SEQUENCE LISTING (112 sequences)

SEQ ID NO: 1 - protein, 133 aa, Homo sapiens [sequence omitted]

SEQ ID NO: 2 - protein, 153 aa, Homo sapiens
  Unprocessed human IL-2
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 3 - protein, 132 aa, Artificial Sequence
  Delta-A1/T3A/F42A/Y45A/L72G/C125S mutations in the human IL-2 sequence
PASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFSQSIISTLT

SEQ ID NO: 4 - protein, 263 aa, Homo sapiens
  exemplary human CD25
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEEKPQASPEGRPESETSCLVTTTDFQIQTEMAATMETSIFTTEYQVAVAGCVFLLISVLLLSGLTWQRRQRKSRRTI

SEQ ID NO: 5 - protein, 760 aa, Homo sapiens
  exemplary human FAP
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 6 - protein, 227 aa, Homo sapiens
  native human IgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 133 aa, Artificial Sequence
  exemplary IL-2 mutants
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 8 - protein, 133 aa, Artificial Sequence
  IL2 C125A
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 9 - protein, 133 aa, Artificial Sequence
  IL2-F42A/Y45A/L72G/C125A
APTSSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 10 - protein, 133 aa, Artificial Sequence
  IL2-R38A/F42A/Y45A/E62A/C125S
APTSSSTKKTQLQLEHLLLDLQMILNGINNYKNPKLTAMLTAKFAMPKKATELKHLQCLEEALKPLEEVLNLAQSKNFHLRPRDLISNINVIVLELKGSETTFMCEYADETATIVEFLNRWITFSQSIISTLT

SEQ ID NO: 11 - protein, 133 aa, Artificial Sequence
  IL2-T3A/R38E/F42A/C125S
APASSSTKKTQLQLEHLLLDLQMILNGINNYKNPKLTEMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 12 - protein, 133 aa, Artificial Sequence
  IL2-T3A/R38E/Y45A/C125S
APASSSTKKTQLQLEHLLLDLQMILNGINNYKNPKLTEMLTFKFAMPKKATELKHLQCLEEELKPLEEVLNLAQSKNFHLRPRDLISNINVIVLELKGSETTFMCEYADETATIVEFLNRWITFSQSIISTLT

SEQ ID NO: 13 - protein, 133 aa, Artificial Sequence
  IL2-T3A/R38E/L72G/C125S
APASSSTKKTQLQLEHLLLDLQMILNGINNYKNPKLTEMLTFKFYMPKKATELKHLQCLEEELKPLEEVLNGAQSKNFHLRPRDLISNINVIVLELKGSETTFMCEYADETATIVEFLNRWITFSQSIISTLT

SEQ ID NO: 14 - protein, 132 aa, Artificial Sequence; identical to SEQ ID NO: 3
  IL2-delta-A2/T3A/F42A/Y45A/L72G/C125S

SEQ ID NO: 15 - protein, 132 aa, Artificial Sequence
  IL2-delta-A2/T3A/K35E/F42A/Y45A/L72G/C125S
PASSSTKKTQLQLEHLLLDLQMILNGINNYKNPELTRMLTAKFAMPKKATELKHLQCLEEELKPLEEVLNGAQSKNFHLRPRDLISNINVIVLELKGSETTFMCEYADETATIVEFLNRWITFSQSIISTLT

SEQ ID NO: 16 - protein, 11 aa, Artificial Sequence
  D001 VL CDR1
RASQVIGSSLN

SEQ ID NO: 17 - protein, 7 aa, Artificial Sequence
  D001,002,003,047,049,872-5, 872-59,872-70 and 872-5v1 VL CDR2
AASSLQS

SEQ ID NO: 18 - protein, 9 aa, Artificial Sequence
  D001 VL CDR3
QQGRSYPYT

SEQ ID NO: 19 - protein, 11 aa, Artificial Sequence
  D002 VL CDR1
RASQSIGNSLN

SEQ ID NO: 20 - protein, 9 aa, Artificial Sequence
  D002 VL CDR3
QQGRRSPFT

SEQ ID NO: 21 - protein, 11 aa, Artificial Sequence
  D029 VL CDR1
RASQDGGNYLN

SEQ ID NO: 22 - protein, 7 aa, Artificial Sequence
  D029 VL CDR2
GASTLYS

SEQ ID NO: 23 - protein, 9 aa, Artificial Sequence
  D029 and D029LV1(D029-VL-Rev1) VL CDR3
QQGRTPPLT

SEQ ID NO: 24 - protein, 11 aa, Artificial Sequence
  D029LV1 (D029-VL-Rev1) and D029LV5(D029-VL-Rev1-P94Y) VL CDR1
RASQALGFYLN

SEQ ID NO: 25 - protein, 7 aa, Artificial Sequence
  D029LV1(D029-VL-Rev1), D029LV2(D029-VL-Rev1-Q27G/P94Y/L96F), D029LV3(D029-VL-Rev1-Q27G/L96F) D029LV4(D029-VL-Rev1-Q27G/P94Y) and D029LV5(D029-VL-Rev1-P94Y) VL CDR2
AASSLSS

SEQ ID NO: 26 - protein, 11 aa, Artificial Sequence
  D029LV2(D029-VL-Rev1-Q27G/P94Y/L96F), D029LV3 (D029-VL-Rev1-Q27G/L96F), and D029LV4(D029-VL-Rev1-Q27G/P94Y) VL CDR1
RASGALGFYLN

SEQ ID NO: 27 - protein, 9 aa, Artificial Sequence
  D029LV2 (D029-VL-Rev1-Q27G/P94Y/L96F) VL CDR3
QQGRTYPFT

SEQ ID NO: 28 - protein, 9 aa, Artificial Sequence
  D029LV3 (D029-VL-Rev1-Q27G/L96F) VL CDR3
QQGRTPPFT

SEQ ID NO: 29 - protein, 9 aa, Artificial Sequence
  D029LV4 (D029-VL-Rev1-Q27G/P94Y) and D029LV5(D029-VL-Rev1-P94Y) VL CDR3
QQGRTYPLT

SEQ ID NO: 30 - protein, 11 aa, Artificial Sequence
  D003, 872-5, 872-59, 872-70 872-5V1, VL CDR1
RASQSISSYLN

SEQ ID NO: 31 - protein, 9 aa, Artificial Sequence
  D003 VL CDR3
QQTRSYLPT

SEQ ID NO: 32 - protein, 11 aa, Artificial Sequence
  D047 VL CDR1
RASQGIKNYLN

SEQ ID NO: 33 - protein, 9 aa, Artificial Sequence
  D047 VL CDR3
QQGSYLQPT

SEQ ID NO: 34 - protein, 11 aa, Artificial Sequence
  D049 VL CDR1
RASQIVRTYLN

SEQ ID NO: 35 - protein, 9 aa, Artificial Sequence
  D049 VL CDR3
QQGAQLQPT

SEQ ID NO: 36 - protein, 5 aa, Artificial Sequence
  D001, D002 and 872-70 VH CDR1
RYSMS

SEQ ID NO: 37 - protein, 17 aa, Artificial Sequence
  D001 VH CDR2
AISPYGGGTDYADSVKG

SEQ ID NO: 38 - protein, 15 aa, Artificial Sequence
  D001, D002, D029, D029HV1 (D029-VH-S55L), D029HV2 (D029-VH-S55L/S32F), D029HV3 (D029-VH-S55L/T30R/S32F), D029HV4 (D029-VH-H1V9), D029HV5 (D029-VH-S55L/T30R/S32A), and 872-70 VH VH CDR3
SQYGRYYSPDYYFDY

SEQ ID NO: 39 - protein, 16 aa, Artificial Sequence
  D002 VH CDR2
DISSYSTTYYADSVKG

SEQ ID NO: 40 - protein, 5 aa, Artificial Sequence
  D029 and D029HV1 (D029-VH-S55L) VH CDR1
WSFMS

SEQ ID NO: 41 - protein, 17 aa, Artificial Sequence
  D029 VH CDR2
WIGPSGSYTDYADSVKG

SEQ ID NO: 42 - protein, 17 aa, Artificial Sequence
  D029HV1(D029-VH-S55L), D029HV2 (D029-VH-S55L/S32F), D029HV4 (D029-VH-H1V9), D029HV5 (D029-VH-S55L/T30R/S32A) and D029HV6 (D029-VH-S55L/S32A) VH CDR2
WIGPSGLYTDYADSVKG

SEQ ID NO: 43 - protein, 5 aa, Artificial Sequence
  D029HV2 (D029-VH-S55L/S32F) and D029HV3 (D029-VH-S55L/T30R/S32F) VH CDR1
WFFMS

SEQ ID NO: 44 - protein, 5 aa, Artificial Sequence
  D029HV4 (D029-VH-H1V9) VH CDR1
RFFMS

SEQ ID NO: 45 - protein, 5 aa, Artificial Sequence
  D029HV5 (D029-VH-S55L/T30R/S32A) and D029HV6 (D029-VH-S55L/S32A) VH CDR1
WAFMS

SEQ ID NO: 46 - protein, 5 aa, Artificial Sequence
  D003 VH CDR1
TYIMS

SEQ ID NO: 47 - protein, 17 aa, Artificial Sequence
  D003 VH CDR2
SIGPSYGDTIYADSVKG

SEQ ID NO: 48 - protein, 15 aa, Artificial Sequence
  D003 and 872-59 VH CDR3
TISYSQYAYGYSFDY

SEQ ID NO: 49 - protein, 5 aa, Artificial Sequence
  D047 VH CDR1
YFSMS

SEQ ID NO: 50 - protein, 17 aa, Artificial Sequence
  D047 VH CDR2
SISPTYGTTDYADSVKG

SEQ ID NO: 51 - protein, 9 aa, Artificial Sequence
  D047, D049 and 872-5V1 VH VH CDR3
LLYGTWFDY

SEQ ID NO: 52 - protein, 5 aa, Artificial Sequence
  D049 VH CDR1
SYWMS

SEQ ID NO: 53 - protein, 17 aa, Artificial Sequence
  D049 VH CDR2
TISPYDSSTAYADSVKG

SEQ ID NO: 54 - protein, 9 aa, Artificial Sequence
  872-5 VL CDR3
QQATLLLPT

SEQ ID NO: 55 - protein, 9 aa, Artificial Sequence
  872-59 VL CDR3
QQAFTSPRT

SEQ ID NO: 56 - protein, 9 aa, Artificial Sequence
  872-70 VL CDR3
QQSRTSPYT

SEQ ID NO: 57 - protein, 10 aa, Artificial Sequence
  872-5V1 VL CDR3
QQGTLLLPTF

SEQ ID NO: 58 - protein, 5 aa, Artificial Sequence
  872-5 and 872-5V1 VH CDR1
GYIMS

SEQ ID NO: 59 - protein, 17 aa, Artificial Sequence
  872-5 VH CDR2
WISPEGSRTGYADSVKG

SEQ ID NO: 60 - protein, 9 aa, Artificial Sequence
  872-5 VH CDR3
LQYGTWFDY

SEQ ID NO: 61 - protein, 5 aa, Artificial Sequence
  872-59 VH CDR1
SYAMS

SEQ ID NO: 62 - protein, 17 aa, Artificial Sequence
  872-59 VH CDR2
SIYSWYGTTSYADSVKG

SEQ ID NO: 63 - protein, 17 aa, Artificial Sequence
  872-70 VH CDR2
YITSTDGTTDYADSVKG

SEQ ID NO: 64 - protein, 17 aa, Artificial Sequence
  872-5V1 VH CDR2
WIGPEGSRTGYADSVKG

SEQ ID NO: 65 - protein, 5 aa, Artificial Sequence
  VHH6 VH CDR1
PTYMY

SEQ ID NO: 66 - protein, 16 aa, Artificial Sequence
  VHH6 VH CDR2
AIDPYGRTEYPDSVKG

SEQ ID NO: 67 - protein, 17 aa, Artificial Sequence
  VHH6 VH CDR3
RVPPGVYTYYRTGPYDY

SEQ ID NO: 68 - protein, 111 aa, Artificial Sequence
  D001 VL
DIQMTQSPSSLSASVGDRVTITCRASQVIGSSLNWYQQKPGKAPKLLIYAASSLQSGVPSRFSGSGSGTDFTLTISSLQPEDFATYYCQQGRSYPYTFGQGTKVEIKRTVA

SEQ ID NO: 69 - protein, 111 aa, Artificial Sequence
  D002 VL
DIQMTQSPSSLSASVGDRVTITCRASQSIGNSLNWYQQKPGKAPKLLIYAASSLQSGVPSRFSGSGSGTDFTLTISSLQPEDFATYYCQQGRRSPFTFGQGTKVEIKRTVA

SEQ ID NO: 70 - protein, 111 aa, Artificial Sequence
  D029 VL
DIQMTQSPSSLSASVGDRVTITCRASQDGGNYLNWYQQKPGKAPKLLIYGASTLYSGVPSRFSGSGSGTDFTLTISSLQPEDFATYYCQQGRTPPLTFGQGTKVEIKRTVA

SEQ ID NO: 71 - protein, 111 aa, Artificial Sequence
  D029LV1 (D029-VL-Rev1) VL
DIQMTQSPSSLSASVGDRVTITCRASQALGFYLNWYQQKPGKAPKLLIYAASSLSSGVPSRFSGSGSGTDFTLTISSLQPEDFATYYCQQGRTPPLTFGQGTKVEIKRTVA

SEQ ID NO: 72 - protein, 111 aa, Artificial Sequence
  D029LV2(D029-VL-Rev1-Q27G/P94Y/L96F) VL
DIQMTQSPSSLSASVGDRVTITCRASGALGFYLNWYQQKPGKAPKLLIYAASSLSSGVPSRFSGSGSGTDFTLTISSLQPEDFATYYCQQGRTYPFTFGQGTKVEIKRTVA

SEQ ID NO: 73 - protein, 111 aa, Artificial Sequence
  D029LV3 (D029-VL-Rev1-Q27G/L96F) VL
DIQMTQSPSSLSASVGDRVTITCRASGALGFYLNWYQQKPGKAPKLLIYAASSLSSGVPSRFSGSGSGTDFTLTISSLQPEDFATYYCQQGRTPPFTFGQGTKVEIKRTVA

SEQ ID NO: 74 - protein, 111 aa, Artificial Sequence
  D029LV4 (D029-VL-Rev1-Q27G/P94Y) VL
DIQMTQSPSSLSASVGDRVTITCRASGALGFYLNWYQQKPGKAPKLLIYAASSLSSGVPSRFSGSGSGTDFTLTISSLQPEDFATYYCQQGRTYPLTFGQGTKVEIKRTVA

SEQ ID NO: 75 - protein, 111 aa, Artificial Sequence
  D029LV5(D029-VL-Rev1-P94Y) VL
DIQMTQSPSSLSASVGDRVTITCRASQALGFYLNWYQQKPGKAPKLLIYAASSLSSGVPSRFSGSGSGTDFTLTISSLQPEDFATYYCQQGRTYPLTFGQGTKVEIKRTVA

SEQ ID NO: 76 - protein, 111 aa, Artificial Sequence
  D003 VL
DIQMTQSPSSLSASVGDRVTITCRASQSISSYLNWYQQKPGKAPKLLIYAASSLQSGVPSRFSGSGSGTDFTLTISSLQPEDFATYYCQQTRSYLPTFGQGTKVEIKRTVA

SEQ ID NO: 77 - protein, 111 aa, Artificial Sequence
  D047 VL
DIQMTQSPSSLSASVGDRVTITCRASQGIKNYLNWYQQKPGKAPKLLIYAASSLQSGVPSRFSGSGSGTDFTLTISSLQPEDFATYYCQQGSYLQPTFGQGTKVEIKRTVA

SEQ ID NO: 78 - protein, 111 aa, Artificial Sequence
  D049 VL
DIQMTQSPSSLSASVGDRVTITCRASQIVRTYLNWYQQKPGKAPKLLIYAASSLQSGVPSRFSGSGSGTDFTLTISSLQPEDFATYYCQQGAQLQPTFGQGTKVEIKRTVA

SEQ ID NO: 79 - protein, 124 aa, Artificial Sequence
  D001 VH
EVQLVESGGGLVQPGGSLRLSCAASGFTFSRYSMSWVRQAPGKGLEWVSAISPYGGGTDYADSVKGRFTISRDNSKNTLYLQMNSLRAEDTAVYYCARSQYGRYYSPDYYFDYWGQGTLVTVSS

SEQ ID NO: 80 - protein, 123 aa, Artificial Sequence
  D002 VH
EVQLVESGGGLVQPGGSLRLSCAASGFTFSRYSMSWVRQAPGKGLEWVSDISSYSTTYYADSVKGRFTISRDNSKNTLYLQMNSLRAEDTAVYYCARSQYGRYYSPDYYFDYWGQGTLVTVSS

SEQ ID NO: 81 - protein, 124 aa, Artificial Sequence
  D029 VH
EVQLVESGGGLVQPGGSLRLSCAASGFTFTWSFMSWVRQAPGKGLEWVSWIGPSGSYTDYADSVKGRFTISRDNSKNTLYLQMNSLRAEDTAVYYCARSQYGRYYSPDYYFDYWGQGTLVTVSS

SEQ ID NO: 82 - protein, 124 aa, Artificial Sequence
  D029HV1 (D029-VH-S55L) VH
EVQLVESGGGLVQPGGSLRLSCAASGFTFTWSFMSWVRQAPGKGLEWVSWIGPSGLYTDYADSVKGRFTISRDNSKNTLYLQMNSLRAEDTAVYYCARSQYGRYYSPDYYFDYWGQGTLVTVSS

SEQ ID NO: 83 - protein, 124 aa, Artificial Sequence
  D029HV2 (D029-VH-S55L/S32F) VH
EVQLVESGGGLVQPGGSLRLSCAASGFTFTWFFMSWVRQAPGKGLEWVSWIGPSGLYTDYADSVKGRFTISRDNSKNTLYLQMNSLRAEDTAVYYCARSQYGRYYSPDYYFDYWGQGTLVTVSS

SEQ ID NO: 84 - protein, 124 aa, Artificial Sequence
  D029HV3 (D029-VH-S55L/T30R/S32F) VH
EVQLVESGGGLVQPGGSLRLSCAASGFTFRWFFMSWVRQAPGKGLEWVSWIGPSGLYTDYADSVKGRFTISRDNSKNTLYLQMNSLRAEDTAVYYCARSQYGRYYSPDYYFDYWGQGTLVTVSS

SEQ ID NO: 85 - protein, 124 aa, Artificial Sequence
  D029HV4 (D029-VH-H1V9) VH
EVQLVESGGGLVQPGGSLRLSCAASGFTFSRFFMSWVRQAPGKGLEWVSWIGPSGLYTDYADSVKGRFTISRDNSKNTLYLQMNSLRAEDTAVYYCARSQYGRYYSPDYYFDYWGQGTLVTVSS

SEQ ID NO: 86 - protein, 124 aa, Artificial Sequence
  D029HV5 (D029-VH-S55L/T30R/S32A) VH
EVQLVESGGGLVQPGGSLRLSCAASGFTFRWAFMSWVRQAPGKGLEWVSWIGPSGLYTDYADSVKGRFTISRDNSKNTLYLQMNSLRAEDTAVYYCARSQYGRYYSPDYYFDYWGQGTLVTVSS

SEQ ID NO: 87 - protein, 124 aa, Artificial Sequence
  D029HV6 (D029-VH-S55L/S32A) VH
EVQLVESGGGLVQPGGSLRLSCAASGFTFTWAFMSWVRQAPGKGLEWVSWIGPSGLYTDYADSVKGRFTISRDNSKNTLYLQMNSLRAEDTAVYYCARSQYGRYYSPDYYFDYWGQGTLVTVSS

SEQ ID NO: 88 - protein, 124 aa, Artificial Sequence
  D003 VH
EVQLVESGGGLVQPGGSLRLSCAASGFTFYTYIMSWVRQAPGKGLEWVSSIGPSYGDTIYADSVKGRFTISRDNSKNTLYLQMNSLRAEDTAVYYCARTISYSQYAYGYSFDYWGQGTLVTVSS

SEQ ID NO: 89 - protein, 118 aa, Artificial Sequence
  D047 VH
EVQLVESGGGLVQPGGSLRLSCAASGFTFHYFSMSWVRQAPGKGLEWVSSISPTYGTTDYADSVKGRFTISRDNSKNTLYLQMNSLRAEDTAVYYCARLLYGTWFDYWGQGTLVTVSS

SEQ ID NO: 90 - protein, 118 aa, Artificial Sequence
  D049 VH
EVQLVESGGGLVQPGGSLRLSCAASGFTFFSYWMSWVRQAPGKGLEWVSTISPYDSSTAYADSVKGRFTISRDNSKNTLYLQMNSLRAEDTAVYYCARLLYGTWFDYWGQGTLVTVSS

SEQ ID NO: 91 - protein, 111 aa, Artificial Sequence
  872-5 VL Domain
DIQMTQSPSSLSASVGDRVTITCRASQSISSYLNWYQQKPGKAPKLLIYAASSLQSGVPSRFSGSGSGTDFTLTISSLQPEDFATYYCQQATLLLPTFGQGTKVEIKRTVA

SEQ ID NO: 92 - protein, 111 aa, Artificial Sequence
  872-59 VL Domain
DIQMTQSPSSLSASVGDRVTITCRASQSISSYLNWYQQKPGKAPKLLIYAASSLQSGVPSRFSGSGSGTDFTLTISSLQPEDFATYYCQQAFTSPRTFGQGTKVEIKRTVA

SEQ ID NO: 93 - protein, 111 aa, Artificial Sequence
  872-70 VL Domain
DIQMTQSPSSLSASVGDRVTITCRASQSISSYLNWYQQKPGKAPKLLIYAASSLQSGVPSRFSGSGSGTDFTLTISSLQPEDFATYYCQQSRTSPYTFGQGTKVEIKRTVA

SEQ ID NO: 94 - protein, 111 aa, Artificial Sequence
  872-5V1 VL Domain
DIQMTQSPSSLSASVGDRVTITCRASQSISSYLNWYQQKPGKAPKLLIYAASSLQSGVPSRFSGSGSGTDFTLTISSLQPEDFATYYCQQGTLLLPTFGQGTKVEIKRTVA

SEQ ID NO: 95 - protein, 118 aa, Artificial Sequence
  872-5 VH Domain
EVQLVESGGGLVQPGGSLRLSCAASGFTFRGYIMSWVRQAPGKGLEWVSWISPEGSRTGYADSVKGRFTISRDNSKNTLYLQMNSLRAEDTAVYYCARLQYGTWFDYWGQGTLVTVSS

SEQ ID NO: 96 - protein, 124 aa, Artificial Sequence
  872-59 VH Domain
EVQLVESGGGLVQPGGSLRLSCAASGFTFTSYAMSWVRQAPGKGLEWVSSIYSWYGTTSYADSVKGRFTISRDNSKNTLYLQMNSLRAEDTAVYYCARTISYSQYAYGYSFDYWGQGTLVTVSS

SEQ ID NO: 97 - protein, 124 aa, Artificial Sequence
  872-70 VH Domain
EVQLVESGGGLVQPGGSLRLSCAASGFTFSRYSMSWVRQAPGKGLEWVSYITSTDGTTDYADSVKGRFTISRDNSKNTLYLQMNSLRAEDTAVYYCARSQYGRYYSPDYYFDYWGQGTLVTVSS

SEQ ID NO: 98 - protein, 118 aa, Artificial Sequence
  872-5V1 VH Domain
EVQLVESGGGLVQPGGSLRLSCAASGFTFRGYIMSWVRQAPGKGLEWVSWIGPEGSRTGYADSVKGRFTISRDNSKNTLYLQMNSLRAEDTAVYYCARLLYGTWFDYWGQGTLVTVSS

SEQ ID NO: 99 - protein, 125 aa, Artificial Sequence
  VHH6 VH Domain
EVQLVESGGGLVQPGGSLRLSCAASGFTFTPTYMYWVRQAPGKGLEWVSAIDPYGRTEYPDSVKGRFTISRDNAKNTLYLQMNSLRPEDTAVYYCAVRVPPGVYTYYRTGPYDYWGQGTLVTVSS

SEQ ID NO: 100 - DNA, 15 bp, Artificial Sequence
  GGGGS linker at 15 amino acids length
ggggsggggsggggs

SEQ ID NO: 101 - protein, 108 aa, Artificial Sequence
  VL of B10 Antibody
DIQMTQSPSSLSASVGDRVTITCRASQNIARYLNWYQQKPGKAPKLLIYAASSLQSGVPSRFSGSGSGTDFTLTISSLQPEDFATYYCQQGRYSPFTFGQGTKVEIKR

SEQ ID NO: 102 - protein, 124 aa, Artificial Sequence
  VH of B10 Antibody
EVQLVESGGGLVQPGGSLRLSCAASGFTFSRYSMGWVRQAPGKGLEWVSTIHSHGSGTRYADSVKGRFTISRDNSKNTLYLQMNSLRAEDTAVYYCARSQYGRYYSPDYYFDYWGQGTLVTVSS

SEQ ID NO: 103 - protein, 11 aa, Artificial Sequence
  B10 Antibody - VL CDR1
RASQNIARYLN

SEQ ID NO: 104 - protein, 9 aa, Artificial Sequence
  B10 Antibody - VL CDR3
QQGRYSPFT

SEQ ID NO: 105 - protein, 6 aa, Artificial Sequence
  B10 Antibody - VH CDR1
SRYSMG

SEQ ID NO: 106 - protein, 17 aa, Artificial Sequence
  B10 Antibody - VH CDR2
TIHSHGSGTRYADSVKG

SEQ ID NO: 107 - protein, 133 aa, Artificial Sequence
  IL2-T3A/K35E/F42A/Y45A/L72G/C125S
APASSSTKKTQLQLEHLLLDLQMILNGINNYKNPELTRMLTAKFAMPKKATELKHLQCLEEELKPLEEVLNGAQSKNFHLRPRDLISNINVIVLELKGSETTFMCEYADETATIVEFLNRWITFSQSIISTLT

SEQ ID NO: 108 - protein, 133 aa, Artificial Sequence
  IL2-T3A/K35E/F42A/C125S
APASSSTKKTQLQLEHLLLDLQMILNGINNYKNPELTRMLTAKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 109 - protein, 133 aa, Artificial Sequence
  IL2-T3A/D20T/K35E/C125S
APASSSTKKTQLQLEHLLLTLQMILNGINNYKNPELTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 110 - protein, 133 aa, Artificial Sequence
  IL2-T3A/H16A/K35E/C125S
APASSSTKKTQLQLEALLLDLQMILNGINNYKNPELTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 111 - protein, 551 aa, Homo sapiens
  an exemplary human CD122
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 112 - protein, 369 aa, Artificial Sequence
  an exemplary human CD132 (with signal sequence)
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

(19) United States
(12) Patent Application Publication
(10) Pub. No.: US 2024/0358849 A1
(43) Pub. Date: Oct. 31, 2024

(54) IMMUNOCONJUGATE MOLECULES AND RELATED METHODS AND COMPOSITIONS THEREOF

(71) Applicant: SUZHOU FUSE BIOSCIENCES LIMITED, Jiangsu (CN)

(72) Inventors: Qufei LI, Jiangsu (CN); Lucas BAILEY, Jiangsu (CN)

(21) Appl. No.: 18/571,703

(22) PCT Filed: May 13, 2022
(86) PCT No.: PCT/CN2022/092831

§ 371 Date: Dec. 18, 2023

(30) Foreign Application Priority Data
Jun. 17, 2021 (WO) PCT/CN2021/100705

(51) Int. Cl. A61K 47/68; A61K 39/00; A61P 35/00; C07K 14/55; C07K 16/24; C07K 16/30; C07K 16/40; C07K 19/00

(52) U.S. Cl. CPC A61K 47/6813; A61K 47/6851; A61K 47/6879; A61P 35/00; C07K 14/55; C07K 16/246; C07K 16/30; C07K 16/40; C07K 19/00; A61K 2039/505; C07K 2317/31; C07K 2317/52; C07K 2317/55; C07K 2317/565; C07K 2317/71; C07K 2317/76; C07K 2317/92; C07K 2317/94; C07K 2319/33

(57) ABSTRACT

The immunoconjugate molecules contain an interleukin-2 (IL-2) polypeptide and a masking moiety capable of inhibiting and activating the IL-2 activity under suitable conditions. Methods for producing the immunoconjugate molecules. The therapeutic uses of the immunoconjugate molecules due to their modulating effects on the immune system for treating diseases such as cancer and other chronic infectious diseases.

### CROSS-REFERENCE TO RELATED APPLICATIONS

This application claims the benefit of priority of PCT/CN2021/100705 filed on Jun. 17, 2021, the content of which is herein incorporated by reference in its entirety.

### REFERENCE TO SEQUENCE LISTING SUBMITTED ELECTRONICALLY

This application contains a sequence listing, which is submitted electronically as an ASCII formatted sequence listing with a file “14625-006-228_SEQLIST.txt” and a creation date of May 10, 2022 and having a size of 90,098 bytes. The sequence listing submitted electronically is part of the specification and is herein incorporated by reference in its entirety.

### 1. FIELD

The present disclosure generally relates to interleukin-2 (IL-2) containing immunoconjugate molecules. More particularly, the present disclosure concerns immunoconjugate molecules exhibited improved properties for use as immunotherapeutic agents due to the ability of modulating the immune system. The present disclosure further relates to therapeutic uses and pharmaceutical compositions of the immunoconjugate molecules for treating diseases such as cancer and other chronic infectious diseases.

### 2. BACKGROUND

Interleukin-2 (IL-2), also known as T cell growth factor (TCGF), is a 15.5 kDa globular glycoprotein playing a central role in lymphocyte generation, survival and homeostasis. The ability of IL-2 to expand lymphocyte populations in vivo and to increase the effector functions of these cells confers antitumor effects to IL-2, making IL-2 immunotherapy an attractive treatment option for certain metastatic cancers. Consequently, high-dose IL-2 treatment has been approved for use in patients with metastatic renal-cell carcinoma and malignant melanoma. However, soluble IL-2 is not optimal for inhibiting tumor growth, because IL-2 has dual function in the immune response that it not only mediates expansion and activity of effector cells, but also is crucially involved in maintaining peripheral immune tolerance. A further concern in relation to IL-2 immunotherapy are the side effects produced by recombinant human IL-2 treatment. For example, patients receiving high-dose IL-2 treatment frequently experience severe cardiovascular, pulmonary, renal, hepatic, gastrointestinal, neurological, cutaneous, haematological and systemic adverse events, which require intensive monitoring and in-patient management. Thus, there remains a need in the art to further enhance the therapeutic usefulness of IL-2 proteins. The present disclosure meets this need.

### 3. SUMMARY

The present disclosure provides immunoconjugate molecules comprising a cytokine polypeptide. The present disclosure also provides, in certain embodiments, polynucleotides and vectors comprising sequences encoding such immunoconjugate molecules, and compositions, reagents, and kits comprising such immunoconjugate molecules. In related aspect, provided herein are also methods for delivery and/or activation of a cytokine activity at a target site, or reduce toxicity and/or other side-effects associated with systemic exposure to the cytokine activity in a subject through the use of the immunoconjugate molecules according to the present disclosure.

The present disclosure also provides, in certain embodiments, peptides or polypeptides, such as antibodies or antigen binding fragments thereof that can form part of such immunoconjugate molecules of the present disclosure. In specific embodiments, provided herein are binding proteins, including antibodies of fragments thereof that bind to fibrosis activation protein (FAP). In specific embodiments, provided herein are bispecific binding proteins, including two-in-one antibodies or fragments thereof that bind to both FAP and interleukin-2 (IL-2).

In some embodiments, an immunoconjugate molecule of the present disclosure comprises a cytokine moiety that comprises a cytokine polypeptide having a cytokine activity and a masking moiety. Such masking moiety comprises a bispecific antibody or antigen binding fragment thereof capable of binding to the cytokine polypeptide and a first target antigen. When binding to the cytokine polypeptide, the masking moiety reduces or inhibits the cytokine activity, and when binding to the second target antigen, the masking moiety disassociates from the cytokine polypeptide, thereby activating the cytokine activity

In some embodiments, the masking moiety comprises an intact antibody, a Fab, a Fab′, a F(ab′)₂, a Fv, a scFv, a dsFv, a diabody, a triabody, a tetrabody, or a VHH formed from antibody fragments. In some embodiments, the bispecific antibody is a two-in-one antibody.

In some embodiments, the first target antigen is not the cytokine polypeptide. In some embodiments, the first target antigen is expressed on a cell surface. In some embodiments, the cell is a cancer cell or a cell in a tumor microenvironment. In some embodiments, the first target antigen is soluble. In some embodiments, the first target antigen is a tumor associated antigen. In some embodiments, the first target antigen is fibrosis activation protein (FAP).

In some embodiments, the cytokine moiety comprises wild-type or mutant interleukin-2 (IL-2). In some embodiments, the cytokine moiety comprises human IL-2 or mutant human IL-2.

In some embodiments, the immunoconjugate molecule further comprises an anchoring moiety comprising an antibody or antigen binding fragment thereof that specifically binds to a second target antigen. In some embodiments, the second target antigen is expressed on a cell surface. In some embodiments, the cell is a cancer cell or a cell in a tumor microenvironment. In some embodiments, the second target antigen is soluble. In some embodiments, the second target antigen is a tumor associated antigen.

In some embodiments, the first and second target antigens are the same. In some embodiments, the bispecific masking moiety and the anchoring moiety bind to the same epitope of the first or second target antigen. In some embodiments, the bispecific masking moiety and the anchoring moiety bind to different epitopes of the first or second target antigen. In some embodiments, the first target antigen and second target antigens are different. In some embodiments, the second target antigen is fibrosis activation protein (FAP).

In some embodiments, the anchoring moiety comprises an intact antibody, a Fab, a Fab′, a F(ab′)₂, a Fv, a scFv, a dsFv, a diabody, a triabody, a tetrabody, or a VHH formed from antibody fragments. In specific embodiments, the bispecific antibody or antigen binding fragment of the masking moiety is a Fab, ScFv or VHH. In specific embodiments, the antibody or antigen binding fragment thereof of the anchoring moiety is a Fab, ScFv or VHH.

In some embodiments, the immunoconjugate molecule further comprises a conjugating moiety, wherein the conjugating moiety operably connects two or more of the cytokine moiety, the masking moiety, and the anchoring moiety of the immunoconjugate molecule.

In some embodiments, the conjugating moiety comprises an immunoglobulin Fc domain or a mutant thereof. In some embodiments, the Fc domain comprises a first subunit and a second subunit that are two non-identical polypeptide chains; and wherein the Fc domain comprises a first modification promoting hetero-dimerization of the two non-identical polypeptide chains. In some embodiments, the first modification is a knob-into-hole modification comprising a knob modification in the first subunit and a hole modification in the second subunit.

In some embodiments, the Fc domain comprises a second modification, wherein the Fc domain has reduced binding affinity to an Fc receptor compared to a native Fc domain without said second modification. In some embodiments, the Fc domain has reduced binding affinity to a Fcγ receptor as compared to the native Fc domain without said second modification. In some embodiments, the Fcγ receptor is an FcγRIIIa, FcγRI or FcγRIIa receptor.

In some embodiments, the Fc domain has reduced binding affinity to a complement component as compared to the native Fc domain without said second modification. In some embodiments, the complement component is C1q.

In some embodiments, the Fc domain has reduced Fc effector function as compared to an Fc domain without said second modification. In some embodiments, the reduced Fc effector function is selected from complement dependent cytotoxicity (CDC), antibody-dependent cell-mediated cytotoxicity (ADCC), antibody-dependent cellular phagocytosis (ADCP), cytokine secretion, downregulation of cell surface receptors, and B cell activation.

In some embodiments, the second modification comprises one or more mutations selected from S228P, E233P, L234V, L234A, L235A, L235E, AG236, D265G, N297A, N297D, P329E, P329S, P329A, P329G, A330S, or P331S, wherein the numbering is that of the EU index as in Kabat. In some embodiments, the second modification comprises one or more mutations selected from E233P, L234V, L234A, L235A, AG236, D265G, P327E, A328S, P329E, A330S, or P331S, wherein the numbering is that of the EU index as in Kabat.

In some embodiments, the cytokine moiety is connected to the C-terminus of one of the first and second subunits of the Fc domain, and the masking moiety is connected to the C-terminus of the other of the first and second subunits of the Fc domain. In some embodiments, the anchoring moiety is connected to the N-terminus of one of the first and second subunits of the Fc domain. In some embodiments, the anchoring moiety and the cytokine moiety are connected to the same subunit of the Fc domain. In some embodiments, the anchoring moiety and the masking moiety are connected to the same subunit of the Fc domain. In some embodiments, the masking moiety is connected to the C-terminus of one of the first and second subunits of the Fc domain; and wherein the cytokine moiety is connected to the masking moiety. In some embodiments, the anchoring moiety is connected to the N-terminus of one of the first and second subunits of the Fc domain. In some embodiments, the anchoring moiety and the masking moiety are connected to the same subunit of the Fc domain; or wherein the anchoring moiety and the masking moiety are connected to different subunits of the Fc domain. In some embodiments, the masking moiety is connected to the N-terminus of one of the first and second subunits of the Fc domain, and the cytokine moiety is connected to the masking moiety. In some embodiments, the masking moiety is connected to the N-terminus of one of the first and second subunits of the Fc domain, and wherein the anchoring moiety is connected to the N-terminus of the other one of the first and second subunits of the Fc domain. In some embodiments, the cytokine moiety is connected to the masking moiety. In some embodiments, the cytokine moiety is connected to the anchoring moiety.

In some embodiments, the two-in-one antibody or antigen binding fragment thereof of the masking moiety is a Fab, a ScFv or a VHH. In some embodiments, the antibody or antigen binding fragment thereof of the anchoring moiety is a Fab, a ScFv, or a VHH. In some embodiments, the connection between two or more of the cytokine moiety, the masking moiety, the anchoring moiety and the conjugating moiety is via a peptidic linker.

In some embodiments, the cytokine is IL-2 polypeptide. In specific embodiments, the cytokine polypeptide comprises an amino acid sequence selected from SEQ ID NOS: 1, 3, 7 to 15, and 107-110. In some embodiments, the first target antigen and the second target antigen are Fibroblast Activation Protein (FAP). In specific embodiments, the first target antigen and the second target antigen are human FAP.

In specific embodiments, the masking moiety comprises an antibody or antigen binding fragment thereof that binds to Fibroblast Activation Protein (FAP) and interleukin-2 (IL-2), wherein the antibody or antigen binding fragment comprises (a) a light chain variable region (VH) comprising VL complementarity determining region 1 (CDR1), VL CDR2, and VL CDR3 of any one of antibodies D001, D002, D029, D029LV1, D029LV2, D029LV3, D029LV4, D029LV5, D003, D047, D049, or B10 as set forth in Table 1; and/or (b) a heavy chain variable region (VH) comprising VH complementarity determining region 1 (CDR1), VH CDR2, and VH CDR3 of any one of antibodies D001, D002, D029, D029HV1, D029HV2, D029HV3, D029HV4, D029HV5, D029HV6, D003, D047, D049, or B10 as set forth in Table 2.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:16, 17, and 18, respectively, and the VH CDR1, VH CDR2, and VH CDR3 comprise amino acid sequences of SEQ ID NOS:36, 37, and 38, respectively.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:19, 17, and 20, respectively, and the VH CDR1, VH CDR2, and VH CDR3 comprise amino acid sequences of SEQ ID NOS:36, 39, and 38, respectively.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:21, 22, and 23, respectively, and the VH CDR1, VH CDR2, and VH CDR3 comprise amino acid sequences of SEQ ID NOS:40, 41, and 38, respectively.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:30, 17, and 31, respectively, and the VH CDR1, VH CDR2, and VH CDR3 comprise amino acid sequences of SEQ ID NOS:46, 47, and 48, respectively.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:32, 17, and 33, respectively, and the VH CDR1, VH CDR2, and VH CDR3 comprise amino acid sequences of SEQ ID NOS:49, 50, and 51, respectively.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:34, 17, and 35, respectively, and the VH CDR1, VH CDR2, and VH CDR3 comprise amino acid sequences of SEQ ID NOS:52, 53, and 51, respectively.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:24, 25, and 23, respectively, and the VH CDR1, VH CDR2, and VH CDR3 comprise amino acid sequences of SEQ ID NOS:40, 42, and 38, respectively.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:26, 25, and 28, respectively, and the VH CDR1, VH CDR2, and VH CDR3 comprise amino acid sequences of SEQ ID NOS:43, 42, and 38, respectively.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:26, 25, and 29, respectively, and the VH CDR1, VH CDR2, and VH CDR3 comprise amino acid sequences of SEQ ID NOS:43, 42, and 38, respectively.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:24, 25, and 29, respectively, and the VH CDR1, VH CDR2, and VH CDR3 comprise amino acid sequences of SEQ ID NOS:40, 42, and 38, respectively.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:26, 25, and 27, respectively, and the VH CDR1, VH CDR2, and VH CDR3 comprise amino acid sequences of SEQ ID NOS:43, 42, and 38, respectively.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:26, 25, and 27, respectively, and the VH CDR1, VH CDR2, and VH CDR3 comprise amino acid sequences of SEQ ID NOS:44, 42, and 38, respectively.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:26, 25, and 27, respectively, and the VH CDR1, VH CDR2, and VH CDR3 comprise amino acid sequences of SEQ ID NOS:45, 42, and 38, respectively.

In some embodiments, the masking moiety comprises an antibody or antigen binding fragment thereof that binds to Fibroblast Activation Protein (FAP) and interleukin-2 (IL-2), wherein the antibody or antigen binding fragment comprises the VL CDR1, VL CDR2, and VL CDR3 comprise amino acid sequences of SEQ ID NOS:103, 17, and 104, respectively, and the VH CDR1, VH CDR2, and VH CDR3 comprise amino acid sequences of SEQ ID NOS:105, 106, and 38, respectively.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: (a) a light chain variable region (VL) comprising VL of any one of antibodies D001, D002, D029, D029LV1, D029LV2, D029LV3, D029LV4, D029LV5, D003, D047, D049, or B10 as set forth in Table 3; and/or (b) a heavy chain variable region (VH) comprising VH of any one of antibodies D001, D002, D029, D029LV1, D029LV2, D029LV3, D029LV4, D029LV5, D003, D047, D049, or B10 as set forth in Table 4.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO: 76, SEQ ID NO:77, SEQ ID NO: 78, or SEQ ID NO: 101.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VH comprising an amino acid sequence of SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO: 87, SEQ ID NO:88, SEQ ID NO: 89, SEQ ID NO: 90, or SEQ ID NO: 102.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 68; and a VH comprising an amino acid sequence of SEQ ID NO: 79.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 69; and a VH comprising an amino acid sequence of SEQ ID NO: 80.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 70; and a VH comprising an amino acid sequence of SEQ ID NO: 81.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 76; and a VH comprising an amino acid sequence of SEQ ID NO: 88.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 77; and a VH comprising an amino acid sequence of SEQ ID NO: 89.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 78; and a VH comprising an amino acid sequence of SEQ ID NO: 90.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 71; and a VH comprising an amino acid sequence of SEQ ID NO: 82.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 73; and a VH comprising an amino acid sequence of SEQ ID NO: 83.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 74; and a VH comprising an amino acid sequence of SEQ ID NO: 83.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 75; and a VH comprising an amino acid sequence of SEQ ID NO: 82.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 72; and a VH comprising an amino acid sequence of SEQ ID NO: 84.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 72; and a VH comprising an amino acid sequence of SEQ ID NO: 85.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 72; and a VH comprising an amino acid sequence of SEQ ID NO: 87.

In some embodiments, the masking moiety comprises an antibody or antigen binding fragment thereof that binds to Fibroblast Activation Protein (FAP) and interleukin-2 (IL-2), wherein the antibody or antigen-binding fragment comprises: a VL comprising an amino acid sequence of SEQ ID NO: 101; and a VH comprising an amino acid sequence of SEQ ID NO: 102.

In some embodiments, the anchoring moiety comprises an antibody or antigen binding fragment thereof that binds to Fibroblast Activation Protein (FAP), wherein the antibody or antigen binding fragment comprises (a) a light chain variable region (VH) comprising VL complementarity determining region 1 (CDR1), VL CDR2, and VL CDR3 of any one of antibodies 872-5, 872-59, 872-70, or 872-5V1 as set forth in Table 5; and/or (b) a heavy chain variable region (VH) comprising VH complementarity determining region 1 (CDR1), VH CDR2, and VH CDR3 of any one of antibodies 872-5, 872-59, 872-70, 872-5V1, or VHH6 as set forth in Table 6.

In some embodiments, the anchoring moiety comprises an antibody or antigen binding fragment thereof that binds to Fibroblast Activation Protein (FAP), wherein the antibody or antigen binding fragment comprises the VL CDR1, VL CDR2, and VL CDR3 comprise amino acid sequences of SEQ ID NOS:30, 17, and 54, respectively, and the VH CDR1, VH CDR2, and VH CDR3 comprise amino acid sequences of SEQ ID NOS:58, 59, and 60, respectively.

In some embodiments, the anchoring moiety comprises an antibody or antigen binding fragment thereof that binds to Fibroblast Activation Protein (FAP), wherein the antibody or antigen binding fragment comprises the VL CDR1, VL CDR2, and VL CDR3 comprise amino acid sequences of SEQ ID NOS:30, 17, and 55, respectively, and the VH CDR1, VH CDR2, and VH CDR3 comprise amino acid sequences of SEQ ID NOS:61, 62, and 48, respectively.

In some embodiments, the anchoring moiety comprises an antibody or antigen binding fragment thereof that binds to Fibroblast Activation Protein (FAP), wherein the antibody or antigen binding fragment comprises the VL CDR1, VL CDR2, and VL CDR3 comprise amino acid sequences of SEQ ID NOS:30, 17, and 56, respectively, and the VH CDR1, VH CDR2, and VH CDR3 comprise amino acid sequences of SEQ ID NOS:36, 63, and 38, respectively.

In some embodiments, the anchoring moiety comprises an antibody or antigen binding fragment thereof that binds to Fibroblast Activation Protein (FAP), wherein the antibody or antigen binding fragment comprises the VL CDR1, VL CDR2, and VL CDR3 comprise amino acid sequences of SEQ ID NOS:30, 17, and 57, respectively, and the VH CDR1, VH CDR2, and VH CDR3 comprise amino acid sequences of SEQ ID NOS:58, 64, and 51, respectively.

In some embodiments, the anchoring moiety comprises an antibody or antigen binding fragment thereof that binds to Fibroblast Activation Protein (FAP), wherein the antibody or antigen binding fragment comprises the antibody is an VHH comprising the VH CDR1, VH CDR2, and VH CDR3 comprise amino acid sequences of SEQ ID NOS:65, 66, and 67, respectively.

In some embodiments, the anchoring moiety comprises an antibody or antigen binding fragment thereof that binds to Fibroblast Activation Protein (FAP), wherein the antibody or antigen binding fragment comprises (a) a light chain variable region (VL) comprising VL of any one of antibodies 872-5, 872-59, 872-70, or 872-5V1 as set forth in Table 7; and/or (b) a heavy chain variable region (VH) comprising VH of any one of antibodies 872-5, 872-59, 872-70, 872-5V1, or VHH6 as set forth in Table 8.

In some embodiments, the anchoring moiety comprises an antibody or antigen binding fragment thereof that binds to Fibroblast Activation Protein (FAP), wherein the antibody or antigen binding fragment comprises a VL comprising an amino acid sequence of SEQ ID NO: 91, SEQ ID NO: 92, SEQ ID NO: 93, or SEQ ID NO: 94.

In some embodiments, the anchoring moiety comprises an antibody or antigen binding fragment thereof that binds to Fibroblast Activation Protein (FAP), wherein the antibody or antigen binding fragment comprises a VH comprising an amino acid sequence of SEQ ID NO: 95, SEQ ID NO: 96, SEQ ID NO: 97, SEQ ID NO: 98, or SEQ ID NO: 99.

In some embodiments, the anchoring moiety comprises an antibody or antigen binding fragment thereof that binds to Fibroblast Activation Protein (FAP), wherein the antibody or antigen binding fragment comprises a VL comprising an amino acid sequence of SEQ ID NO: 91; and a VH comprising an amino acid sequence of SEQ ID NO: 95.

In some embodiments, the anchoring moiety comprises an antibody or antigen binding fragment thereof that binds to Fibroblast Activation Protein (FAP), wherein the antibody or antigen binding fragment comprises a VL comprising an amino acid sequence of SEQ ID NO: 92; and a VH comprising an amino acid sequence of SEQ ID NO: 96.

In some embodiments, the anchoring moiety comprises an antibody or antigen binding fragment thereof that binds to Fibroblast Activation Protein (FAP), wherein the antibody or antigen binding fragment comprises a VL comprising an amino acid sequence of SEQ ID NO: 93; and a VH comprising an amino acid sequence of SEQ ID NO: 97.

In some embodiments, the anchoring moiety comprises an antibody or antigen binding fragment thereof that binds to Fibroblast Activation Protein (FAP), wherein the antibody or antigen binding fragment comprises a VL comprising an amino acid sequence of SEQ ID NO: 94; and a VH comprising an amino acid sequence of SEQ ID NO: 98.

In some embodiments, the anchoring moiety comprises an antibody or antigen binding fragment thereof that binds to Fibroblast Activation Protein (FAP), wherein the antibody or antigen binding fragment comprises a VHH comprising an amino acid sequence of SEQ ID NO: 99.

The present disclosure provides, in certain embodiments, a composition comprising the immunoconjugate molecule according to the present disclosure, and a pharmaceutical acceptable carrier.

The present disclosure provides, in certain embodiments, a polynucleotide encoding the immunoconjugate molecule according to the present disclosure, or a subunit or a fragment thereof. In some embodiments, the polynucleotide is operably linked to a promoter. Also provided herein is a population of polynucleotides encoding the immunoconjugate molecule according to the present disclosure, or a subunit or a fragment thereof. For example, in some embodiments, a first polynucleotide encodes a first subunit or polypeptide forming part of the immunoconjugate molecule, and a second polynucleotide encodes a second subunit or polypeptide forming part of the immunoconjugate molecule. In some embodiments, the first polynucleotide is operably linked to a first promoter and the second polynucleotide is operably linked to a second promoter.

The present disclosure provides, in certain embodiments, a vector comprising the polynucleotide according to the present disclosure. The present disclosure further provides, in certain embodiments a population of vectors comprising: (a) a first vector comprising nucleotide sequences encoding a first subunit or polypeptide forming part of the immunoconjugate molecule provided herein operably linked to a first promoter, and (b) a second vector comprising nucleotide sequences encoding a second subunit or polypeptide forming part of the immunoconjugate molecule provided herein operably linked to a second promoter.

The present disclosure provides, in certain embodiments, a cell comprising the polynucleotide according to the present disclosure. Also provided herein is a cell comprising a vector or a population of vectors according to the present disclosure. The present disclosure provides, in certain embodiments, an isolated cell producing the immunoconjugate molecule according to the present disclosure.

Also provided herein is a population of cells comprising: (a) a first host cell comprising a polynucleotide comprising nucleotide sequences encoding a first subunit of polypeptide forming part of an immunoconjugate molecule provided herein, and (b) a second host cell comprising a polynucleotide comprising nucleotide sequences encoding a second subunit of polypeptide forming part of an immunoconjugate molecule provided herein.

Further provided herein is a population of cells comprising: (a) a first host cell comprising a polynucleotide comprising nucleotide sequences encoding a first subunit of polypeptide forming part of an immunoconjugate molecule provided herein operably linked to a first promoter, and (b) a second host cell comprising a polynucleotide comprising nucleotide sequences encoding a second subunit of polypeptide forming part of an immunoconjugate molecule provided herein operably linked to a second promoter.

The present disclosure provides, in certain embodiments, a kit comprising the immunoconjugate molecule according to the present disclosure.

Also provided herein is a method of making an immunoconjugate molecule according to the present disclosure or a subunit or fragment thereof. In certain embodiments, the method comprises culturing a cell provided herein to express the immunoconjugate molecule or a subunit or fragment thereof. In other embodiments, the method comprises expressing a polynucleotide provided herein.

In a related aspect, provided herein is a method for activating a cytokine-mediated effect at a target site, the method comprising delivering to the target site an immunoconjugate molecule comprising the cytokine and a masking moiety; wherein the masking moiety comprises a two-in-one antibody or antigen binding fragment thereof that binds to the cytokine through intramolecular interaction and inhibits the cytokine-mediated effect; wherein the two-in-one antibody or antigen binding fragment is capable of binding to a first target antigen in the target site; wherein when the immunoconjugate molecule is at the target site, the two-in-one antibody binds to the first target antigen and disassociate from the cytokine; and wherein the cytokine-mediated effect is activated at the target site.

In some embodiments, the immunoconjugate molecule further comprises an anchoring moiety; wherein the anchoring moiety comprises an antibody or antigen binding fragment thereof capable of binding to a second target antigen in the target site.

In some embodiments, when the immunoconjugate molecule is at the target site, the antibody or antigen binding fragment of the anchoring moiety binds to the second target antigen; and wherein the immunoconjugate molecule is immobilized at the target site.

In some embodiments, delivering the immunoconjugate molecule to the target site comprises administering the immunoconjugate molecule to a subject. In some embodiments, the cytokine activity is at least about 10%, 20%, 30%, 40%, 45%, 50%, 55%, 60%, 65%, 70%, 75%, 80%, 85%, 90%, 95%, or 98% lower at a non-target site as compared to the cytokine activity at the target site after administration the immunoconjugate molecule to a subject.

In a related aspect, provided herein is a method for enriching a cytokine at a target site, the method comprising delivering to the target site an immunoconjugate molecule comprising the cytokine and an anchoring moiety; wherein the anchoring moiety comprises an antibody or antigen binding fragment thereof capable of binding to a second target antigen in the target site; wherein when the immunoconjugate molecule is at the target site, the anchoring moiety binds to the second target antigen; and wherein the cytokine is distributed at a higher concentration at the target site as compared to a non-target site.

In some embodiments, delivering the immunoconjugate molecule to the target site comprises administering the immunoconjugate molecule to a subject. In some embodiments, the cytokine concentration is at least about 10%, 20%, 30%, 40%, 45%, 50%, 55%, 60%, 65%, 70%, 75%, 80%, 85%, 90%, 95%, or 98% lower at a non-target site as compared to the cytokine activity at the target site after administration the immunoconjugate molecule to a subject.

In some embodiments, the immunoconjugate molecule further comprises a masking moiety; wherein the masking moiety comprises a two-in-one antibody or antigen binding fragment thereof that binds to the cytokine through intramolecular interaction and inhibits an cytokine-mediated effect; wherein the two-in-one antibody or antigen binding fragment is capable of binding to a first target antigen in the target site; wherein when the immunoconjugate molecule is at the target site, the two-in-one antibody binds to the first target antigen and disassociate from the cytokine; and wherein the cytokine-mediated effect is activated at the target site.

In some embodiments, administration of the immunoconjugate molecule to a subject reduces toxicity or side-effect associated with the cytokine in the subject. In some embodiments, the cytokine toxicity or side-effect is reduced at least about 10%, 20%, 30%, 40%, 45%, 50%, 55%, 60%, 65%, 70%, 75%, 80%, 85%, 90%, 95%, or 98% in the present method as compared to administration to the subject an equivalent amount of the cytokine in an unconjugated form. In some embodiments, the reduction in toxicity or side-effect associated with the cytokine is measured as the elongation of life span of the administered subject. In some embodiments, the reduction in toxicity or side-effect associated with the cytokine is measured as reduction in loss of body weight of the administered subject. In some embodiments, the reduction in toxicity or side-effect associated with the cytokine is measured as change in the level of an immune response in the administered subject. In some embodiments, the reduction in toxicity or side-effect associated with the cytokine is measured as a change in an inflammatory response in the administered subject.

In some embodiments of the present method, the first antigen and second antigen are the same antigen or different antigens. In some embodiments, the target site is tumor microenvironment. In some embodiments, the target site is a cancerous cell. In some embodiments, the first and/or second antigen is expressed on the surface of cancer cells. In some embodiments, the first and/or second antigen is expressed by cells in the tumor microenvironment. In some embodiments, the first and/or second antigen is fibrosis activation protein (FAP). In some embodiments, the immunoconjugate molecule further comprises conjugating moiety configured for operably connecting two or more of the cytokine polypeptide, the masking moiety and the anchoring moiety. In some embodiments, the conjugating moiety is an immunoglobulin Fc domain comprising a first subunit and a second subunit that are two non-identical polypeptide chains; and wherein the Fc domain comprises a first modification promoting hetero-dimerization of the two non-identical polypeptide chains. In some embodiments, the immunoglobulin domain comprises a second modification, wherein the Fc domain has reduced binding affinity to an Fc receptor compared to a native Fc domain without said second modification. In some embodiments, the immunoconjugate molecule used in the present method is the immunoconjugate molecule according to the present disclosure.

The present disclosure provides, in certain embodiments, antibody or antigen binding fragments thereof that can form part of the immunoconjugate molecules of the present disclosure. In some embodiments, provided herein is a two-in-one antibody or antigen binding fragment thereof that binds to Fibroblast Activation Protein (FAP) and interleukin-2 (IL-2), wherein the antibody or antigen binding fragment comprises (a) a light chain variable region (VH) comprising VL complementarity determining region 1 (CDR1), VL CDR2, and VL CDR3 of any one of antibodies D001, D002, D029, D029LV1, D029LV2, D029LV3, D029LV4, D029LV5, D003, D047, D049, or B10 as set forth in Table 1; and/or (b) a heavy chain variable region (VH) comprising VH complementarity determining region 1 (CDR1), VH CDR2, and VH CDR3 of any one of antibodies D001, D002, D029, D029HV1, D029HV2, D029HV3, D029HV4, D029HV5, D029HV6, D003, D047, D049, or B10 as set forth in Table 2.

In some embodiments, the two-in-one antibody or antigen binding fragment thereof that binds to Fibroblast Activation Protein (FAP) and interleukin-2 (IL-2) comprises the VL CDR1, VL CDR2, and VL CDR3 comprise amino acid sequences of SEQ ID NOS:16, 17, and 18, respectively, and the VH CDR1, VH CDR2, and VH CDR3 comprise amino acid sequences of SEQ ID NOS:36, 37, and 38, respectively.

In some embodiments, the two-in-one antibody or antigen binding fragment thereof that binds to Fibroblast Activation Protein (FAP) and interleukin-2 (IL-2) comprises the VL CDR1, VL CDR2, and VL CDR3 comprise amino acid sequences of SEQ ID NOS:19, 17, and 20, respectively, and the VH CDR1, VH CDR2, and VH CDR3 comprise amino acid sequences of SEQ ID NOS:36, 39, and 38, respectively.

In some embodiments, the two-in-one antibody or antigen binding fragment thereof that binds to Fibroblast Activation Protein (FAP) and interleukin-2 (TL-2) comprises the VL CDR1, VL CDR2, and VL CDR3 comprise amino acid sequences of SEQ ID NOS:21, 22, and 23, respectively, and the VH CDR1, VH CDR2, and VH CDR3 comprise amino acid sequences of SEQ ID NOS:40, 41, and 38, respectively.

In some embodiments, the two-in-one antibody or antigen binding fragment thereof that binds to Fibroblast Activation Protein (FAP) and interleukin-2 (TL-2) comprises the VL CDR1, VL CDR2, and VL CDR3 comprise amino acid sequences of SEQ ID NOS:30, 17, and 31, respectively, and the VH CDR1, VH CDR2, and VH CDR3 comprise amino acid sequences of SEQ ID NOS:46, 47, and 48, respectively.

In some embodiments, the two-in-one antibody or antigen binding fragment thereof that binds to Fibroblast Activation Protein (FAP) and interleukin-2 (TL-2) comprises the VL CDR1, VL CDR2, and VL CDR3 comprise amino acid sequences of SEQ ID NOS:32, 17, and 33, respectively, and the VH CDR1, VH CDR2, and VH CDR3 comprise amino acid sequences of SEQ ID NOS:49, 50, and 51, respectively.

In some embodiments, the two-in-one antibody or antigen binding fragment thereof that binds to Fibroblast Activation Protein (FAP) and interleukin-2 (TL-2) comprises the VL CDR1, VL CDR2, and VL CDR3 comprise amino acid sequences of SEQ ID NOS:34, 17, and 35, respectively, and the VH CDR1, VH CDR2, and VH CDR3 comprise amino acid sequences of SEQ ID NOS:52, 53, and 51, respectively.

In some embodiments, the two-in-one antibody or antigen binding fragment thereof that binds to Fibroblast Activation Protein (FAP) and interleukin-2 (TL-2) comprises the VL CDR1, VL CDR2, and VL CDR3 comprise amino acid sequences of SEQ ID NOS:24, 25, and 23, respectively, and the VH CDR1, VH CDR2, and VH CDR3 comprise amino acid sequences of SEQ ID NOS:40, 42, and 38, respectively.

In some embodiments, the two-in-one antibody or antigen binding fragment thereof that binds to Fibroblast Activation Protein (FAP) and interleukin-2 (TL-2) comprises the VL CDR1, VL CDR2, and VL CDR3 comprise amino acid sequences of SEQ ID NOS:26, 25, and 28, respectively, and the VH CDR1, VH CDR2, and VH CDR3 comprise amino acid sequences of SEQ ID NOS:43, 42, and 38, respectively.

In some embodiments, the two-in-one antibody or antigen binding fragment thereof that binds to Fibroblast Activation Protein (FAP) and interleukin-2 (TL-2) comprises the VL CDR1, VL CDR2, and VL CDR3 comprise amino acid sequences of SEQ ID NOS:26, 25, and 29, respectively, and the VH CDR1, VH CDR2, and VH CDR3 comprise amino acid sequences of SEQ ID NOS:43, 42, and 38, respectively.

In some embodiments, the two-in-one antibody or antigen binding fragment thereof that binds to Fibroblast Activation Protein (FAP) and interleukin-2 (TL-2) comprises the VL CDR1, VL CDR2, and VL CDR3 comprise amino acid sequences of SEQ ID NOS:24, 25, and 29, respectively, and the VH CDR1, VH CDR2, and VH CDR3 comprise amino acid sequences of SEQ ID NOS:40, 42, and 38, respectively.

In some embodiments, the two-in-one antibody or antigen binding fragment thereof that binds to Fibroblast Activation Protein (FAP) and interleukin-2 (TL-2) comprises the VL CDR1, VL CDR2, and VL CDR3 comprise amino acid sequences of SEQ ID NOS:26, 25, and 27, respectively, and the VH CDR1, VH CDR2, and VH CDR3 comprise amino acid sequences of SEQ ID NOS:43, 42, and 38, respectively.

In some embodiments, the two-in-one antibody or antigen binding fragment thereof that binds to Fibroblast Activation Protein (FAP) and interleukin-2 (TL-2) comprises the VL CDR1, VL CDR2, and VL CDR3 comprise amino acid sequences of SEQ ID NOS:26, 25, and 27, respectively, and the VH CDR1, VH CDR2, and VH CDR3 comprise amino acid sequences of SEQ ID NOS:44, 42, and 38, respectively.

In some embodiments, the two-in-one antibody or antigen binding fragment thereof that binds to Fibroblast Activation Protein (FAP) and interleukin-2 (TL-2) comprises the VL CDR1, VL CDR2, and VL CDR3 comprise amino acid sequences of SEQ ID NOS:26, 25, and 27, respectively, and the VH CDR1, VH CDR2, and VH CDR3 comprise amino acid sequences of SEQ ID NOS:45, 42, and 38, respectively.

In some embodiments, the two-in-one antibody or antigen binding fragment thereof that binds to Fibroblast Activation Protein (FAP) and interleukin-2 (IL-2) comprises the VL CDR1, VL CDR2, and VL CDR3 comprise amino acid sequences of SEQ ID NOS:103, 17, and 104, respectively, and the VH CDR1, VH CDR2, and VH CDR3 comprise amino acid sequences of SEQ ID NOS:105, 106, and 38, respectively.

In some embodiments, the two-in-one antibody or antigen binding fragment thereof that binds to Fibroblast Activation Protein (FAP) and interleukin-2 (IL-2) comprises: (a) a light chain variable region (VL) comprising VL of any one of antibodies D001, D002, D029, D029LV1, D029LV2, D029LV3, D029LV4, D029LV5, D003, D047, D049, or B10 as set forth in Table 3; and/or (b) a heavy chain variable region (VH) comprising VH of any one of antibodies D001, D002, D029, D029LV1, D029LV2, D029LV3, D029LV4, D029LV5, D003, D047, D049, or B10 as set forth in Table 4.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 68, SEQ ID NO: 69, SEQ ID NO: 70, SEQ ID NO: 71, SEQ ID NO: 72, SEQ ID NO: 73, SEQ ID NO: 74, SEQ ID NO: 75, SEQ ID NO:76, SEQ ID NO: 77, SEQ ID NO: 78, or SEQ ID NO: 101.

In some embodiments, the two-in-one antibody or antigen binding fragment thereof that binds to Fibroblast Activation Protein (FAP) and interleukin-2 (IL-2) comprises a VH comprising an amino acid sequence of SEQ ID NO: 79, SEQ ID NO: 80, SEQ ID NO: 81, SEQ ID NO: 82, SEQ ID NO: 83, SEQ ID NO: 84, SEQ ID NO: 85, SEQ ID NO: 86, SEQ ID NO:87, SEQ ID NO: 88, SEQ ID NO: 89, SEQ ID NO: 90, or SEQ ID NO: 102.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 68; and a VH comprising an amino acid sequence of SEQ ID NO: 79.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 69; and a VH comprising an amino acid sequence of SEQ ID NO: 80.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 70; and a VH comprising an amino acid sequence of SEQ ID NO: 81.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 76; and a VH comprising an amino acid sequence of SEQ ID NO: 88.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 77; and a VH comprising an amino acid sequence of SEQ ID NO: 89.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 78; and a VH comprising an amino acid sequence of SEQ ID NO: 90.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 71; and a VH comprising an amino acid sequence of SEQ ID NO: 82.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 73; and a VH comprising an amino acid sequence of SEQ ID NO: 83.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 74; and a VH comprising an amino acid sequence of SEQ ID NO: 83.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 75; and a VH comprising an amino acid sequence of SEQ ID NO: 82.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 72; and a VH comprising an amino acid sequence of SEQ ID NO: 84.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 72; and a VH comprising an amino acid sequence of SEQ ID NO: 85.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 72; and a VH comprising an amino acid sequence of SEQ ID NO: 87.

In some embodiments, the two-in-one antibody or antigen binding fragment thereof that binds to Fibroblast Activation Protein (FAP) and interleukin-2 (IL-2) comprises a VL comprising an amino acid sequence of SEQ ID NO: 101; and a VH comprising an amino acid sequence of SEQ ID NO: 102.

The present disclosure provides, in certain embodiments, an immunoconjugate molecule comprising the two-in-one antibody or antigen binding fragment thereof that binds to Fibroblast Activation Protein (FAP) and interleukin-2 (IL-2) disclosed herein and an IL-2 polypeptide. In some embodiments, the IL-2 polypeptide is human IL-2. In some embodiments, IL-2 polypeptide is wild-type or mutant IL-2 as described herein.

The present disclosure also provides, in certain embodiments, an antibody or antigen binding fragment thereof that binds to Fibroblast Activation Protein (FAP), wherein the antibody or antigen binding fragment comprises (a) a light chain variable region (VH) comprising VL complementarity determining region 1 (CDR1), VL CDR2, and VL CDR3 of any one of antibodies 872-5, 872-59, 872-70, or 872-5V1 as set forth in Table 5; and/or (b) a heavy chain variable region (VH) comprising VH complementarity determining region 1 (CDR1), VH CDR2, and VH CDR3 of any one of antibodies 872-5, 872-59, 872-70, 872-5V1, or VHH6 as set forth in Table 6.

In some embodiments, the antibody or antigen binding fragment thereof that binds to Fibroblast Activation Protein (FAP) comprises the VL CDR1, VL CDR2, and VL CDR3 comprise amino acid sequences of SEQ ID NOS:30, 17, and 54, respectively, and the VH CDR1, VH CDR2, and VH CDR3 comprise amino acid sequences of SEQ ID NOS:58, 59, and 60, respectively.

In some embodiments, the antibody or antigen binding fragment thereof that binds to Fibroblast Activation Protein (FAP) comprises the VL CDR1, VL CDR2, and VL CDR3 comprise amino acid sequences of SEQ ID NOS:30, 17, and 55, respectively, and the VH CDR1, VH CDR2, and VH CDR3 comprise amino acid sequences of SEQ ID NOS:61, 62, and 48, respectively.

In some embodiments, the antibody or antigen binding fragment thereof that binds to Fibroblast Activation Protein (FAP) comprises the VL CDR1, VL CDR2, and VL CDR3 comprise amino acid sequences of SEQ ID NOS:30, 17, and 56, respectively, and the VH CDR1, VH CDR2, and VH CDR3 comprise amino acid sequences of SEQ ID NOS:36, 63, and 38, respectively.

In some embodiments, the antibody or antigen binding fragment thereof that binds to Fibroblast Activation Protein (FAP) comprises the VL CDR1, VL CDR2, and VL CDR3 comprise amino acid sequences of SEQ ID NOS:30, 17, and 57, respectively, and the VH CDR1, VH CDR2, and VH CDR3 comprise amino acid sequences of SEQ ID NOS:58, 64, and 51, respectively.

In some embodiments, the antibody or antigen binding fragment thereof that binds to Fibroblast Activation Protein (FAP) comprises the antibody is an VHH comprising the VH CDR1, VH CDR2, and VH CDR3 comprise amino acid sequences of SEQ ID NOS:65, 66, and 67, respectively.

In some embodiments, the antibody or antigen binding fragment thereof that binds to Fibroblast Activation Protein (FAP) comprises (a) a light chain variable region (VL) comprising VL of any one of antibodies 872-5, 872-59, 872-70, or 872-5V1 as set forth in Table 7; and/or (b) a heavy chain variable region (VH) comprising VH of any one of antibodies 872-5, 872-59, 872-70, 872-5V1, or VHH6 as set forth in Table 8.

In some embodiments, the antibody or antigen binding fragment thereof that binds to Fibroblast Activation Protein (FAP) comprises a VL comprising an amino acid sequence of SEQ ID NO: 91, SEQ ID NO: 92, SEQ ID NO: 93, or SEQ ID NO: 94.

In some embodiments, the antibody or antigen binding fragment thereof that binds to Fibroblast Activation Protein (FAP) comprises a VH comprising an amino acid sequence of SEQ ID NO: 95, SEQ ID NO: 96, SEQ ID NO: 97, SEQ ID NO: 98, or SEQ ID NO: 99.

In some embodiments, the antibody or antigen binding fragment thereof that binds to Fibroblast Activation Protein (FAP) comprises a VL comprising an amino acid sequence of SEQ ID NO: 91; and a VH comprising an amino acid sequence of SEQ ID NO: 95.

In some embodiments, the antibody or antigen binding fragment thereof that binds to Fibroblast Activation Protein (FAP) comprises a VL comprising an amino acid sequence of SEQ ID NO: 92; and a VH comprising an amino acid sequence of SEQ ID NO: 96.

In some embodiments, the antibody or antigen binding fragment thereof that binds to Fibroblast Activation Protein (FAP) comprises a VL comprising an amino acid sequence of SEQ ID NO: 93; and a VH comprising an amino acid sequence of SEQ ID NO: 97.

In some embodiments, the antibody or antigen binding fragment thereof that binds to Fibroblast Activation Protein (FAP) comprises a VL comprising an amino acid sequence of SEQ ID NO: 94; and a VH comprising an amino acid sequence of SEQ ID NO: 98.

In some embodiments, the antibody or antigen binding fragment thereof that binds to Fibroblast Activation Protein (FAP) comprises an VHH comprising an amino acid sequence of SEQ ID NO: 99.

The present disclosure provides, in certain embodiments, an immunoconjugate molecule comprising the antibody or antigen binding fragment thereof that binds to Fibroblast Activation Protein (FAP) disclosed herein and an IL-2 polypeptide. In some embodiments, the IL-2 polypeptide is human IL-2. In some embodiments, IL-2 polypeptide is wild-type or mutant IL-2 as described herein.

In another aspect, provided herein is an immunoconjugate molecule comprising an IL-2 polypeptide conjugated to a masking moiety, wherein the masking moiety comprises a two-in-one antibody or antigen binding fragment thereof capable of binding to the IL-2 polypeptide and a first target antigen; wherein when binding to the IL-2 polypeptide, the masking moiety blocks binding of the IL-2 polypeptide to a first IL-2 receptor (IL-2R) subunit; and wherein when binding to the first target antigen, the masking moiety disassociates from the IL-2 polypeptide, thereby releasing the IL-2 polypeptide for binding with the first IL-2R subunit. In some embodiments, the IL-2 polypeptide comprises one or more mutations that attenuate binding of the IL-2 polypeptide to a second IL-2R subunit.

In some embodiments, the first IL-2R subunit is the IL-2R a-chain (IL-2Rα), and the second IL-2R subunit is the IL-2R β-chain (IL-2Rβ). In some embodiments, the binding of the IL-2 polypeptide to the second IL-2R subunit is reduced about 10%, about 20%, about 30%, about 40%, about 45%, about 50%, about 55%, about 60%, about 65%, about 70%, about 75%, about 80%, about 85%, about 90%, about 95%, or about 99% comparing to wild-type IL-2.

In some embodiments, the one or more mutations that attenuate binding of the IL-2 polypeptide to IL-2Rβ are selected from D20T, D20G, D20A, H16E, H16R, H16A, N88D, N88S, N88R, V91G, V91A, V91R, and V91S, or a combination thereof. In some embodiments, the masking moiety binds to an epitope of IL-2 comprising one or more of the residues P34, K35, R38, T41, F42, K43, F44, Y45, E61, E62, K64, P65, E68, V69, N71, L72, Q74, Y107, and D109 of IL-2.

In some embodiments, the masking moiety binds to an epitope of IL-2 recognized by an antibody comprising a light chain variable region having an amino acid sequence of SEQ ID NO: 101 and a heavy chain variable region having an amino acid sequence of SEQ ID NO: 102. In some embodiments, the masking moiety competes for binding with IL-2 with an antibody comprising a light chain variable region having an amino acid sequence of SEQ ID NO: 101 and a heavy chain variable region having an amino acid sequence of SEQ ID NO:102.

In some embodiments, the masking moiety comprises (a) a light chain variable region (VL) comprising VL complementarity determining region 1 (CDR1), VL CDR2, and VL CDR3 of antibody B10 as set forth in Table 1; and/or (b) a heavy chain variable region (VH) comprising VH complementarity determining region 1 (CDR1), VH CDR2, and VH CDR3 of antibody B10 as set forth in Table 2.

In some embodiments, wherein the masking moiety comprises (a) the VL CDR1, VL CDR2, and VL CDR3 comprising amino acid sequences of SEQ ID NOS:103, 17, and 104, respectively, and (b) the VH CDR1, VH CDR2, and VH CDR3 comprising amino acid sequences of SEQ ID NOS:105, 106, and 38, respectively.

In some embodiments, wherein the masking moiety comprises: (a) a light chain variable region (VL) comprising VL of antibody B10 as set forth in Table 3; and/or (b) a heavy chain variable region (VH) comprising VH of antibody B10 as set forth in Table 4.

In some embodiments, wherein the masking moiety comprises a VL comprising an amino acid sequence of SEQ ID NO: 101. In some embodiments, wherein the masking moiety comprises a VH comprising an amino acid sequence of SEQ ID NO: 102. In some embodiments, wherein the masking moiety comprises (a) a VL comprising an amino acid sequence of SEQ ID NO: 101; and (b) a VH comprising an amino acid sequence of SEQ ID NO:102.

In some embodiments, wherein the first IL-2R subunit is the IL-2Rβ, and the second IL-2R subunit is the IL-2Rα. In some embodiments, wherein binding of the IL-2 polypeptide to the IL-2Rα is reduced about 10%, about 20%, about 30%, about 40%, about 45%, about 50%, about 55%, about 60%, about 65%, about 70%, about 75%, about 80%, about 85%, about 90%, about 95%, or about 99% comparing to wild-type IL-2.

In some embodiments, the one or more mutations that attenuate binding of the IL-2 polypeptide to IL-2Rα are selected from K35E, R38A, R38E, R38D, F42A, F42K, K43E, Y45A, E61R, E62A, L72G, or a combination thereof. In some embodiments, the one or more mutations that attenuate binding of the IL-2 polypeptide to IL-2Rα are (a) F42A; or (b) K35E and F42A. In some embodiments, the masking moiety binds to an epitope of IL-2 comprising one or more of the residues L12, Q13, E15, H16, L19, D20, M23, R81, D84, D87, N88, V91, 192, and E95 or IL-2.

In some embodiments, the masking moiety binds to an epitope of IL-2 recognized by the antibody 5UTZ. In some embodiments, the masking moiety competes for binding with IL-2 with antibody 5UTZ.

In some embodiments, the IL-2 polypeptide further comprises one or more mutations that modifying binding of the IL-2 polypeptide to IL-2R y-chain (IL-2Rγ). In some embodiments, the one or more mutations modifying binding of the IL-2 polypeptide to IL-2Rγ is selected from L18R, Q22E, T123A, Q126T, I129V, S130A, S130R, or a combination thereof.

In some embodiments, the immunoconjugate further comprises an anchoring moiety, wherein the anchoring moiety comprises an antibody or antigen binding fragment thereof that specifically binds to a second target antigen. In some embodiments, wherein the masking moiety disassociate from the IL-2 polypeptide in the presence of the first target antigen expressed on the surface of a first cell.

In some embodiments, wherein the second target antigen is expressed on the surface of the first cell or a second cell in proximity of the first cell. In some embodiments, the first target antigen and the second target antigen are the same or different. In some embodiments, the first target antigen and/or the second target antigen is a tumor associated antigen. In some embodiments, the first target antigen and the second target antigen are each independently selected from FAP, Her2, Her3, CD19, CD20, BCMA, PSMA, CEA, cMET, EGFR, CA-125, MUC-1, EpCAM, or Trop-2. In some embodiments, the first target antigen is FAP.

In another aspect, provided herein is a method for activating an IL-2R comprising contacting the IL-2R with an effective amount of an immunoconjugate molecule comprising an IL-2 polypeptide as provided herein. In some embodiments, the IL-2R comprises IL-2Rβ. In some embodiments, the IL-2R comprises IL-2Rα. In some embodiments, the IL-2R comprises IL-2Rγ.

In some embodiments, the IL-2R comprises the IL-2Rβ, and wherein the IL-2Rβ is expressed on the surface of a first cell. In some embodiments, the IL-2R further comprises the IL-2Rγ, and wherein the IL-2Rγ is expressed on the surface of the first cell.

In some embodiments, the IL-2R further comprises the IL-2Rα. In some embodiments, the IL-2Rα is associated on a cell surface. In some embodiments, the IL-2Rα is associated on the surface of the first cell (cis-presentation). In some embodiments, the IL-2Rα is associated on the surface of a second cell (trans-presentation). In some embodiments, the IL-2Rα is not associated on a cell surface. In some embodiments, the IL-2R does not comprises the IL-2Rα.

In some embodiments, the first cell and/or the second cell is an immune cell, and wherein upon activation of the IL-2R, the immune cell is activated. In some embodiments, activation of the immune cell is measured as increased proliferation or maturation of the immune cell. In some embodiments, proliferation or maturation of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%. In some embodiments, activation of the immune cell is measured as prolonged survival time of the immune cell. In some embodiments, survival time of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

In some embodiments, the immune cell is an effector T cell, memory T cell, or a combination thereof. In some embodiments, the immune cell is CD4+ T cells, CD8+ T cells, helper T cells, cytotoxic T cells, SLECs (short-lived effector cells), MPEC (memory precursor effector cells), TEs (terminal effector cells), NKs (natural killer cells), NKTs (natural killer T cells), innate lymphoid cells (Types I-III), or a combination thereof.

In some embodiments, the immune cell is a regulatory T cell (Treg). In some embodiments, the immune cell is natural Treg (nTreg) cells, induced Treg (iTreg) cells, or a combination thereof.

In some embodiments, the first cell and/or the second cell is a diseased cell, and wherein upon activation of the IL-2R, the diseased cell dies. In some embodiments, the diseased cell is a cancer cell. In some embodiments, the diseased cell is a cell infected by an infectious pathogen. In some embodiments, the infectious pathogen is a virus, a bacteria, a fungus, a parasite, or a combination thereof.

In one aspect, provided herein is a method of activating a target cell expressing an IL-2R, comprising contacting the target cell with an effective amount of the immunoconjugate molecule of comprising an IL-2 polypeptide as described herein, wherein upon binding of the IL-2 polypeptide with the IL-2R, the target cell is activated. In some embodiments, the target cell is an immune cell. In some embodiments, the target cell is an effector T cell, memory T cell, regulatory T cell, or a combination thereof. In some embodiments, the target cell is CD4+ T cells, CD8+ T cells, helper T cells, cytotoxic T cells, SLECs (short-lived effector cells), MPEC (memory precursor effector cells), TEs (terminal effector cells), NKs (natural killer cells), NKTs (natural killer T cells), innate lymphoid cells (Types I-III), or a combination thereof. In some embodiments, the target cell is natural Treg (nTreg) cells, included Treg (iTreg) cells, or a combination thereof.

In some embodiments, activation of the target cell is measured as increased proliferation or maturation of the target cell. In some embodiments, proliferation or maturation of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

In some embodiments, activation of the target cell is measured as prolonged survival time of the target cell. In some embodiments, survival time of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

In some embodiments, wherein the contacting further comprises administering a pharmaceutical composition comprising a pharmaceutically acceptable carrier and immunoconjugate molecule comprising an IL-2 polypeptide as described herein. In some embodiments, the contacting enhances an anti-neoplastic immune response. In some embodiments, the contacting enhances an anti-infection immune response.

In one aspect, provided herein is a method of enhancing an antigen-specific immune response of a population of T cells, comprising contacting the population of T cells with an effective amount of the immunoconjugate molecule comprising an IL-2 polypeptide as described herein. 141 In some embodiments, the contacting enhances proliferation or maturation of antigen-specific effector T cells. In some embodiments, the contacting enhances formation of antigen-specific memory T cells. In some embodiments, the contacting is performed in the presence of the antigen. In some embodiments, the antigen is an antigen of a cancer, tumor, pathogen, or allergen.

In one aspect, provided herein is a method of increasing secretion of pro-inflammatory cytokines by a population of T cells, comprising contacting the population of T cells with an immunoconjugate molecule comprising an IL-2 polypeptide as described herein, wherein said IL-2 polypeptide activates the T cells upon binding. In some embodiments, the cytokine is IL-1, IL-2, IL-6, IL-12, IL-17, IL-22, IL-23, GM-CSF, TNF-α, IFN-γ, or any combination thereof. In some embodiments, the cytokine production is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

In one aspect, provided herein is a method of increasing assembly of IL-2R on the surface of a target cell, comprising contacting the target cell with an effective amount of the immunoconjugate molecule comprising an IL-2 polypeptide as described herein. In some embodiments, the IL-2R comprises IL-2Rα, IL-2Rβ, IL-2Rγ, or a combination thereof on the surface of the target cell. In some embodiments, the IL-2R comprises IL-2Rβ and IL-2Rγ on the surface of the target cell, and IL-2Rα on the surface of a second cell in proximity of the target cell. In some embodiments, the IL-2R comprises IL-2Rβ and IL-2Rγ on the surface of the target cell, and IL-2Rα not associated with a cell surface. In some embodiments, assembly of IL-2R on the surface of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%. In some embodiments, the target cell is an immune cell. In some embodiments, the target cell is an effector T cell, memory T cell, regulatory T cell, or a combination thereof. In some embodiments, the target cell is CD4+ T cells, CD8+ T cells, helper T cells, cytotoxic T cells, SLECs (short-lived effector cells), MPEC (memory precursor effector cells), TEs (terminal effector cells), NKs (natural killer cells), NKTs (natural killer T cells), innate lymphoid cells (Types I-III), or a combination thereof. In some embodiments, the target cell is natural Treg (nTreg) cells, included Treg (iTreg) cells, or a combination thereof.

In one aspect, provided herein is a method of forming a pro-inflammatory milieu in a tissue surrounding a population of diseased cells, comprising contacting the tissue with an effective amount of the immunoconjugate molecule comprising an IL-2 polypeptide as described herein. In some embodiments, concentration of activated B cells, CD4+ effector T cells, CD8+ effector T cells, dendritic cells, macrophages, natural killer cells, monocytes, granulocytes, eosinophil and/or neutrophils in the tissue is increased. In some embodiments, concentration of regulatory T cells in the tissue is reduced. In some embodiments, concentration of a pro-inflammatory cytokine is increased in the tissue. In some embodiments, the pro-inflammatory cytokine is IL-1, IL-2, IL-6, IL-12, IL-17, IL-22, IL-23, GM-CSF, TNF-α, IFN-γ, or any combination thereof. In some embodiments, concentration of antibodies binding to antigens originated or derived from the diseased cells is increased in the tissue. In some embodiments, presentation of antigens originated or derived from the diseased cells by antigen presentation cells is increased in the tissue. In some embodiments, phagocytosis of the diseased cells is increased in the tissue. In some embodiments, apoptosis of the diseased cells induced by cell-mediated cytotoxicity is increased in the tissue. In some embodiments, apoptosis of the diseased cells induced by antibody-dependent cellular cytotoxicity is increased in the tissue. In some embodiments, the population of the diseased cells is reduced in the tissue. In some embodiments, the population of the diseased cells is reduced by about 10%, about 20%, about 30%, about 40%, about 45%, about 50%, about 55%, about 60%, about 65%, about 70%, about 75%, about 80%, about 85%, about 90%, about 95%, or about 99% in the tissue.

In one aspect, provided herein is a method of eliminating a diseased cell in a subject, comprising administering to the subject an effective amount of the immunoconjugate molecule comprising an IL-2 polypeptide as described herein. In some embodiments, the diseased cell is a cancer cell. In some embodiments, the diseased cell is a cell infected by an infectious pathogen. In some embodiments, the infectious pathogen is a virus, a bacteria, a fungus, a parasite, or a combination thereof.

In one aspect, provided herein is a method of treating cancer in a subject in need thereof, comprising administering to the subject an effective amount of the immunoconjugate molecule comprising an IL-2 polypeptide as described herein. In some embodiments, the treatment enhances an innate, humoral or cell-mediated anti-neoplastic immune response. In some embodiments, the method further comprises co-administration of a second therapy.

In one aspect, provided herein is a method of treating an infection in a subject in need thereof, comprising administering to the subject an effective amount of the immunoconjugate molecule comprising an IL-2 polypeptide as described herein. In some embodiments, the treatment enhances an innate, humoral, or cell-mediated anti-infective immune response. In some embodiments, the subject is co-administered with a vaccine composition for preventing the infection in the subject. In some embodiments, the vaccine composition is co-administered simultaneously or sequentially.

In one aspect, provided herein is a method of increasing the response to an antigen in a subject in need thereof, comprising administering to the subject an effective amount of the immunoconjugate molecule comprising an TL-2 polypeptide as described herein. In some embodiments, the antigen is an antigen of a cancer, tumor, pathogen, or allergen. In some embodiments, the antigen is originated or derived from an infectious pathogen. In some embodiments, the infectious pathogen is a virus, a bacteria, a fungus, a parasite, or a combination thereof. In some embodiments, the antigen is originated or derived from a diseased cell. In some embodiments, the antigen is originated or derived from a cell infected by an infectious pathogen. In some embodiments, the infectious pathogen is a virus, a bacteria, a fungus, a parasite, or a combination thereof. In some embodiments, the antigen is originated or derived from a cancer cell.

In one aspect, provided herein is a method of increasing a response to a vaccine in a subject in need thereof, comprising administering to the subject the vaccine and an effective amount of the immunoconjugate molecule comprising an IL-2 polypeptide as described herein. In some embodiments, the vaccine is a vaccine against a tumor, cancer, pathogen or allergen. In some embodiments, the immunoconjugate molecule is formulated as an adjuvant composition for the vaccine.

In one aspect, provided herein is a method of establishing immune tolerance of an antigen in a tissue surrounding the antigen, comprising contacting the tissue with an effective amount of the immunoconjugate molecule comprising an IL-2 polypeptide as described herein. In some embodiments, concentration of activated B cells, CD4+ effector T cells, CD8+ effector T cells, dendritic cells, macrophages, natural killer cells, monocytes, granulocytes, eosinophil and/or neutrophils in the tissue is reduced. In some embodiments, concentration of regulatory T cells in the tissue is increased. In some embodiments, concentration of a pro-inflammatory cytokine is reduced in the tissue. In some embodiments, the pro-inflammatory cytokine is IL-1, IL-2, IL-6, IL-12, IL-17, IL-22, IL-23, GM-CSF, TNF-α, IFN-γ or any combination thereof. In some embodiments, concentration of antibodies binding to the antigen is reduced in the tissue. In some embodiments, presentation of the antigen by antigen presentation cells is reduced in the tissue. In some embodiments, phagocytosis of cells expressing the antigen is reduced in the tissue. In some embodiments, apoptosis of cells expressing the antigen is reduced in the tissue. In some embodiments, wherein the tissue is in a subject, and wherein the antigen is a self-antigen of the subject. In some embodiments, the subject is suffering from an autoimmune disease.

In yet another aspect, provided herein is a method for treating an autoimmune disease in a subject in need thereof, comprising administering to the subject an effective amount of the immunoconjugate molecule comprising an IL-2 polypeptide as described herein. In some embodiments, the treatment reduces an innate, humoral or cell-mediated immune response towards a self-antigen. In some embodiments, the method further comprises co-administration of a second therapy.

### 4. BRIEF DESCRIPTION OF THE FIGURES

FIG. 1 is a schematic illustration of an antibody-cytokine immunoconjugate molecule according to one embodiment of the present disclosure. In the exemplary embodiment, the immunoconjugate comprises (i) a cytokine polypeptide capable of mediating a cellular effect, (ii) an masking moiety capable of (a) binding to the cytokine and inhibits the cellular effect of the cytokine, and (b) binding to an antigen (e.g., a TAA) in the environment, and upon such binding release the cytokine, (iii) an anchoring moiety capable of binding to the antigen, thereby immobilizing the immunoconjugate in an environment enriched of the antigen; and (iv) a conjugation moiety connecting the portions described in (i), (ii), and (iii) of the immunoconjugate.

FIG. 2 is a schematic illustration of an IL-2 containing immunoconjugate molecule according one embodiment of the present disclosure, and the operation of this immunoconjugate in the absence or presence of Fibroblast Activation Protein (FAP). In this exemplary embodiment, the immunoconjugate comprises (i) an anti-IL-2/anti-FAP two-in-one Fab antibody fused to the N terminus of the immunoglobulin Fc domain, (ii) an IL-2 polypeptide fused to the N terminus of this two-in-one antibody, (iii) an anti-FAP antibody or binding fragment thereof fused to the N terminus of the immunoglobulin Fc domain. Upper panel illustrates that in the absence of FAP in the nearby environment, the equilibrium of the two-in-one antibody shifts towards binding with IL-2 due to the prevalence of intramolecular interaction, thereby preventing IL-2 from binding with cell surface receptors and inhibiting IL-2 cellular effects. Lower panel illustrates that when immobilized in an environment enriched of FAP via the binding of the anti-FAP antibody to FAP, the equilibrium of the two-in-one antibody shifts towards disassociation from IL-2 and binding with FAP, thereby releasing the tethered IL-2 to bind with cell surface receptors and elicit cellular effects.

FIG. 3A shows binding kinetics an anti-FAP antibody designated as 872-5 to biotinylated FAP immobilized on Streptavidin sensor and measured by bio-layer interferometry. The K_{D} values was 6.6 nM for 872-5.

FIG. 3B shows binding kinetics an anti-FAP antibody designated as 872-59 to biotinylated FAP immobilized on Streptavidin sensor and measured by bio-layer interferometry. The K_{D} values was 15.5 nM for 872-59.

FIG. 3C shows binding kinetics an anti-FAP antibody designated as 872-70 to biotinylated FAP immobilized on Streptavidin sensor and measured by bio-layer interferometry. The K_{D} values was <1 nM for 872-70.

FIG. 4A shows binding kinetics of the monovalent Fab-Fc fusion of D002 to biotinylated IL-2 immobilized on Streptavidin sensor and measured by bio-layer interferometry.

FIG. 4B shows the K_{D} value was 3.4 μM for the interaction of D002 with IL-2, determined by equilibrium binding analysis.

FIG. 4C shows binding kinetics of the monovalent Fab-Fc fusion of D002 to FAP immobilized on Streptavidin sensor and measured by bio-layer interferometry. The K_{D} value was 50 nM for the interaction of D002 with FAP (data not shown).

FIG. 5A is a schematic illustration of a soluble cytokine polypeptide.

FIGS. 5B to 5U are schematic illustrations of antibody-cytokine immunoconjugates of different molecular configurations according to the present disclosure. Particularly, FIG. 5B shows an immunoconjugate containing a cytokine polypeptide fused to the C-terminus of one of the two heavy chain fragments in an immunoglobulin Fc domain (e.g., Fc-knob).

FIG. 5C shows an immunoconjugate containing (a) anti-cytokine/anti-TAA two-in-one Fab antibody fused at the N-terminus of its heavy chain fragment to the C-terminus of one of the heavy chain fragments of the immunoglobulin Fc domain (e.g., Fc-hole), and (b) a cytokine polypeptide fused to the C-terminus of the other heavy chain fragment in the immunoglobulin Fc domains (e.g., Fc-knob), and.

FIG. 5D shows an immunoconjugate containing (a) an anti-cytokine/anti-TAA two-in-one Fab antibody fused at the N-terminus of its heavy chain fragment to the C-terminus of one of the heavy chain fragments of the immunoglobulin Fc domain (e.g., the Fc-hole), (b) a cytokine polypeptide fused to the C-terminus of the other one of the two heavy chain fragments of an immunoglobulin Fc domain (e.g., the Fc-knob), and (c) an anti-TAA scFv antibody fused to the N terminus of one of the two heavy chain fragments in the immunoglobulin Fc domain (e.g., the Fc-knob).

FIG. 5E shows an immunoconjugate containing (a) an anti-cytokine/anti-TAA two-in-one Fab antibody fused at the N-terminus of its heavy chain fragment to the C-terminus of one of the two heavy chain fragments of the immunoglobulin Fc domain (e.g., Fc-hole), and (b) a cytokine polypeptide fused to the N terminus of the light chain fragment of the Fab antibody.

FIG. 5F shows an immunoconjugate containing (a) an anti-cytokine/anti-TAA two-in-one Fab antibody fused at the N-terminus of its heavy chain fragment to the C-terminus of one of the heavy chain fragments of an immunoglobulin Fc domain (e.g., Fc-knob); (b) a cytokine polypeptide fused to the C-terminus of the other heavy chain fragment of the immunoglobulin Fc domain (e.g., Fc-hole), and (c) an anti-TAA single domain antibody fused to the N-terminus of one of the two heavy chain fragments in the immunoglobulin Fc domain (e.g., Fc-knob).

FIG. 5G shows an immunoconjugate containing (a) an anti-cytokine/anti-TAA two-in-one scFv antibody fused at the C-terminus of its heavy chain fragment to the N-terminus of one of the heavy chain fragments of an immunoglobulin Fc domain (e.g., Fc-hole), (b) a cytokine polypeptide fused to the C-terminus of the other heavy chain fragment of the immunoglobulin Fc domain (e.g., Fc-knob), and (c) an anti-TAA Fab antibody fused to the N-terminus of one of the two heavy chain fragments in the immunoglobulin Fc domain (e.g., Fc-knob).

FIG. 5H shows an immunoconjugate containing (a) an anti-cytokine/anti-TAA two-in-one Fab antibody fused at the N-terminus of its heavy chain fragment to the C-terminus of one of the two heavy chain fragments of the immunoglobulin Fc domain (e.g., the Fc-knob), (b) a cytokine polypeptide fused to the N-terminus of the light chain fragment of the Fab antibody, and (c) an anti-TAA scFv antibody fused to the N-terminus of one of the two heavy chain fragments in the immunoglobulin Fc domain (e.g., Fc-hole).

FIG. 51 shows an immunoconjugate containing (a) an anti-cytokine/anti-TAA two-in-one Fab antibody fused at the N-terminus of its heavy chain fragment to the C-terminus of one of the two heavy chain fragments of the immunoglobulin Fc domain (e.g., Fc-hole), (b) a cytokine peptide fused to the C-terminus of the other heavy chain fragments in an immunoglobulin Fe domains (e.g., Fc-knob), and (c) an anti-TAA Fab fused to the N-terminus of one of the two heavy chain fragments in the immunoglobulin Fc domain (e.g., Fc-knob).

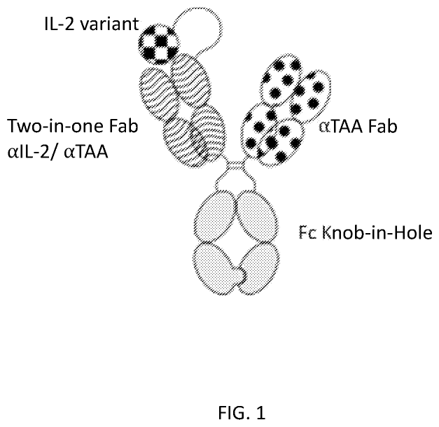

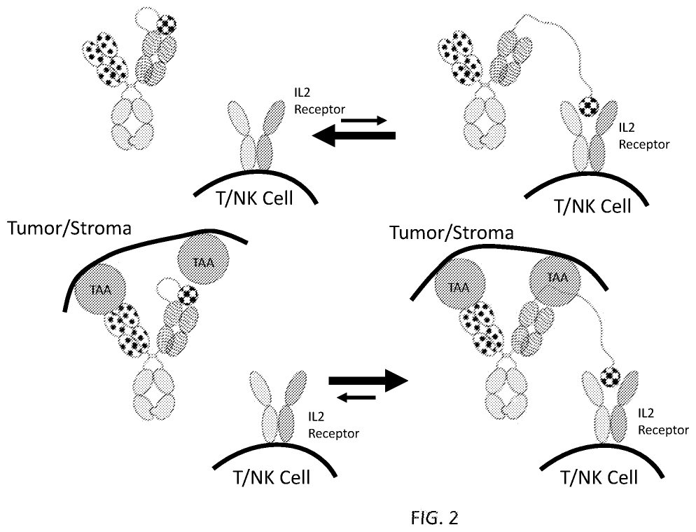

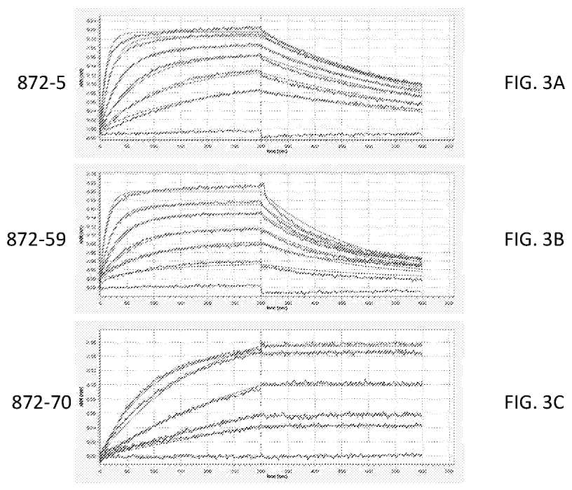

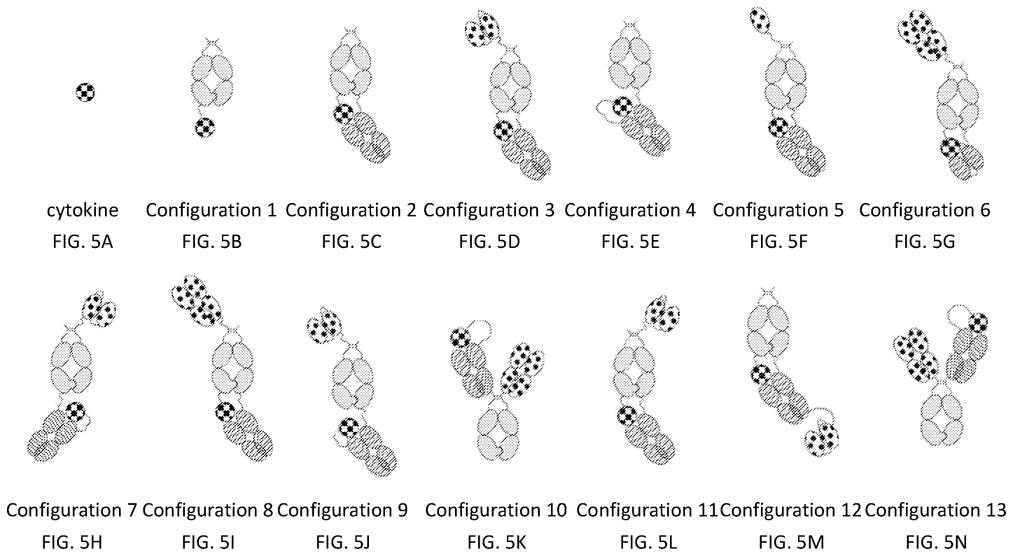

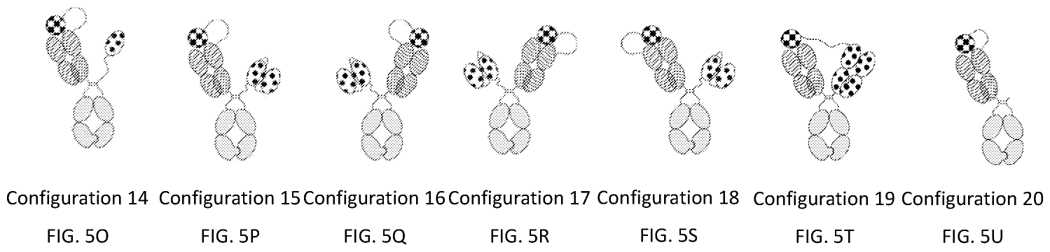

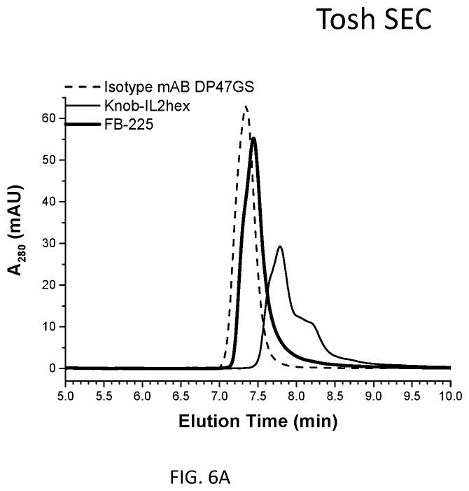

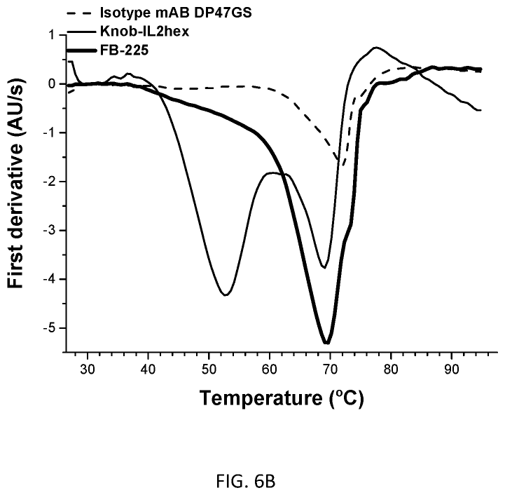

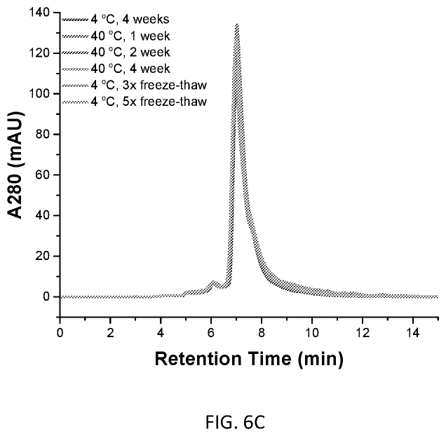

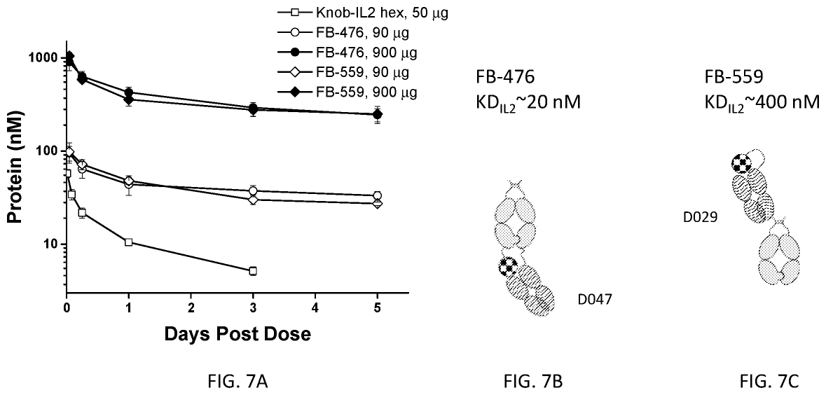

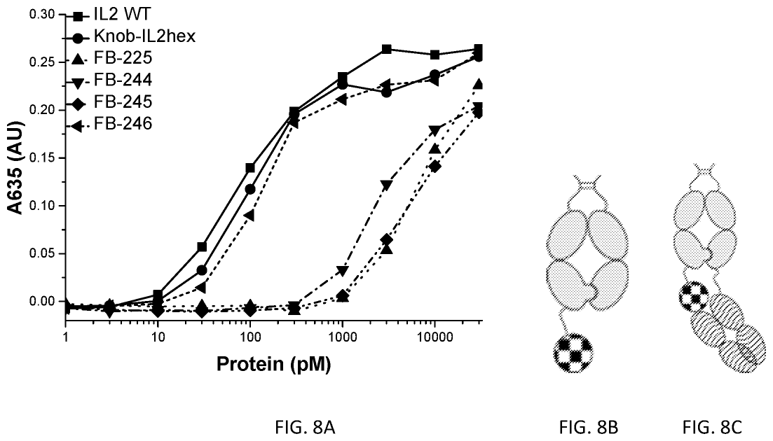

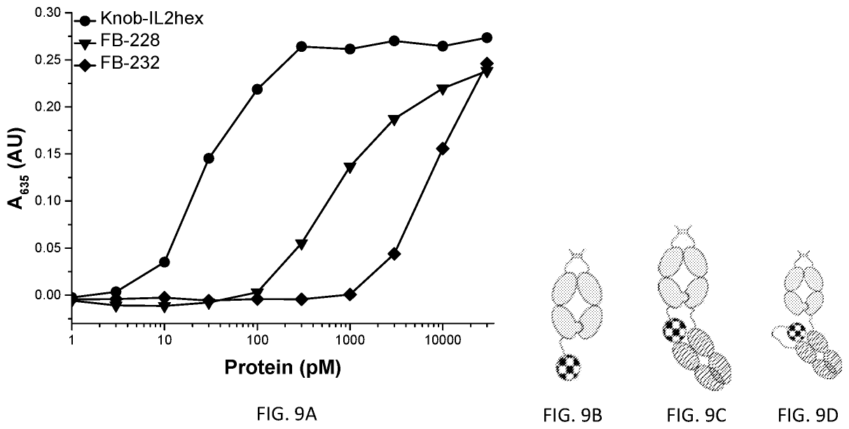

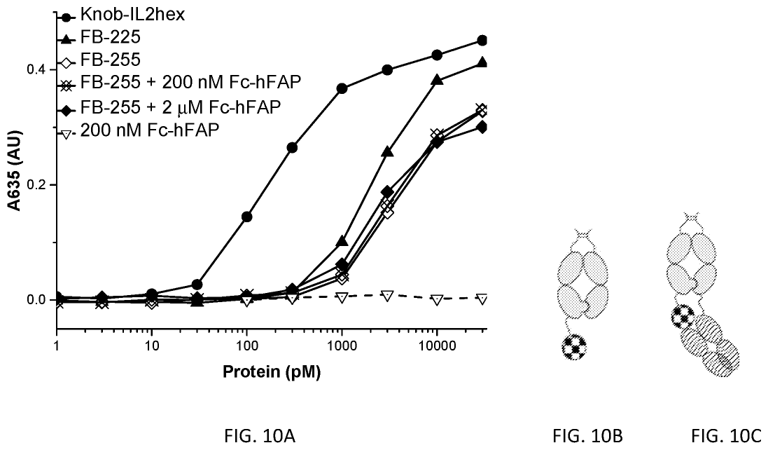

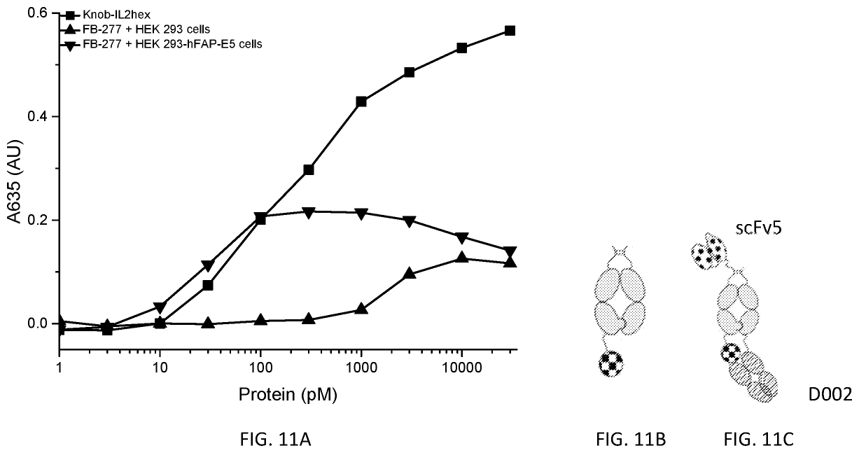

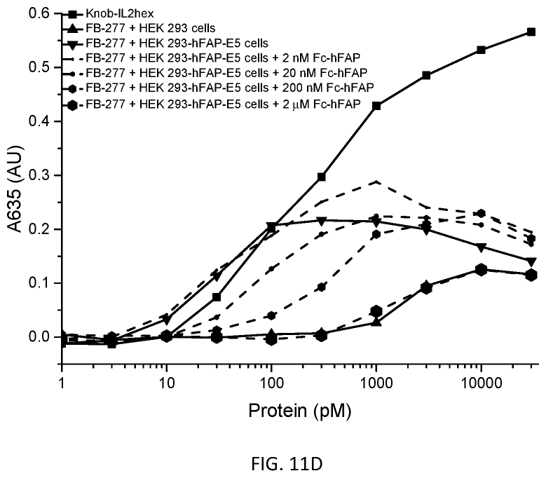

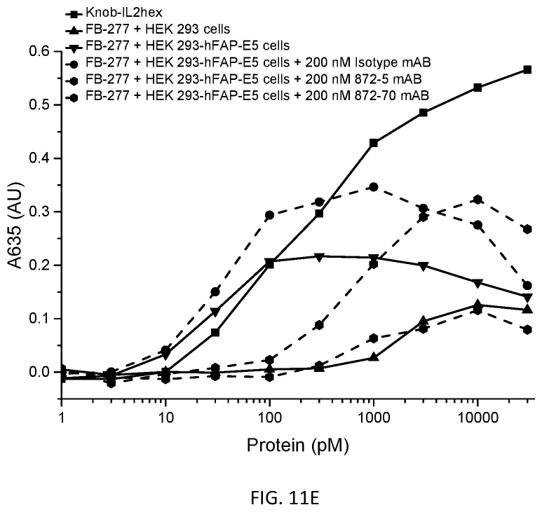

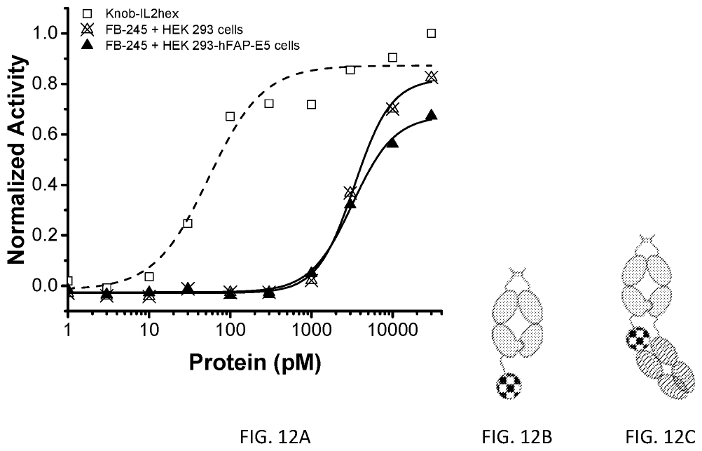

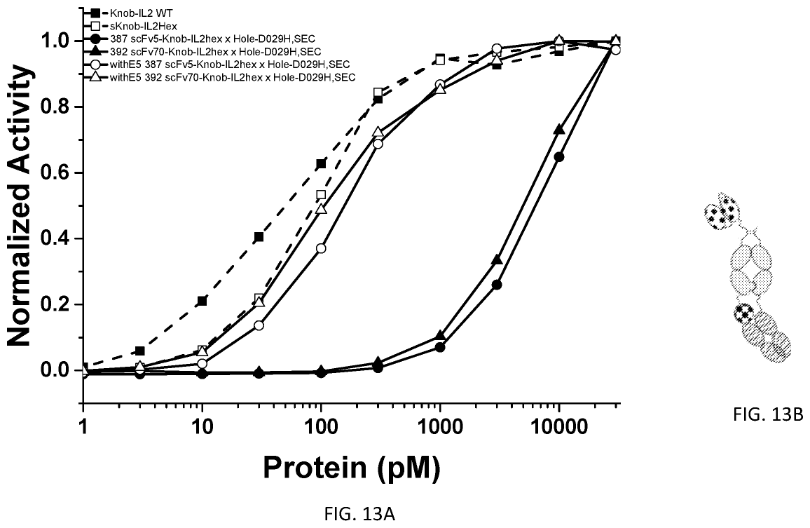

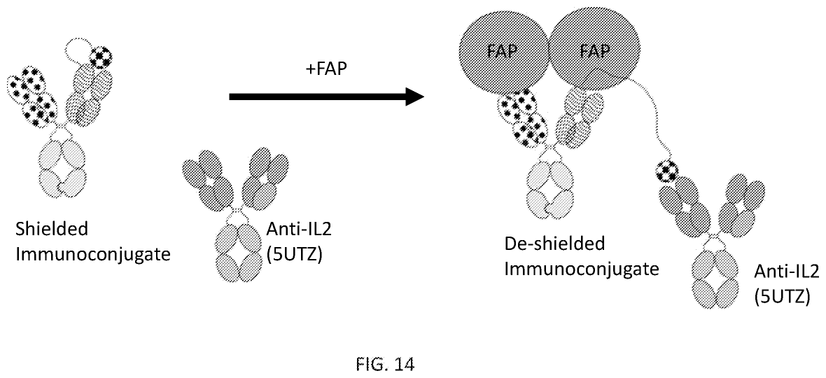

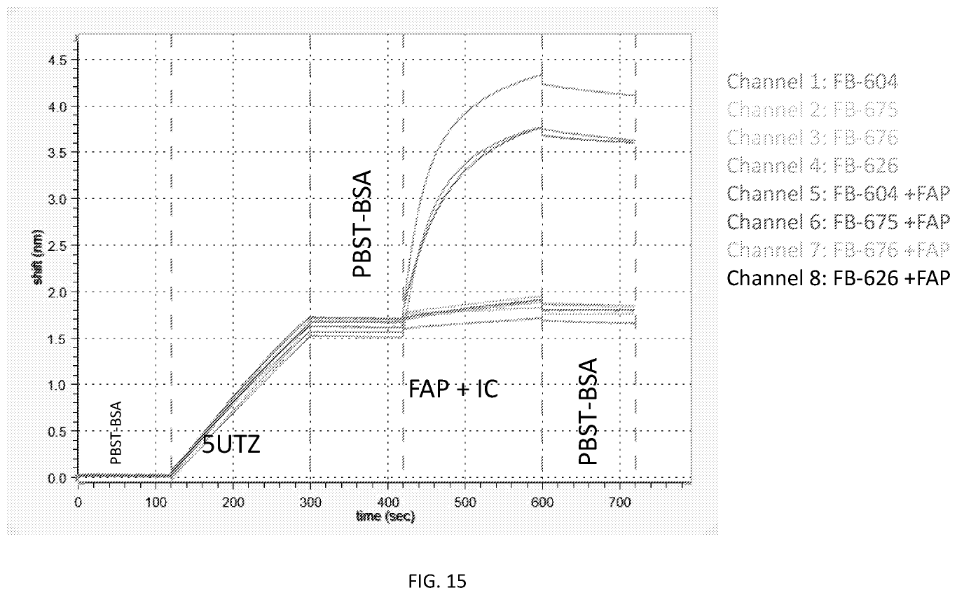

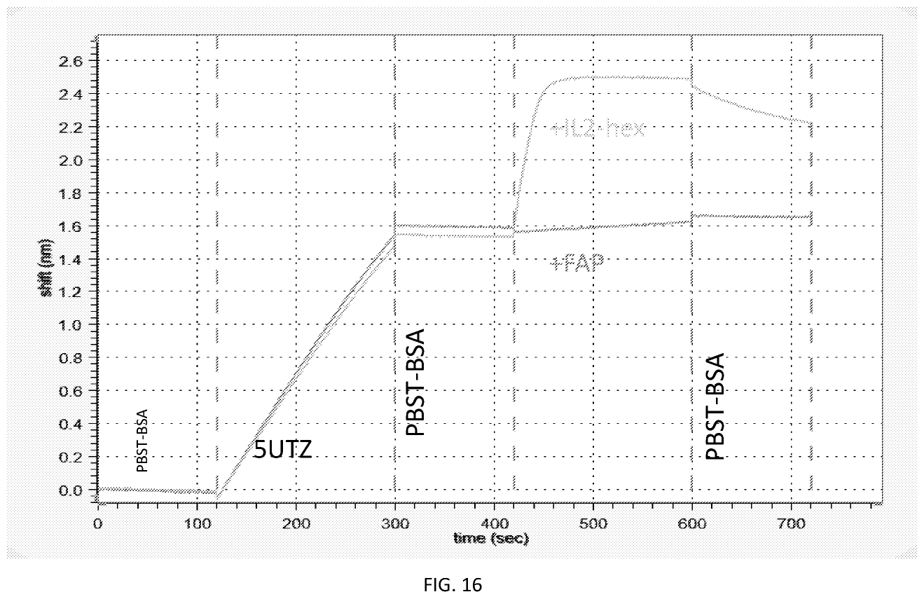

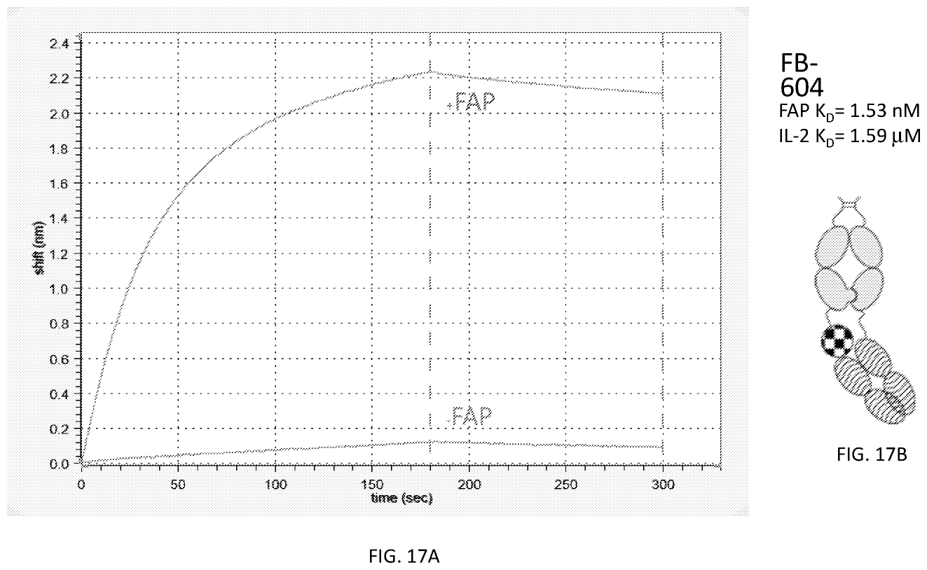

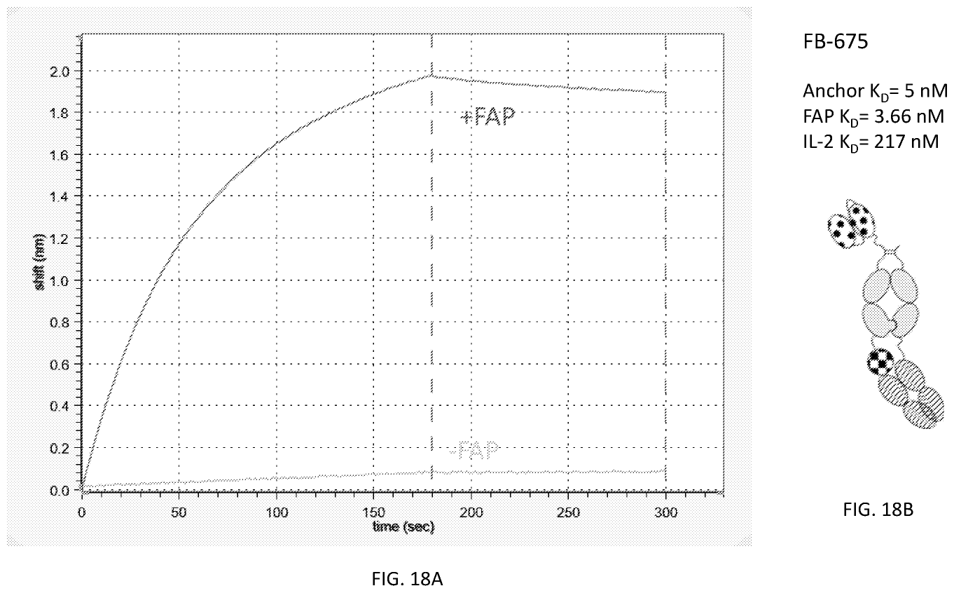

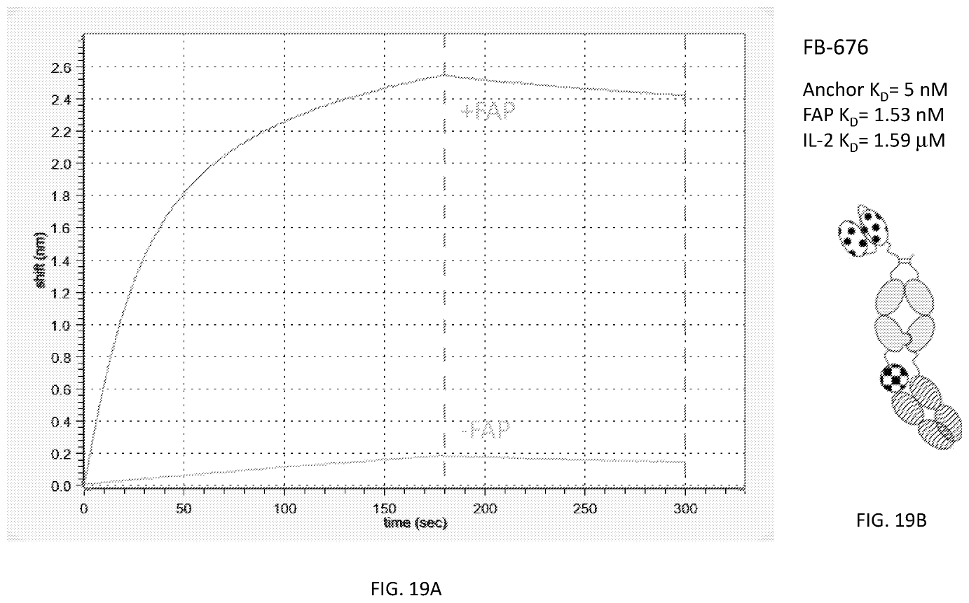

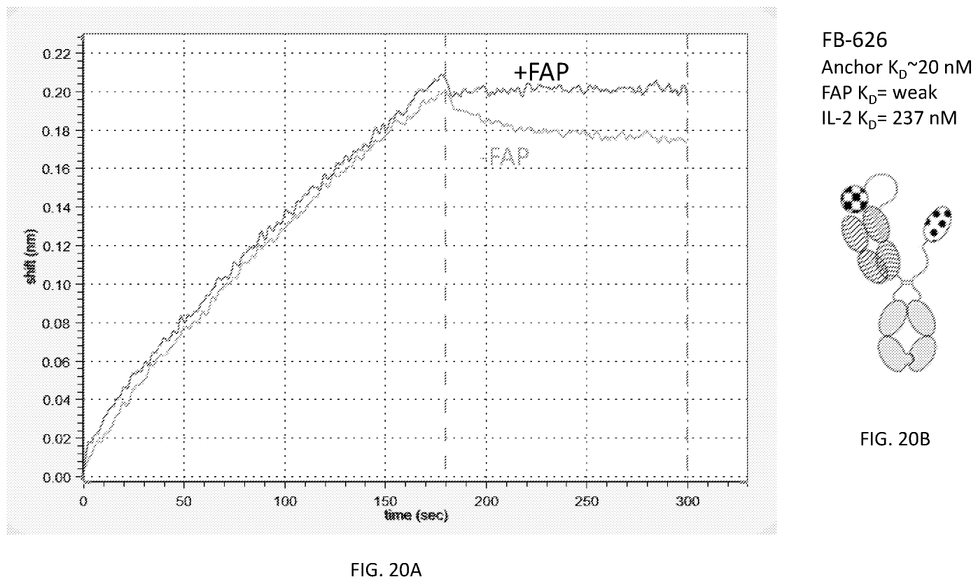

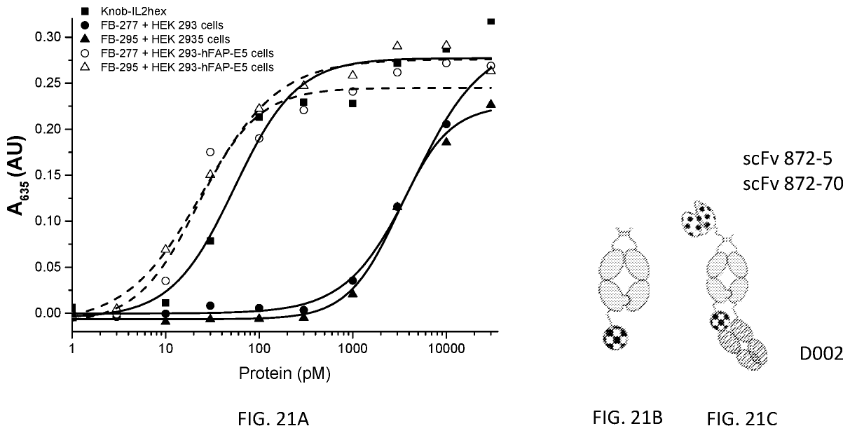

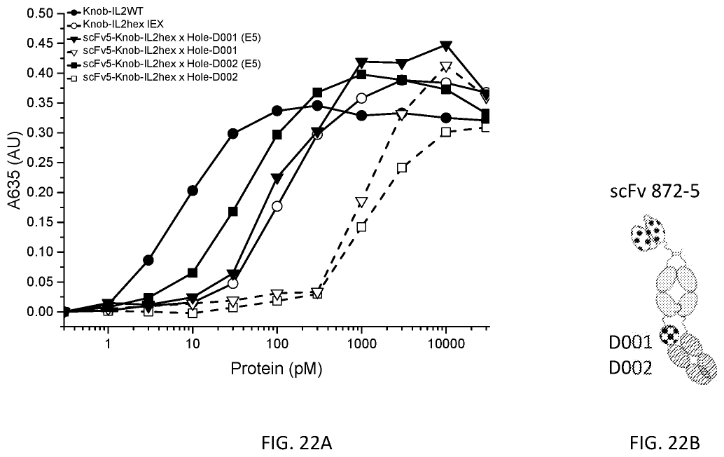

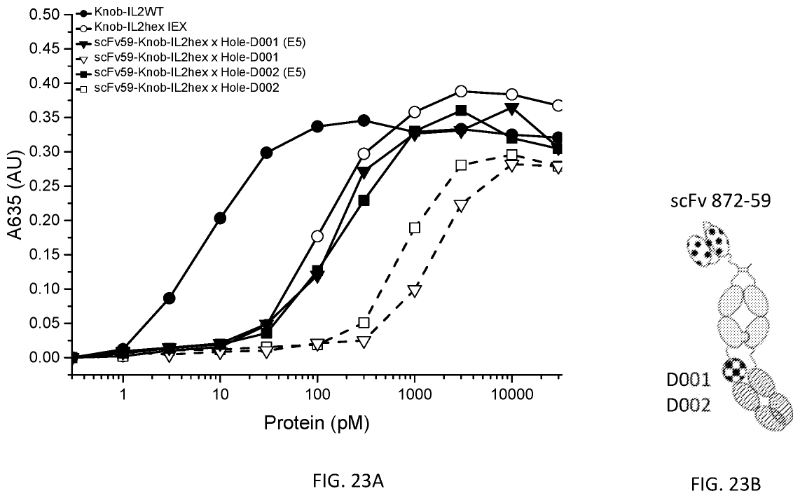

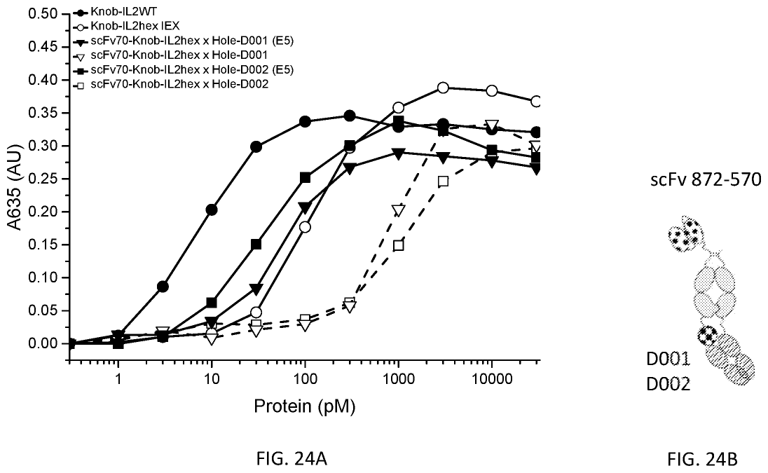

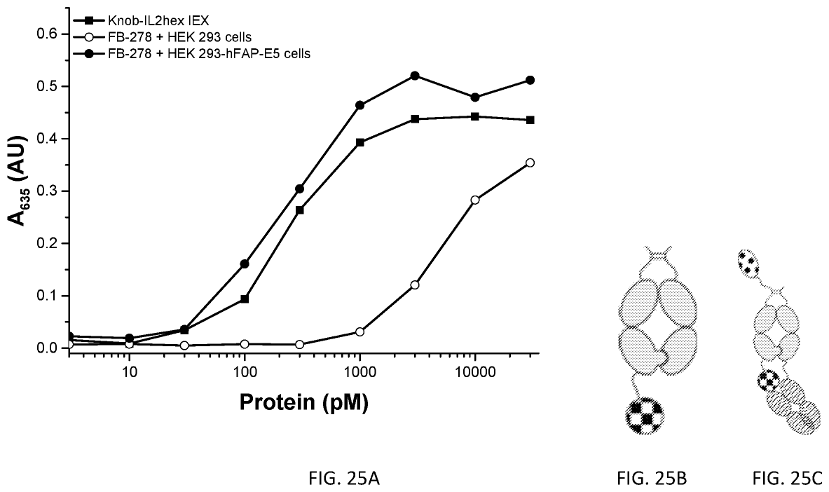

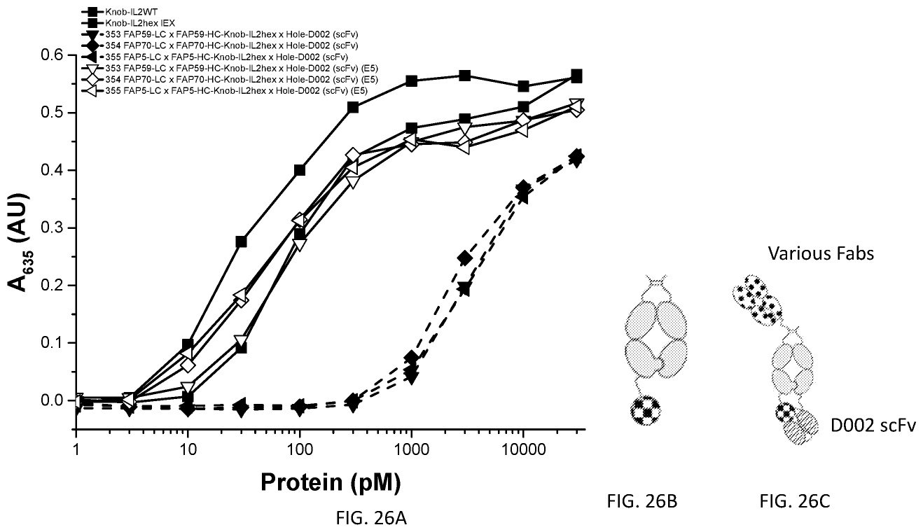

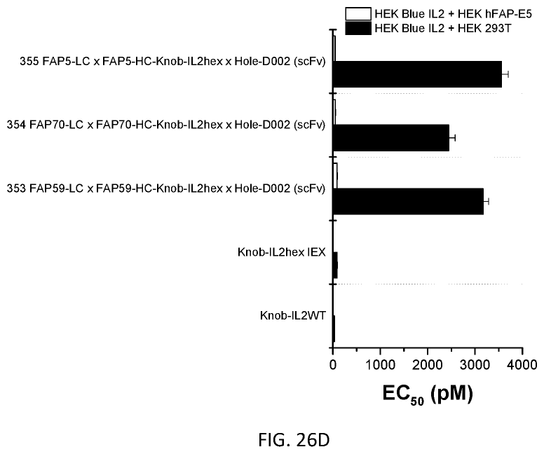

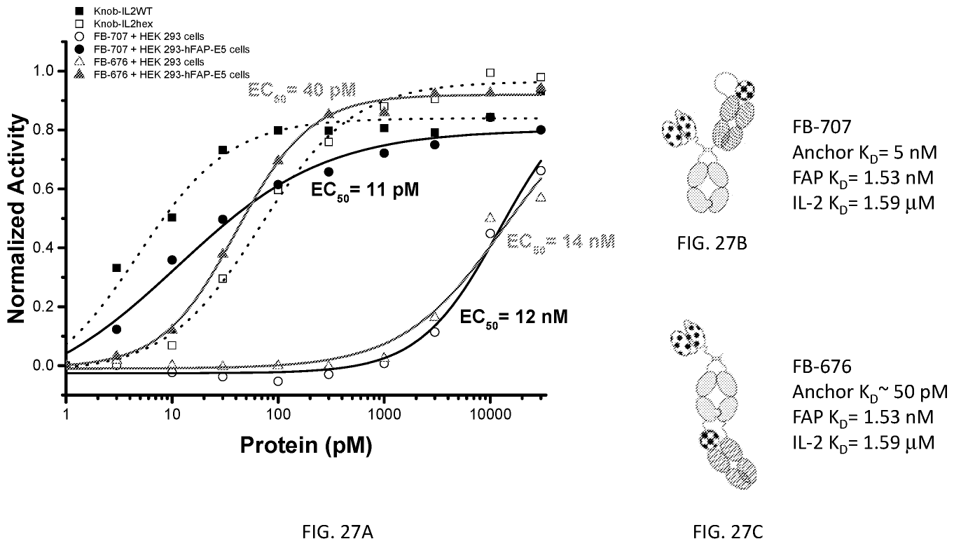

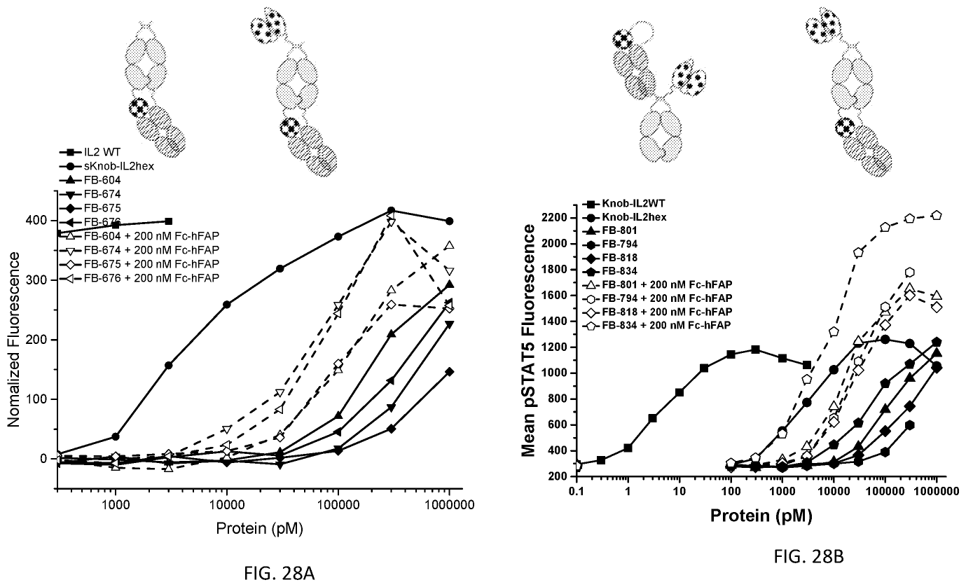

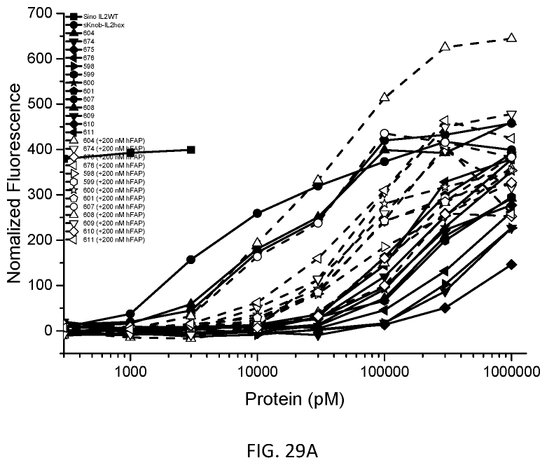

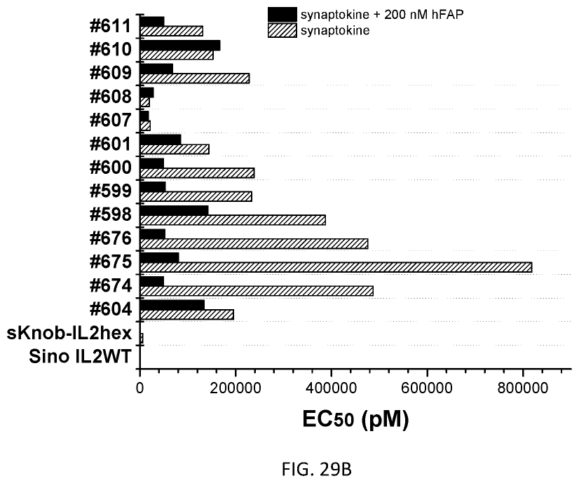

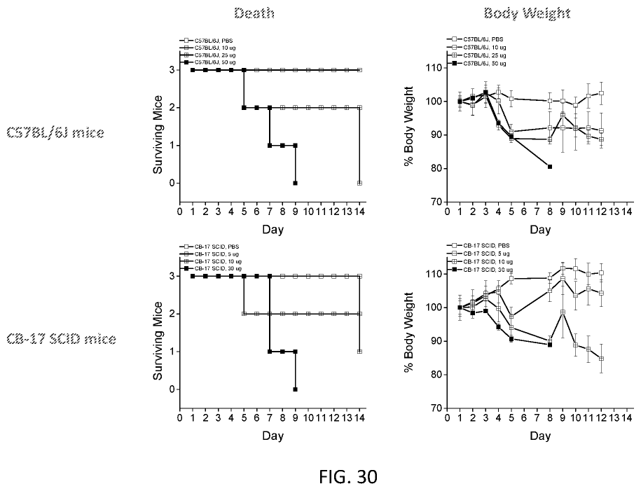

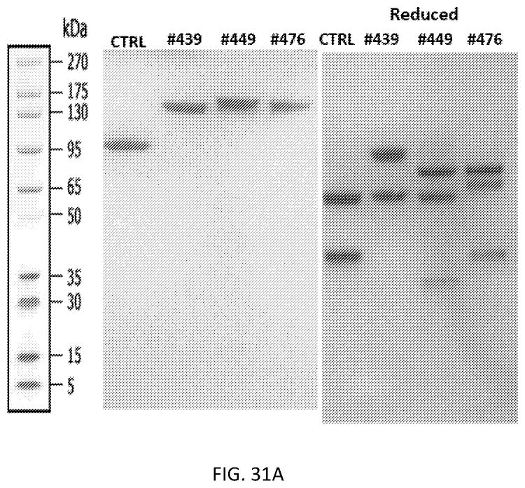

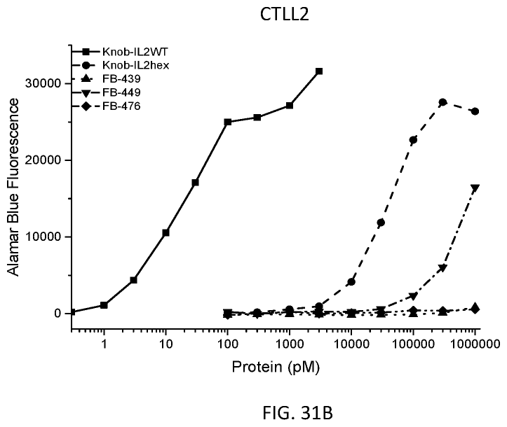

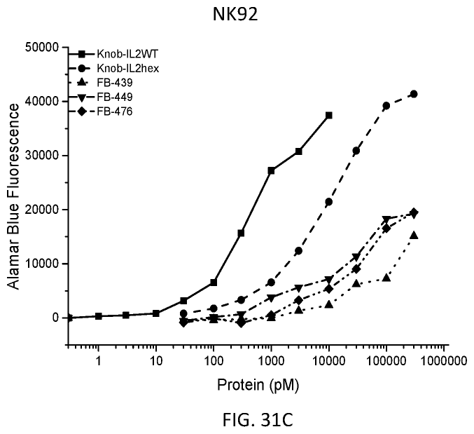

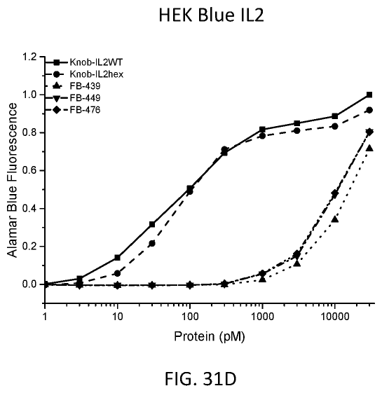

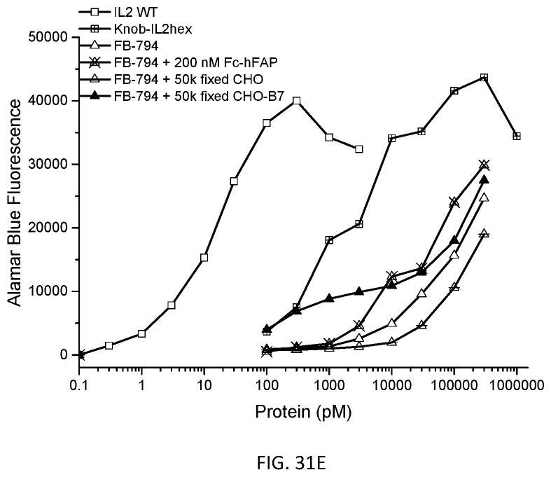

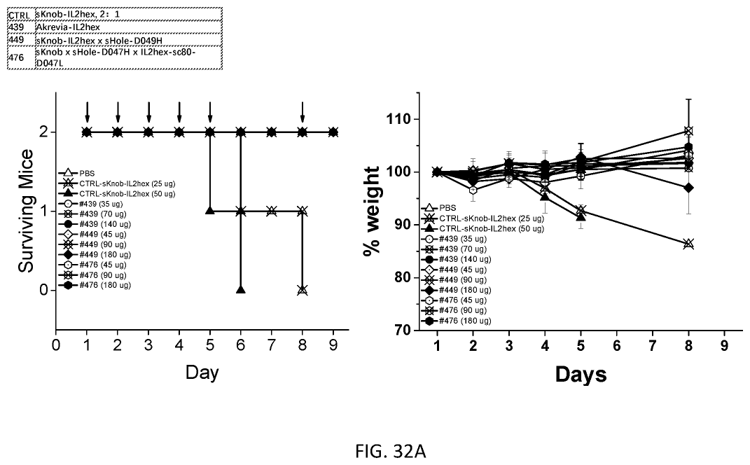

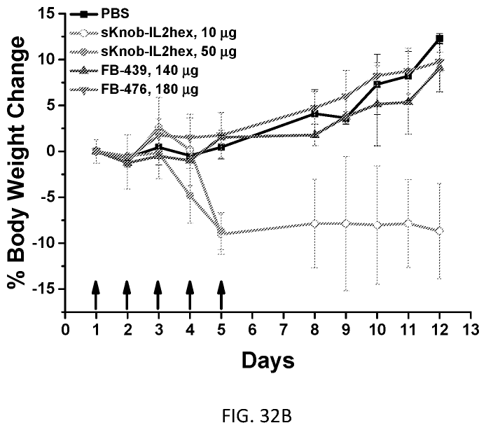

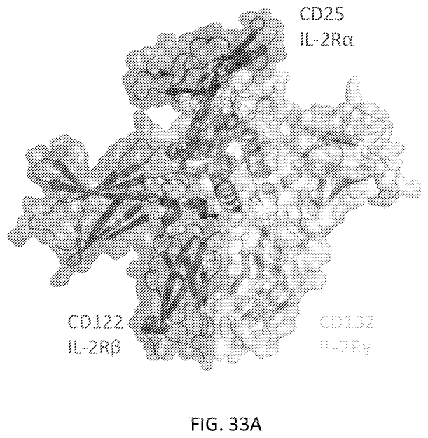

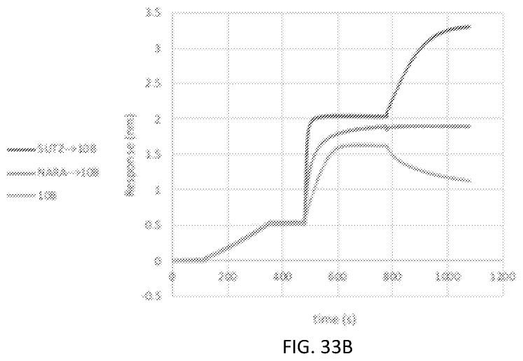

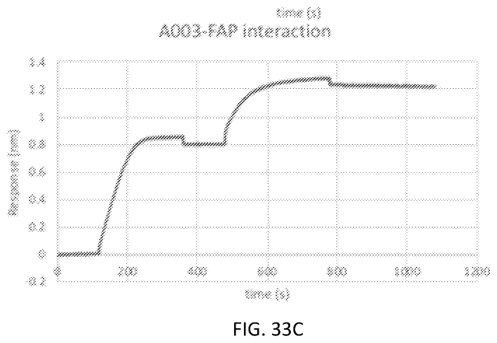

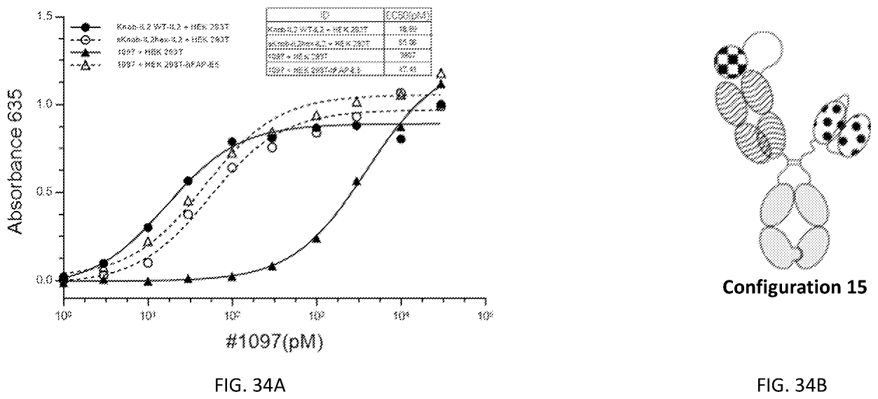

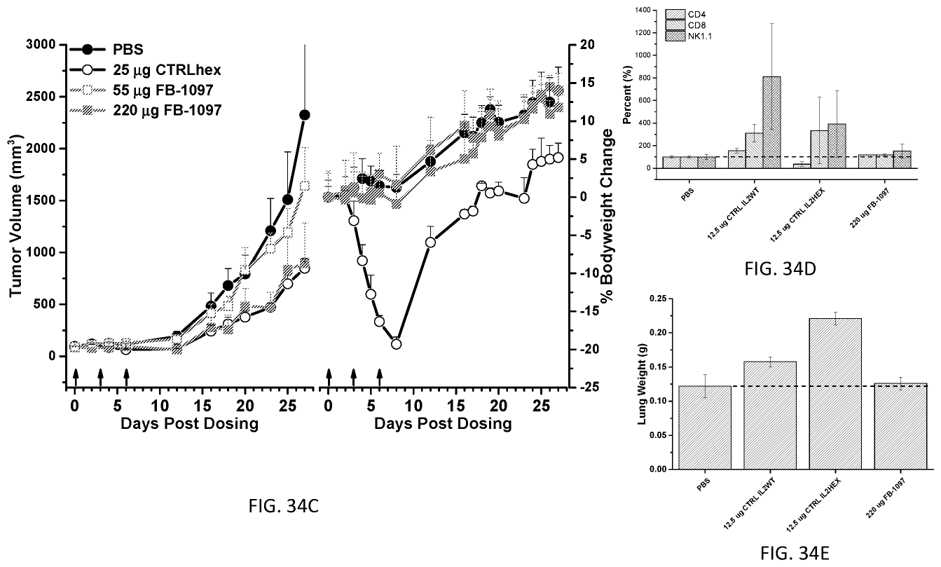

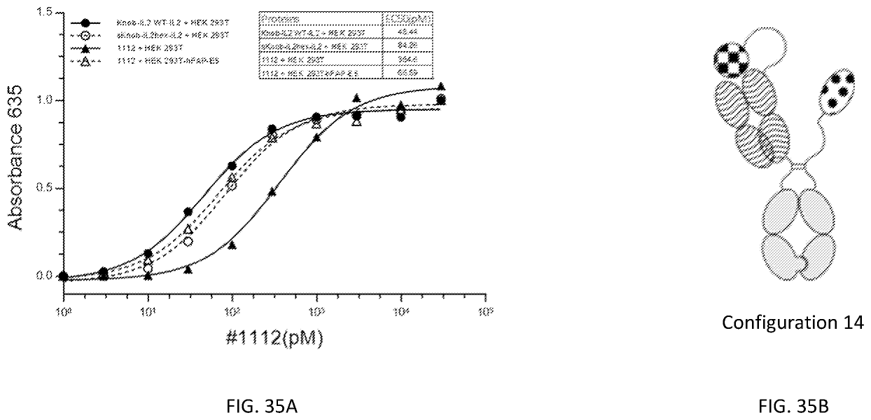

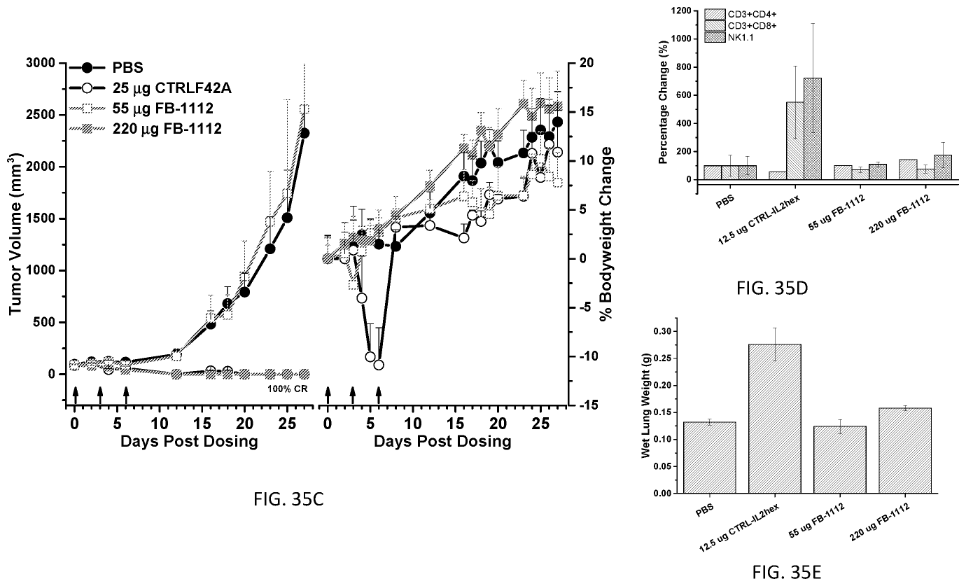

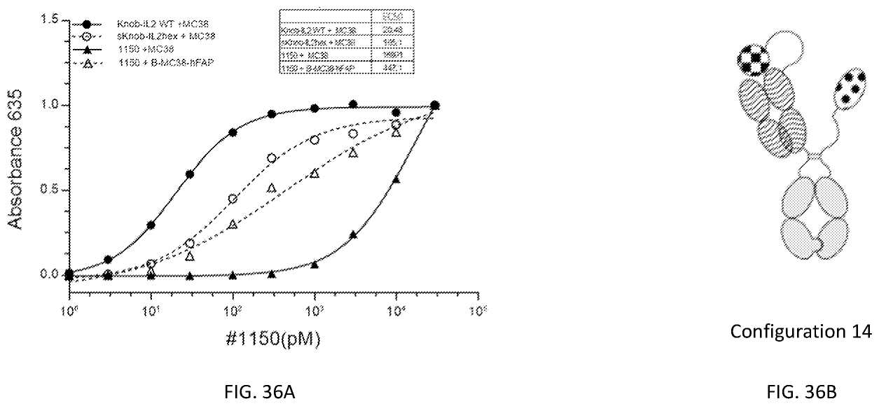

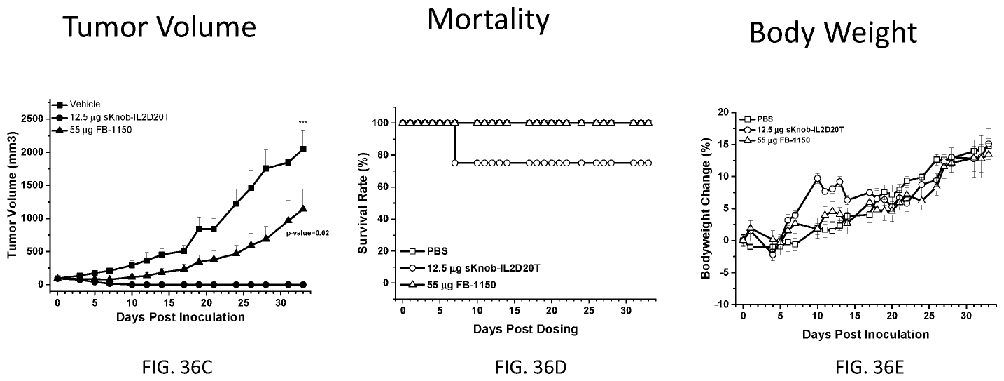

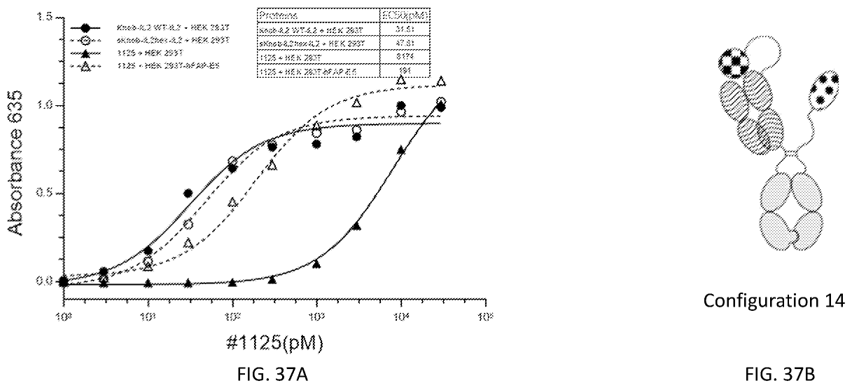

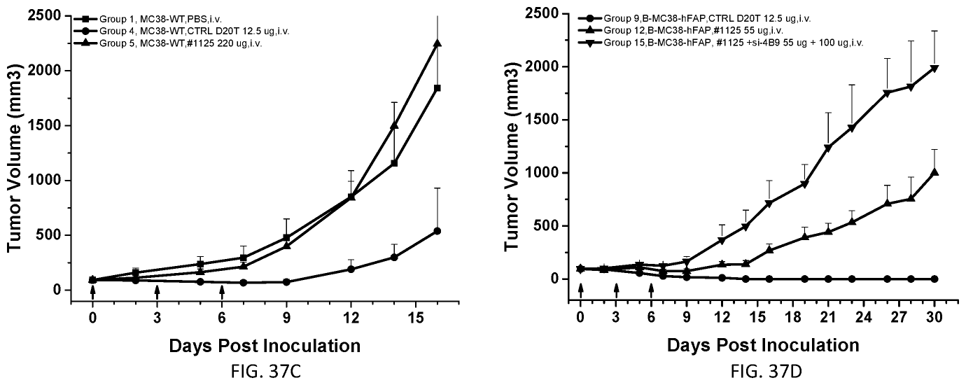

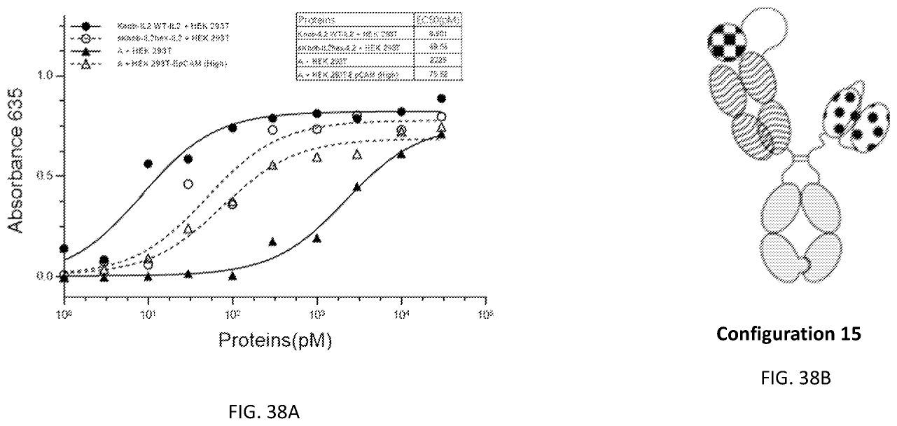

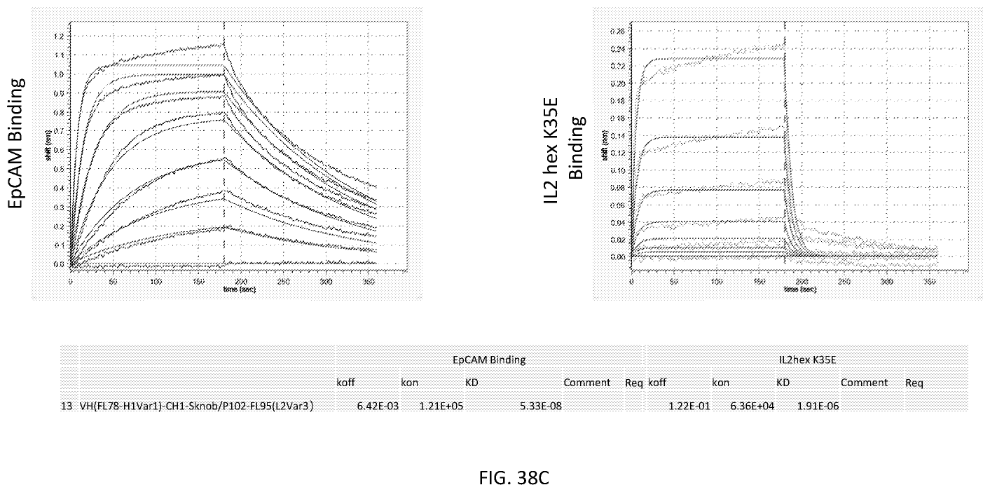

FIG. 5J shows an immunoconjugate containing (a) an anti-cytokine/anti-TAA two-in-one Fab antibody fused at the N-terminus of its heavy chain fragment to the C-terminus of one of the two heavy chain fragments of the immunoglobulin Fc domain (e.g., the Fc-hole), (b) a cytokine peptide fused to the N-terminus of the light chain fragment of the Fab antibody, and (c) an anti-TAA scFv antibody fused to the N-terminus of one of the two heavy chain fragments in the immunoglobulin Fc domain (e.g., Fc-knob).

FIG. 5K shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., the Fc-knob), (b) a cytokine peptide fused to the N-terminus of the heavy chain fragment of a Fab antibody, and (c) an anti-TAA Fab antibody fused at the C-terminus of its heavy chain fragment to the N-terminus of the other heavy chain fragment of the immunoglobulin Fc domain (e.g., the Fc-hole).

FIG. 5L shows an immunoconjugate containing (a) an anti-cytokine/anti-TAA two-in-one Fab antibody fused at the N-terminus of its heavy chain fragment to the C-terminus of one of the heavy chain fragments of the immunoglobulin Fc domain (e.g., the Fc-hole), (b) a cytokine polypeptide fused to the C-terminus of the other heavy chain fragment of the immunoglobulin Fc domain (e.g., the Fc-knob), and (c) an anti-TAA scFv antibody fused to the N-terminus of one of the two heavy chain fragments in the immunoglobulin Fc domain (e.g., the Fc-hole).

FIG. 5M shows an immunoconjugate containing (a) an anti-cytokine/anti-TAA two-in-one Fab antibody fused at the N-terminus of its heavy chain fragment to the C-terminus of one of the heavy chain fragments of the immunoglobulin Fc domain (e.g., the Fc-hole), (b) a cytokine peptide fused to the C-terminus of the other heavy chain fragment of the immunoglobulin Fc domain (Fc-knob), and (c) and anti-TAA scFv fused to the C-terminus of the heavy chain fragment of the anti-cytokine/anti-TAA two-in-one Fab antibody.

FIG. 5N shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., the Fc-hole), (b) a cytokine peptide fused to the N-terminus of the heavy chain fragment of a Fab antibody, and (c) an anti-TAA Fab antibody fused at the C-terminus of its heavy chain to the N-terminus of the other heavy chain fragment of the immunoglobulin Fc domain (e.g., the Fc-knob).

FIG. 50 shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-knob), (b) a cytokine peptide fused to the N-terminus of the heavy chain fragment of the Fab antibody, and (c) an anti-TAA single domain antibody fused to the N-terminus of the other one of the two heavy chain fragments in the immunoglobulin Fc domain (e.g., Fc-hole).

FIG. 5P shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-knob), (b) a cytokine peptide fused to the N-terminus of the heavy chain fragment of the Fab antibody, and (c) an anti-TAA scFv antibody fused to the N-terminus of the other one of the two heavy chain fragments in the immunoglobulin Fc domain (e.g., Fc-hole).

FIG. 5Q shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-hole), (b) a cytokine peptide fused to the N-terminus of the heavy chain fragment of the Fab antibody, and (c) an anti-TAA scFv antibody fused to the N-terminus of the other one of the two heavy chain fragments in the immunoglobulin Fc domain (e.g., Fc-knob).

FIG. 5R shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-hole), (b) a cytokine peptide fused to the N-terminus of the light chain fragment of the Fab antibody, and (c) an anti-TAA scFv antibody fused to the N-terminus of the other one of the two heavy chain fragments in the immunoglobulin Fc domain (e.g., Fc-knob).

FIG. 5S shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-knob), (b) a cytokine peptide fused to the N-terminus of the light chain fragment of the Fab antibody, and (c) an anti-TAA scFv antibody fused to the N-terminus of the other one of the two heavy chain fragments in the immunoglobulin Fc domain (e.g., Fc-hole).

FIG. 5T shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-knob), (b) an anti-TAA Fab antibody fused at the C-terminus of its heavy chain fragment to the N-terminus of the other heavy chain fragment in the immunoglobulin Fc domain (e.g., Fc-hole), and (c) a cytokine peptide fused to the N-terminus of the heavy chain fragment of the Fab antibody.

FIG. 5U shows an immunoconjugate containing (a) an anti-cytokine/anti-TAA two-in-one Fab antibody fused at the C-terminus of its heavy chain fragment to the N-terminus of one of the heavy chain fragments of the immunoglobulin Fc domain (e.g., Fc-knob), and (b) a cytokine peptide fused to the N-terminus of the heavy chain fragment of the Fab antibody.

FIG. 6A shows the homogeneity of isotype control antibody (DP47GS), immunoconjugate molecule having configuration 2 (FB-225) and naked cytokine (Knob-IL2hex), by HPLC with TOSH SW3000 column. As shown in the figure, homogeneity was significantly improved comparing naked cytokine Knob-IL2hex to the immunoconjugate (FB-255) containing an IL-2 binding antibody which stabilized the cytokine.

FIG. 6B shows the thermostability of control antibody (DP47GS), immunoconjugate molecule having configuration 2 (FB-FB225), naked cytokine (Knob-IL2hex) as measured by differential scan fluorimetry. The peak at 53° C. indicates denaturation of IL-2hex which was significantly right shifted, indicating that the IL-2 was stabilized by the two-in-one masking antibody in the form of the immunocytokine molecule (FB-225).

FIG. 6C shows accelerated stability of an IL-2 containing immunoconjugate as described herein measured using size-exclusion chromatography (SEC). As shown in the figure, the protein remained stable after storage at 40° C. for four weeks, or 5 rounds of freeze-thaw cycle.

FIG. 7A shows pharmacokinetics of naked cytokine (Knob-IL2hex) control and immunoconjugate molecules having configuration 2 (FB-476) and configuration 20 (FB-559), respectively, upon administration to mice in a single dose at various dosages. The protein concentrations were determined by anti-human Fc ELISA.

FIG. 7B is a schematic illustration of immunocytokine FB-476, in configuration 2 as shown in FIG. 5C. FB-476 contains an anti-cytokine/anti-hFAP two-in-one Fab antibody D047 which has affinity to IL2hex of a K_{D} of about 20 nM.

FIG. 7C is a schematic illustration of immunocytokine FB-559, in configuration 20 as shown in FIG. 5U. FB-559 contains an anti-cytokine/anti-hFAP two-in-one Fab antibody D029 mutant which has affinity to IL2hex of a K_{D} of about 400 nM.

FIG. 8A shows IL-2 activities measured using an IL-2 reporter cell line in the presence of IL-2 containing immunoconjugates having configuration 1 (circle) or configuration 2 (up triangle; down triangle; diamond; and left triangle) as shown in FIGS. 6B and 6C, respectively. Assays performed in the presence of naked IL-2 (square) were included as the positive control. X-axis shows the concentration (pM) of IL-2 or IL-2 containing immunoconjugates; Y-axis shows absorbance at 635 nm (A₆₃₅) determined using a TECAN plate reader, which reflected secreted embryonic alkaline phosphatase (SEAP) level and responses to IL2. FIG. 8B is a schematic illustration of the immunoconjugate molecule of configuration 1 according to the present disclosure. FIG. 8C is a schematic illustration of the immunoconjugate molecule of configuration 2 according to the present disclosure.

FIG. 9A shows IL-2 activities measured using an IL-2 reporter cell line in the presence of IL-2 containing immunoconjugates having configuration 1 (square), configuration 2 (circle) or configuration 4 (triangle) as shown in FIGS. 7B, 7C, and 7D, respectively. X-axis shows the concentration (pM) of the IL-2 containing immunoconjugates; Y-axis shows absorbance at 635 nm (A635) determined using a TECAN plate reader, which reflected secreted embryonic alkaline phosphatase (SEAP) level and responses to IL2. FIG. 9B is a schematic illustration of the immunoconjugate molecule of configuration 1 according to the present disclosure. FIG. 9C is a schematic illustration of the immunoconjugate molecule of configuration 2 according to the present disclosure. FIG. 9D is a schematic illustration of the immunoconjugate molecule of configuration 4 according to the present disclosure.

FIG. 10A shows IL-2 activities measured using an IL-2 reporter cell line in the presence of IL-2 containing immunoconjugates having configuration 1 (open square) or configuration 2 (open square with cross; blue square; pink square, red square) as shown in FIGS. 8B and 8C, respectively. The assays were performed in the presence (pink square, red square) or absence (open square, open square with cross; blue square) of soluble human Fibroblast Activation Protein (hFAP). Assays performed in the presence of naked IL-2 (closed square) were included as the positive control; assays performed in the presence of soluble FAP but without any immunoconjugate molecule (open square with dashed line) were included as the negative control. X-axis shows the concentration (pM) of the IL-2 containing immunoconjugates; Y-axis shows absorbance at 635 nm (A₆₃₅) determined using a TECAN plate reader, which reflected secreted embryonic alkaline phosphatase (SEAP) level and responses to IL-2. FIG. 10B is a schematic illustration of the immunoconjugate molecule of configuration 1 according to the present disclosure. FIG. 10C is a schematic illustration of the immunoconjugate molecule of configuration 2 according to the present disclosure.

FIG. 11A shows IL-2 activities measured using an IL-2 reporter cell line in the presence of IL-2 containing immunoconjugates having configuration 1 (square) or configuration 3 (triangle; circle) as shown in FIGS. 9B and 9C, respectively. The assays were performed in the presence (triangle) or absence (square, circle) of cells expressing human Fibroblast Activation Protein (hFAP) on the cell surfaces. X-axis shows the concentration (pM) of the IL-2 containing immunoconjugates; Y-axis shows absorbance at 635 nm (A₆₃₅) determined using a TECAN plate reader, which reflected secreted embryonic alkaline phosphatase (SEAP) level and responses to IL-2. FIG. 11B is a schematic illustration of the immunoconjugate molecule of configuration 1 according to the present disclosure. FIG. 11C is a schematic illustration of the immunoconjugate molecule of configuration 3 according to the present disclosure.

FIG. 11D shows IL-2 activities measured using an IL-2 reporter cell line in the presence of IL-2 containing immunoconjugates having configuration 1 (square) or configuration 3 (triangle; circle) as shown in FIGS. 9B and 9C, respectively. The assays were performed with (blue triangle, red triangle, hexagons of sizes 1-4) or without (square, circle) cells expressing human Fibroblast Activation Protein (hFAP) on the cell surfaces, and with (red triangle, hexagons of sizes 1-4) or without (square, circle, up triangle, blue triangle) soluble FAP molecules. X-axis shows the concentration (pM) of the IL-2 containing immunoconjugates; Y-axis shows absorbance at 635 nm (A₆₃₅) determined using a TECAN plate reader, which reflected secreted embryonic alkaline phosphatase (SEAP) level and responses to IL-2.

FIG. 11E shows IL-2 activities measured using an IL-2 reporter cell line in the presence of IL-2 containing immunoconjugates having configuration 1 (square) or configuration 3 (triangle; circle) as shown in FIGS. 9B and 9C, respectively. The assays were performed with (down triangle, diamond, pentagon, hexagon) or without (square, circle, up triangle) cells expressing human Fibroblast Activation Protein (hFAP) on the cell surfaces, and with (diamond, pentagon, hexagon) or without (square, circle, up triangle, down triangle) soluble antibodies. X-axis shows the concentration (pM) of the IL-2 containing immunoconjugates; Y-axis shows absorbance at 635 nm (A₆₃₅) determined using a TECAN plate reader, which reflected secreted embryonic alkaline phosphatase (SEAP) level and responses to IL-2.

FIG. 12A shows IL-2 activities measured using an IL-2 reporter cell line in the presence of IL-2 containing immunoconjugates having configuration 1 (closed square; open square) or configuration 2 (closed triangle; open triangle) as shown in FIGS. 10B and 10C, respectively. The assays were performed in the presence of unmodified HEK293T cells (closed square, open square, open triangle) or HEK293T cells expressing human Fibroblast Activation Protein (hFAP) on the cell surfaces (closed triangle). X-axis shows the concentration (pM) of the IL-2 containing immunoconjugates; Y-axis shows absorbance at 635 nm (A₆₃₅) determined using a TECAN plate reader, which reflected secreted embryonic alkaline phosphatase (SEAP) level and responses to IL-2. FIG. 12B is a schematic illustration of the immunoconjugate molecule of configuration 1 according to the present disclosure. FIG. 12C is a schematic illustration of the immunoconjugate molecule of configuration 2 according to the present disclosure.

FIG. 13A shows IL-2 activities measured using an IL-2 reporter cell line in the presence of IL-2 containing immunoconjugates having configuration 3 as shown in FIG. 13B. The assays were performed in the presence (solid line, open circle and open triangle) or absence (solid line, solid circle and solid triangle) of cells expressing human Fibroblast Activation Protein (hFAP) on the cell surfaces. X-axis shows the concentration (pM) of the IL-2 containing immunoconjugates; Y-axis shows absorbance at 635 nm (A₆₃₅) determined using a TECAN plate reader, which reflected secreted embryonic alkaline phosphatase (SEAP) level and responses to IL-2. Both tested immunoconjugate molecules (FB-387) and (FB-392) were in configuration 3 containing the same two-in-one Fab D029. The anchoring moiety of FB-387 was scFv5 having an K_{D} to hFAP of about 5 nM and binding to a different epitope on hFAP from D029; the anchoring moiety of FB-392 was scFv70 having an K_{D} to hFAP of about 1 nM and binding to the same epitope of hFAP as D029. Both molecules showed similar activities in presence or absence of hFAP expression cells.

FIG. 13B shows a schematic illustration of the immunoconjugate molecule of configuration 3 according to the present disclosure.

FIG. 14 is a schematic illustration of soluble FAP induced de-shielding of IL2 contained in an immunoconjugate molecule of the present disclosure. The simultaneous engagement of two FAP binding moieties (anchoring moiety and two-in-one masking moiety) enables the disassociation of the cytokine peptide from the masking moiety, and become capable of binding to the 5UTZ which is Human L-2/Fab complex shown in the figure.

FIG. 15 shows Biolayer interferometry (BLI) binding curves of immobilized 5UTZ to de-shielded IL2hex in four immunoconjugate molecules FB-604, FB-675, FB-676, and FB-626.

FIG. 16 shows Biolayer interferometry (BLI)binding curve of immobilized 5UTZ molecule to soluble Fc-hFAP and Knob-IL2hex.

FIG. 17A shows Biolayer interferometry (BLI)curves of immunoconjugate molecule FB-604 which was able to bind to immobilized 5UTZ molecule in the presence of soluble Fc-hFAP, but not in the absence of soluble Fc-hFAP.

FIG. 17B is a schematic illustration of immunoconjugate molecule FB-604 in configuration 2. The two-in-one antibody within FB-604 binds to FAP with a K_{D} value of about 1.53 nM, and IL2hex with an K_{D} value of about 1.59 μM.

FIG. 18A shows Biolayer interferometry (BLI)curves of immunoconjugate molecule FB-675 which was able to bind to immobilized 5UTZ molecule in presence of soluble Fc-hFAP, but not in the absence of soluble Fc-hFAP.

FIG. 18B is a schematic illustration of immunoconjugate molecule FB-675 in configuration 3. The two-in-one antibody within FB-675 binds to FAP with a K_{D} value of about 3.66 nM, and IL2hex with a K_{D} value of about 217 nM. The anchoring moiety in FB-675 binds to FAP with a K_{D} of about 5 nM.

FIG. 19A shows Biolayer interferometry (BLI)curves of immunoconjugate molecule FB-676 which was able to bind to immobilized 5UTZ molecule in presence of soluble Fc-hFAP, but not in the absence of soluble Fc-hFAP.

FIG. 19B is a schematic illustration of immunoconjugate molecule FB-676 in configuration 3. The two-in-one antibody within FB-675 binds to FAP with a K_{D} of about 1.53 nM, and IL2hex with a K_{D} of about 1.59 μM. The anchoring moiety binds to FAP with a K_{D} of about 5 nM.

FIG. 20A shows Biolayer interferometry (BLI) curves of immunoconjugate molecule FB-626 which was not able to bind to immobilized 5UTZ molecule either in the presence or absence of soluble Fc-hFAP.

FIG. 20B is a schematic illustration of immunoconjugate molecule FB-626 in configuration 14. The Two-in-one antibody within FB-626 binds to FAP with a K_{D} of great than about 5 μM, and to IL2hex with a K_{D} of about 237 μM.

FIG. 21A shows IL-2 activities measured using an IL-2 reporter cell line in the presence of IL-2 containing immunoconjugates having configuration 1 (square) or configuration 3 (closed circle, closed triangle, open circle, open triangle) as shown in FIGS. 21B and 21C. The assays were performed with (open circle, open triangle) or without (square, closed circle, closed triangle) HEK293T cells expressing human Fibroblast Activation Protein (hFAP) on the cell surface. X-axis shows the concentration (pM) of the IL-2 containing immunoconjugates; Y-axis shows absorbance at 635 nm (A₆₃₅) determined using a TECAN plate reader, which reflected secreted embryonic alkaline phosphatase (SEAP) level and responses to IL-2. FIG. 21B is a schematic illustration of the immunoconjugate molecule of configuration 1 according to the present disclosure. FIG. 21C is a schematic illustration of the immunoconjugate molecule of configuration 3 according to the present disclosure.

FIG. 22A shows TL-2 activities measured using an IL-2 reporter cell line in the presence or absence of FAP expression cells HEK 293T-hFAP-E5. Both tested immunoconjugate molecules had configuration 3 as shown in FIG. 22B and contain the same anchor moiety having scFv872-5. The two tested immunoconjugate molecules had different masking moieties containing two-in-one antibodies of D001 and D002, respectively. As shown in the figure, both immunoconjugate molecules had similar masking effect on the cytokine in the absence of hFAP expression cells. Further, both immunoconjugate molecules were able to de-shield and activate the cytokine activity in the presence of hFAP expression cells.

FIG. 22B is a schematic illustration of the immunoconjugate molecule of configuration 3 according to the present disclosure.

FIG. 23A shows TL-2 activities measured using an IL-2 reporter cell line in the presence or absence of FAP expression cells HEK 293T-hFAP-E5. Both tested immunoconjugate molecules had configuration 3 as shown in FIG. 23B and contained the same anchoring moiety comprising scFv872-59. The two tested immunoconjugate molecules had different masking moieties containing two-in-one antibodies D001 and D002, respectively. As shown in the figure, both immunoconjugate molecules had similar masking effect on the cytokine in the absence of hFAP expression cells. Further, both immunoconjugate molecules were able to de-shield and activate the cytokine activity in the presence of hFAP expression cells.

FIG. 23B is a schematic illustration of the immunoconjugate molecule of configuration 3 according to the present disclosure.

FIG. 24A shows IL-2 activities measured using an IL-2 reporter cell line in the presence or absence of FAP expression cells HEK 293T-hFAP-E5. Both of tested immunoconjugate molecules had configuration 3 as shown in FIG. 24B and contained the same anchoring moiety comprising scFv872-70. The two tested immunoconjugate molecules had different masking moieties containing two-in-one antibodies D001 and D002, respectively. As shown in the figure, both immunoconjugate molecules had similar masking effect on the cytokine in the absence of hFAP expression cells. Further, both immunoconjugate molecules were able to de-shield and activate the cytokine activity in the presence of hFAP expression cells.

FIG. 24B is a schematic illustration of the immunoconjugate molecule of configuration 3 according to the present disclosure.

FIG. 25A shows IL-2 activities measured using an IL-2 reporter cell line in the presence of IL-2 containing immunoconjugates having configuration 1 (circle, square) or configuration 5 (open diamond, closed diamond) as shown in FIGS. 12B and 12C, respectively. The assays were performed with (closed diamond) or without (square, circle, open diamond) HEK293T cells expressing human Fibroblast Activation Protein (hFAP) on the cell surface. X-axis shows the concentration (pM) of the IL-2 containing immunoconjugates; Y-axis shows absorbance at 635 nm (A₆₃₅) determined using a TECAN plate reader, which reflected secreted embryonic alkaline phosphatase (SEAP) level and responses to IL-2. FIG. 25B is a schematic illustration of the immunoconjugate molecule of configuration 1 according to the present disclosure. FIG. 25C is a schematic illustration of the immunoconjugate molecule of configuration 5 according to the present disclosure.

FIG. 26A shows IL-2 activities measured using an IL-2 reporter cell line in the presence of IL-2 containing immunoconjugates having configuration 1 (circle, square) or configuration 6 (open diamond, open down triangle, open left triangle, closed diamond, closed down triangle, closed left triangle) as shown in FIGS. 13B and 13C, respectively. The assays were performed with (open diamond, open down triangle, open left triangle) or without (square, circle, closed diamond, closed down triangle, closed left triangle) HEK293T cells expressing human Fibroblast Activation Protein (hFAP) on the cell surface. X-axis shows the concentration (pM) of the IL-2 containing immunoconjugates; Y-axis shows absorbance at 635 nm (A₆₃₅) determined using a TECAN plate reader, which reflected secreted embryonic alkaline phosphatase (SEAP) level and responses to IL-2. FIG. 26B is a schematic illustration of the immunoconjugate molecule of configuration 1 according to the present disclosure. FIG. 26C is a schematic illustration of the immunoconjugate molecule of configuration 6 according to the present disclosure. FIG. 26D is a bar graph showing the quantitated EC₅₀ (pM) values for the assays in the study.

FIG. 27A shows IL-2 activities measured using an IL-2 reporter cell line in the presence or absence of FAP expression cells HEK 293T-hFAP-E5 for the two immunoconjugate molecules FB-676 and FB-707. The EC₅₀ was about 14 nM for shielded FB-676 and about 40 μM for de-shielded FB-676; The EC₅₀ was about 12 nM for shielded FB-707 and about 11 μM for deshielded FB-707. The TL-2 potency increased about 700 to 1000 folds in the presence of FAP expression cells as compared to in the absence of FAP expression cells.

FIG. 27B is a schematic illustration of immunoconjugate molecule FB-707 in configuration 15. The two-in-one antibody in FB-707 binds to FAP with a K_{D} of about 1.53 nM, and to IL2hex with a K_{D} of about 1.59 μM. The anchoring moiety binds to FAP with a K_{D} of about 5 nM.

FIG. 27C is a schematic illustration of immunoconjugate molecule FB-676 in configuration 3. The two-in-one antibody within FB-675 binds to FAP with a K_{D} of about 1.53 nM, and to IL2hex with a K_{D} of about 1.59 μM. The anchoring moiety binds to FAP with a K_{D} of about 50 nM.

FIG. 28A shows human CD4+ T cell activation with immunoconjugate molecules of the present disclosure as measured using a pSTAT5 staining assay. The ability of immunoconjugate molecules FB-604, FB-674, FB-675 and FB-676 to stimulate pre-activated human CD4+ cells were measured in the presence or absence of 200 nM Fc-hFAP. As shown in the figure, the potency of IL2hex increased about 2 folds with immunoconjugate molecule FB-604 that does not have an anchoring moiety, and for about 10 folds for all other tested immunoconjugate molecules that have an anchoring moiety.

FIG. 28B shows human CD4+ T cell activation with immunoconjugate molecules of the present disclosure as measured using a pSTAT5 staining assay. The ability of immunoconjugate molecule FB-801, FB-794, FB-818 and FB-834 to stimulate pre-activated human CD4+ cells were measured in the presence or absence of 200 nM Fc-hFAP. As shown in the figure, the potency of IL2hex increased about 30 folds for all tested immunoconjugate molecules that have an anchoring moiety.

FIG. 29A shows human CD4+ T cell activation with immunoconjugate molecules of the present disclosure as measured using a pSTAT5 staining assay. The ability of immunoconjugate molecules FB-611, FB-610, FB-609, FB-608, FB-607, FB-601, FB-600, FB-599, FB-598, FB-676, FB-675, FB-674 and FB-604 to stimulate pre-activated human CD4+ cells were measured in presence or absence of 200 nM Fc-hFAP.

FIG. 29B shows quantitation of the EC₅₀ values as measured by the assay of FIG. Q-A.

FIG. 30 shows the acute toxicity of Knob-IL2hex on C57BL/6J and CB-17 SCID mice as measured in death (left) and body weight loss (right).

FIG. 31A shows the purified immunoconjugate molecule in non-reduced and reduced SDS-PAGE gel for four protein samples: Control (Knob-IL2hex, MW=66.8 kDa), FB-439 (MW=92.3 kDa), FB-449 (MW=120 kDa), FB-476 (MW=116 kDa).

FIGS. 31B to 31D show potency of immunoconjugate molecules FB-439, FB-449, and FB-476 as measured by the CTLL2 proliferation assay, NK92 proliferation assay and HEK Blue IL2 activation assay, respectively.

FIG. 31E shows human CD4+ T cell proliferation with immunoconjugate molecules of the present disclosure as measured Alarma Blue fluorescence. The ability of immunoconjugate molecules FB-794 stimulate pre-activated human CD4+ cells were measured by in presence or absence of 200 nM Fc-hFAP, and co-cultured with 40k fixed ExpiCHO cells with or without hFAP receptor on the surfaces.

FIG. 32A shows measurement of death (left) and body weight loss (right) in mice administered with immunoconjugate molecules: Control (Knob-IL2hex), FB-439, FB-449, FB-476.

FIG. 32B shows measurement of body weight loss in mice administered with immunoconjugate molecules sKnob-IL2hex (control), FB-439, FB-476, or PBS (control).

FIG. 33A is a 3D illustration of an IL-2 molecule binding with the IL-2R a, 3, and γ subunits (PDB: 2ERJ).

FIG. 33B and FIG. 33C show binding kinetics of a two-in-one antibody (B10) to IL-2 and FAP, respectively, comparing to two other IL-2 antibodies (namely 5UTZ that blocks IL-2 binding to IL-2Rβ (CD122) and NARA1 that blocks IL-2 binding to IL-2Rα (CD25)). B10 binds to IL2 on an overlapping epitope as NARA1, but not 5UTZ.

FIG. 34A shows IL-2 activities measured using an IL-2 reporter cell line in the presence or absence of FAP expressing cells for the immunoconjugate molecule FB-1097. The immunoconjugate tested has Configuration 15 as shown in FIG. 34B. The immunoconjugate contained point mutations (T3A, K35E, F42A, Y45A, L72G, C125S) in IL-2. The immunoconjugate also contained a variant of the D029 Fab as the masking moiety and a variant of scFv 872-5 as the anchoring moiety. IL-2 Fc fusion proteins having configuration 1 with both wild-type IL-2 (closed circle) and mutant IL-2hex (square) were included as controls. The assay was performed in the presence of cells that expressed human Fibroblast Activation Protein (hFAP) on the cell surface (HEK 293T-hFAP-E5; down triangle), or in the presence of cells that did not express FAP (HEK 293T up triangle). X-axis shows the concentration (pM) of the IL-2 containing immunoconjugate; Y-axis shows the activity of the immunoconjugate using a TECAN plate reader, which reflects secreted embryonic alkaline phosphatase (SEAP) level and responses to IL-2. FIG. 34C shows the tumor size and bodyweight of a MC38-FAP tumor model in C57BL/6 mice that were administered vehicle (PBS), CTRL-IL2hex, 55 μg FB-1097, or 220 μg FB1097.

FIG. 34D shows the systemic expansion of CD3+CD4+, CD3+CD8+, and NK cells in a MC38-FAP tumor model in C57BL/6 mice that were administered vehicle (PBS), 12.5 μg CTRL-TL2WT, 12.5 μg CTRL-IL2hex, or 220 μg FB-1097. FIG. 34E shows the lung weight in a MC38-FAP tumor model in C57BL/6 mice that were administered vehicle (PBS), 12.5 μg CTRL-IL2WT, 12.5 μg CTRL-IL2hex, or 220 μg FB-1097.

FIG. 35A shows TL-2 activities measured using an IL-2 reporter cell line in the presence or absence of FAP expressing cells for the immunoconjugate molecule #1112. The immunoconjugate tested has configuration 14 as shown in FIG. 35B. The immunoconjugate contained a point mutation (T3A, K35E, F42A, C125S) in IL-2. The immunoconjugate also contained the D029H and D029L masking moiety and the anchoring moiety VHH-E33. IL-2 Fc fusion protein having configuration 1 with both wild-type IL-2 (circle) and mutant IL-2hex (closed square) were included as controls. The assay was performed in the presence of cells that expressed human Fibroblast Activation Protein (hFAP) on the cell surface (HEK 293T-hFAP-E5; open square), or in the presence of cells that did not express FAP (HEK 293T triangle). X-axis shows the concentration (pM) of the IL-2 containing immunoconjugate; Y-axis shows the activity of the immunoconjugate using a TECAN plate reader, which reflects secreted embryonic alkaline phosphatase (SEAP) level and responses to IL-2.

FIG. 35C shows tumor size and bodyweight in a MC38-FAP tumor model in C57BL/6 mice that were administered vehicle (PBS), 25 μg CTRL-IL2 F42A, 55 μg FB-1112, or 220 μg FB-1112.

FIG. 35D shows the systemic expansion of CD3+CD4+, CD3+CD8+, and NK cells in a MC38-FAP tumor model in C57BL/6 mice that were administered vehicle (PBS), 12.5 μg CTRL-IL2hex, 55 μg FB-1112, or 220 μg FB-1112.

FIG. 35E shows the lung weight in a MC38-FAP tumor model in C57BL/6 mice that were administered vehicle (PBS), 12.5 μg CTRL-IL2hex, 55 μg FB-1112, or 220 μg FB-1112.

FIG. 36A shows IL-2 activities measured using an IL-2 reporter cell line in the presence or absence of FAP expressing cells for the immunoconjugate molecule #1150. The immunoconjugate tested has Configuration 14 as shown in FIG. 36B. The immunoconjugate also contained a Fab derived from antibody B10 as the masking moiety and the anchoring moiety VHH-E33. IL-2 Fc fusion proteins having Configuration 1 with both wild-type IL-2 (solid circle) and mutant IL-2hex (open circle) were included as controls. The assay was performed in the presence of cells that expressed human Fibroblast Activation Protein (hFAP) on the cell surface (B-MC38-FAP; open up triangle), or in the presence of cells that did not express FAP (MC38 solid up triangle). X-axis shows the concentration (pM) of the IL-2 containing immunoconjugate; Y-axis shows the activity of the immunoconjugate using a TECAN plate reader, which reflects secreted embryonic alkaline phosphatase (SEAP) level and responses to IL-2.

FIG. 36C to FIG. 36D show tumor size (FIG. 36C), survival rate (FIG. 36D) and body weight change (FIG. 36E) were measured in a MC38-FAP tumor model in C57BL/6 mice that were administered vehicle (PBS), 12.5 μg CTRL-IL2D20T, or 55 μg FB-1150.

FIG. 37A shows IL-2 activities measured using an IL-2 reporter cell line in the presence or absence of FAP expressing cells for the immunoconjugate molecule #1125. The immunoconjugate tested has Configuration 14 as shown in FIG. 37B. The immunoconjugate also contained a Fab derived from antibody B10 as the masking moiety and the anchoring moiety scFv872-5. IL-2 Fc fusion proteins having Configuration 1 with both wild-type IL-2 (solid circle) and mutant IL-2hex (open circle) were included as controls. The assay was performed in the presence of cells that expressed human Fibroblast Activation Protein (hFAP) on the cell surface (B-MC38-FAP; open up triangle), or in the presence of cells that did not express FAP (MC38 solid up triangle). X-axis shows the concentration (pM) of the IL-2 containing immunoconjugate; Y-axis shows the activity of the immunoconjugate using a TECAN plate reader, which reflects secreted embryonic alkaline phosphatase (SEAP) level and responses to IL-2.

FIG. 37C shows tumor volume in a MC38 tumor model in C57BL/6 mice that were administered PBS, 12.5 μg CTRL D20T, or 220 μg FB-1125.

FIG. 37D shows tumor volume in a MC38-FAP tumor model in C57BL/6 mice that were administered 12.5 μg CTRL D20T, 55 μg FB-1125, or 55 μg FB-1125 and 100 μg si-4B9.

FIG. 38A and FIG. 38B show TL-2 activity measured using an TL-2 reporter cell line in various cells by screening immunoconjugate molecule A and the corresponding molecular configuration. Immunoconjugate molecule A contains an IL-2 moiety, a two-in-one masking moiety capable of binding to IL-2 and EpCAM (a Fab derived from antibody FL78), and an anti-EpCAM anchoring moiety (scFv derived from MOC31). The assays were performed in the presence of HEK 293T EpCAM(high) cells expressing EpCAM on the cell surface. HEK 293T cells that did not express EpCAM. Molecule A has the same scaffold as configuration 15. X-axis shows the concentration (pM) of the IL-2 containing immunoconjugate; Y-axis shows absorbance at 635 nm (A₆₃₅) determined using a TECAN plate reader, which reflects secreted embryonic alkaline phosphatase (SEAP) level and response to I1L2.

FIG. 38C shows Biolayer interferometry (BLI) binding curves of immobilized EpCAM and mutant IL2 IL-2hex (K35E) molecules to immunoconjugate molecule A shown in FIG. 38A.

### 5. DETAILED DESCRIPTION

The present disclosure provides immunoconjugate molecules comprising a cytokine polypeptide. The present disclosure also provides, in certain embodiments, polynucleotides and vectors comprising sequences encoding such immunoconjugate molecules, and compositions, reagents, and kits comprising such immunoconjugate molecules. In related aspect, provided herein are also methods for delivery and/or activation of a cytokine activity at a target site, or reduce toxicity and/or other side-effects associated with systemic exposure to the cytokine activity in a subject through the use of the immunoconjugate molecules according to the present disclosure.

The present disclosure also provides, in certain embodiments, peptides or polypeptides, such as antibodies or antigen binding fragments thereof that can form part of such immunoconjugate molecules of the present disclosure. In specific embodiments, provided herein are binding proteins, including antibodies of fragments thereof that bind to fibrosis activation protein (FAP). In specific embodiments, provided herein are bispecific binding proteins, including two-in-one antibodies or fragments thereof that bind to both FAP and interleukin-2 (IL-2).

### 5.1 General Techniques

Techniques and procedures described or referenced herein include those that are generally well understood and/or commonly employed using conventional methodology by those skilled in the art, such as, for example, the widely utilized methodologies described in Sambrook et al., Molecular Cloning: A Laboratory Manual (3d ed. 2001); Current Protocols in Molecular Biology (Ausubel et al. eds., 2003); Therapeutic Monoclonal Antibodies: From Bench to Clinic (An ed. 2009); Monoclonal Antibodies: Methods and Protocols (Albitar ed. 2010); Phage Display in Biotechnology and Drug Discovery (Sidhu and Geyer eds., 2d ed. 2005); Phage Display: a Laboratory Manual (Barbas et al. eds., 2004); and Antibody Engineering Vols 1 and 2 (Kontermann and Dubel eds., 2d ed. 2010).

### 5.2 Terminology

Unless described otherwise, all technical and scientific terms used herein have the same meaning as is commonly understood by one of ordinary skill in the art. For purposes of interpreting this specification, the following description of terms will apply and whenever appropriate, terms used in the singular will also include the plural and vice versa. All patents, applications, published applications, and other publications are incorporated by reference in their entirety. In the event that any description of terms set forth conflicts with any document incorporated herein by reference, the description of term set forth below shall control.

As used herein, the singular terms “a,” “an,” and “the” include the plural reference unless the context clearly indicates otherwise.

Unless otherwise indicated, the terms “oligonucleotides” and “nucleic acids” are used interchangeably and are written left to right in 5′ to 3′ orientation; amino acid sequences are written left to right in amino to carboxy orientation, respectively. Therefore, in general, the codon at the 5′-terminus of an oligonucleotide will correspond to the N-terminal amino acid residue that is incorporated into a translated protein or peptide product. Similarly, in general, the codon at the 3′-terminus of an oligonucleotide will correspond to the C-terminal amino acid residue that is incorporated into a translated protein or peptide product. It is to be understood that this present disclosure is not limited to the particular methodology, protocols, and reagents described, as these may vary, depending upon the context they are used by those of skill in the art.

The term “interleukin-2” or “IL-2” as used herein, refers to any native IL-2 from any vertebrate source, including mammals such as primates (e.g. humans) and rodents (e.g., mice and rats), unless otherwise indicated. The term encompasses unprocessed IL-2 as well as any form of IL-2 that results from processing in the cell. The term also encompasses naturally occurring variants of IL-2, e.g. splice variants or allelic variants. The amino acid sequence of an exemplary human IL-2 is APTSSSTKKTQLQLEHLLLDLQMILNGINNYKNPKLTRMLTFKFYMPKKATELKHLQ CLEEELKPLEEVLNLAQSKNFHLRPRDLISNINVIVLELKGSETTFMCEYADETATIVE FLNRWITFCQSIISTLT (SEQ ID NO: 1). Unprocessed human IL-2 additionally comprises an N-terminal 20 amino acid signal peptide (underlined, absent in the matured IL-2 molecule) and has the sequence as shown below

(SEQ ID NO: 2)
MYRMQLLSCIALSLALVTNSAPTSSSTKKTQLQLEHLLLDLQMILNGINN
YKNPKLTRMLTFKFYMPKKATELKHLQCLEEELKPLEEVLNLAQSKNFHL
RPRDLISNINVIVLELKGSETTFMCEYADETATIVEFLNRWITFCQSIIS
TLT

Without being bound by the theory, it is contemplated that an IL-2 polypeptide binds to the IL-2 receptor (IL-2R) at the α-, β-, and/or γ-subunit(s) of the IL-2R receptor complex. Further, it is contemplated that the regions of IL-2 that participate in binding to IL-2Rα (CD-25) include: P34 (3.2 Å²), K35 (37 Å²), R38 (130 Å²), T41(25 Å²), F42 (95 Å²), K43 (61 Å²), F44 (4 Å²), Y45 (90 Å²), E61 (67 Å²), E62 (15 Å²), K64 (46 Å²), P65 (46 Å²) E68 (78 Å²), V69 (6 Å²), N71 (3 Å²), L72 (49 Å²), Q74 (43 Å²), Y107 (35 Å²), D109 (3 Å²) the regions of IL-2 participating in binding to IL-2Rβ (CD122) include: L12 (40 Å²), Q13 (29 Å²), E15 (37 Å²), H16 (89 Å²), L19 (68 Å²), D20 (24 Å²), M23 (33 Å²), R81 (51 Å²), D84 (57 Å²), D87 (16 Å²), N88 (62 Å²), V91 (85 Å²), 192 (34 Å²), E95 (45 Å²); and the regions of IL-2 that participate in binding to IL-2rγ (CD-132) include: Q11 (9 Å²), L12 (3 Å²), E15 (41 Å²), L18 (19 Å²), L19 (12 Å²), Q22 (28 Å²), K48 (8 Å²), T51 (1 Å²), E110 (26 Å²), N119 (59 Å²), R120 (5 Å²), 1122 (5 Å²), T123 (59 Å²), Q126 (82 Å²), S127 (32 Å²), 1129 (40 Å²), S130 (55 Å²), T131 (<1 Å²), T133 (29 Å²), where the number in parenthesis is the calculated buried surface area from the IL-2 receptor protein complex with the Protein Databank ID 2B51.

The term “IL-2 mutant” or “mutant IL-2 polypeptide” as used herein is intended to encompass any mutant forms of various forms of the IL-2 molecule including full-length IL-2, truncated forms of IL-2 and forms where IL-2 polypeptide containing one or more amino acid mutations in its sequence. “Full-length” when used in reference to IL-2 is intended to mean the mature, natural length IL-2 molecule. For example, full-length human IL-2 refers to a molecule that has 133 amino acids (see e.g., SEQ ID NO: 1). The various forms of IL-2 mutants are characterized in having at least one amino acid mutation affecting the interaction of IL-2 with CD25. This mutation may involve substitution, deletion, truncation or modification of the wild-type amino acid residue normally located at that position. Unless otherwise indicated, an IL-2 mutant may be referred to herein as an IL-2 mutant peptide sequence, an IL-2 mutant polypeptide, IL-2 mutant protein or IL-2 mutant analog. Designation of various forms of IL-2 is herein made with respect to the sequence shown in SEQ ID NO: 1. Various designations may be used herein to indicate the same mutation. For example, a mutation from phenylalanine at position 42 to alanine can be indicated as 42A, A42, A₄₂, F42A, or Phe42Ala. The designation of “IL-2hex” refers to a mutant form of human IL-2 as shown below, which contains the ΔA1/T3A/F42A/Y45A/L72G/C125S mutations in the human IL-2 sequence (amino acid substitutions are underlined and bolded): PASSSTKKTQLQLEHLLLDLQMILNGINNYKNPKLTRMLTAKFAMPKKATELKHLQ CLEEELKPLEEVLNGAQSKNFHLRPRDLISNINVIVLELKGSETTFMCEYADETATIVE FLNRWITFSQSIISTLT (SEQ ID NO: 3). The numbering of the positions of mutated amino acid residues is according to the wild-type human IL-2 sequence (SEQ ID NO: 1). Without being bound by the theory, it is contemplated that mutation AA1 removes the N-terminal residue of the mature form of IL-2; mutation T3A removes a potential glycosylation site; the F42A/Y45A/L72G mutations diminish binding of IL-2 to CD25; and the C125S mutation removes an unpaired cysteine within IL-2.

Without being bound by the theory, it is contemplated that mutations in a region of the IL-2 polypeptide responsible for IL-2 interaction with one IL-2R subunit may impact IL-2 binding to that IL-2R subunit, while not affecting IL-2R binding to another IL-2R subunit. For example, various IL-2 mutations are known to negatively impact binding of IL-2 to IL-2Rα (CD25), including but not limited to K35E, R38A, R38D, R38E, F42A, F42K, K43E, Y45A, E61R, E62A, L72G, or a combination thereof. For example, the F42A single mutation has been demonstrated to reduce binding to IL-2 to IL-2a for approximately 100-fold, whereas the combination of (a) F42A/Y45A/L72G, (b) R38D/K43E/E61R or (c) R38A/F42A/Y45A/E62A have been demonstrated to completely abolish binding of IL-2 to IL-2a. Various IL-2 mutations are known to negatively impact binding of IL-2 to IL-2Rβ (CD122), including but not limited to D20T, D20G, D20A, H16E, H16R, H16A, N88D, N88S, N88R, V91G, V91A, V91R, V91S, or a combination thereof. Various IL-2 mutations are known to impact binding of IL-2 to IL-2Rγ (CD132), including but not limited to L18R, Q22E, T123A, Q126X where X=H, M, K, R, E, S, G, A, C, D, I or T, 1129V, S130A, S130R, or a combination thereof. Combinations of IL-2 mutations impacting binding to IL-2Rγ have been used to create agonists and inhibitors of IL-2 signaling. For example, the Q126T mutation in combination with the Q74H/L80F/R81D/L85V/I92F mutations has been demonstrated to enhance binding of IL-2 to IL-2Rγ and can act as a partial agonist of IL-2 receptor signaling. For another example, the L18R/Q22E/Q126T/S130R mutation combination of IL-2 has been demonstrated to abolish IL-2 signaling and can serve to inhibit signaling of wild-type IL-2.

Additional exemplary IL-2 mutants that can be used in connection with the present disclosure also include: IL2 C125S (residues 1-153, no signal peptide, amino acid substitutions underlined and bolded) having the sequence of

(SEQ ID NO: 7)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFSQSIISTLT;

IL2 C125A (residues 1-153, no signal peptide, amino acid substitutions underlined and bolded) having the sequence of

(SEQ ID NO: 8)
APTSSSTKKTQLQLEHLLLDLQMILNGINNYKNPKLTRMLTFKFYMPKKA
TELKHLQCLEEELKPLEEVLNLAQSKNFHLRPRDLISNINVIVLELKGSE
TTFMCEYADETATIVEFLNRWITFAQSIISTLT;

IL2-F42A/Y45A/L72G/C125A (residues 1-153, no signal peptide, amino acid substitutions underlined and bolded) having the sequence of

(SEQ ID NO: 9)
APTSSSTKKTQLQLEHLLLDLQMILNGINNYKNPKLTRMLTAKFAMPKKA
TELKHLQCLEEELKPLEEVLNGAQSKNFHLRPRDLISNINVIVLELKGSE
TTFMCEYADETATIVEFLNRWITFAQSIISTLT;

IL2-R38A/F42A/Y45A/E62A/C125S (residues 1-153, amino acid substitutions underlined and bolded) having the sequence of

(SEQ ID NO: 10)
APTSSSTKKTQLQLEHLLLDLQMILNGINNYKNPKLTAMLTAKFAMPKKA
TELKHLQCLEEALKPLEEVLNLAQSKNFHLRPRDLISNINVIVLELKGSE
TTFMCEYADETATIVEFLNRWITFSQSIISTLT;

IL2-T3A/R38E/F42A/C125S (residues 1-153, amino acid substitutions underlined and bolded) having the sequence of

(SEQ ID NO: 11)
APASSSTKKTQLQLEHLLLDLQMILNGINNYKNPKLTEMLTAKFYMPKKA
TELKHLQCLEEELKPLEEVLNLAQSKNFHLRPRDLISNINVIVLELKGSE
TTFMCEYADETATIVEFLNRWITFSQSIISTLT;

IL2-T3A/R38E/Y45A/C125S (residues 1-153, amino acid substitutions underlined and bolded) having the sequence of

(SEQ ID NO: 12)
APASSSTKKTQLQLEHLLLDLQMILNGINNYKNPKLTEMLTFKFAMPKKA
TELKHLQCLEEELKPLEEVLNLAQSKNFHLRPRDLISNINVIVLELKGSE
TTFMCEYADETATIVEFLNRWITFSQSIISTLT;

IL2-T3A/R38E/L72G/C125S (residues 1-153, amino acid substitutions underlined and bolded) having the sequence of

(SEQ ID NO: 13)
APASSSTKKTQLQLEHLLLDLQMILNGINNYKNPKLTEMLTFKFYMPKKA
TELKHLQCLEEELKPLEEVLNGAQSKNFHLRPRDLISNINVIVLELKGSE
TTFMCEYADETATIVEFLNRWITFSQSIISTLT;

IL2-ΔA2/T3A/F42A/Y45A/L72G/C125S, residues 2-153, no signal peptide “hex”, amino acid substitutions underlined and bolded) having the sequence of

(SEQ ID NO: 14)
PASSSTKKTQLQLEHLLLDLQMILNGINNYKNPKLTRMLTAKFAMPKKAT
ELKHLQCLEEELKPLEEVLNGAQSKNFHLRPRDLISNINVIVLELKGSET
TFMCEYADETATIVEFLNRWITFSQSIISTLT;

IL2-AA2/T3A/K35E/F42A/Y45A/L72G/C125S, residues 2-153, no signal peptide “hex/K35E” amino acid substitutions underlined and bolded having the sequence of

(SEQ ID NO: 15)
PASSSTKKTQLQLEHLLLDLQMILNGINNYKNPELTRMLTAKFAMPKKAT
ELKHLQCLEEELKPLEEVLNGAQSKNFHLRPRDLISNINVIVLELKGSET
TFMCEYADETATIVEFLNRWITFSQSIISTLT;

IL2-T3A/K35E/F42A/Y45A/L72G/C125S (residues 1-153, no signal peptide, amino acid substitutions underlined and bolded) having the sequence of

(SEQ ID NO: 107)
APASSSTKKTQLQLEHLLLDLQMILNGINNYKNPELTRMLTAKFAMPKKA
TELKHLQCLEEELKPLEEVLNGAQSKNFHLRPRDLISNINVIVLELKGSE
TTFMCEYADETATIVEFLNRWITFSQSIISTLT;

IL2-T3A/K35E/F42A/C125S (residues 1-153, no signal peptide, amino acid substitutions underlined and bolded) having the sequence of

(SEQ ID NO: 108)
APASSSTKKTQLQLEHLLLDLQMILNGINNYKNPELTRMLTAKFYMPKKA
TELKHLQCLEEELKPLEEVLNLAQSKNFHLRPRDLISNINVIVLELKGSE
TTFMCEYADETATIVEFLNRWITFSQSIISTLT;

IL2-T3A/D20T/K35E/C125S (residues 1-153, no signal peptide, amino acid substitutions underlined and bolded) having the sequence of

(SEQ ID NO: 109)
APASSSTKKTQLQLEHLLLTLQMILNGINNYKNPELTRMLTFKFYMPKK
ATELKHLQCLEEELKPLEEVLNLAQSKNFHLRPRDLISNINVIVLELKG
SETTFMCEYADETATIVEFLNRWITFSQSIISTLT;

and

IL2-T3A/H16A/K35E/C125S (residues 1-153, no signal peptide, amino acid substitutions underlined and bolded) having the sequence of

(SEQ ID NO: 110)
APASSSTKKTQLQLEALLLDLQMILNGINNYKNPELTRMLTFKFYMPKK
ATELKHLQCLEEELKPLEEVLNLAQSKNFHLRPRDLISNINVIVLELKG
SETTFMCEYADETATIVEFLNRWITFSQSIISTLT.

Additional Mutant IL-2 polypeptides that can be used in connection with the present disclosure include those described in, for example, U.S. Pat. Nos.:10,184,009 and 5,229,109 and International Patent Publication No. WO2012107417A1, the disclosure of each of which is enclosed herein by reference in its entirety.

As used herein, a “wild-type” form of IL-2 is a form of IL-2 that is otherwise the same as the mutant IL-2 polypeptide except that the wild-type form has a wild-type amino acid at each amino acid position of the mutant IL-2 polypeptide. For example, if the IL-2 mutant is the full-length IL-2 (i.e. IL-2 not fused or conjugated to any other molecule), the wild-type form of this mutant is full-length native IL-2. If the IL-2 mutant is a fusion between IL-2 and another polypeptide encoded downstream of IL-2 (e.g., an antibody chain) the wild-type form of this IL-2 mutant is IL-2 with a wild-type amino acid sequence fused to the same downstream polypeptide. Furthermore, if the IL-2 mutant is a truncated form of IL-2 (the mutated or modified sequence within the non-truncated portion of IL-2) then the wild-type form of this IL-2 mutant is a similarly truncated IL-2 that has a wild-type sequence. For the purpose of comparing IL-2 receptor binding affinity or biological activity of various forms of IL-2 mutants to the corresponding wild-type form of IL-2, the term wild-type encompasses forms of IL-2 comprising one or more amino acid mutation that does not affect IL-2 receptor binding compared to the naturally occurring, native IL-2, such as e.g., a substitution of cysteine at a position corresponding to residue 125 of human IL-2 to alanine. In certain embodiments according to the invention the wild-type IL-2 polypeptide to which the mutant IL-2 polypeptide is compared comprises the amino acid sequence of SEQ ID NO: 1.

The term “CD25” or “a-subunit of the IL-2 receptor” or “IL-2Rα” as used herein, refers to any native CD25 from any vertebrate source, including mammals such as primates (e.g., humans) and rodents (e.g., mice and rats), unless otherwise indicated. The term encompasses “full-length”, unprocessed CD25 as well as any form of CD25 that results from processing in the cell. The term also encompasses naturally occurring variants of CD25, e.g., splice variants or allelic variants. In certain embodiments CD25 is human CD25. The amino acid sequence of an exemplary human CD25 (with signal sequence, underlined) is shown below:

(SEQ ID NO: 4)
MDSYLLMWGLLTFIMVPGCQAELCDDDPPEIPHATFKAMAYKEGTMLNC
ECKRGFRRIKSGSLYMLCTGNSSHSSWDNQCQCTSSATRNTTKQVTPQP
EEQKERKTTEMQSPMQPVDQASLPGHCREPPPWENEATERIYHFVVGQM
VYYQCVQGYRALHRGPAESVCKMTHGKTRWTQPQLICTGEEKPQASPEG
RPESETSCLVTTTDFQIQTEMAATMETSIFTTEYQVAVAGCVFLLISVL
LLSGLTWQRRQRKSRRTI.

The term “CD122” or “p-subunit of the IL-2 receptor” or “IL-2R β” as used herein, refers to any native CD122 from any vertebrate source, including mammals such as primates (e.g. humans) and rodents (e.g., mice and rats), unless otherwise indicated. The term encompasses “full-length”, unprocessed CD122 as well as any form of CD122 that results from processing in the cell. The term also encompasses naturally occurring variants of CD122, e.g., splice variants or allelic variants. In certain embodiments CD122 is human CD122. The amino acid sequence of an exemplary human CD122 is shown below:

(SEQ ID NO: 111)
MAAPALSWRLPLLILLLPLATSWASAAVNGTSQFTCFYNSRANISCVWS
QDGALQDTSCQVHAWPDRRRWNQTCELLPVSQASWACNLILGAPDSQKL
TTVDIVTLRVLCREGVRWRVMAIQDFKPFENLRLMAPISLQVVHVETHR
CNISWEISQASHYFERHLEFEARTLSPGHTWEEAPLLTLKQKQEWICLE
TLTPDTQYEFQVRVKPLQGEFTTWSPWSQPLAFRTKPAALGKDTIPWLG
HLLVGLSGAFGFIILVYLLINCRNTGPWLKKVLKCNTPDPSKFFSQLSS
EHGGDVQKWLSSPFPSSSFSPGGLAPEISPLEVLERDKVTQLLLQQDKV
PEPASLSSNHSLTSCFTNQGYFFFHLPDALEIEACQVYFTYDPYSEEDP
DEGVAGAPTGSSPQPLQPLSGEDDAYCTFPSRDDLLLFSPSLLGGPSPP
STAPGGSGAGEERMPPSLQERVPRDWDPQPLGPPTPGVPDLVDFQPPPE
LVLREAGEEVPDAGPREGVSFPWSRPPGQGEFRALNARLPLNTDAYLSL
QELQGQDPTHLV.

The term “CD132” or “γ-subunit of the IL-2 receptor” or “IL-2Rγ” as used herein, refers to any native CD132 from any vertebrate source, including mammals such as primates (e.g., humans) and rodents (e.g., mice and rats), unless otherwise indicated. The term encompasses “full-length”, unprocessed CD132 as well as any form of CD132 that results from processing in the cell. The term also encompasses naturally occurring variants of CD132, e.g., splice variants or allelic variants. In certain embodiments CD132 is human CD132. The amino acid sequence of an exemplary human CD132 (with signal sequence, underlined) is shown below:

(SEQ ID NO: 112)
MLKPSLPFTSLLFLQLPLLGVGLNTTILTPNGNEDTTADFFLTTMPTDS
LSVSTLPLPEVQCFVFNVEYMNCTWNSSSEPQPTNLTLHYWYKNSDNDK
VQKCSHYLFSEEITSGCQLQKKEIHLYQTFVVQLQDPREPRRQATQMLK
LQNLVIPWAPENLTLHKLSESQLELNWNNRFLNHCLEHLVQYRTDWDHS
WTEQSVDYRHKFSLPSVDGQKRYTFRVRSRFNPLCGSAQHWSEWSHPIH
WGSNTSKENPFLFALEAVVISVGSMGLIISLLCVYFWLERTMPRIPTLK
NLEDLVTEYHGNFSAWSGVSKGLAESLQPDYSERLCLVSEIPPKGGALG
EGPGASPCNQHSPYWAPPCYTLKPET.

The term “high-affinity IL-2 receptor” as used herein refers to the heterotrimeric form of the IL-2 receptor, consisting of the receptor γ-subunit (also known as common cytokine receptor γ-subunit, γ_{c}, or CD132), the receptor β-subunit (also known as CD122 or p70) and the receptor a-subunit (also known as CD25 or p55), or a functional variant thereof. The term “intermediate-affinity IL-2 receptor” by contrast refers to the IL-2 receptor including only the γ-subunit and the β-subunit, without the α-subunit, or a functional variant thereof (for a review see e.g., Olejniczak and Kasprzak, Med Sci Monit 14, RA179-189 (2008)).

The term “tumor associated antigen” or “TAA”, as used herein, refers to an antigen expressed by a cancer cell or in the stroma of a solid tumor. The TAA can be a protein, nucleic acid, lipid or other antigen. In certain embodiments, the TAA can be a cell-surface expressed TAA. In the context of a solid tumor, the TAA can be expressed in the stroma of a solid tumor mass. The term “stroma” as used herein refers to components in a solid tumor mass other than a cancer cell. For example, the stroma can include fibroblasts, epithelial cells, other blood vessel components or extracellular matrix components. As used herein, the term “stroma” does not include components of the immune system, such as immune cells (e.g., B-cells, T-cells, dendritic cells, macrophages, natural killer cells, and the like). Various TAAs are known in the art. Identifying TAA can be performed using methods known in the art, such as disclosed in Zhang et al., Methods Mol. Biol., 520:1-10 (2009); the content of which is enclosed herein by reference.

The term “fibroblast activation protein” or “FAP” as used herein, refers to any native FAP from any vertebrate source, including mammals such as primates (e.g., humans) and rodents (e.g., mice and rats), unless otherwise indicated. The term encompasses unprocessed FAP as well as any form of FAP that results from processing in the cell. The term also encompasses naturally occurring variants of FAP, e.g., splice variants or allelic variants. The amino acid sequence of an exemplary human FAP is shown below

(SEQ ID NO: 5)
MKTWVKIVFGVATSAVLALLVMCIVLRPSRVHNSEENTMRALTLKDILN
GTFSYKTFFPNWISGQEYLHQSADNNIVLYNIETGQSYTILSNRTMKSV
NASNYGLSPDRQFVYLESDYSKLWRYSYTATYYIYDLSNGEFVRGNELP
RPIQYLCWSPVGSKLAYVYQNNIYLKQRPGDPPFQITFNGRENKIFNGI
PDWVYEEEMLATKYALWWSPNGKFLAYAEFNDTDIPVIAYSYYGDEQYP
RTINIPYPKAGAKNPVVRIFIIDTTYPAYVGPQEVPVPAMIASSDYYFS
WLTWVTDERVCLQWLKRVQNVSVLSICDFREDWQTWDCPKTQEHIEESR
TGWAGGFFVSTPVFSYDAISYYKIFSDKDGYKHIHYIKDTVENAIQITS
GKWEAINIFRVTQDSLFYSSNEFEEYPGRRNIYRISIGSYPPSKKCVTC
HLRKERCQYYTASFSDYAKYYALVCYGPGIPISTLHDGRTDQEIKILEE
NKELENALKNIQLPKEEIKKLEVDEITLWYKMILPPQFDRSKKYPLLIQ
VYGGPCSQSVRSVFAVNWISYLASKEGMVIALVDGRGTAFQGDKLLYAV
YRKLGVYEVEDQITAVRKFIEMGFIDEKRIAIWGWSYGGYVSSLALASG
TGLFKCGIAVAPVSSWEYYASVYTERFMGLPTKDDNLEHYKNSTVMARA
EYFRNVDYLLIHGTADDNVHFQNSAQIAKALVNAQVDFQAMWYSDQNHG
LSGLSTNHLYTHMTHFLKQCFSLSD.

The term “tumor microenvironment” refers to any and all elements of the neoplasia milieu that creates a structural and/or functional environment for the neoplastic process to survive, expand, and/or spread. As a non-limiting example, a tumor microenvironment is constituted by the cells, molecules, fibroblasts, extracellular matrix and/or blood vessels that surround and/or feed one or more neoplastic cells, such as a solid tumor. In certain embodiments, the neoplastic disease is a solid tumor. Exemplary cells or tissue within the tumor microenvironment include, but are not limited to, tumor vasculature, tumor infiltrating lymphocytes, fibroblast reticular cells, endothelial progenitor cells (EPC), cancer-associated fibroblasts, pericytes, other stromal cells, components of the extracellular matrix (ECM), dendritic cells, antigen presenting cells, T-cells, regulatory T-cells, macrophages, neutrophils, and other immune cells located proximal to a tumor. Exemplary cellular functions affecting the tumor microenvironment include, but are not limited to, production of cytokines and/or chemokines, response to cytokines, antigen processing and presentation of peptide antigen, regulation of leukocyte chemotaxis and migration, regulation of gene expression, complement activation, regulation of signaling pathways, cell-mediated cytotoxicity, cell-mediated immunity, humoral immune responses, and innate immune responses, etc.

The term “antibody,” “immunoglobulin,” or “Ig” is used interchangeably herein, and is used in the broadest sense and specifically encompasses, for example, individual monoclonal antibodies (including agonist, antagonist, neutralizing antibodies, full length or intact monoclonal antibodies), antibody compositions with polyepitopic or monoepitopic specificity, polyclonal or monovalent antibodies, multivalent antibodies, multispecific antibodies (e.g., bispecific antibodies so long as they exhibit the desired biological activity), formed from at least two intact antibodies, single chain antibodies, and fragments of antibodies, as described below. An antibody can be human, humanized, chimeric and/or affinity matured, as well as an antibody from other species, for example, mouse and rabbit, etc. The term “antibody” is intended to include a polypeptide product of B cells within the immunoglobulin class of polypeptides that is able to bind to a specific molecular antigen and is composed of two identical pairs of polypeptide chains, wherein each pair has one heavy chain (about 50-70 kDa) and one light chain (about 25 kDa), each amino-terminal portion of each chain includes a variable region of about 100 to about 130 or more amino acids, and each carboxy-terminal portion of each chain includes a constant region. See, e.g., Antibody Engineering (Borrebaeck ed., 2d ed. 1995); and Kuby, Immunology (3d ed. 1997). In specific embodiments, the specific molecular antigen can be bound by an antibody provided herein, such as a IL-2 polypeptide, a IL-2 fragment, or a IL-2 epitope. Antibodies also include, but are not limited to, synthetic antibodies, recombinantly produced antibodies, camelized antibodies, intrabodies, anti-idiotypic (anti-Id) antibodies, and functional fragments (e.g., antigen-binding fragments such as IL-2-binding fragments) of any of the above, which refers to a portion of an antibody heavy or light chain polypeptide that retains some or all of the binding activity of the antibody from which the fragment was derived. Non-limiting examples of functional fragments (e.g., antigen-binding fragments such as IL-2-binding fragments) include single-chain Fvs (scFv) (e.g., including monospecific, bispecific, etc.), Fab fragments (e.g., including monospecific, bispecific, etc.), F(ab′) fragments, F(ab)₂ fragments, F(ab′)₂ fragments, disulfide-linked Fvs (dsFv), Fd fragments, Fv fragments, diabody, triabody, tetrabody, minibody, and single domain antibody (VHH or nanobody). In particular, antibodies provided herein include immunoglobulin molecules and immunologically active portions of immunoglobulin molecules, for example, antigen-binding domains or molecules that contain an antigen-binding site that binds to an IL-2 antigen (e.g., one or more CDRs of an anti-IL-2 antibody). Such antibody fragments can be found in, for example, Harlow and Lane, Antibodies: A Laboratory Manual (1989); Mol. Biology and Biotechnology: A Comprehensive Desk Reference (Myers ed., 1995); Huston et al., 1993, Cell Biophysics 22:189-224; Pluckthun and Skerra, 1989, Meth. Enzymol. 178:497-515; and Day, Advanced Immunochemistry (2d ed. 1990). The antibodies provided herein can be of any class (e.g., IgG, IgE, IgM, IgD, and IgA) or any subclass (e.g., IgG1, IgG2, IgG3, IgG4, IgA1, and IgA2) of immunoglobulin molecule.

The term “monoclonal antibody” as used herein refers to an antibody obtained from a population of substantially homogeneous antibodies, e.g., the individual antibodies comprising the population are identical except for possible naturally occurring mutations that may be present in minor amounts, and each monoclonal antibody will typically recognize a single epitope on the antigen. In specific embodiments, a “monoclonal antibody,” as used herein, is an antibody produced by a single hybridoma or other cell, wherein the antibody binds to only one epitope as determined, for example, by ELISA or other antigen-binding or competitive binding assay known in the art. The term “monoclonal” is not limited to any particular method for making the antibody. For example, the monoclonal antibodies useful in the present disclosure may be prepared by the hybridoma methodology first described by Kohler et al., 1975, Nature 256:495, or may be made using recombinant DNA methods in bacterial or eukaryotic animal or plant cells (see, e.g., U.S. Pat. No. 4,816,567). The “monoclonal antibodies” may also be isolated from phage antibody libraries using the techniques described in Clackson et al., 1991, Nature 352:624-28 and Marks et al., 1991, J. Mol. Biol. 222:581-97, for example. Other methods for the preparation of clonal cell lines and of monoclonal antibodies expressed thereby are well known in the art. See, e.g., Short Protocols in Molecular Biology (Ausubel et al. eds., 5th ed. 2002). Exemplary methods of producing monoclonal antibodies are provided in the Examples herein.

“Polyclonal antibodies” as used herein refer to an antibody population generated in an immunogenic response to a protein having many epitopes and thus includes a variety of different antibodies directed to the same or different epitopes within the protein. Methods for producing polyclonal antibodies are known in the art (See, e.g., Short Protocols in Molecular Biology (Ausubel et al. eds., 5th ed. 2002)).

An “antigen” is a predetermined antigen to which an antibody can selectively bind. A target antigen may be a polypeptide, carbohydrate, nucleic acid, lipid, hapten, or other naturally occurring or synthetic compound. In some embodiments, the target antigen is a polypeptide.

The terms “antigen-binding fragment,” “antigen-binding domain,” “antigen-binding region,” and similar terms refer to that portion of an antibody, which comprises the amino acid residues that interact with an antigen and confer on the binding agent its specificity and affinity for the antigen (e.g., the CDRs).

A “bispecific antibody” as used herein refers to an antibody or antigen binding fragment thereof that is capable of binding with two different target antigens. A “two-in-one antibody” as used herein refers to a bispecific antibody that is capable of binding with two different target antigens via a single antigen binding domain. In some embodiments, the target antigens compete with one another for binding with the single antigen binding domain of the two-in-one antibody, such that the two-in-one antibody, upon binding with one target antigen, dissociates from the other target antigen.

An “epitope” is the site on the surface of an antigen molecule to which a single antibody molecule binds, such as a localized region on the surface of an antigen, such as a IL-2 polypeptide or a IL-2 polypeptide fragment, that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, including, for example, by an immunoassay. Antigenic epitopes need not necessarily be immunogenic. Epitopes often consist of chemically active surface groupings of molecules such as amino acids or sugar side chains and have specific three dimensional structural characteristics as well as specific charge characteristics. Antibody epitopes may be linear epitopes or conformational epitopes. Linear epitopes are formed by a continuous sequence of amino acids in a protein. Conformational epitopes are formed of amino acids that are discontinuous in the protein sequence, but which are brought together upon folding of the protein into its three-dimensional structure. Induced epitopes are formed when the three dimensional structure of the protein is in an altered conformation, such as following activation or binding of another protein or ligand. Generally an antigen has several or many different epitopes and may react with many different antibodies. In certain embodiments, an antigen (e.g., FAP) can have more than one epitopes that are recognized and bound by different anti-FAP antibodies. In certain embodiments, different anti-FAP antibodies compete with one another for binding with the same epitope of FAP.

An antibody binds “an epitope,” “essentially the same epitope,” or “the same epitope” as a reference antibody, when the two antibodies recognize identical, overlapping, or adjacent epitopes in a three-dimensional space. The most widely used and rapid methods for determining whether two antibodies bind to identical, overlapping, or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent, or enzyme labels.

“Epitope mapping” is the process of identifying the binding sites, or epitopes, of antibodies on their target antigens. “Epitope binning” is the process of grouping antibodies based on the epitopes they recognize. More particularly, epitope binning comprises methods and systems for discriminating the epitope recognition properties of different antibodies, using competition assays combined with computational processes for clustering antibodies based on their epitope recognition properties and identifying antibodies having distinct binding specificities.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between a single antigen-binding site on an antibody and a single epitope of a target molecule, such as IL-2, is the affinity of the antibody or functional fragment for that epitope. The ratio of dissociation rate (k_{off}) to association rate (kₒₙ) of an antibody to a monovalent antigen (k_{off}/kₒₙ) is the dissociation constant K_{D}, which is inversely related to affinity. The lower the K_{D} value, the higher the affinity of the antibody. The value of K_{D} varies for different complexes of antibody and antigen and depends on both kₒₙ and k_{off}. The dissociation constant K_{D} for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent IL-2, come in contact with antibodies containing multiple binding sites, the interaction of antibody with antigen at one site will increase the probability of a reaction at a second site. The strength of such multiple interactions between a multivalent antibody and antigen is called the avidity. The avidity of an antibody can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively.

The terms “antibodies that specifically bind to an antigen,” “antibodies that specifically bind to an epitope” and analogous terms are also used interchangeably herein and refer to antibodies that specifically bind to the antigen, or fragment, or epitope of the antigen. An antibody that specifically binds to an antigen can be identified, for example, by immunoassays, Biacore©, or other techniques known to those of skill in the art. An antibody binds specifically to an antigen when it binds to the antigen with higher affinity than to any cross-reactive antigen as determined using experimental techniques, such as radioimmunoassays (RIA) and enzyme linked immunosorbent assays (ELISAs). Typically, a specific or selective reaction will be at least twice background signal or noise and may be more than 10 times background. See, e.g., Fundamental Immunology 332-36 (Paul ed., 2d ed. 1989) for a discussion regarding antibody specificity. An antibody which “binds an antigen of interest” (e.g., a target antigen such as IL-2) is one that binds the antigen with sufficient affinity such that the antibody is useful as a therapeutic agent in targeting a cell or tissue expressing the antigen, and does not significantly cross-react with other proteins. In such embodiments, the extent of binding of the antibody to a “non-target” protein will be less than about 10% of the binding of the antibody to its particular target protein, for example, as determined by fluorescence activated cell sorting (FACS) analysis or RIA. With regard to the binding of an antibody to a target molecule, the term “specific binding,” “specifically binds to,” or “is specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. The term “specific binding,” “specifically binds to,” or “is specific for” a particular polypeptide or an epitope on a particular polypeptide target as used herein refers to binding where a molecule binds to a particular polypeptide or epitope on a particular polypeptide without substantially binding to any other polypeptide or polypeptide epitope. In certain embodiments, an antibody that binds to an antigen of the present disclosure has a dissociation constant (K_{D}) of less than or equal to 10 nM, 5 nM, 4 nM, 3 nM, 2 nM, 1 nM, 0.9 nM, 0.8 nM, 0.7 nM, 0.6 nM, 0.5 nM, 0.4 nM, 0.3 nM, 0.2 nM, or 0.1 nM.

The term “compete” when used in the context of antibodies (e.g., antibodies and binding proteins that bind to a cell surface antigen and compete for the same epitope or binding site on a target) means competition as determined by an assay in which the antibody (or binding fragment) thereof under study prevents or inhibits the specific binding of a reference molecule (e.g., a reference ligand or reference antigen-binding protein, such as a reference antibody) to a common antigen (e.g., FAP or a fragment thereof). Numerous types of competitive binding assays can be used to determine if a test antibody competes with a reference antibody for binding to an antigen (e.g., human FAP). Examples of assays that can be employed include solid phase direct or indirect RIA, solid phase direct or indirect enzyme immunoassay (EIA), sandwich competition assay (see, e.g., Stahli et al., 1983, Methods in Enzymology 9:242-53), solid phase direct biotin-avidin EIA (see, e.g., Kirkland et al., 1986, J. Immunol. 137:3614-19), solid phase direct labeled assay, solid phase direct labeled sandwich assay (see, e.g., Harlow and Lane, Antibodies, A Laboratory Manual (1988)), solid phase direct label RIA using I-125 label (see, e.g., Morel et al., 1988, Mol. Immunol. 25:7-15), and direct labeled RIA (Moldenhauer et al., 1990, Scand. J. Immunol. 32:77-82). Typically, such an assay involves the use of a purified antigen (e.g., IL-2) bound to a solid surface, or cells bearing either of an unlabelled test antigen-binding protein (e.g., test anti-IL-2 antibody) or a labeled reference antigen-binding protein (e.g., reference anti-IL-2 antibody). Competitive inhibition may be measured by determining the amount of label bound to the solid surface or cells in the presence of the test antigen-binding protein. Usually the test antigen-binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur. Additional details regarding methods for determining competitive binding are described herein. Usually, when a competing antibody protein is present in excess, it will inhibit specific binding of a reference antibody to a common antigen by at least 30%, for example 40%, 45%, 50%, 55%, 60%, 65%, 70%, or 75%. In some instance, binding is inhibited by at least 80%, 85%, 90%, 95%, 96%, 97%, 98%, 99%, or more.

The term “heavy chain” when used in reference to an antibody refers to a polypeptide chain of about 50-70 kDa, wherein the amino-terminal portion includes a variable region of about 120 to 130 or more amino acids, and a carboxy-terminal portion includes a constant region. The constant region can be one of five distinct types, (e.g., isotypes) referred to as alpha (a), delta (6), epsilon (F), gamma (γ), and mu (p), based on the amino acid sequence of the heavy chain constant region. The distinct heavy chains differ in size: a, 6, and γ contain approximately 450 amino acids, while p and F contain approximately 550 amino acids. When combined with a light chain, these distinct types of heavy chains give rise to five well known classes (e.g., isotypes) of antibodies, IgA, IgD, IgE, IgG, and IgM, respectively, including four subclasses of IgG, namely IgG1, IgG2, IgG3, and IgG4. A heavy chain can be a human heavy chain.

The term “light chain” when used in reference to an antibody refers to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, referred to as kappa (x) or lambda (k) based on the amino acid sequence of the constant domains. Light chain amino acid sequences are well known in the art. A light chain can be a human light chain.

The term “variable region,” “variable domain,” “V region,” or “V domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” that are each about 9-12 amino acids long. The variable regions of heavy and light chains each comprise four FRs, largely adopting a R sheet configuration, connected by three hypervariable regions, which form loops connecting, and in some cases form part of, the R sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest (5th ed. 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. In specific embodiments, the variable region is a human variable region.

The term “variable region residue numbering as in Kabat” or “amino acid position numbering as in Kabat”, and variations thereof, refer to the numbering system used for heavy chain variable regions or light chain variable regions of the compilation of antibodies in Kabat et al., supra. Using this numbering system, the actual linear amino acid sequence may contain fewer or additional amino acids corresponding to a shortening of, or insertion into, an FR or CDR of the variable domain. For example, a heavy chain variable domain may include a single amino acid insert (residue 52a according to Kabat) after residue 52 and three inserted residues (e.g., residues 82a, 82b, and 82c, etc. according to Kabat) after residue 82. The Kabat numbering of residues may be determined for a given antibody by alignment at regions of homology of the sequence of the antibody with a “standard” Kabat numbered sequence. The Kabat numbering system is generally used when referring to a residue in the variable domain (approximately residues 1-107 of the light chain and residues 1-113 of the heavy chain) (e.g., Kabat et al., supra). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human IgG 1 EU antibody. Other numbering systems have been described, for example, by AbM, Chothia, Contact, IMGT, and AHon.

A “CDR” refers to one of three hypervariable regions (H1, H2 or H3) within the non-framework region of the immunoglobulin (Ig or antibody) VH 3-sheet framework, or one of three hypervariable regions (L1, L2 or L3) within the non-framework region of the antibody VL 3-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al., 1997, J. Biol. Chem. 252:6609-16; Kabat, 1978, Adv. Prot. Chem. 32:1-75). CDR region sequences also have been defined structurally by Chothia as those residues that are not part of the conserved p-sheet framework, and thus are able to adapt different conformations (Chothia and Lesk, 1987, J. Mol. Biol. 196:901-17). Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art.

The term “hypervariable region,” “HVR,” or “HV,” when used herein refers to the regions of an antibody variable region that are hypervariable in sequence and/or form structurally defined loops. Generally, antibodies comprise six hypervariable regions, three in the VH (H1, H2, H3) and three in the VL (L1, L2, L3). A number of hypervariable region delineations are in use and are encompassed herein. The Kabat Complementarity Determining Regions (CDRs) are based on sequence variability and are the most commonly used (see, e.g., Kabat et al., supra). Chothia refers instead to the location of the structural loops (see, e.g., Chothia and Lesk, 1987, J. Mol. Biol. 196:901-17). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Antibody Engineering Vol. 2 (Kontermann and Dubel eds., 2d ed. 2010)). The “contact” hypervariable regions are based on an analysis of the available complex crystal structures. The residues from each of these hypervariable regions or CDRs are noted below.

Recently, a universal numbering system has been developed and widely adopted, ImMunoGeneTics (IMGT) Information System® (Lafranc et al., 2003, Dev. Comp. Immunol. 27(1):55-77). IMGT is an integrated information system specializing in immunoglobulins (IG), T cell receptors (TCR), and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues are readily identified. This information can be used in grafting and replacement of CDR residues from immunoglobulins of one species into an acceptor framework from, typically, a human antibody. An additional numbering system (AHon) has been developed by Honegger and Pluckthun, 2001, J. Mol. Biol. 309: 657-70. Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra). In some embodiments, the CDRs are as defined by the IMGT numbering system. In other embodiments, the CDRs are as defined by the Kabat numbering system. In certain embodiments, the CDRs are as defined by the AbM numbering system. In other embodiments, the CDRs are as defined by the Chothia system. In yet other embodiments, the CDRs are as defined by the Contact numbering system.

| | IMGT | Kabat | AbM | Chothia | Contact |
|---|---|---|---|---|---|
| V_{H} CDR1 | 27-38 | 31-35 | 26-35 | 26-32 | 30-35 |
| V_{H} CDR2 | 56-65 | 50-65 | 50-58 | 53-55 | 47-58 |
| V_{H} CDR3 | 105-117 | 95-102 | 95-102 | 96-101 | 93-101 |
| V_{L} CDR1 | 27-38 | 24-34 | 24-34 | 26-32 | 30-36 |
| V_{L} CDR2 | 56-65 | 50-56 | 50-56 | 50-52 | 46-55 |
| V_{L} CDR3 | 105-117 | 89-97 | 89-97 | 91-96 | 89-96 |

Hypervariable regions may comprise “extended hypervariable regions” as follows: 24-36 or 24-34 (L1), 46-56 or 50-56 (L2), and 89-97 or 89-96 (L3) in the VL, and 26-35 or 26-35A (H1), 50-65 or 49-65 (H2), and 93-102, 94-102, or 95-102 (H3) in the VH. As used herein, the terms “HVR” and “CDR” are used interchangeably.

The term “constant region” or “constant domain” refers to a carboxyl terminal portion of the light and heavy chain which is not directly involved in binding of the antibody to antigen but exhibits various effector function, such as interaction with the Fc receptor. The term refers to the portion of an immunoglobulin molecule having a more conserved amino acid sequence relative to the other portion of the immunoglobulin, the variable region, which contains the antigen binding site. The constant region may contain the CH1, CH2, and CH3 regions of the heavy chain and the CL region of the light chain.

The term “framework” or “FR” refers to those variable region residues flanking the CDRs. FR residues are present, for example, in chimeric, humanized, human, domain antibodies, diabodies, linear antibodies, and bispecific antibodies. FR residues are those variable domain residues other than the hypervariable region residues or CDR residues.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue.

A “functional Fc region” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); antibody-dependent cellular phagocytosis (ADCP); cytokine secretion, downregulation of cell surface receptors (e.g., B cell receptor), and B cell activation, etc. Such effector functions generally require the Fc region to be combined with a binding region or binding domain (e.g., an antibody variable region or domain) and can be assessed using various assays as disclosed.

An “activating Fc receptor” is an Fc receptor that following engagement by an Fc region of an antibody elicits signaling events that stimulate the receptor-bearing cell to perform effector functions. Exemplary activating Fc receptors include FcγRIIIa (CD16α), FcγRI (CD64), FcγRIIa (CD32), and FcαRI (CD89).

A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human IgG1 Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof. For example, a native human IgG1 Fc region amino acid sequence is provided below:

(SEQ ID NO: 6)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSPGK

A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith. For example, a variant.

A “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution), insertion of one or more (e.g., generally fewer than 5, 4, or 3) amino acids adjacent to said residue/position, and/or deletion of said residue/position.

A “modification promoting heterodimerization” is a manipulation of the peptide backbone or the post-translational modifications of a polypeptide, e.g., an immunoglobulin heavy chain, that reduces or prevents the association of the polypeptide with an identical polypeptide to form a homodimer. A modification promoting heterodimerization as used herein particularly includes separate modifications made to each of two polypeptides desired to form a dimer, wherein the modifications are complementary to each other so as to promote association of the two polypeptides. For example, a modification promoting heterodimerization may alter the structure or charge of one or both of the polypeptides desired to form a dimer so as to make their association sterically or electrostatically favorable, respectively. Heterodimerization occurs between two non-identical polypeptides, such as two immunoglobulin heavy chains wherein further immunoconjugate components fused to each of the heavy chains (e.g., IL-2 polypeptide) are not the same. In the immunoconjugates of the present disclosure, the modification promoting heterodimerization is in the heavy chain(s), specifically in the Fc domain, of an immunoglobulin molecule. In some embodiments the modification promoting heterodimerization comprises an amino acid mutation, specifically an amino acid substitution. In a particular embodiment, the modification promoting heterodimerization comprises a separate amino acid mutation, specifically an amino acid substitution, in each of the two immunoglobulin heavy chains.

The term “Fc domain” herein is used to define the C-terminal portion of an immunoglobulin composed of the Fc regions of both heavy chains of the immunoglobulin. Each heavy chain Fc region in an Fc domain is herein referred to as a subunit of the Fc domain. The two subunits of a Fc domain can be both native sequence Fc regions, or both variant Fc regions, or one native sequence Fc region and one variant Fc region. In certain embodiments, the Fc domain comprises a modification promoting hetero-dimerization of two non-identical immunoglobulin heavy chains. The site of most extensive protein-protein interaction between the two polypeptide chains of a human IgG Fc domain is in the CH3 domain of the Fc regions. Thus, in one embodiment, said modification is in the CH3 domain of the Fc regions. In a specific embodiment said modification is a knob-into-hole modification, comprising a knob modification in one of the Fc subunits, referred to as “Fc-Knob,” and a hole modification in the other one of the Fc subunits, referred to as “Fc-hole.” The knob-into-hole technology is described e.g., in U.S. Pat. Nos. 5,731,168; 7,695,936; Ridgway et al., Prat Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). The protuberance and cavity can be made by altering the nucleic acid encoding the polypeptides, e.g., by site-specific mutagenesis, or by peptide synthesis. In a specific embodiment a knob modification comprises the amino acid substitution T366W in one of the two Fc subunits, and the hole modification comprises the amino acid substitutions T366S, L368A and Y407V in the other one of the two Fc subunits. In a further specific embodiment, the Fc subunit comprising the knob modification additionally comprises the amino acid substitution S354C, and the immunoglobulin heavy chain comprising the hole modification additionally comprises the amino acid substitution Y349C. Introduction of these two cysteine residues results in formation of a disulfide bridge between the two heavy chains, further stabilizing the dimer (Carter, J. Immunol Methods 248, 7-15 (2001)).

The term “variant” when used in relation to a peptide or polypeptide, to an antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a IL-2 variant may result from one or more (such as, for example, about 1 to about 25, about 1 to about 20, about 1 to about 15, about 1 to about 10, or about 1 to about 5) changes to an amino acid sequence of a native IL-2. Also by way of example, a variant of an anti-FAP antibody may result from one or more (such as, for example, about 1 to about 25, about 1 to about 20, about 1 to about 15, about 1 to about 10, or about 1 to about 5) changes to an amino acid sequence of a native or previously unmodified anti-FAP antibody. Variants may be naturally occurring, such as allelic or splice variants, or may be artificially constructed. Polypeptide variants may be prepared from the corresponding nucleic acid molecules encoding the variants. In specific embodiments, the IL-2 variant or anti-FAP antibody variant at least retains IL-2 or anti-FAP antibody functional activity, respectively. In specific embodiments, an anti-FAP antibody variant is a bispecific antibody that binds to both FAP and IL-2. In certain embodiments, the variant is encoded by a single nucleotide polymorphism (SNP) variant of a nucleic acid molecule that encodes IL-2 or anti-FAP antibody VH or VL regions or subregions, such as one or more CDRs.

An “intact” antibody is one comprising an antigen-binding site as well as a CL and at least heavy chain constant regions, CH1, CH2 and CH3. The constant regions may include human constant regions or amino acid sequence variants thereof. In certain embodiments, an intact antibody has one or more effector functions.

“Antibody fragments” comprise a portion of an intact antibody, such as the antigen-binding or variable region of the intact antibody. Examples of antibody fragments include, without limitation, Fab, Fab′, F(ab′)₂, and Fv fragments; diabodies and di-diabodies (see, e.g., Holliger et al., 1993, Proc. Natl. Acad. Sci. 90:6444-48; Lu et al., 2005, J. Biol. Chem. 280:19665-72; Hudson et al., 2003, Nat. Med. 9:129-34; WO 93/11161; and U.S. Pat. Nos. 5,837,242 and 6,492,123); single-chain antibody molecules (see, e.g., U.S. Pat. Nos. 4,946,778; 5,260,203; 5,482,858; and 5,476,786); dual variable domain antibodies (see, e.g., U.S. Pat. No. 7,612,181); single domain antibodies (sdAbs) (see, e.g., Woolven et al., 1999, Immunogenetics 50: 98-101; and Streltsov et al., 2004, Proc Natl Acad Sci USA. 101:12444-49); and multispecific antibodies formed from antibody fragments.

A “functional fragment,” “binding fragment,” or “antigen-binding fragment” of a therapeutic antibody will exhibit at least one if not some or all of the biological functions attributed to the intact antibody, the function comprising at least binding to the target antigen (e.g., an IL-2 binding fragment or fragment that binds to IL-2).

As used herein, the term “immunoconjugate” refers to a polypeptide molecule that includes at least one cytokine moiety and at least one antigen binding moiety. In certain embodiments, the immunoconjugate comprises at least one cytokine moiety (e.g., IL-2), and at least two antigen binding moieties (e.g., a masking moiety and an anchoring moiety as described herein). Particularly, in certain embodiments, immunoconjugates according to the present disclosure comprise one cytokine moiety and two antigen binding moieties joined by one or more linker sequences. In certain embodiments, immunoconjugates according to the present disclosure comprises one cytokine moiety and two antigen binding moieties joined by an Fc domain of immunoglobulin. In various embodiments of the present disclosure, the antigen binding moiety can be joined to the cytokine moiety by a variety of interactions and in a variety of configurations as described herein.

The term “fusion,” “fuse” or other grammatical variants thereof when used in relation to a peptide or polypeptide, or to an antibody refers to the joining of a peptide or polypeptide, or fragment, variant, and/or derivative thereof, with a heterologous peptide or polypeptide.

An “affinity matured” antibody is one with one or more alterations (e.g., amino acid sequence variations, including changes, additions, and/or deletions) in one or more HVRs thereof which result in an improvement in the affinity of the antibody for antigen, compared to a parent antibody which does not possess those alteration(s). Affinity matured antibodies can have nanomolar or even picomolar affinities for the target antigen. Affinity matured antibodies are produced by procedures known in the art. For review, see Hudson and Souriau, 2003, Nature Medicine 9:129-34; Hoogenboom, 2005, Nature Biotechnol. 23:1105-16; Quiroz and Sinclair, 2010, Revista Ingeneria Biomedia 4:39-51.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. Specific illustrative embodiments include the following. In one embodiment, the “K_{D}” or “K_{D} value” may be measured by assays known in the art, for example by a binding assay. The K_{D} may be measured in a RIA, for example, performed with the Fab version of an antibody of interest and its antigen (Chen et al., 1999, J. Mol Biol 293:865-81). The K_{D} or K_{D} value may also be measured by using surface plasmon resonance assays by Biacore®, using, for example, a Biacore® TM-2000 or a Biacore® TM-3000, or by biolayer interferometry using, for example, a Octet® QK384 or Gator™ system. An “on-rate” or “rate of association” or “association rate” or “kₒₙ” may also be determined with the same surface plasmon resonance or biolayer interferometry techniques described above using, for example, a Biacore® TM-2000 or a Biacore® TM-3000, or a Octet® QK384, or Gator™ system.

The term “inhibition” or “inhibit,” when used herein, refers to partial (such as, 1%, 2%, 5%, 10%, 20%, 25%, 50%, 75%, 90%, 95%, 99%) or complete (i.e., 100%) inhibition.

“Fc receptor” or “FcR” describes a receptor that binds to the Fc region of an antibody. An exemplary FcR is a native sequence human FcR. Moreover, an exemplary FcR is one that binds an IgG antibody (e.g., a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof (see, e.g., Daeron, 1997, Annu. Rev. Immunol. 15:203-34). Various FcRs are known (see, e.g., Ravetch and Kinet, 1991, Annu. Rev. Immunol. 9:457-92; Capel et al., 1994, Immunomethods 4:25-34; and de Haas et al., 1995, J. Lab. Clin. Med. 126:330-41). Other FcRs, including those to be identified in the future, are encompassed by the term “FcR” herein. The term also includes the neonatal receptor, FcRn, which is responsible for the transfer of maternal IgGs to the fetus (see, e.g., Guyer et al., 1976, J. Immunol. 117:587-93; and Kim et al., 1994, Eu. J. Immunol. 24:2429-34). Antibody variants with improved or diminished binding to FcRs have been described (see, e.g., WO 2000/42072; U.S. Pat. Nos. 7,183,387; 7,332,581; and 7.335,742; Shields et al. 2001, J. Biol. Chem. 9(2):6591-604).

The term “vector” refers to a substance that is used to carry or include a nucleic acid sequence, including for example, a nucleic acid sequence encoding an antibody or a cytokine polypeptide as described herein, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed (e.g., both an antibody heavy and light chain or an antibody VH and VL), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., an anti-FAP antibody as described herein), and it is further understood that expression levels can be optimized to obtain sufficient expression using methods well known in the art.

An “isolated nucleic acid” is a nucleic acid, for example, an RNA, DNA, or a mixed nucleic acids, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a specific embodiment, one or more nucleic acid molecules encoding an antibody as described herein are isolated or purified. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction. A polynucleotide may comprise modified nucleotides, such as methylated nucleotides and their analogs. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. A cell that produces an antibody of the present disclosure may include a parent hybridoma cell, as well as bacterial and eukaryotic host cells into which nucleic acids encoding the antibodies have been introduced. Suitable host cells are disclosed below.

Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “encoding nucleic acid” or grammatical equivalents thereof as it is used in reference to nucleic acid molecule refers to a nucleic acid molecule in its native state or when manipulated by methods well known to those skilled in the art that can be transcribed to produce mRNA, which is then translated into a polypeptide and/or a fragment thereof. The antisense strand is the complement of such a nucleic acid molecule, and the encoding sequence can be deduced therefrom.

The term “recombinant antibody” refers to an antibody that is prepared, expressed, created, or isolated by recombinant means. Recombinant antibodies can be antibodies expressed using a recombinant expression vector transfected into a host cell, antibodies isolated from a recombinant, combinatorial antibody library, antibodies isolated from an animal (e.g., a mouse or cow) that is transgenic and/or transchromosomal for human immunoglobulin genes (see, e.g., Taylor et al., 1992, Nucl. Acids Res. 20:6287-95), or antibodies prepared, expressed, created, or isolated by any other means that involves splicing of immunoglobulin gene sequences to other DNA sequences. Such recombinant antibodies can have variable and constant regions, including those derived from human germline immunoglobulin sequences (See Kabat et al., supra). In certain embodiments, however, such recombinant antibodies may be subjected to in vitro mutagenesis (or, when an animal transgenic for human Ig sequences is used, in vivo somatic mutagenesis), thus the amino acid sequences of the VH and VL regions of the recombinant antibodies are sequences that, while derived from and related to human germline VH and VL sequences, may not naturally exist within the human antibody germline repertoire in vivo.

The term “composition” is intended to encompass a product containing the specified ingredients (e.g., an immunoconjugate molecule provided herein) in, optionally, the specified amounts.

“Carriers” as used herein include pharmaceutically acceptable carriers, excipients, or stabilizers that are nontoxic to the cell or mammal being exposed thereto at the dosages and concentrations employed. Often the physiologically acceptable carrier is an aqueous pH buffered solution. Examples of physiologically acceptable carriers include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The term “carrier” can also refer to a diluent, adjuvant (e.g., Freund's adjuvant (complete or incomplete)), excipient, or vehicle. Such carriers, including pharmaceutical carriers, can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard carriers such as pharmaceutical grades of mannitol, lactose, starch, magnesium stearate, sodium saccharine, cellulose, magnesium carbonate, etc. Examples of suitable pharmaceutical carriers are described in Remington and Gennaro, Remington's Pharmaceutical Sciences (18th ed. 1990). Compositions, including pharmaceutical compounds, may contain an antibody, for example, in isolated or purified form, together with a suitable amount of carriers.

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

The term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.), and polyols (e.g., mannitol, sorbitol, etc.). See, also, Remington and Gennaro, Remington's Pharmaceutical Sciences (18th ed. 1990), which is hereby incorporated by reference in its entirety.

The terms “subject” and “patient” may be used interchangeably. As used herein, in certain embodiments, a subject is a mammal, such as a non-primate (e.g., cow, pig, horse, cat, dog, rat, etc.) or a primate (e.g., monkey and human). In specific embodiments, the subject is a human.

“Administer” or “administration” refers to the act of injecting or otherwise physically delivering a substance as it exists outside the body (e.g., an immunoconjugate molecule as described herein) into a patient, such as by mucosal, intradermal, intravenous, intramuscular delivery, and/or any other method of physical delivery described herein or known in the art.

The term “effective amount” as used herein refers to the amount of an antibody or pharmaceutical composition provided herein which is sufficient to result in the desired outcome.

The terms “about” and “approximately” mean within 20%, within 15%, within 10%, within 9%, within 8%, within 7%, within 6%, within 5%, within 4%, within 3%, within 2%, within 1%, or less of a given value or range.

“Substantially all” refers to at least about 60%, at least about 65%, at least about 70%, at least about 75%, at least about 80%, at least about 85%, at least about 90%, at least about 95%, at least about 98%, at least about 99%, or about 100%.

The phrase “substantially similar” or “substantially the same” denotes a sufficiently high degree of similarity between two numeric values (e.g., one associated with an antibody of the present disclosure and the other associated with a reference antibody) such that one of skill in the art would consider the difference between the two values to be of little or no biological and/or statistical significance within the context of the biological characteristic measured by the values (e.g., K_{D} values). For example, the difference between the two values may be less than about 50%, less than about 40%, less than about 30%, less than about 20%, less than about 10%, or less than about 5%, as a function of the value for the reference antibody.

The phrase “substantially increased,” “substantially reduced,” or “substantially different,” as used herein, denotes a sufficiently high degree of difference between two numeric values (e.g., one associated with an antibody of the present disclosure and the other associated with a reference antibody) such that one of skill in the art would consider the difference between the two values to be of statistical significance within the context of the biological characteristic measured by the values. For example, the difference between said two values can be greater than about 10%, greater than about 20%, greater than about 30%, greater than about 40%, or greater than about 50%, as a function of the value for the reference antibody.

### 5.3 Compositions and Methods of Making the Same

In one aspect of the present disclosure, provided herein are cytokine-containing immunoconjugate molecules. In some embodiments, the immunoconjugate molecules are fusion proteins comprising a cytokine moiety and a non-cytokine portion operably linked to one another. According to the present disclosure, the cytokine-containing immunoconjugate molecules are capable of delivery and activation of cellular activities of the cytokine at particular tissue or cellular location in a subject. For example, in some embodiments, the cytokine activity is reduced or blocked when the immunoconjugate molecules are present in an environment lacking an activation signal for the cytokine. In some embodiments, the cytokine activity is activated or enhanced when the immunoconjugate molecule are present in an environment containing or enriched of the activation signal for the cytokine. For example, in some embodiments, the immunoconjugate molecules are configured for tissue-specific distribution upon administration to a subject. In particular embodiments, the immunoconjugate molecules are capable of being enriched in certain tissue or cellular environment providing the activation signal for the cytokine, thereby activating the cytokine activity specifically in such tissue or cellular environment.

In specific embodiments, the activation signal for the cytokine is the presence of a signal molecule in the target tissue or cellular environment where the cytokine activity is activated. In some embodiments, the signal molecule is enriched in the target tissue or cellular environment, while present at other non-target tissue or cellular environment at a lower amount or concentration. In some embodiments, the activation signal for the cytokine is the presence of a signal molecule in the target tissue or cellular environment at a concentration above a threshold. In some embodiments, the signal molecule is capable of interacting with the immunoconjugate molecule, thereby activates the cytokine activity. In some embodiments, the signal molecule is a peptidic molecule.

In specific embodiments, the immunoconjugate molecules are configured for the targeted delivery and activation of the cytokine activity in cancerous tissues, such as a tumor. In those embodiments, the signal molecule for activating the cytokine can be an antigen that is expressed or enriched in the cancerous tissue, such as in the tumor microenvironment. In specific embodiments, the activation signal for the cytokine is an antigen expressed on the tumor cells. In other embodiments, the activation signal for the cytokine is an antigen expressed on the cells in the tumor microenvironment, such as tumor stromal cells. In specific embodiments, the activation signal for the cytokine is a tumor associated antigen.

In some embodiments, the non-cytokine portion of the immunoconjugate molecule comprises a masking moiety capable of binding with the cytokine moiety, and upon the binding, the masking moiety reduces or blocks the cytokine activity. In some embodiments, the immunoconjugate molecule comprises an antibody or antigen binding fragment thereof that is fused to a cytokine polypeptide, and the antibody or antigen binding fragment thereof is capable of binding with the cytokine polypeptide and reduces or blocks the cytokine activity.

In some embodiments, the intramolecular binding between the cytokine moiety and the masking moiety of an immunoconjugate molecule is reversible. Accordingly, in some embodiments, the immunoconjugate molecules can switch between cytokine active and inactive states, through the reversible binding and disassociation between the cytokine moiety and the masking moiety.

In some embodiments, the masking moiety is a bispecific two-in-one antibody or a binding fragment thereof, which is capable of binding to the cytokine moiety and a second target antigen that is different from the cytokine. In specific embodiments, when the immunoconjugate molecule is in an environment where the second target antigen is absent, the masking moiety comprising the two-in-one antibody or antigen binding fragment thereof binds with the cytokine moiety of the immunoconjugate molecule, thereby inhibiting the cytokine activity. In specific embodiments, when the immunoconjugate molecule is in an environment where the second target antigen is present at an amount or concentration below a certain threshold, the masking moiety comprising the two-in-one antibody or antigen binding fragment thereof binds with the cytokine moiety of the immunoconjugate molecule, thereby inhibiting the cytokine activity. In various embodiments, the environment is a cellular environment or a tissue-specific environment. In particular embodiments, the environment is a cancerous tissue or a tumor microenvironment. In particular embodiments, the second target antigen is an antigen expressed by the cancer cells. In other embodiments, the second target antigen is an antigen expressed by the cells in the tumor microenvironment, such as tumor stromal cells. In some embodiments, the second target antigen is a tumor associated antigen.

In some embodiments, the masking moiety is a bispecific two-in-one antibody or a binding fragment thereof, which is capable of binding to the cytokine moiety and a second target antigen that is different from the cytokine. In specific embodiments, when the immunoconjugate molecule is in an environment where the second target antigen is present, the masking moiety comprising the two-in-one antibody or antigen binding fragment thereof binds with the second antigen and disassociates from the cytokine moiety of the immunoconjugate molecule, thereby activating the cytokine activity. In specific embodiments, when the immunoconjugate molecule is in an environment where the second target antigen is present at an amount or concentration above a certain threshold, the masking moiety comprising the two-in-one antibody or antigen binding fragment thereof binds with the second antigen and disassociates from the cytokine moiety of the immunoconjugate molecule, thereby activating the cytokine activity. In various embodiments, the environment is a cellular environment or a tissue-specific environment. In particular embodiments, the environment is a cancerous tissue or a tumor microenvironment. In particular embodiments, the second target antigen is an antigen expressed by the tumor cells. In other embodiments, the second target antigen is an antigen expressed by the cells in the tumor microenvironment, such as tumor stromal cells. In some embodiments, the second target antigen is a tumor associated antigen.

In specific embodiments, the immunoconjugate molecules of the present disclosure comprises a cytokine moiety and a non-cytokine portion, where the cytokine moiety comprises an interleukin-2 (IL-2) polypeptide, and the non-cytokine portion comprises a bispecific two-in-one antibody capable of binding to both the IL-2 polypeptide in the immunoconjugate molecule and an second target antigen that is not IL-2. In particular embodiments, the second target antigen is an antigen expressed by the tumor cells. In other embodiments, the second target antigen is an antigen expressed by the cells in the tumor microenvironment, such as tumor stromal cells. In some embodiments, the second target antigen is a tumor associated antigen. In specific embodiments, the second target antigen is fibroblast activation protein (FAP). In yet specific embodiments, the IL-2 polypeptide is wild-type IL-2 polypeptide. In other embodiments, the IL-2 polypeptide is a mutant IL-2 polypeptide. In some embodiments, the IL-2 polypeptide is a human IL-2 polypeptide. In some embodiments, the IL-2 polypeptide is a monkey IL-2 polypeptide. In some embodiments, the IL-2 polypeptide is a mouse IL-2 polypeptide. In some embodiments, the IL-2 polypeptide is a mutant IL-2 polypeptide as described herein. In specific embodiments, the mutant IL-2 polypeptide is IL-2hex. Additional mutant IL-2 polypeptides that can be used in connection with the present disclosure can be found in U.S. Pat. Nos.:10,184,009 and 5,229,109 and International Patent Publication No. WO2012107417A1, the disclosure of each of which is enclosed herein by reference in its entirety.

In some embodiments, the non-cytokine portion of the immunoconjugate molecule comprises an anchoring moiety configured to tether the immunoconjugate molecule to a target location of delivery. Hence, in some embodiments, immunoconjugate molecules of the present disclosure having the anchoring moiety can achieve tissue-specific distribution after being administered to a subject, such as after systemic administration to a subject. In some embodiments, the anchoring moiety of the immunoconjugate molecule is capable of specific binding to a target molecule that is present in the target location of delivery. In some embodiments, the anchoring moiety of the immunoconjugate molecule comprises an antibody or antigen binding fragment thereof capable of binding to an antigen present in the target location of delivery, thereby tethering the immunoconjugate molecule to the target location of delivery.

In some embodiments, the target location of delivery is a cellular environment, or a tissue-specific environment. In some embodiments, the target location of delivery also contains an activation signal for the cytokine of the immunoconjugate molecule, such that the cytokine activity can be activated in the target location.

In particular embodiments, the target location of delivery is a cancerous tissue or a tumor microenvironment. In some embodiments, the target location of delivery is a particular type of tissue or population of cells in a subject. In some embodiments, the anchoring moiety of the immunoconjugate molecule comprises an antibody or antigen binding fragment thereof that bind to an antigen expressed on cancer cells. Accordingly, in those embodiments, the immunoconjugate molecule, upon administration to a subject having cancer, can bind to a population of cancer cells in the subject. In some embodiments, the anchoring moiety of the immunoconjugate molecule comprises an antibody or antigen binding fragment thereof that bind to an antigen present in the tumor microenvironment, such as an antigen expressed on surface of a tumor cells or antigen secreted by cells in the tumor microenvironment, such as tumor stromal cells. Accordingly, in those embodiments, the immunoconjugate molecule, upon administration to a subject having a solid tumor, can enrich in the tumor microenvironment in the subject.

In some embodiments, the immunoconjugate molecules of the present disclosure comprises a cytokine moiety, a masking moiety and an anchoring moiety that are operably connected with one another. In specific embodiments, the masking moiety is a bispecific two-in-one antibody or antigen binding fragment thereof capable of binding to both the cytokine moiety and a second target antigen that is not the cytokine. In specific embodiments, the anchoring moiety is an antibody or antigen binding fragment thereof capable of binding to a third target antigen, such as an antigen present in a target location of delivery for the immunoconjugate molecule. In some embodiments, the target location of delivery also contains the second target antigen in a sufficient amount to compete with the cytokine for binding with the masking moiety, resulting in disassociation of the masking moiety from the cytokine and activation of cytokine activity at the target location of delivery.

In some embodiments, the immunoconjugate molecules, upon administration to a subject, can achieve tissue-specific distribution and enrich in a target tissue or cellular environment in the subject that contains sufficient amount of the third antigen. In specific embodiments, the target tissue or cellular environment also contains the second target antigen in a sufficient amount to compete with the cytokine for binding with the masking moiety, resulting in disassociation of the masking moiety from the cytokine and activation of cytokine activity in the target tissue or cellular environment.

In specific embodiments, the second and the third target antigens respectively recognized by the masking moiety and the anchoring moiety of the immunoconjugate are the same antigen. In alternative embodiments, the second and the third target antigens respectively recognized by the masking moiety and the anchoring moiety of the immunoconjugate are different antigens.

In specific embodiments, the cytokine moiety comprises an interleukin-2 (IL-2) polypeptide, and the non-cytokine portion of the immunoconjugate molecule comprises a masking moiety comprising a bispecific two-in-one antibody capable of binding to both the IL-2 polypeptide in the immunoconjugate molecule and a second target antigen that is not IL-2. In particular embodiments, the second target antigen is an antigen expressed by the tumor cells. In other embodiments, the second target antigen is an antigen expressed by the cells in the tumor microenvironment, such as tumor stromal cells. In some embodiments, the second target antigen is a tumor associated antigen. In specific embodiments, the second target antigen is fibroblast activation protein (FAP). In specific embodiments, the non-cytokine portion of the immunoconjugate molecule further comprises an anchoring moiety comprising an antibody or antigen binding fragment capable of binding to a third target antigen that is not IL-2. In particular embodiments, the third target antigen is an antigen expressed by the tumor cells. In some embodiments, the third target antigen is an antigen expressed by the cells in the tumor microenvironment, such as tumor stromal cells. In some embodiments, the third target antigen is a tumor associated antigen. In specific embodiments, the third target antigen is fibroblast activation protein (FAP). In yet specific embodiments, the IL-2 polypeptide is wild-type IL-2 polypeptide.). In yet specific embodiments, the IL-2 polypeptide is wild-type IL-2 polypeptide. In other embodiments, the IL-2 polypeptide is a mutant IL-2 polypeptide. In some embodiments, the IL-2 polypeptide is a human IL-2 polypeptide. In some embodiments, the IL-2 polypeptide is a monkey IL-2 polypeptide. In some embodiments, the IL-2 polypeptide is a mouse IL-2 polypeptide. In some embodiments, the IL-2 polypeptide is a mutant IL-2 polypeptide as described herein. In specific embodiments, the mutant IL-2 polypeptide is IL-2hex. Additional mutant IL-2 polypeptides that can be used in connection with the present disclosure can be found in U.S. Pat. Nos.:10,184,009 and 5,229,109 and International Patent Publication No. WO2012107417A1, the disclosure of each of which is enclosed herein by reference in its entirety.

In some embodiments, the present immunoconjugate molecule comprises an anchoring moiety, a masking moiety and a cytokine moiety that are operably linked to one another via a conjugating moiety. In some embodiments, the conjugating moiety comprises an immunoglobulin Fc domain composed of the Fc regions of both heavy chains of the immunoglobulin (each a subunit of the Fc domain). In some embodiments, the Fc domain is the Fc domain of an IgG molecule (e.g., IgG1, IgG2, IgG3, or IgG4).

In some embodiments, the two subunits of the Fc domain can be both native sequence Fc regions. In some embodiments, the two subunits of the Fc domain can be both variant Fc regions. In some embodiments, the two subunits of the Fc domain can be one native sequence Fc region and one variant Fc region. In certain embodiments, the Fc domain comprises a modification promoting hetero-dimerization of two non-identical immunoglobulin heavy chains. The site of most extensive protein-protein interaction between the two polypeptide chains of a human IgG Fc domain is in the CH3 domain of the Fc regions. Thus, in one embodiment, said modification is in the CH3 domain of the Fc regions. In a specific embodiment said modification is a knob-into-hole modification, comprising a knob modification in one of the Fc subunits and a hole modification in the other one of the Fc subunits. The knob-into-hole technology is described e.g., in U.S. Pat. Nos. 5,731,168; 7,695,936; Ridgway et al., Prat Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g., tyrosine or tryptophan). Compensatory cavities of identical or similar size to the protuberances are created in the interface of the second polypeptide by replacing large amino acid side chains with smaller ones (e.g., alanine or threonine). The protuberance and cavity can be made by altering the nucleic acid encoding the polypeptides, e.g., by site-specific mutagenesis, or by peptide synthesis. In a specific embodiment, a knob modification comprises the amino acid substitution T366W in one of the two Fc subunits, and the hole modification comprises the amino acid substitutions T366S, L368A and Y407V in the other one of the two Fc subunits. In a further specific embodiment, the Fc subunit comprising the knob modification additionally comprises the amino acid substitution S354C, and the immunoglobulin heavy chain comprising the hole modification additionally comprises the amino acid substitution Y349C. Introduction of these two cysteine residues results in formation of a disulfide bridge between the two heavy chains, further stabilizing the dimer (Carter, J. Immunol Methods 248, 7-15 (2001)).

In an alternative embodiment a modification promoting heterodimerization of two non-identical polypeptide chains comprises a modification mediating electrostatic steering effects, e.g., as described in PCT publication WO 2009/089004. Generally, this method involves replacement of one or more amino acid residues at the interface of the two polypeptide chains by charged amino acid residues so that homodimer formation becomes electro statically unfavorable but heterodimerization electrostatically favorable.

Without being bound by the theory, it is contemplated that an Fc domain confers to the immunoconjugate molecule favorable pharmacokinetic properties, including a long serum half-life which contributes to good accumulation in the target tissue and a favorable tissue-blood distribution ratio. At the same time an Fc domain may lead to undesirable targeting of the immunoconjugate molecules to cells expressing Fc receptors rather than to the target antigen-bearing cells. Moreover, the co-activation of Fc receptor signaling pathways may lead to cytokine release which, in combination with the cytokine polypeptide in the immunoconjugate molecule and the long half-life of the immunoconjugate, results in excessive activation of cytokine receptors and severe side effects upon systemic administration. In line with this, conventional IgG-IL-2 immunoconjugates have been described to be associated with infusion reactions (see e.g., King et al., J Clin Oneal 22, 4463-4473 (2004)).

In certain embodiments, the modification to the Fc region of the antibody results in the decrease or elimination of an effector function of the antibody. In certain embodiments, the effector function is ADCC, ADCP, and/or CDC. In some embodiments, the effector function is ADCC. In other embodiments, the effector function is ADCP. In other embodiments, the effector function is CDC. In one embodiment, the effector function is ADCC and ADCP. In one embodiment, the effector function is ADCC and CDC. In one embodiment, the effector function is ADCP and CDC. In one embodiment, the effector function is ADCC, ADCP and CDC. This may be achieved by introducing one or more amino acid substitutions in an Fc region of the antibody. For example, substitutions into human IgG1 using IgG2 residues at positions 233-236 and IgG4 residues at positions 327, 330, and 331 were shown to greatly reduce ADCC and CDC (see, e.g., Armour et al., 1999, Eur. J. Immunol. 29(8):2613-24; and Shields et al., 2001, J. Biol. Chem. 276(9): 6591-604). Other Fc variants are provided elsewhere herein.

To increase the serum half-life of the antibody, one may incorporate a salvage receptor binding epitope into the antibody (especially an antibody fragment), for example, as described in U.S. Pat. No. 5,739,277. Term “salvage receptor binding epitope” refers to an epitope of the Fc region of an IgG molecule (e.g., IgG1, IgG2, IgG3, or IgG4) that is responsible for increasing the in vivo serum half-life of the IgG molecule.

Accordingly, in some embodiments, the Fc domain forming part of the immunoconjugate molecule according to the present disclosure is engineered to have reduced binding affinity to an Fc receptor. In one such embodiment the Fc domain comprises one or more amino acid mutation that reduces the binding affinity of the Fc domain to an Fc receptor. In one such embodiment, the one or more such amino acid mutations are present in one of the two Fc subunits of the Fc domain. In another such embodiment, the one or more such amino acid mutations are present in both of the two Fc subunits of the Fc domain. In various embodiments, such amino acid mutations reduce the binding affinity of the immunoconjugate to the Fc receptor by at least 2-fold, at least 5-fold, or at least 10-fold.

In some embodiments where there is more than one amino acid mutation that reduces the binding affinity of the Fc domain composed of the present immunoconjugate molecule to the Fc receptor, the combination of these amino acid mutations can reduce the binding affinity of the Fc domain to the Fc receptor by at least 10-fold, at least 20-fold, or even at least 50-fold. In one embodiment the immunoconjugate comprising an engineered immunoglobulin molecule exhibits less than 20%, particularly less than 10%, more particularly less than 5% of the binding affinity to an Fc receptor as compared to an immunoconjugate comprising a non-engineered immunoglobulin molecule.

In some embodiments, the Fc receptor is an activating Fc receptor. In a specific embodiment the Fc receptor is an Fcγ receptor. More specifically, in some embodiments, the Fc receptor is an FcγRIIIa, FcγRI or FcγRIIa receptor. In some embodiments, binding of the Fc domain to each of these exemplary receptors is reduced. In some embodiments, binding affinity of the Fc domain to a complement component is reduced. Specifically in some embodiments, binding affinity of the Fc domain to C1q is reduced. In one embodiment, binding affinity to neonatal Fc receptor (FcRn) is not reduced. Substantially similar binding to FcRn, i.e. preservation of the binding affinity of the Fc domain to said receptor, is achieved when the immunoconjugate comprising said Fc domain exhibits greater than about 70% of the binding affinity of a non-engineered form of the immunoconjugate molecule comprising said non-engineered form of the Fc to FcRn. Immunoglobulins, or immunoconjugates comprising said immunoglobulins, may exhibit greater than about 80% and even greater than about 90% of such affinity.

In some embodiments, the Fc domain forming part of the present immunoconjugate molecule is not a native sequence Fc domain and has at least one amino acid mutation in one of its Fc subunits. In some embodiments, the Fc domain forming part of the present immunoconjugate molecule is not a native sequence Fc domain and has at least one amino acid mutation in both of its Fc subunits. In some embodiments, the amino acid mutations in both Fc subunits of an Fc domain are the same mutations. In some embodiments, the amino acid mutations in the two Fc subunits of an Fc domain are different mutations. In some embodiments, the amino acid mutation is selected from amino acid substitution, amino acid deletion and amino acid insertion. In particular embodiments, one or both of the Fc subunits in the Fc domain of the immunoconjugate molecule comprise one or more amino acid mutations at any one or more amino acid positions 228, 233, 234, 235, 236, 265, 297, 329, 330, and 331 of the Fc subunit, where the number of the residues in the Fc subunit is that of the EU index as in Kabat. In particular embodiments, such one or more amino acid substitutions comprise S228P. In particular embodiments, such one or more amino acid substitutions comprise E233P. In particular embodiments, such one or more amino acid substitutions comprise L234V or L234A. In particular embodiments, such one or more amino acid substitutions comprise L235A or L235E. In particular embodiments, such one or more amino acid deletion comprises AG236. In particular embodiments, such one or more amino acid substitutions comprise D265G. In particular embodiments, such one or more amino acid substitutions comprise N297A or N297D. In particular embodiments, such one or more amino acid substitutions comprise P329E, P329A or P329G, particularly P329E. In particular embodiments, such one or more amino acid substitutions comprise A330S. In particular embodiments, such one or more amino acid substitutions comprise P331S.

In particular embodiments, the Fc domain comprises amino acid mutations at positions E233, L234, L235, G236, A330, and P331. In specific embodiments, both of the two Fc subunits comprises amino acid mutations at positions E233, L234, L235, G236, A330, and P331. In particular embodiments, the Fc domain comprises amino acid mutations of E233P, L234V, L235A, AG236, A330S, and P331S. In specific embodiments, both of the two Fc subunits comprises amino acid mutations of E233P, L234V, L235A, AG236, P329S, A330S, and P331S.

In particular embodiments, the Fc domain comprises amino acid mutations at positions L234, L235, A330, and P331. In specific embodiments, both of the two Fc subunits comprise amino acid mutations at positions L234, L235, A330, and P331. In particular embodiments, the Fc domain comprises amino acid mutations of L234A, L235A, A330S, and P331S. In specific embodiments, both of the two Fc subunits comprise amino acid mutations of L234A, L235A, A330S, and P331S.

In particular embodiments, the Fc domain comprises amino acid mutations at positions E233, L234, L235, G236, P329, A330, and P331. In specific embodiments, both of the two Fc subunits comprise amino acid mutations at positions E233, L234, L235, G236, P329, A330, and P331. In particular embodiments, the Fc domain comprises amino acid mutations of E233P, L234V, L235A, AG236, P329E, A330S, and P331S. In specific embodiments, both of the two Fc subunits comprise amino acid mutations of E233P, L234V, L235A, AG236, P329E, A330S, and P331S.

In particular embodiments, the Fc domain comprises amino acid mutations at positions L234, L235, P329, A330, and P331. In specific embodiments, both of the two Fc subunits comprise amino acid mutations at positions L234, L235, P329, A330, and P331. In particular embodiments, the Fc domain comprises amino acid mutations of L234A, L235A, P329E, A330S, and P331S. In specific embodiments, both of the two Fc subunits comprise amino acid mutations of L234A, L235A, P329E, A330S, and P331S.

In particular embodiments, the Fc domain comprises amino acid mutations at positions E233, L234, L235, G236, and P329. In specific embodiments, both of the two Fc subunits comprise amino acid mutations at positions E233, L234, L235, G236, and P329. In particular embodiments, the Fc domain comprises amino acid mutations of E233P, L234V, L235A, AG236, and P329E. In specific embodiments, both of the two Fc subunits comprise amino acid mutations of E233P, L234V, L235A, AG236, and P329E.

In particular embodiments, the Fc domain comprises amino acid mutations at positions L234, L235, P329. In specific embodiments, both of the two Fc subunits comprise amino acid mutations at positions L234, L235, P329. In particular embodiments, the Fc domain comprises amino acid mutations of L234A, L235A, and P329E. In specific embodiments, both of the two Fc subunits comprise amino acid mutations of L234A, L235A, and P329E.

In particular embodiments, the Fc domain comprises amino acid mutations at positions E233, L234, L235, G236, D265, A330, and P331. In specific embodiments, both of the two Fc subunits comprise amino acid mutations at positions E233, L234, L235, G236, D265, A330, and P331. In particular embodiments, the Fc domain comprises amino acid mutations of E233P, L234V, L235A, AG236, D265G, A330S, and P331S. In specific embodiments, both of the two Fc subunits comprise amino acid mutations of E233P, L234V, L235A, AG236, D265G, A330S, and P331S. In these embodiments, the Fc domain has reduced binding affinity to the Fcγ receptor.

In particular embodiments, the Fc domain comprises amino acid mutations at positions L234, L235, D265, A330, and P331. In specific embodiments, both of the two Fc subunits comprise amino acid mutations at positions L234, L235, D265, A330, and P331. In particular embodiments, the Fc domain comprises amino acid mutations of L234A, L235A, D265G, A330S, and P331S. In specific embodiments, both of the two Fc subunits comprise amino acid mutations of L234A, L235A, D265G, A330S, and P331S.

In particular embodiments, the Fc domain comprises amino acid mutations at positions E233, L234, L235, G236, D265, P329, A330, and P331. In specific embodiments, both of the two Fc subunits comprise amino acid mutations at positions E233, L234, L235, G236, D265, P329, A330, and P331. In particular embodiments, the Fc domain comprises amino acid mutations of E233P, L234V, L235A, AG236, D265G, P329E, A330S, and P331S. In specific embodiments, both of the two Fc subunits comprise amino acid mutations of E233P, L234V, L235A, AG236, D265G, P329E, A330S, and P331S.

In particular embodiments, the Fc domain comprises amino acid mutations at positions L234, L235, D265, P329, A330, and P331. In specific embodiments, both of the two Fc subunits comprise amino acid mutations at positions L234, L235, D265, P329, A330, and P331. In particular embodiments, the Fc domain comprises amino acid mutations of L234A, L235A, D265G, P329E, A330S, and P331S. In specific embodiments, both of the two Fc subunits comprise amino acid mutations of L234A, L235A, D265G, P329E, A330S, and P331S.

In particular embodiments, the Fc domain comprises amino acid mutations at positions E233, L234, L235, G236, D265, and P329. In specific embodiments, both of the two Fc subunits comprise amino acid mutations at positions E233, L234, L235, G236, D265, and P329. In particular embodiments, the Fc domain comprises amino acid mutations of E233P, L234V, L235A, AG236, D265G, and P329E. In specific embodiments, both of the two Fc subunits comprise amino acid mutations of E233P, L234V, L235A, AG236, D265G, and P329E.

In particular embodiments, the Fc domain comprises amino acid mutations at positions L234, L235, D265, and P329. In specific embodiments, both of the two Fc subunits comprise amino acid mutations at positions L234, L235, D265, and P329. In particular embodiments, the Fc domain comprises amino acid mutations of L234A, L235A, D265G, and P329E. In specific embodiments, both of the two Fc subunits comprise amino acid mutations of L234A, L235A, D265G, and P329E.

In particular embodiments, the Fc domain comprises amino acid mutations at positions L234, L235, and P329. In specific embodiments, both of the two Fc subunits comprise amino acid mutations at positions L234, L235, and P329. In particular embodiments, the Fc domain comprises amino acid mutations of L234A, L235A, and P329G. In specific embodiments, both of the two Fc subunits comprise amino acid mutations of L234A, L235A, and P329G.

According to the present disclosure, the present immunoconjugate molecule comprises an anchoring moiety, a masking moiety and a cytokine moiety that are operably linked to one another via a conjugating moiety. In specific embodiments, the cytokine moiety comprises a cytokine polypeptide. In specific embodiments, the masking moiety comprises a bispecific two-in-one antibody or antigen binding fragment capable of binding to the cytokine polypeptide and a second target antigen. In specific embodiments, the anchoring moiety comprises an antibody or antigen binding fragment thereof capable of binding to a third target antigen. In specific embodiments, the conjugating moiety comprises an immunoglobulin Fc domain composed of two Fc regions of immunoglobulin heavy chains (each Fc region is referred to as a subunit of the Fc domain or “Fc subunit”). In some embodiments, the Fc domain comprises a modification promoting hetero-dimerization of the two Fc subunits. In specific embodiments, said modification is a knob-into-hole modification, comprising a knob modification in one of the Fc subunits (Fc-knob) and a hole modification in the other one of the Fc subunits (Fc-hole).

According to the present disclosure, in these embodiments, the cytokine moiety, the masking moiety, and the anchoring moiety of the immunoconjugate molecule can be operably linked to one another via the conjugating moiety in a variety of different configurations. In one exemplary embodiment, the cytokine moiety comprises a cytokine polypeptide that is fused to the C-terminus of one Fc subunit. In one exemplary embodiment, the masking moiety comprises an antibody or antigen binding fragment thereof that is fused to the C-terminus of one Fc subunit. In one exemplary embodiment, the cytokine moiety comprises a cytokine polypeptide that is fused to the C-terminus of one subunit of the Fc domain, and the masking moiety comprises an antibody or antigen binding fragment thereof that is fused to the C-terminus of the other Fc subunit. In some embodiments, the masking moiety is fused to the C-terminus of the Fc subunit. In some embodiments, the Fc domain comprises a modification promoting hetero-dimerization of the two Fc subunits. In specific embodiments, said modification is a knob-into-hole modification, comprising a knob modification in one of the Fc subunits (Fc-knob subunit) and a hole modification in the other one of the Fc subunits (Fc-hole subunit).

In one exemplary embodiment, the cytokine moiety comprises a cytokine polypeptide that is fused to the C-terminus of one Fc subunit. In one exemplary embodiment, the masking moiety comprises a bispecific two-in-one antibody or antigen binding fragment thereof that is fused to the C-terminus of one Fc subunit. In one exemplary embodiment, the anchoring moiety comprises an antibody or antigen binding fragment thereof that is fused to the N-terminus of one Fc subunit. In one exemplary embodiment, the cytokine moiety comprises a cytokine polypeptide that is fused to the C-terminus of one subunit of the Fc domain, and the masking moiety comprises a bispecific two-in-one antibody or antigen binding fragment thereof that is fused to the C-terminus of the other Fc subunit. In one exemplary embodiment, the cytokine moiety comprises a cytokine polypeptide that is fused to the C-terminus of one subunit of the Fc domain, the masking moiety comprises a bispecific two-in-one antibody or antigen binding fragment thereof that is fused to the C-terminus of the other Fc subunit, and the anchoring moiety comprises an antibody or antigen binding fragment thereof that is fused to the N-terminus of one subunit of the Fc domain. In specific embodiments, the anchoring moiety and the cytokine moiety are fused to the N- and C-terminus of the same Fc subunit, respectively. In specific embodiment, the masking moiety and the cytokine moiety are fused to the N- and C-terminus of the same Fc subunit, respectively. In some embodiments, the Fc domain comprises a modification promoting hetero-dimerization of the two Fc subunits. In specific embodiments, said modification is a knob-into-hole modification, comprising a knob modification in one of the Fc subunits (Fc-knob subunit) and a hole modification in the other one of the Fc subunits (Fc-hole subunit).

In one exemplary embodiment, the masking moiety comprises a bispecific two-in-one antibody or antigen binding fragment thereof that is fused to the N-terminus of one Fc subunit. In one exemplary embodiment, the cytokine moiety comprises a cytokine polypeptide that is fused to the masking moiety. In one exemplary embodiment, the masking moiety comprises a bispecific two-in-one antibody or antigen binding fragment thereof that is fused to the N-terminus of one Fc subunit, and the cytokine moiety comprises a cytokine polypeptide that is fused to the masking moiety. In one exemplary embodiment, the anchoring moiety comprises an antibody or antigen binding fragment thereof that is fused to the N-terminus of one Fc subunit. In one exemplary embodiment, the cytokine moiety comprises a cytokine polypeptide that is fused to the anchoring moiety. In one exemplary embodiment, the masking moiety comprises a bispecific two-in-one antibody or antigen binding fragment thereof that is fused to the N-terminus of one Fc subunit, the anchoring moiety comprises an antibody or antigen binding fragment thereof that is fused to the N-terminus of the other Fc subunit, and the cytokine moiety comprises a cytokine polypeptide that is fused to the masking moiety. In one exemplary embodiment, the masking moiety comprises a bispecific two-in-one antibody or antigen binding fragment thereof that is fused to the N-terminus of one Fc subunit, the anchoring moiety comprises an antibody or antigen binding fragment thereof that is fused to the N-terminus of the other Fc subunit, and the cytokine moiety comprises a cytokine polypeptide that is fused to the anchoring moiety. In some embodiments, the Fc domain comprises a modification promoting hetero-dimerization of the two Fc subunits. In specific embodiments, said modification is a knob-into-hole modification, comprising a knob modification in one of the Fc subunits (Fc-knob subunit) and a hole modification in the other one of the Fc subunits (Fc-hole subunit).

In one exemplary embodiment, the masking moiety comprises a bispecific two-in-one antibody or an antigen binding fragment thereof that is fused to the C-terminus of one Fc subunit. In one exemplary embodiment, the cytokine moiety comprises a cytokine polypeptide that is fused to the masking moiety. In one exemplary embodiment, the masking moiety comprises a bispecific two-in-one antibody or an antigen binding fragment thereof that is fused to the C-terminus of one Fc subunit, and the cytokine moiety comprises a cytokine polypeptide that is fused to the masking moiety. In one exemplary embodiment, the anchoring moiety comprising an antibody or antigen binding fragment thereof that is fused to the N terminus of one Fc subunit. In one exemplary embodiment, the masking moiety comprises a bispecific two-in-one antibody or an antigen binding fragment thereof that is fused to the C-terminus of one Fc subunit, the anchoring moiety comprises an antibody or antigen binding fragment thereof that is fused to the N-terminus of the other Fc subunit, and the cytokine moiety comprises a cytokine polypeptide fused to the masking moiety. In specific embodiments, the masking moiety and the anchoring moiety bind to the same Fc subunit. In specific embodiments, the masking moiety and the anchoring moiety bind to different Fc subunits. In some embodiments, the Fc domain comprises a modification promoting hetero-dimerization of the two Fc subunits. In specific embodiments, said modification is a knob-into-hole modification, comprising a knob modification in one of the Fc subunits (Fc-knob subunit) and a hole modification in the other one of the Fc subunits (Fc-hole subunit).

In one exemplary embodiment, the masking moiety comprises a bispecific two-in-one antibody or an antigen binding fragment thereof that is fused to the C-terminus of one Fc subunit. In one exemplary embodiment, the cytokine moiety comprises a cytokine polypeptide that is fused to the C-terminus of one Fc subunit. In one exemplary embodiment, the anchoring moiety comprises an antibody or antigen binding fragment thereof that is fused to the masking moiety. In some embodiments, the Fc domain comprises a modification promoting hetero-dimerization of the two Fc subunits. In specific embodiments, said modification is a knob-into-hole modification, comprising a knob modification in one of the Fc subunits (Fc-knob subunit) and a hole modification in the other one of the Fc subunits (Fc-hole subunit).

In one exemplary embodiment, the masking moiety comprises a bispecific two-in-one antibody or an antigen binding fragment thereof that is fused to the N-terminus of one Fc subunit. In one exemplary embodiment, the cytokine moiety comprises a cytokine polypeptide that is fused to the N-terminus of one Fc subunit. In one exemplary embodiment, the anchoring moiety comprises an antibody or antigen binding fragment thereof that is fused to the masking moiety. In some embodiments, the Fc domain comprises a modification promoting hetero-dimerization of the two Fc subunits. In specific embodiments, said modification is a knob-into-hole modification, comprising a knob modification in one of the Fc subunits (Fc-knob subunit) and a hole modification in the other one of the Fc subunits (Fc-hole subunit).

According to the present disclosure, in any of the embodiments described herein, the different moieties of the immunoconjugate molecule can be connected with a peptidic linker sequence. In some embodiments, the peptidic linker has at least 5 amino acid residues. In some embodiments, the peptidic linker has at least 7 amino acid residues. In some embodiments, the peptidic linker has at least 10 amino acid residues. In some embodiments, the peptidic linker has at least 15 amino acid residues. In some embodiments, the peptidic linker has at least 20 amino acid residues.

According to the present disclosure, in any of the embodiments described herein, non-limiting examples of an antibody forming part of the immunoconjugate molecule can be synthetic antibodies, recombinantly produced antibodies, camelized antibodies, intrabodies, anti-idiotypic (anti-Id) antibodies. In some embodiments, an antibody forming part of the immunoconjugate molecule is a monoclonal antibody. In any of the embodiments described herein, an antigen binding fragment forming part of the immunoconjugate molecule can be functional fragments of an antibody that retains some or all of the binding activity of the antibody from which the fragment was derived. Non-limiting examples of functional fragments (e.g., antigen-binding fragments such as IL-2-binding fragments) include single-chain Fvs (scFv) (e.g., including monospecific, bispecific, etc.), Fab fragments (e.g., including monospecific, bispecific, etc.), F(ab′) fragments, F(ab)₂ fragments, F(ab′)₂ fragments, disulfide-linked Fvs (dsFv), Fd fragments, Fv fragments, diabody, triabody, tetrabody, minibody, and single domain antibody (VHH or nanobody). In specific embodiments, the immunoconjugate molecule can have any of the configurations 1 to 20 as shown in FIG. 5.

For example, in specific embodiments, the bispecific two-in-one antibody in the masking moiety of the present immunoconjugate molecule is a Fab fragment. For example, in specific embodiments, the bispecific two-in-one antibody in the masking moiety of the present immunoconjugate molecule is a ScFv fragment. For example, in specific embodiments, the bispecific two-in-one antibody in the masking moiety of the present immunoconjugate molecule is a single domain (VHH) antibody.

For example, in specific embodiments, the antibody in the anchoring moiety of the present immunoconjugate molecule is a Fab fragment. For example, in specific embodiments, the antibody in the anchoring moiety of the present immunoconjugate molecule is a ScFv fragment. For example, in specific embodiments, the antibody in the anchoring moiety of the present immunoconjugate molecule is a single domain (VHH) antibody.

For example, in specific embodiments, the bispecific two-in-one antibody in the masking moiety of the present immunoconjugate molecule is a Fab fragment, and the antibody in the anchoring moiety of the immunoconjugate molecule is also a Fab fragment. For example, in specific embodiments, the bispecific two-in-one antibody in the masking moiety of the present immunoconjugate molecule is a Fab fragment, and the antibody in the anchoring moiety of the immunoconjugate molecule is a ScFv fragment. For example, in specific embodiments, the bispecific two-in-one antibody in the masking moiety of the present immunoconjugate molecule is a Fab fragment, and the antibody in the anchoring moiety of the immunoconjugate molecule is a single domain (VHH) fragment.

For example, in specific embodiments, the bispecific two-in-one antibody in the masking moiety of the present immunoconjugate molecule is a ScFv fragment, and the antibody in the anchoring moiety of the immunoconjugate molecule is a Fab fragment. For example, in specific embodiments, the bispecific two-in-one antibody in the masking moiety of the present immunoconjugate molecule is a ScFv fragment, and the antibody in the anchoring moiety of the immunoconjugate molecule is also ScFv fragment. For example, in specific embodiments, the bispecific two-in-one antibody in the masking moiety of the present immunoconjugate molecule is a ScFv fragment, and the antibody in the anchoring moiety of the immunoconjugate molecule is a single domain (VHH) fragment.

For example, in specific embodiments, the bispecific two-in-one antibody in the masking moiety of the present immunoconjugate molecule is a single domain (VHH) antibody, and the antibody in the anchoring moiety of the immunoconjugate molecule is a Fab fragment. For example, in specific embodiments, the bispecific two-in-one antibody in the masking moiety of the present immunoconjugate molecule is a single domain (VHH) antibody, and the antibody in the anchoring moiety of the immunoconjugate molecule is ScFv fragment. For example, in specific embodiments, the bispecific two-in-one antibody in the masking moiety of the present immunoconjugate molecule is a single domain (VHH) antibody, and the antibody in the anchoring moiety of the immunoconjugate molecule is also a single domain (VHH) fragment.

In specific embodiments, the bispecific two-in-one antibody or antigen binding fragment thereof forming part of the present immunoconjugate molecule is capable of binding to both an IL-2 polypeptide and fibrosis activation protein (FAP). In specific embodiments, the bispecific two-in-one antibody comprises a VH region, VL region, VH CDR1, VH CDR2, VH CDR3, VL CDR1, VL CDR2, and/or VL CDR3 of an amino acid sequence depicted in Tables 1-4. Accordingly, in some embodiments, the two-in-one antibody or functional fragment thereof provided herein comprises one, two, and/or three heavy chain CDRs and/or one, two, and/or three light chain CDRs from: (a) the antibody D001, (b) the antibody D002, (c) the antibody D029, (d) the antibody D003, (e) the antibody D047, (f) the antibody D049, (g) any one of the light chain variants D029LV1, D029LV2, D029LV3, D029LV4, and D029LV5, (h) any one of the heavy chain variants D029HV1, D029HV2, D029HV3, D029HV4, D029HV5, and D029HV6, or (i) the antibody B10 as shown in Tables 1-2. In specific embodiments, the two-in-one antibody or functional fragment thereof provided herein comprises one, two, and/or three heavy chain CDRs and/or one, two, and/or three light chain CDRs from the antibody D029-HV1LV1, the antibody D029-HV2LV3, the antibody D029-HV2LV4, the antibody D029-HV1LV5, the antibody D029-HV3LV2, the antibody D029-HV4LV2, or the antibody D029-HV6LV2. In some embodiments, the two-in-one antibody or functional fragment thereof provided herein comprises VH and VL regions selected from: (a) the antibody D001, (b) the antibody D002, (c) the antibody D029, (d) the antibody D003, (e) the antibody D047, (f) the antibody D049, (g) any one of the light chain variants D029LV1, D029LV2, D029LV3, D029LV4, and D029LV5, (h) any one of the heavy chain variants D029HV1, D029HV2, D029HV3, D029HV4, D029HV5, and D029HV6, or (i) the antibody B10 as shown in Tables 3-4. In specific embodiments, the two-in-one antibody or functional fragment thereof provided herein comprises VH and VL regions from the antibody D029-HV1LV1, the antibody D029-HV2LV3, the antibody D029-HV2LV4, the antibody D029-HV1LV5, the antibody D029-HV3LV2, the antibody D029-HV4LV2, or the antibody D029-HV6LV2. The nomenclature “D029-HVxLVx” refers to an antibody comprising the combination of VH and VL domain sequences of the corresponding numbers as shown in Tables 3-4. For example, “D029-HV2LV3” refers to an antibody comprising the VH domain sequence of D029HV2 and the VL domain sequence D029LV3 as shown in Tables 3-4).

TABLE 1
Two-In-One VL CDR Amino Acid Sequences

| | VL CDR1 | VL CDR2 | VL CDR3 |
|---|---|---|---|
| Antibody | (SEQ ID NO:) | (SEQ ID NO:) | (SEQ ID NO:) |
| D001 | RASQVIGSSLN | AASSLQS (SEQ ID | QQGRSYPYT (SEQ ID |
| | (SEQ ID NO: 6) | NO: 17) | NO: 18) |
| D002 | RASQSIGNSLN | AASSLQS (SEQ ID | QQGRRSPFT (SEQ ID |
| | (SEQ ID NO: 19) | NO: 17) | NO: 20) |
| D029 | RASQDGGNYLN | GASTLYS (SEQ ID | QQGRTPPLT (SEQ ID |
| | (SEQ ID NO: 21) | NO: 22) | NO: 23) |
| D029LV1 | RASQALGFYLN | AASSLSS (SEQ ID | QQGRTPPLT (SEQ ID |
| (D029-VL-Rev1) | (SEQ ID NO: 24) | NO: 25) | NO: 23) |
| D029LV2 | RASGALGFYLN | AASSLSS (SEQ ID | QQGRTYPFT (SEQ ID |
| (D029-VL-Rev1- | (SEQ ID NO: 26) | NO: 25) | NO: 27) |
| Q27G/P94Y/L96F) | | | |
| D029LV3 | RASGALGFYLN | AASSLSS (SEQ ID | QQGRTPPFT (SEQ ID |
| (D029-VL-Rev1- | (SEQ ID NO: 26) | NO: 25) | NO: 28) |
| Q27G/L96F) | | | |
| D029LV4 | RASGALGFYLN | AASSLSS (SEQ ID | QQGRTYPLT (SEQ ID |
| (D029-VL-Rev1- | (SEQ ID NO: 26) | NO: 25) | NO: 29) |
| Q27G/P94Y) | | | |
| D029LV5 | RASQALGFYLN | AASSLSS (SEQ ID | QQGRTYPLT (SEQ ID |
| (D029-VL-Rev1- | (SEQ ID NO: 24) | NO: 25) | NO: 29) |
| P94Y) | | | |
| D003 | RASQSISSYLN | AASSLQS (SEQ ID | QQTRSYLPT (SEQ ID |
| | (SEQ ID NO: 30) | NO: 17) | NO: 31) |
| D047 | RASQGI KNYLN | AASSLQS (SEQ ID | QOGSYLQPT (SEQ ID |
| | (SEQ ID NO: 32) | NO: 17) | NO: 33) |
| D049 | RASQIVRTYLN | AASSLQS (SEQ ID | QQGAQLQPT (SEQ ID |
| | (SEQ ID NO: 34) | NO: 17) | NO: 35) |
| B10 | RASQNIARYLN | AASSLQS (SEQ ID | QQGRYSPFT (SEQ ID |
| | (SEQ ID No: 103) | NO: 17) | NO: 104) |

TABLE 2
Two-In-One VH CDR Amino Acid Sequences

| | VH CDR1 | VH CDR2 | VH CDR3 |
|---|---|---|---|
| Antibody | (SEQ ID NO:) | (SEQ ID NO:) | (SEQ ID NO:) |
| D001 | RYSMS (SEQ ID | AISPYGGGTDYADSVKG | SQYGRYYSPDYYFDY |
| | NO: 36) | (SEQ ID NO: 37) | (SEQ ID NO: 38) |
| D002 | RYSMS (SEQ ID | DISSYSTTYYADSVKG | SQYGRYYSPDYYFDY |
| | NO: 36) | (SEQ ID NO: 39) | (SEQ ID NO: 38) |
| D029 | WSFMS (SEQ ID | WIGPSGSYTDYADSVKG | SQYGRYYSPDYYFDY |
| | NO: 40) | (SEQ ID NO: 41) | (SEQ ID NO: 38) |
| D029HV1 | WSFMS (SEQ ID | WIGPSGLYTDYADSVKG | SQYGRYYSPDYYFDY |
| (D029-VH-S55L) | NO: 40) | (SEQ ID NO: 42) | (SEQ ID NO: 38) |
| D029HV2 | WFFMS (SEQ ID | WIGPSGLYTDYADSVKG | SQYGRYYSPDYYFDY |
| (D029-VH- | NO: 43) | (SEQ ID NO: 42) | (SEQ ID NO: 38) |
| S55L/S32F) | | | |
| D029HV3 | WFFMS (SEQ ID | WIGPSGLYTDYADSVKG | SQYGRYYSPDYYFDY |
| (D029-VH- | NO: 43) | (SEQ ID NO: 42) | (SEQ ID NO: 38) |
| S55L/T30R/S32F) | | | |
| D029HV4 | RFFMS (SEQ ID | WIGPSGLYTDYADSVKG | SQYGRYYSPDYYFDY |
| (D029-VH-H1V9) | NO: 44) | (SEQ ID NO: 42) | (SEQ ID NO: 38) |
| D029HV5 | WAFMS (SEQ ID | WIGPSGLYTDYADSVKG | SQYGRYYSPDYYFDY |
| (D029-VH- | NO: 45) | (SEQ ID NO: 42) | (SEQ ID NO: 38) |
| S55L/T30R/S32A) | | | |
| D029HV6 | WAFMS (SEQ ID | WIGPSGLYTDYADSVKG | SQYGRYYSPDYYFDY |
| (D029-VH- | NO: 45) | (SEQ ID NO: 42) | (SEQ ID NO: 38) |
| S55L/S32A) | | | |
| D003 | TYIMS (SEQ ID | SIGPSYGDTIYADSVKG | TISYSQYAYGYSFDY |
| | NO: 46) | (SEQ ID NO: 47) | (SEQ ID NO: 48) |
| D047 | YFSMS (SEQ ID | SISPTYGTTDYADSVKG | LLYGTWFDY (SEQ ID |
| | NO: 49) | (SEQ ID NO: 50) | NO: 51) |
| D049 | SYWMS (SEQ ID | TISPYDSSTAYADSVKG | LLYGTWFDY (SEQ ID |
| | NO: 52) | (SEQ ID NO: 53) | NO: 51) |
| B10 | SRYSMG (SEQ ID | TIHSHGSGTRYADSVKG | SQYGRYYSPDYYFDY |
| | NO: 105) | (SEQ ID NO: 106) | (SEQ ID NO: 38) |

TABLE 3
Two-In-One VL Domain Amino Acid Sequences

| Antibody | VL (SEQ ID NO:) |
|---|---|
| D001 | DIQMTQSPSSLSASVGDRVTITCRASQVIGSSLNWYQQKPGKAPKLLIYAASSLQSGVP |
| | SRFSGSGSGTDFTLTISSLQPEDFATYYCQQGRSYPYTFGQGTKVEIKRTVA (SEQ |
| | ID NO: 68) |
| D002 | DIQMTQSPSSLSASVGDRVTITCRASQSIGNSLNWYQQKPGKAPKLLIYAASSLQSGVP |
| | SRFSGSGSGTDFTLTISSLQPEDFATYYCQQGRRSPFTFGQGTKVEIKRTVA (SEQ |
| | ID NO: 69) |
| D029 | DIQMTQSPSSLSASVGDRVTITCRASQDGGNYLNWYQQKPGKAPKLLIYGASTLYSGVP |
| | SRFSGSGSGTDFTLTISSLQPEDFATYYCQQGRTPPLTFGQGTKVEIKRTVA (SEQ |
| | ID NO: 70) |
| D029LV1 | DIQMTQSPSSLSASVGDRVTITCRASQALGFYLNWYQQKPGKAPKLLIYAASSLSSGVP |
| (D029-VL- | SRFSGSGSGTDFTLTISSLQPEDFATYYCQQGRTPPLTFGQGTKVEIKRTVA (SEQ |
| Rev1) | ID NO: 71) |
| D029LV2 | DIQMTQSPSSLSASVGDRVTITCRASGALGFYLNWYQQKPGKAPKLLIYAASSLSSGVP |
| (D029-VL- | SRFSGSGSGTDFTLTISSLQPEDFATYYCQQGRTYPFTFGQGTKVEIKRTVA (SEQ |
| Rev1-Q27G/ | ID NO: 72) |
| P94Y/L96F) | |
| D029LV3 | DIQMTQSPSSLSASVGDRVTITCRASGALGFYLNWYQQKPGKAPKLLIYAASSLSSGVP |
| (D029-VL- | SRFSGSGSGTDFTLTISSLQPEDFATYYCQQGRTPPFTFGQGTKVEIKRTVA (SEQ |
| Rev1-Q27G/ | ID NO: 73) |
| L96F) | |
| D029LV4 | DIQMTQSPSSLSASVGDRVTITCRASGALGFYLNWYQQKPGKAPKLLIYAASSLSSGVP |
| (D029-VL- | SRFSGSGSGTDFTLTISSLQPEDFATYYCQQGRTYPLTFGQGTKVEIKRTVA (SEQ |
| Rev1-Q27G/ | ID NO: 74) |
| P94Y) | |
| D029LV5 | DIQMTQSPSSLSASVGDRVTITCRASQALGFYLNWYQQKPGKAPKLLIYAASSLSSGVP |
| (D029-VL- | SRFSGSGSGTDFTLTISSLQPEDFATYYCQQGRTYPLTFGQGTKVEIKRTVA (SEQ |
| Rev1-P94Y) | ID NO: 75) |
| D003 | DIQMTQSPSSLSASVGDRVTITCRASQSISSYLNWYQQKPGKAPKLLIYAASSLQSGVP |
| | SRFSGSGSGTDFTLTISSLQPEDFATYYCQQTRSYLPTFGQGTKVEIKRTVA (SEQ |
| | ID NO: 76) |
| D047 | DIQMTQSPSSLSASVGDRVTITCRASQGIKNYLNWYQQKPGKAPKLLIYAASSLQSGVP |
| | SRFSGSGSGTDFTLTISSLQPEDFATYYCQQGSYLQPTFGQGTKVEIKRTVA (SEQ |
| | ID NO: 77) |
| D049 | DIQMTQSPSSLSASVGDRVTITCRASQIVRTYLNWYQQKPGKAPKLLIYAASSLQSGVP |
| | SRFSGSGSGTDFTLTISSLQPEDFATYYCQQGAQLQPTFGQGTKVEIKRTVA (SEQ |
| | ID NO: 78) |
| B10 | DIQMTQSPSSLSASVGDRVTITCRASQNIARYLNWYQQKPGKAPKLLIYAASSLQSGVP |
| | SRFSGSGSGTDFTLTISSLQPEDFATYYCQQGRYSPFTFGQGTKVEIKR (SEQ ID |
| | NO: 101) |

TABLE 4
Two-In-One VH Domain Amino Acid Sequences

| Antibody | VH (SEQ ID NO:) |
|---|---|
| D001 | EVQLVESGGGLVQPGGSLRLSCAASGFTFSRYSMSWVRQAPGKGLEWVSAISPYGGGTD |
| | YADSVKGRFTISRDNSKNTLYLQMNSLRAEDTAVYYCARSQYGRYYSPDYYFDYWGQGT |
| | LVTVSS (SEQ ID NO: 79) |
| D002 | EVQLVESGGGLVQPGGSLRLSCAASGFTFSRYSMSWVRQAPGKGLEWVSDISSYSTTYY |
| | ADSVKGRFTISRDNSKNTLYLQMNSLRAEDTAVYYCARSQYGRYYSPDYYFDYWGQGTL |
| | VTVSS (SEQ ID NO: 80) |
| D029 | EVQLVESGGGLVQPGGSLRLSCAASGFTFTWSFMSWVRQAPGKGLEWVSWIGPSGSYTD |
| | YADSVKGRFTISRDNSKNTLYLQMNSLRAEDTAVYYCARSQYGRYYSPDYYFDYWGQGT |
| | LVTVSS (SEQ ID NO: 81) |
| D029HV1 | EVQLVESGGGLVQPGGSLRLSCAASGFTFTWSFMSWVRQAPGKGLEWVSWIGPSGLYTD |
| (D029-VH- | YADSVKGRFTISRDNSKNTLYLQMNSLRAEDTAVYYCARSQYGRYYSPDYYFDYWGQGT |
| S55L) | LVTVSS (SEQ ID NO: 82) |
| D029HV2 | EVQLVESGGGLVQPGGSLRLSCAASGFTFTWFFMSWVRQAPGKGLEWVSWIGPSGLYTD |
| (D029-VH- | YADSVKGRFTISRDNSKNTLYLQMNSLRAEDTAVYYCARSQYGRYYSPDYYFDYWGQGT |
| S55L/S32F) | LVTVSS (SEQ ID NO: 83) |
| D029HV3 | EVQLVESGGGLVQPGGSLRLSCAASGFTFRWFFMSWVRQAPGKGLEWVSWIGPSGLYTD |
| (D029-VH- | YADSVKGRFTISRDNSKNTLYLQMNSLRAEDTAVYYCARSQYGRYYSPDYYFDYWGQGT |
| S55L/T30R/ | LVTVSS (SEQ ID NO: 84) |
| S32F) | |
| D029HV4 | EVQLVESGGGLVQPGGSLRLSCAASGFTFSRFFMSWVRQAPGKGLEWVSWIGPSGLYTD |
| (D029-VH- | YADSVKGRFTISRDNSKNTLYLQMNSLRAEDTAVYYCARSQYGRYYSPDYYFDYWGQGT |
| H1V9) | LVTVSS (SEQ ID NO: 85) |
| D029HV5 | EVQLVESGGGLVQPGGSLRLSCAASGFTFRWAFMSWVRQAPGKGLEWVSWIGPSGLYTD |
| (D029-VH- | YADSVKGRFTISRDNSKNTLYLQMNSLRAEDTAVYYCARSQYGRYYSPDYYFDYWGQGT |
| S55L/T30R/ | LVTVSS (SEQ ID NO: 86) |
| S32A) | |
| D029HV6 | EVQLVESGGGLVQPGGSLRLSCAASGFTFTWAFMSWVRQAPGKGLEWVSWIGPSGLYTD |
| (D029-VH- | YADSVKGRFTISRDNSKNTLYLQMNSLRAEDTAVYYCARSQYGRYYSPDYYFDYWGQGT |
| S55L/S32A) | LVTVSS (SEQ ID NO: 87) |
| D003 | EVQLVESGGGLVQPGGSLRLSCAASGFTFYTYIMSWVRQAPGKGLEWVSSIGPSYGDTI |
| | YADSVKGRFTISRDNSKNTLYLQMNSLRAEDTAVYYCARTISYSQYAYGYSFDYWGQGT |
| | LVTVSS (SEQ ID NO: 88) |
| D047 | EVQLVESGGGLVQPGGSLRLSCAASGFTFHYFSMSWVRQAPGKGLEWVSSISPTYGTTD |
| | YADSVKGRFTISRDNSKNTLYLQMNSLRAEDTAVYYCARLLYGTWFDYWGQGTLVTVSS |
| | (SEQ ID NO: 89) |
| D049 | EVQLVESGGGLVQPGGSLRLSCAASGFTFFSYWMSWVRQAPGKGLEWVSTISPYDSSTA |
| | YADSVKGRFTISRDNSKNTLYLQMNSLRAEDTAVYYCARLLYGTWFDYWGQGTLVTVSS |
| | (SEQ ID NO: 90) |
| B10 | EVQLVESGGGLVQPGGSLRLSCAASGFTFSRYSMGWVRQAPGKGLEWVSTIHSHGSGTR |
| | YADSVKGRFTISRDNSKNTLYLQMNSLRAEDTAVYYCARSQYGRYYSPDYYFDYWGQGT |
| | LVTVSS (SEQ ID NO: 102) |

In specific embodiments, the anchoring moiety of the present immunoconjugate molecule comprises an antibody or antigen binding fragment thereof that binds to fibrosis activation protein (FAP). In specific embodiments, the anti-FAP antibody comprises a VH region, VL region, VH CDR1, VH CDR2, VH CDR3, VL CDR1, VL CDR2, and/or VL CDR3 of an amino acid sequence depicted in Tables 5-8. Accordingly, in some embodiments, the anti-FAP antibody or functional fragment thereof provided herein comprises one, two, and/or three heavy chain CDRs and/or one, two, and/or three light chain CDRs from: (a) the antibody 872-5, (b) the antibody 872-59, (c) 872-70, (d) 872-5V1, or (e) VHH6 as shown in Tables 5-6. In some embodiments, the anti-FAP antibody or functional fragment thereof provided herein comprises VH and VL regions from: (a) the antibody 872-5, (b) the antibody 872-59, (c) 872-70, (d) 872-5V1, or (e) VHH6 as shown in Tables 7-8.

TABLE 5
Anti-FAP VL CDR Amino Acid Sequences

| | VL CDR1 | VL CDR2 | VL CDR3 |
|---|---|---|---|
| Antibody | (SEQ ID NO:) | (SEQ ID NO:) | (SEQ ID NO:) |
| 872-5 | RASQSISSYLN | AASSLQS | QQATLLLPT |
| | (SEQ ID | (SEQ ID | (SEQ ID |
| | NO: 30) | NO: 17) | NO: 54) |
| 872-59 | RASQSISSYLN | AASSLQS | QQAFTSPRT |
| | (SEQ ID | (SEQ ID | (SEQ ID |
| | NO: 30) | NO: 17) | NO: 55) |
| 872-70 | RASQSISSYLN | AASSLQS | QQSRTSPYT |
| | (SEQ ID | (SEQ ID | (SEQ ID |
| | NO: 30) | NO: 17) | NO: 56) |
| 872-5V1 | RASQSISSYLN | AASSLQS | QQGTLLLPTF |
| | (SEQ ID | (SEQ ID | (SEQ ID |
| | NO: 30) | NO: 17) | NO: 57) |

TABLE 6
Anti-FAP VH CDR Amino Acid Sequences

| | VH CDR1 | | VH CDR3 |
|---|---|---|---|
| Antibody | (SEQ ID NO:) | VH CDR2 (SEQ ID NO:) | (SEQ ID NO:) |
| 872-5 | GYIMS | WISPEGSRTGYADSVKG | LQYGTWFDY (SEQ ID |
| | (SEQ ID NO: 58) | (SEQ ID NO: 59) | NO: 60) |
| 872-59 | SYAMS | SIYSWYGTTSYADSVKG | TISYSQYAYGYSFDY (SEQ |
| | (SEQ ID NO: 61) | (SEQ ID NO: 62) | ID NO: 48) |
| 872-70 | RYSMS | YITSTDGTTDYADSVKG | SQYGRYYSPDYYFDY (SEQ |
| | (SEQ ID NO: 36) | (SEQ ID NO: 63) | ID NO: 38) |
| 872-5V1 | GYIMS | WIGPEGSRTGYADSVKG | LLYGTWFDY (SEQ ID |
| | (SEQ ID NO: 58) | (SEQ ID NO: 64) | NO: 51) |
| VHH6 | PTYMY | AIDPYGRTEYPDSVKG | RVPPGVYTYYRTGPYDY |
| | (SEQ ID NO: 65) | (SEQ ID NO: 66) | (SEQ ID NO: 67) |

TABLE 7
Anti-FAP VL Domain Amino Acid Sequences

| Antibody | VL (SEQ ID NO:) |
|---|---|
| 872-5 | DIQMTQSPSSLSASVGDRVTITCRASQSISSYLNWYQQKPGKAPKLLIYAASSLQSGVPSRFSGSG |
| | SGTDFTLTISSLQPEDFATYYCQQATLLLPTFGQGTKVEIKRTVA (SEQ ID NO: 91) |
| 872-59 | DIQMTQSPSSLSASVGDRVTITCRASQSISSYLNWYQQKPGKAPKLLIYAASSLQSGVPSRFSGSG |
| | SGTDFTLTISSLQPEDFATYYCQQAFTSPRTFGQGTKVEIKRTVA (SEQ ID NO: 92) |
| 872-70 | DIQMTQSPSSLSASVGDRVTITCRASQSISSYLNWYQQKPGKAPKLLIYAASSLQSGVPSRFSGSG |
| | SGTDFTLTISSLQPEDFATYYCQQSRTSPYTFGQGTKVEIKRTVA (SEQ ID NO: 93) |
| 872-5V1 | DIQMTQSPSSLSASVGDRVTITCRASQSISSYLNWYQQKPGKAPKLLIYAASSLQSGVPSRFSGSG |
| | SGTDFTLTISSLQPEDFATYYCQQGTLLLPTFGQGTKVEIKRTVA (SEQ ID NO: 94) |

TABLE 8
Anti-FAP VH Domain Amino Acid Sequences

| Antibody | VH (SEQ ID NO:) |
|---|---|
| 872-5 | EVQLVESGGGLVQPGGSLRLSCAASGFTFRGYIMSWVRQAPGKGLEWVSWISPEGSRTGYADSVKG |
| | RFTISRDNSKNTLYLQMNSLRAEDTAVYYCARLQYGTWFDYWGQGTLVTVSS (SEQ ID |
| | NO: 95) |
| 872-59 | EVQLVESGGGLVQPGGSLRLSCAASGFTFTSYAMSWVRQAPGKGLEWVSSIYSWYGTTSYADSVKG |
| | RFTISRDNSKNTLYLQMNSLRAEDTAVYYCARTISYSQYAYGYSFDYWGQGTLVTVSS (SEQ ID |
| | NO: 96) |
| 872-70 | EVQLVESGGGLVQPGGSLRLSCAASGFTFSRYSMSWVRQAPGKGLEWVSYITSTDGTTDYADSVKG |
| | RFTISRDNSKNTLYLQMNSLRAEDTAVYYCARSQYGRYYSPDYYFDYWGQGTLVTVSS (SEQ ID |
| | NO: 97) |
| 872-5V1 | EVQLVESGGGLVQPGGSLRLSCAASGFTFRGYIMSWVRQAPGKGLEWVSWIGPEGSRTGYADSVKG |
| | RFTISRDNSKNTLYLQMNSLRAEDTAVYYCARLLYGTWFDYWGQGTLVTVSS (SEQ ID |
| | NO: 98) |
| VHH6 | EVQLVESGGGLVQPGGSLRLSCAASGFTFTPTYMYWVRQAPGKGLEWVSAIDPYGRTEYPDSVKGR |
| | FTISRDNAKNTLYLQMNSLRPEDTAVYYCAVRVPPGVYTYYRTGPYDYWGQGTLVTVSS (SEQ |
| | ID NO: 99) |

In one particular aspect, provided herein are IL-2 containing immunoconjugate molecules that modulate IL-2 activity by reversible binding and disassociation from the IL-2 region responsible for binding with a particular IL-2R subunit. In some embodiments, the IL-2 polypeptide in the immunoconjugate molecule further comprises one or more mutations that modifying binding activity of the IL-2 polypeptide to a particular IL-2R subunit.

In some embodiments, the immunoconjugate molecule comprises an IL-2 polypeptide conjugated to a masking moiety, wherein the masking moiety comprises a two-in-one antibody or antigen binding fragment thereof capable of binding to the IL-2 polypeptide and a first target antigen; wherein when binding to the IL-2 polypeptide, the masking moiety blocks binding of the IL-2 polypeptide to IL-2 receptor a subunit (IL-2Rα); and wherein when binding to the first target antigen, the masking moiety disassociates from the IL-2 polypeptide, thereby releasing the IL-2 polypeptide for binding with IL-2Rα, and wherein the IL-2 polypeptide comprises one or more mutations that attenuate binding of the IL-2 polypeptide to the IL-2Rβ. In some embodiments, the IL-2 polypeptide further comprises one or more mutations that modifying binding of the IL-2 polypeptide to IL-2Rγ.

In some embodiments, the immunoconjugate molecule comprises an IL-2 polypeptide conjugated to a masking moiety, wherein the masking moiety comprises a two-in-one antibody or antigen binding fragment thereof capable of binding to the IL-2 polypeptide and a first target antigen; wherein when binding to the IL-2 polypeptide, the masking moiety blocks binding of the IL-2 polypeptide to IL-2 receptor a subunit (IL-2Rβ); and wherein when binding to the first target antigen, the masking moiety disassociates from the IL-2 polypeptide, thereby releasing the IL-2 polypeptide for binding with IL-2Rβ, and wherein the IL-2 polypeptide comprises one or more mutations that attenuate binding of the IL-2 polypeptide to the IL-2Rα. In some embodiments, the IL-2 polypeptide further comprises one or more mutations that modifying binding of the IL-2 polypeptide to IL-2Rγ.

In some embodiments, the masking moiety blocks binding of the IL-2 polypeptide to the IL-2Rα subunit. In some embodiments, the masking moiety binds to an epitope of IL-2 comprising one or more of the residues P34, K35, R38, T41, F42, K43, F44, Y45, E61, E62, K64, P65, E68, V69, N71, L72, Q74, Y107, and D109 of IL-2.

In some embodiments, the masking moiety blocks binding of the IL-2 polypeptide to the IL-2Rα subunit. In specific embodiments, the masking moiety binds to an epitope of IL-2 recognized by an antibody comprising a light chain variable region having an amino acid sequence of SEQ ID NO: 101 and a heavy chain variable region having an amino acid sequence of SEQ ID NO: 102. In some embodiments, the masking moiety competes for binding with IL-2 with an antibody comprising a light chain variable region having an amino acid sequence of SEQ ID NO: 101 and a heavy chain variable region having an amino acid sequence of SEQ ID NO: 102. In some embodiments, the masking moiety comprises (a) a light chain variable region (VL) comprising VL complementarity determining region 1 (CDR1), VL CDR2, and VL CDR3 of antibody B10 as set forth in Table 1; and/or (b) a heavy chain variable region (VH) comprising VH complementarity determining region 1 (CDR1), VH CDR2, and VH CDR3 of antibody B10 as set forth in Table 2. In some embodiments, wherein the masking moiety comprises (a) the VL CDR1, VL CDR2, and VL CDR3 comprising amino acid sequences of SEQ ID NOS:103, 17, and 104, respectively, and (b) the VH CDR1, VH CDR2, and VH CDR3 comprising amino acid sequences of SEQ ID NOS:105, 106, and 38, respectively. In some embodiments, wherein the masking moiety comprises: (a) a light chain variable region (VL) comprising VL of antibody B10 as set forth in Table 3; and/or (b) a heavy chain variable region (VH) comprising VH of antibody B10 as set forth in Table 4. In some embodiments, wherein the masking moiety comprises a VL comprising an amino acid sequence of SEQ ID NO: 101. In some embodiments, wherein the masking moiety comprises a VH comprising an amino acid sequence of SEQ ID NO: 102. In some embodiments, wherein the masking moiety comprises (a) a VL comprising an amino acid sequence of SEQ ID NO: 101; and (b) a VH comprising an amino acid sequence of SEQ ID NO: 102.

In some embodiments, the masking moiety blocks binding of the IL-2 polypeptide to IL-2Rβ. In some embodiments, the masking moiety binds to an epitope of IL-2 comprising one or more of the residues L12, Q13, E15, H16, L19, D20, M23, R81, D84, D87, N88, V91, 192, and E95 or IL-2. In some embodiments, the masking moiety binds to an epitope of IL-2 recognized by the antibody 5UTZ. In some embodiments, the masking moiety competes for binding with IL-2 with antibody 5UTZ.

In some embodiments, the IL-2 polypeptide of the immunoconjugate molecule comprises one or more mutations that attenuate binding of the IL-2 polypeptide to the IL-2Rα. In some embodiments, the one or more mutations that attenuate binding of the IL-2 polypeptide to IL-2Rα are selected from K35E, R38A, R38E, R38D, F42A, F42K, K43E, Y45A, E61R, E62A, L72G, or a combination thereof. In some embodiments, the one or more mutations that attenuate binding of the IL-2 polypeptide to IL-2Rα comprise any one, two, three, four, five, six, seven or eight mutations selected from K35E, R38A, R38E, R38D, F42A, F42K, K43E, Y45A, E61R, E62A, L72G. For example, in some embodiments, the one or more mutations that attenuate binding of the IL-2 polypeptide to IL-2Rα comprise F42A. In some embodiments, the one or more mutations that attenuate binding of the IL-2 polypeptide to IL-2Rα comprise K35E and F42A. In some embodiments, the one or more mutations that attenuate binding of the IL-2 polypeptide to IL-2Rα comprises F42A, Y45A, and L72G. In some embodiments, the one or more mutations that attenuate binding of the IL-2 polypeptide to IL-2Rα comprise R38D, K43E, E61R. In some embodiments, the one or more mutations that attenuate binding of the IL-2 polypeptide to IL-2Rα comprise R38A, F42A, Y45A, and E62A. In some embodiments, the binding of the IL-2 polypeptide to IL-2Rα subunit is reduced about 10%, about 20%, about 30%, about 40%, about 45%, about 50%, about 55%, about 60%, about 65%, about 70%, about 75%, about 80%, about 85%, about 90%, about 95%, or about 99% comparing to wild-type IL-2. In some embodiments, the binding of the IL-2 polypeptide to IL-2Rα subunit is reduced about 0.5% to 10%, about 10% to 20%, about 20% to 30%, about 30% to 40%, about 40% to 45%, about 45% to 50%, about 50% to 55%, about 55% to 60%, about 60% to 65%, about 65% to 70%, about 70% to 75%, about 75% to 80%, about 80% to 85%, about 85% to 90%, about 90% to 95%, or 95% to about 99% comparing to wild-type IL-2.

In some embodiments, the IL-2 polypeptide of the immunoconjugate molecule comprises one or more mutations that attenuate binding of the IL-2 polypeptide to the IL-2RP. In some embodiments, the one or more mutations that attenuate binding of the IL-2 polypeptide to IL-2Rβ are selected from H16E, H16R, H16A, D20T, D20G, D20A, N88D, N88S, N88R, V91G, V91A, V91R, and V91S, or a combination thereof. In some embodiments, the one or more mutations that attenuate binding of the IL-2 polypeptide to IL-2RP comprise any one, two, three or four mutations selected from H16E, H16R, H16A, D20T, D20G, D20A, N88D, N88S, N88R, V91G, V91A, V91R, and V91S. In some embodiments, the binding of the IL-2 polypeptide to IL-2Rβ subunit is reduced about 10%, about 20%, about 30%, about 40%, about 45%, about 50%, about 55%, about 60%, about 65%, about 70%, about 75%, about 80%, about 85%, about 90%, about 95%, or about 99% comparing to wild-type IL-2. In some embodiments, the binding of the IL-2 polypeptide to IL-2Rα subunit is reduced about 0.5% to 10%, about 10% to 20%, about 20% to 30%, about 30% to 40%, about 40% to 45%, about 45% to 50%, about 50% to 55%, about 55% to 60%, about 60% to 65%, about 65% to 70%, about 70% to 75%, about 75% to 80%, about 80% to 85%, about 85% to 90%, about 90% to 95%, or 95% to about 99% comparing to wild-type IL-2.

In some embodiments, the IL-2 polypeptide further comprises one or more mutations that modifying binding of the IL-2 polypeptide to IL-2R y-chain (IL-2Rγ). In some embodiments, the one or more mutations modifying binding of the IL-2 polypeptide to IL-2Rγ is selected from L18R, Q22E, Q74H, L80F, R81D, L85V, I92F, T123A, Q126X where X=H, M, K, R, E, S, G, A, C, D, I or T, 1129V, S130A, S130R, or a combination thereof. In some embodiments, the one or more mutations modifying binding of the IL-2 polypeptide to IL-2Rγ comprises any one, two, three, four, five, six, seven, eight, night ten, or eleven mutations selected from L18R, Q22E, Q74H, L80F, R81D, L85V, I92F, T123A, Q126X where X=H, M, K, R, E, S, G, A, C, D, I or T, 1129V, S130A, and S130R. For example, in some embodiments, the one or more mutations modifying binding of the IL-2 polypeptide to IL-2Rγ comprises Q126T, Q74H, L80F, R81D, L85V, and I92F. In some embodiments, the one or more mutations modifying binding of the IL-2 polypeptide to IL-2Rγ comprises L18R, Q22E, Q126T, and S130R. In some embodiments, the binding of the IL-2 polypeptide to IL-2Rγ subunit is enhanced or reduced about 10%, about 20%, about 30%, about 40%, about 45%, about 50%, about 55%, about 60%, about 65%, about 70%, about 75%, about 80%, about 85%, about 90%, about 95%, or about 99% comparing to wild-type IL-2. In some embodiments, the binding of the IL-2 polypeptide to IL-2Rα subunit is reduced about 0.5% to 10%, about 10% to 20%, about 20% to 30%, about 30% to 40%, about 40% to 45%, about 45% to 50%, about 50% to 55%, about 55% to 60%, about 60% to 65%, about 65% to 70%, about 70% to 75%, about 75% to 80%, about 80% to 85%, about 85% to 90%, about 90% to 95%, or 95% to about 99% comparing to wild-type IL-2.

In some embodiments, the IL-2 containing immunoconjugate molecule as describe herein further comprises an anchoring moiety as described herein. In some embodiments, the anchoring moiety comprises an antibody or antigen binding fragment thereof that specifically binds to a second target antigen. In some embodiments, wherein the masking moiety disassociate from the IL-2 polypeptide in the presence of the first target antigen expressed on the surface of a first cell.

In some embodiments, wherein the second target antigen is expressed on the surface of the first cell or a second cell in proximity of the first cell. In some embodiments, the first target antigen and the second target antigen are the same or different. In some embodiments, the first target antigen and/or the second target antigen is a tumor associated antigen. In some embodiments, the first target antigen and the second target antigen are each independently selected from FAP, Her2, Her3, CD19, CD20, BCMA, PSMA, CEA, cMET, EGFR, CA-125, MUC-1, EpCAM, or Trop-2. In some embodiments, the first target antigen is FAP.

In some embodiments, the IL-2 containing immunoconjugate molecule as describe herein further comprises a conjugating moiety as described herein.

### 5.3.1 Polyclonal Antibodies

The antibodies forming part of the immunoconjugate molecules of the present disclosure may comprise polyclonal antibodies. Methods of preparing polyclonal antibodies are known to the skilled artisan. Polyclonal antibodies can be raised in a mammal, for example, by one or more injections of an immunizing agent and, if desired, an adjuvant. Typically, the immunizing agent and/or adjuvant will be injected in the mammal by multiple subcutaneous or intraperitoneal injections. The immunizing agent may include a polypeptide or a fusion protein thereof (e.g., IL-2 polypeptide or FAP polypeptide). It may be useful to conjugate the immunizing agent to a protein known to be immunogenic in the mammal being immunized or to immunize the mammal with the protein and one or more adjuvants. Examples of such immunogenic proteins include, but are not limited to, keyhole limpet hemocyanin, serum albumin, bovine thyroglobulin, and soybean trypsin inhibitor. Examples of adjuvants which may be employed include Ribi, CpG, Poly 1C, Freund's complete adjuvant, and MPL-TDM adjuvant (monophosphoryl Lipid A, synthetic trehalose dicorynomycolate). The immunization protocol may be selected by one skilled in the art without undue experimentation. The mammal can then be bled, and the serum assayed for antibody titer. If desired, the mammal can be boosted until the antibody titer increases or plateaus. Additionally or alternatively, lymphocytes may be obtained from the immunized animal for fusion and preparation of monoclonal antibodies from hybridoma as described below.

### 5.3.2 Monoclonal Antibodies

The antibodies forming part of the immunoconjugate molecules of the present disclosure may alternatively be monoclonal antibodies. Monoclonal antibodies may be made using the hybridoma method first described by Kohler et al., 1975, Nature 256:495-97, or may be made by recombinant DNA methods (see, e.g., U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice 59-103 (1986)).

The hybridoma cells thus prepared are seeded and grown in a suitable culture medium which, in certain embodiments, contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which prevent the growth of HGPRT-deficient cells.

Exemplary fusion partner myeloma cells are those that fuse efficiently, support stable high-level production of antibody by the selected antibody-producing cells, and are sensitive to a selective medium that selects against the unfused parental cells. Exemplary myeloma cell lines are murine myeloma lines, such as SP-2 and derivatives, for example, X63-Ag8-653 cells available from the American Type Culture Collection (Manassas, VA), and those derived from MOPC-21 and MPC-11 mouse tumors available from the Salk Institute Cell Distribution Center (San Diego, CA). Human myeloma and mouse-human heteromyeloma cell lines also have been described for the production of human monoclonal antibodies (Kozbor, 1984, Immunol. 133:3001-05; and Brodeur et al., Monoclonal Antibody Production Techniques and Applications 51-63 (1987)).

Culture medium in which hybridoma cells are growing is assayed for production of monoclonal antibodies directed against the antigen. The binding specificity of monoclonal antibodies produced by hybridoma cells is determined by immunoprecipitation or by an in vitro binding assay, such as RIA or ELISA. The binding affinity of the monoclonal antibody can, for example, be determined by the Scatchard analysis described in Munson et al., 1980, Anal. Biochem. 107:220-39.

Once hybridoma cells that produce antibodies of the desired specificity, affinity, and/or activity are identified, the clones may be subcloned by limiting dilution procedures and grown by standard methods (Goding, supra). Suitable culture media for this purpose include, for example, DMEM or RPMI-1640 medium. In addition, the hybridoma cells may be grown in vivo as ascites tumors in an animal, for example, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells can serve as a source of such DNA. Once isolated, the DNA may be placed into expression vectors, which are then transfected into host cells, such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., 1993, Curr. Opinion in Immunol. 5:256-62 and Pluckthun, 1992, Immunol. Revs. 130:151-88.

In some embodiments, an antibody that binds an epitope comprises an amino acid sequence of a VH domain and/or an amino acid sequence of a VL domain encoded by a nucleotide sequence that hybridizes to (1) the complement of a nucleotide sequence encoding any one of the VH and/or VL domain described herein under stringent conditions (e.g., hybridization to filter-bound DNA in 6X sodium chloride/sodium citrate (SSC) at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art. See, e.g., Current Protocols in Molecular Biology Vol. I, 6.3.1-6.3.6 and 2.10.3 (Ausubel et al. eds., 1989).

In some embodiments, an antibody that binds a FAP epitope comprises an amino acid sequence of a VH CDR or an amino acid sequence of a VL CDR encoded by a nucleotide sequence that hybridizes to the complement of a nucleotide sequence encoding any one of the VH CDRs and/or VL CDRs depicted in Tables 5-6 under stringent conditions (e.g., hybridization to filter-bound DNA in 6×SSC at about 45° C. followed by one or more washes in 0.2×SSC/0.1% SDS at about 50-65° C.), under highly stringent conditions (e.g., hybridization to filter-bound nucleic acid in 6×SSC at about 45° C. followed by one or more washes in 0.1×SSC/0.2% SDS at about 68° C.), or under other stringent hybridization conditions which are known to those of skill in the art (see, e.g., Ausubel et al., supra).

In a further embodiment, monoclonal antibodies or antibody fragments can be isolated from antibody phage libraries generated using the techniques described in, for example, Antibody Phage Display: Methods and Protocols (O'Brien and Aitken eds., 2002). In principle, synthetic antibody clones are selected by screening phage libraries containing phages that display various fragments of antibody variable region (Fv) fused to phage coat protein. Such phage libraries are screened against the desired antigen. Clones expressing Fv fragments capable of binding to the desired antigen are adsorbed to the antigen and thus separated from the non-binding clones in the library. The binding clones are then eluted from the antigen and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., 1994, Ann. Rev. Immunol. 12:433-55.

Repertoires of VH and VL genes can be separately cloned by PCR and recombined randomly in phage libraries, which can then be searched for antigen-binding clones as described in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described by Griffiths et al., 1993, EMBO J 12:725-34. Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, 1992, J. Mol. Biol. 227:381-88.

Screening of the libraries can be accomplished by various techniques known in the art. For example, an antigen (e.g., an IL-2 polypeptide, fragment, or epitope) can be used to coat the wells of adsorption plates, expressed on host cells affixed to adsorption plates or used in cell sorting, conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., 1990, Proteins 8:309-14 and WO 92/09690, and by use of a low coating density of antigen as described in Marks et al., 1992, Biotechnol. 10:779-83.

Antibodies that form part of the immunoconjugate molecules described herein can be obtained by designing a suitable antigen screening procedure to select for the phage clone of interest followed by construction of a full-length antibody clone using VH and/or VL sequences (e.g., the Fv sequences), or various CDR sequences from VH and VL sequences, from the phage clone of interest and suitable constant region (e.g., Fc) sequences described in Kabat et al., supra.

In another embodiment, antibodies that form part of the immunoconjugate molecules is generated by using methods as described in Bowers et al., 2011, Proc Natl Acad Sci USA. 108:20455-60, e.g., the SHM-XHL™ platform (AnaptysBio, San Diego, CA). Briefly, in this approach, a fully human library of IgGs is constructed in a mammalian cell line (e.g., HEK293) as a starting library. Mammalian cells displaying immunoglobulin that binds to a target peptide or epitope are selected (e.g., by FACS sorting), then activation-induced cytidine deaminase (AID)-triggered somatic hypermutation is reproduced in vitro to expand diversity of the initially selected pool of antibodies. After several rounds of affinity maturation by coupling mammalian cell surface display with in vitro somatic hypermutation, high affinity, high specificity antibodies are generated. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

### 5.3.2.1 Antibody Fragments

The present disclosure provides antibodies and antibody fragments that form parts of an immunoconjugate molecule. In certain circumstances there are advantages of using antibody fragments, rather than whole antibodies. The smaller size of the fragments allows for rapid clearance, and may lead to improved access to cells, tissues, or organs. For a review of certain antibody fragments, see Hudson et al., 2003, Nature Med. 9:129-34.

Various techniques have been developed for the production of antibody fragments. Traditionally, these fragments were derived via proteolytic digestion of intact antibodies (see, e.g., Morimoto et al., 1992, J. Biochem. Biophys. Methods 24:107-17; and Brennan et al., 1985, Science 229:81-83). However, these fragments can now be produced directly by recombinant host cells. Fab, Fv, and scFv antibody fragments can all be expressed in and secreted from E. coli or yeast cells, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., 1992, Bio/Technology 10:163-67). According to another approach, F(ab′)₂ fragments can be isolated directly from recombinant host cell culture. Fab and F(ab′)₂ fragment with increased in vivo half-life comprising salvage receptor binding epitope residues are described in, for example, U.S. Pat. No. 5,869,046. Other techniques for the production of antibody fragments will be apparent to the skilled practitioner. In certain embodiments, an antibody is a single chain Fv fragment (scFv) (see, e.g., WO 93/16185; U.S. Pat. Nos. 5,571,894 and 5,587,458). Fv and scFv have intact combining sites that are devoid of constant regions; thus, they may be suitable for reduced nonspecific binding during in vivo use. scFv fusion proteins may be constructed to yield fusion of an effector protein at either the amino or the carboxy terminus of an scFv (See, e.g., Borrebaeck ed., supra). The antibody fragment may also be a “linear antibody,” for example, as described in the references cited above. Such linear antibodies may be monospecific or multi-specific, such as bispecific.

Smaller antibody-derived binding structures are the separate variable domains (V domains) also termed single variable domain antibodies (sdAbs). Certain types of organisms, the camelids and cartilaginous fish, possess high affinity single V-like domains mounted on an Fc equivalent domain structure as part of their immune system. (Woolven et al., 1999, Immunogenetics 50: 98-101; and Streltsov et al., 2004, Proc Natl Acad Sci USA. 101:12444-49). The V-like domains (called VhH in camelids and V-NAR in sharks) typically display long surface loops, which allow penetration of cavities of target antigens. They also stabilize isolated VH domains by masking hydrophobic surface patches.

These VhH and V-NAR domains have been used to engineer sdAbs. Human V domain variants have been designed using selection from phage libraries and other approaches that have resulted in stable, high binding VL- and VH-derived domains.

Antibodies provided herein include, but are not limited to, immunoglobulin molecules and immunologically active portions of immunoglobulin molecules, for example, molecules that contain an antigen binding site that bind to an epitope (e.g., IL-2 epitope or FAP epitope). The immunoglobulin molecules provided herein can be of any class (e.g., IgG, IgE, IgM, IgD, and IgA) or any subclass (e.g., IgG1, IgG2, IgG3, IgG4, IgA1, and IgA2) of immunoglobulin molecule.

Variants and derivatives of antibodies include antibody functional fragments that retain the ability to bind to an epitope (e.g., IL-2 epitope or FAP epitope). Exemplary functional fragments include Fab fragments (e.g., an antibody fragment that contains the antigen-binding domain and comprises a light chain and part of a heavy chain bridged by a disulfide bond); Fab′(e.g., an antibody fragment containing a single antigen-binding domain comprising an Fab and an additional portion of the heavy chain through the hinge region); F(ab′)₂ (e.g., two Fab′molecules joined by interchain disulfide bonds in the hinge regions of the heavy chains; the Fab′molecules may be directed toward the same or different epitopes); a bispecific Fab (e.g., a Fab molecule having two antigen binding domains, each of which may be directed to a different epitope); a single chain comprising a variable region, also known as, scFv (e.g., the variable, antigen-binding determinative region of a single light and heavy chain of an antibody linked together by a chain of 10-25 amino acids); a disulfide-linked Fv, or dsFv (e.g., the variable, antigen-binding determinative region of a single light and heavy chain of an antibody linked together by a disulfide bond); a camelized VH (e.g., the variable, antigen-binding determinative region of a single heavy chain of an antibody in which some amino acids at the VH interface are those found in the heavy chain of naturally occurring camel antibodies); a bispecific scFv (e.g., an scFv or a dsFv molecule having two antigen-binding domains, each of which may be directed to a different epitope); a diabody (e.g., a dimerized scFv formed when the VH domain of a first scFv assembles with the VL domain of a second scFv and the VL domain of the first scFv assembles with the VH domain of the second scFv; the two antigen-binding regions of the diabody may be directed towards the same or different epitopes); and a triabody (e.g., a trimerized scFv, formed in a manner similar to a diabody, but in which three antigen-binding domains are created in a single complex; the three antigen-binding domains may be directed towards the same or different epitopes).

### 5.3.2.2 Humanized Antibodies

In some embodiments, antibodies forming part of an immunoconjugate molecule provided herein can be humanized antibodies that bind, including human and/or cynomolgus antigen (such as human IL-2 or human FAP). For example, humanized antibodies of the present disclosure may comprise one or more CDRs as shown in Tables 1-2 and 5-6. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanization may be performed, for example, following the method of Jones et al., 1986, Nature 321:522-25; Riechmann et al., 1988, Nature 332:323-27; and Verhoeyen et al., 1988, Science 239:1534-36), by substituting hypervariable region sequences for the corresponding sequences of a human antibody.

In some cases, the humanized antibodies are constructed by CDR grafting, in which the amino acid sequences of the six CDRs of the parent non-human antibody (e.g., rodent) are grafted onto a human antibody framework. For example, Padlan et al. determined that only about one third of the residues in the CDRs actually contact the antigen, and termed these the “specificity determining residues,” or SDRs (Padlan et al., 1995, FASEB J. 9:133-39). In the technique of SDR grafting, only the SDR residues are grafted onto the human antibody framework (see, e.g., Kashmiri et al., 2005, Methods 36:25-34).

The choice of human variable domains, both light and heavy, to be used in making the humanized antibodies can be important to reduce antigenicity. For example, according to the so-called “best-fit” method, the sequence of the variable domain of a non-human (e.g., rodent) antibody is screened against the entire library of known human variable-domain sequences. The human sequence that is closest to that of the rodent may be selected as the human framework for the humanized antibody (Sims et al., 1993, J. Immunol. 151:2296-308; and Chothia et al., 1987, J. Mol. Biol. 196:901-17). Another method uses a particular framework derived from the consensus sequence of all human antibodies of a particular subgroup of light or heavy chains. The same framework may be used for several different humanized antibodies (Carter et al., 1992, Proc. Natl. Acad. Sci. USA 89:4285-89; and Presta et al., 1993, J. Immunol. 151:2623-32). In some cases, the framework is derived from the consensus sequences of the most abundant human subclasses, V_{L}6 subgroup I (VL6I) and V_{H} subgroup III (V_{H}III). In another method, human germline genes are used as the source of the framework regions.

In an alternative paradigm based on comparison of CDRs, called superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., 2002, J. Immunol. 169:1119-25).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, 2000, Protein Eng. 13:819-24), Modeller (Sali and Blundell, 1993, J. Mol. Biol. 234:779-815), and Swiss PDB Viewer (Guex and Peitsch, 1997, Electrophoresis 18:2714-23). Inspection of these displays permits analysis of the likely role of the residues in the functioning of the candidate immunoglobulin sequence, e.g., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes, and the differences are scored as HSC. The target sequence is then humanized by maximizing its HSC rather than using a global identity measure to generate multiple diverse humanized variants (Lazar et al., 2007, Mol. Immunol. 44:1986-98).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome, and yeast display libraries as well as by bacterial colony screening (see, e.g., Hoogenboom, 2005, Nat. Biotechnol. 23:1105-16; Dufner et al., 2006, Trends Biotechnol. 24:523-29; Feldhaus et al., 2003, Nat. Biotechnol. 21:163-70; and Schlapschy et al., 2004, Protein Eng. Des. Sel. 17:847-60).

In the FR library approach, a collection of residue variants are introduced at specific positions in the FR followed by screening of the library to select the FR that best supports the grafted CDR. The residues to be substituted may include some or all of the “Vernier” residues identified as potentially contributing to CDR structure (see, e.g., Foote and Winter, 1992, J. Mol. Biol. 224:487-99), or from the more limited set of target residues identified by Baca et al. (1997, J. Biol. Chem. 272:10678-84).

In FR shuffling, whole FRs are combined with the non-human CDRs instead of creating combinatorial libraries of selected residue variants (see, e.g., Dall'Acqua et al., 2005, Methods 36:43-60). The libraries may be screened for binding in a two-step process, first humanizing VL, followed by VH. Alternatively, a one-step FR shuffling process may be used. Such a process has been shown to be more efficient than the two-step screening, as the resulting antibodies exhibited improved biochemical and physicochemical properties including enhanced expression, increased affinity, and thermal stability (see, e.g., Damschroder et al., 2007, Mol. Immunol. 44:3049-60).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple subclasses with distinct human V-segment CDRs. Humaneering allows for isolation of antibodies that are 91-96% homologous to human germline gene antibodies (see, e.g., Alfenito, Cambridge Healthtech Institute's Third Annual PEGS, The Protein Engineering Summit, 2007).

The “human engineering” method involves altering a non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk,” “moderate risk,” or “high risk” residues. The classification is performed using a global risk/reward calculation that evaluates the predicted benefits of making particular substitution (e.g., for immunogenicity in humans) against the risk that the substitution will affect the resulting antibody's folding. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low or moderate risk positions in the non-human sequence can be substituted for the corresponding residues in the human antibody sequence according to the alignment. Techniques for making human engineered proteins are described in greater detail in Studnicka et al., 1994, Protein Engineering 7:805-14; U.S. Pat. Nos. 5,766,886; 5,770,196; 5,821,123; and 5,869,619; and PCT Publication WO 93/11794.

### 5.3.2.3 Human Antibodies

Human antibodies can be constructed by combining Fv clone variable domain sequence(s) selected from human-derived phage display libraries with known human constant domain sequences(s). Alternatively, human monoclonal antibodies of the present disclosure can be made by the hybridoma method. Human myeloma and mouse-human heteromyeloma cell lines for the production of human monoclonal antibodies have been described, for example, by Kozbor, 1984, J. Immunol. 133:3001-05; Brodeur et al., Monoclonal Antibody Production Techniques and Applications 51-63 (1987); and Boerner et al., 1991, J. Immunol. 147:86-95.

It is also possible to produce transgenic animals (e.g., mice) that are capable, upon immunization, of producing a full repertoire of human antibodies in the absence of endogenous immunoglobulin production. Transgenic mice that express human antibody repertoires have been used to generate high-affinity human sequence monoclonal antibodies against a wide variety of potential drug targets (see, e.g., Jakobovits, A., 1995, Curr. Opin. Biotechnol. 6(5):561-66; Bruggemann and Taussing, 1997, Curr. Opin. Biotechnol. 8(4):455-58; U.S. Pat. Nos. 6,075,181 and 6,150,584; and Lonberg et al., 2005, Nature Biotechnol. 23:1117-25).

Alternatively, the human antibody may be prepared via immortalization of human B lymphocytes producing an antibody directed against a target antigen (e.g., such B lymphocytes may be recovered from an individual or may have been immunized in vitro) (see, e.g., Cole et al., Monoclonal Antibodies and Cancer Therapy (1985); Boerner et al., 1991, J. Immunol. 147(1):86-95; and U.S. Pat. No. 5,750,373).

Gene shuffling can also be used to derive human antibodies from non-human, for example, rodent, antibodies, where the human antibody has similar affinities and specificities to the starting non-human antibody. According to this method, which is also called “epitope imprinting” or “guided selection,” either the heavy or light chain variable region of a non-human antibody fragment obtained by phage display techniques as described herein is replaced with a repertoire of human V domain genes, creating a population of non-human chain/human chain scFv or Fab chimeras. Selection with antigen results in isolation of a non-human chain/human chain chimeric scFv or Fab wherein the human chain restores the antigen binding site destroyed upon removal of the corresponding non-human chain in the primary phage display clone (e.g., the epitope guides (imprints) the choice of the human chain partner). When the process is repeated in order to replace the remaining non-human chain, a human antibody is obtained (see, e.g., PCT WO 93/06213; and Osbourn et al., 2005, Methods 36:61-68). Unlike traditional humanization of non-human antibodies by CDR grafting, this technique provides completely human antibodies, which have no FR or CDR residues of non-human origin. Examples of guided selection to humanize mouse antibodies towards cell surface antigens include the folate-binding protein present on ovarian cancer cells (see, e.g., Figini et al., 1998, Cancer Res. 58:991-96) and CD147, which is highly expressed on hepatocellular carcinoma (see, e.g., Bao et al., 2005, Cancer Biol. Ther. 4:1374-80).

A potential disadvantage of the guided selection approach is that shuffling of one antibody chain while keeping the other constant could result in epitope drift. In order to maintain the epitope recognized by the non-human antibody, CDR retention can be applied (see, e.g., Klimka et al., 2000, Br. J. Cancer. 83:252-60; and Beiboer et al., 2000, J. Mol. Biol. 296:833-49). In this method, the non-human VH CDR3 is commonly retained, as this CDR may be at the center of the antigen-binding site and may be the most important region of the antibody for antigen recognition. In some instances, however, VH CDR3 and VL CDR3, as well as VH CDR2, VL CDR2, and VL CDR1 of the non-human antibody may be retained.

### 5.3.3 Antibody Variants

In some embodiments, amino acid sequence modification(s) of the antibodies that form part of the immunoconjugate molecules described herein are contemplated. For example, it may be desirable to improve the binding affinity and/or other biological properties of the antibody, including but not limited to specificity, thermostability, expression level, effector functions, glycosylation, reduced immunogenicity, or solubility. Thus, in addition to the specific antibodies provided herein, it is contemplated that antibody variants can be prepared. For example, antibody variants can be prepared by introducing appropriate nucleotide changes into the encoding DNA, and/or by synthesis of the desired antibody or polypeptide. Those skilled in the art who appreciate that amino acid changes may alter post-translational processes of the antibody, such as changing the number or position of glycosylation sites or altering the membrane anchoring characteristics.

In some embodiments, antibodies provided herein are chemically modified, for example, by the covalent attachment of any type of molecule to the antibody. The antibody derivatives may include antibodies that have been chemically modified, for example, by increase or decrease of glycosylation, acetylation, pegylation, phosphorylation, amidation, derivatization by known protecting/blocking groups, chemical cleavage, proteolytic cleavage, linkage to a cellular ligand or other protein, etc. Additionally, the antibody may contain one or more non-classical amino acids.

Variations may be a substitution, deletion, or insertion of one or more codons encoding the antibody or polypeptide that results in a change in the amino acid sequence as compared with the native sequence antibody or polypeptide. Amino acid substitutions can be the result of replacing one amino acid with another amino acid having similar structural and/or chemical properties, such as the replacement of a leucine with a serine, e.g., conservative amino acid replacements. Insertions or deletions may optionally be in the range of about 1 to 5 amino acids. In certain embodiments, the substitution, deletion, or insertion includes fewer than 25 amino acid substitutions, fewer than 20 amino acid substitutions, fewer than 15 amino acid substitutions, fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the full-length or mature native sequence.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for antibody-directed enzyme prodrug therapy) or a polypeptide which increases the serum half-life of the antibody.

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Alternatively, conservative (e.g., within an amino acid group with similar properties and/or side chains) substitutions may be made, so as to maintain or not significantly change the properties. Amino acids may be grouped according to similarities in the properties of their side chains (see, e.g., Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His(H).

Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

Non-conservative substitutions entail exchanging a member of one of these classes for another class. Such substituted residues also may be introduced into the conservative substitution sites or, into the remaining (non-conserved) sites. Accordingly, in one embodiment, an antibody or fragment thereof that binds to an epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a murine monoclonal antibody provided herein. In one embodiment, an antibody or fragment thereof that binds to an epitope comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to an amino acid sequence depicted in Tables 1-8. In yet another embodiment, an antibody or fragment thereof forming part of the immunoconjugate molecule as described herein comprises a VH CDR and/or a VL CDR amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to a VH CDR amino acid sequence depicted in Table 2 and/or a VL CDR amino acid sequence depicted in Table 1. In yet another embodiment, an antibody or fragment thereof forming part of the immunoconjugate molecule as described herein comprises a VH CDR and/or a VL CDR amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to a VH CDR amino acid sequence depicted in Table 6 and/or a VL CDR amino acid sequence depicted in Table 5. The variations can be made using methods known in the art such as oligonucleotide-mediated (site-directed) mutagenesis, alanine scanning, and PCR mutagenesis. Site-directed mutagenesis (see, e.g., Carter, 1986, Biochem J. 237:1-7; and Zoller et al., 1982, Nucl. Acids Res. 10:6487-500), cassette mutagenesis (see, e.g., Wells et al., 1985, Gene 34:315-23), or other known techniques can be performed on the cloned DNA to produce the antibody variant DNA.

Any cysteine residue not involved in maintaining the proper conformation of the antibody also may be substituted, for example, with another amino acid, such as alanine or serine, to improve the oxidative stability of the molecule and to prevent aberrant crosslinking. Conversely, cysteine bond(s) may be added to the antibody to improve its stability (e.g., where the antibody is an antibody fragment such as an Fv fragment).

In some embodiments, an antibody or antigen binding fragment thereof forming part of the immunoconjugate molecule of the present disclosure is a “de-immunized” antibody. A “de-immunized” antibody is an antibody derived from a humanized or chimeric antibody, which has one or more alterations in its amino acid sequence resulting in a reduction of immunogenicity of the antibody, compared to the respective original non-de-immunized antibody. One of the procedures for generating such antibody mutants involves the identification and removal of T cell epitopes of the antibody molecule. In a first step, the immunogenicity of the antibody molecule can be determined by several methods, for example, by in vitro determination of T cell epitopes or in silico prediction of such epitopes, as known in the art. Once the critical residues for T cell epitope function have been identified, mutations can be made to remove immunogenicity and retain antibody activity. For review, see, for example, Jones et al., 2009, Methods in Molecular Biology 525:405-23.

5.3.3.1 In vitro Affinity Maturation

In some embodiments, antibody variants having an improved property such as affinity, stability, or expression level as compared to a parent antibody may be prepared by in vitro affinity maturation. Like the natural prototype, in vitro affinity maturation is based on the principles of mutation and selection. Libraries of antibodies are displayed as Fab, scFv, or V domain fragments either on the surface of an organism (e.g., phage, bacteria, yeast, or mammalian cell) or in association (e.g., covalently or non-covalently) with their encoding mRNA or DNA. Affinity selection of the displayed antibodies allows isolation of organisms or complexes carrying the genetic information encoding the antibodies. Two or three rounds of mutation and selection using display methods such as phage display usually results in antibody fragments with affinities in the low nanomolar range. Affinity matured antibodies can have nanomolar or even picomolar affinities for the target antigen.

Phage display is a widespread method for display and selection of antibodies. The antibodies are displayed on the surface of Fd or M13 bacteriophages as fusions to the bacteriophage coat protein. Selection involves exposure to antigen to allow phage-displayed antibodies to bind their targets, a process referred to as “panning.” Phage bound to antigen are recovered and used to infect bacteria to produce phage for further rounds of selection. For review, see, for example, Hoogenboom, 2002, Methods. Mol. Biol. 178:1-37; and Bradbury and Marks, 2004, J. Immunol. Methods 290:29-49.

In a yeast display system (see, e.g., Boder et al., 1997, Nat. Biotech. 15:553-57; and Chao et al., 2006, Nat. Protocols 1:755-68), the antibody may be displayed as single-chain variable fusions (scFv) in which the heavy and light chains are connected by a flexible linker. The scFv is fused to the adhesion subunit of the yeast agglutinin protein Aga2p, which attaches to the yeast cell wall through disulfide bonds to Aga1p. Display of a protein via Aga2p projects the protein away from the cell surface, minimizing potential interactions with other molecules on the yeast cell wall. Magnetic separation and flow cytometry are used to screen the library to select for antibodies with improved affinity or stability. Binding to a soluble antigen of interest is determined by labeling of yeast with biotinylated antigen and a secondary reagent such as streptavidin conjugated to a fluorophore. Variations in surface expression of the antibody can be measured through immunofluorescence labeling of either the hemagglutinin or c-Myc epitope tag flanking the scFv. Expression has been shown to correlate with the stability of the displayed protein, and thus antibodies can be selected for improved stability as well as affinity (see, e.g., Shusta et al., 1999, J. Mol. Biol. 292:949-56). An additional advantage of yeast display is that displayed proteins are folded in the endoplasmic reticulum of the eukaryotic yeast cells, taking advantage of endoplasmic reticulum chaperones and quality-control machinery. Once maturation is complete, antibody affinity can be conveniently “titrated” while displayed on the surface of the yeast, eliminating the need for expression and purification of each clone. A theoretical limitation of yeast surface display is the potentially smaller functional library size than that of other display methods; however, a recent approach uses the yeast cells' mating system to create combinatorial diversity estimated to be 10¹⁴ in size (see, e.g., U.S. Pat. Publication 2003/0186374; and Blaise et al., 2004, Gene 342:211-18).

In ribosome display, antibody-ribosome-mRNA (ARM) complexes are generated for selection in a cell-free system. The DNA library coding for a particular library of antibodies is genetically fused to a spacer sequence lacking a stop codon. This spacer sequence, when translated, is still attached to the peptidyl tRNA and occupies the ribosomal tunnel, and thus allows the protein of interest to protrude out of the ribosome and fold. The resulting complex of mRNA, ribosome, and protein can bind to surface-bound ligand, allowing simultaneous isolation of the antibody and its encoding mRNA through affinity capture with the ligand. The ribosome-bound mRNA is then reverse transcribed back into cDNA, which can then undergo mutagenesis and be used in the next round of selection (see, e.g., Fukuda et al., 2006, Nucleic Acids Res. 34:e127). In mRNA display, a covalent bond between antibody and mRNA is established using puromycin as an adaptor molecule (Wilson et al., 2001, Proc. Natl. Acad. Sci. USA 98:3750-55).

As these methods are performed entirely in vitro, they provide two main advantages over other selection technologies. First, the diversity of the library is not limited by the transformation efficiency of bacterial cells, but only by the number of ribosomes and different mRNA molecules present in the test tube. Second, random mutations can be introduced easily after each selection round, for example, by non-proofreading polymerases, as no library must be transformed after any diversification step.

In a mammalian cell display system (see, e.g., Bowers et al., 2011, Proc Natl Acad Sci USA. 108:20455-60), a fully human library of IgGs is constructed based on germline sequence V-gene segments joined to prerecombined D(J) regions. Full-length V regions for heavy chain and light chain are assembled with human heavy chain and light chain constant regions and transfected into a mammalian cell line (e.g., HEK293). The transfected library is expanded and subjected to several rounds of negative selection against streptavidin (SA)-coupled magnetic beads, followed by a round of positive selection against SA-coupled magnetic beads coated with biotinylated target protein, peptide fragment, or epitope. Positively selected cells are expanded, and then sorted by rounds of FACS to isolate single cell clones displaying antibodies that specifically bind to the target protein, peptide fragment, or epitope. Heavy and light chain pairs from these single cell clones are retransfected with AID for further maturation. Several rounds of mammalian cell display, coupled with AID-triggered somatic hypermutation, generate high specificity, high affinity antibodies.

Diversity may also be introduced into the CDRs or the whole V genes of the antibody libraries in a targeted manner or via random introduction. The former approach includes sequentially targeting all the CDRs of an antibody via a high or low level of mutagenesis or targeting isolated hot spots of somatic hypermutations (see, e.g., Ho et al., 2005, J. Biol. Chem. 280:607-17) or residues suspected of affecting affinity on experimental basis or structural reasons. In a specific embodiment, somatic hypermutation is performed by AID-triggered somatic hypermutation, e.g., using the SHM-XEL platform (AnaptysBio, San Diego, CA). Random mutations can be introduced throughout the whole V gene using E. coli mutator strains, error-prone replication with DNA polymerases (see, e.g., Hawkins et al., 1992, J. Mol. Biol. 226:889-96), or RNA replicases. Diversity may also be introduced by replacement of regions that are naturally diverse via DNA shuffling or similar techniques (see, e.g., Lu et al., 2003, J. Biol. Chem. 278:43496-507; U.S. Pat. Nos. 5,565,332 and 6,989,250). Alternative techniques target hypervariable loops extending into framework-region residues (see, e.g., Bond et al., 2005, J. Mol. Biol. 348:699-709) employ loop deletions and insertions in CDRs or use hybridization-based diversification (see, e.g., U.S. Pat. Publication No. 2004/0005709). Additional methods of generating diversity in CDRs are disclosed, for example, in U.S. Pat. No. 7,985,840. Further methods that can be used to generate antibody libraries and/or antibody affinity maturation are disclosed, e.g., in U.S. Pat. Nos. 8,685,897 and 8,603,930, and U.S. Publ. Nos. 2014/0170705, 2014/0094392, 2012/0028301, 2011/0183855, and 2009/0075378, each of which are incorporated herein by reference.

Screening of the libraries can be accomplished by various techniques known in the art. For example, a target antigen (such as IL-2 or FAP polypeptide can be immobilized onto solid supports, columns, pins, or cellulose/poly(vinylidene fluoride) membranes/other filters, expressed on host cells affixed to adsorption plates or used in cell sorting, or conjugated to biotin for capture with streptavidin-coated beads or used in any other method for panning display libraries.

For review of in vitro affinity maturation methods, see, e.g., Hoogenboom, 2005, Nature Biotechnology 23:1105-16; Quiroz and Sinclair, 2010, Revista Ingeneria Biomedia 4:39-51; and references therein.

### 5.3.3.2 Modifications of Antibodies

Covalent modifications of antibodies forming part of the immunoconjugate molecule of the present disclosure are included within the scope of the present disclosure. Covalent modifications include reacting targeted amino acid residues of an antibody with an organic derivatizing agent that is capable of reacting with selected side chains or the N- or C-terminal residues of the antibody. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (see, e.g., Creighton, Proteins: Structure and Molecular Properties 79-86 (1983)), acetylation of the N-terminal amine, and amidation of any C-terminal carboxyl group.

Other types of covalent modification of the antibody included within the scope of this present disclosure include altering the native glycosylation pattern of the antibody or polypeptide (see, e.g., Beck et al., 2008, Curr. Pharm. Biotechnol. 9:482-501; and Walsh, 2010, Drug Discov. Today 15:773-80), and linking the antibody to one of a variety of nonproteinaceous polymers, e.g., polyethylene glycol (PEG), polypropylene glycol, or polyoxyalkylenes, in the manner set forth, for example, in U.S. Pat. Nos. 4,640,835; 4,496,689; 4,301,144; 4,670,417; 4,791,192; or 4,179,337.

An antibody forming part of the immunoconjugate molecule of the present disclosure may also be modified to form chimeric molecules comprising an antibody fused to another, heterologous polypeptide or amino acid sequence, for example, a cytokine polypeptide (see, e.g., Terpe, 2003, Appl. Microbiol. Biotechnol. 60:523-33) or the Fc region of an IgG molecule (see, e.g., Aruffo, Antibody Fusion Proteins 221-42 (Chamow and Ashkenazi eds., 1999)).

Also provided herein are fusion proteins comprising an antibody provided herein that binds to a target antigen and a heterologous polypeptide. In some embodiments, the antibody is useful to deliver and/or immobilize the heterologous polypeptide to which the antibody is fused to cells having cell surface-expressed target antigen.

Also provided herein are panels of antibodies that bind to a target antigen (e.g., IL-2 or FAP). In specific embodiments, the panels of antibodies have different association rates, different dissociation rates, different affinities for the target antigen, and/or different specificities for a target antigen. In some embodiments, the panels comprise or consist of about 10, about 25, about 50, about 75, about 100, about 125, about 150, about 175, about 200, about 250, about 300, about 350, about 400, about 450, about 500, about 550, about 600, about 650, about 700, about 750, about 800, about 850, about 900, about 950, or about 1000 antibodies or more. Panels of antibodies can be used, for example, in 96-well or 384-well plates, for assays such as ELISAs.

### 5.3.4 Preparation of Antibodies and Immunoconjugate Molecules

Antibodies and other peptidic components (e.g., cytokine polypeptide) forming part of the present immunoconjugate molecules may be produced by culturing cells transformed or transfected with a vector containing the encoding nucleic acids. Polynucleotide sequences encoding polypeptide components of the antibody of the present disclosure can be obtained using standard recombinant techniques. Desired polynucleotide sequences may be isolated and sequenced from antibody producing cells such as hybridomas cells. Alternatively, polynucleotides can be synthesized using nucleotide synthesizer or PCR techniques. Once obtained, sequences encoding the polypeptides are inserted into a recombinant vector capable of replicating and expressing heterologous polynucleotides in host cells. Many vectors that are available and known in the art can be used for the purpose of the present disclosure. Selection of an appropriate vector will depend mainly on the size of the nucleic acids to be inserted into the vector and the particular host cell to be transformed with the vector. Host cells suitable for expressing antibodies of the present disclosure include prokaryotes such as Archaebacteria and Eubacteria, including Gram-negative or Gram-positive organisms, eukaryotic microbes such as filamentous fungi or yeast, invertebrate cells such as insect or plant cells, and vertebrate cells such as mammalian host cell lines. Host cells are transformed with the above-described expression vectors and cultured in conventional nutrient media modified as appropriate for inducing promoters, selecting transformants, or amplifying the genes encoding the desired sequences. Antibodies produced by the host cells are purified using standard protein purification methods as known in the art.

Methods for antibody production including vector construction, expression, and purification are further described in Pltckthun et al., Antibody Engineering: Producing antibodies in Escherichia coli: From PCR to fermentation 203-52 (McCafferty et al. eds., 1996); Kwong and Rader, E. coli Expression and Purification of Fab Antibody Fragments, in Current Protocols in Protein Science (2009); Tachibana and Takekoshi, Production of Antibody Fab Fragments in Escherischia coli, in Antibody Expression and Production (Al-Rubeai ed., 2011); and Therapeutic Monoclonal Antibodies: From Bench to Clinic (An ed., 2009).

It is, of course, contemplated that alternative methods, which are well known in the art, may be employed to prepare antibodies. For instance, the appropriate amino acid sequence, or portions thereof, may be produced by direct peptide synthesis using solid-phase techniques (see, e.g., Stewart et al., Solid-Phase Peptide Synthesis (1969); and Merrifield, 1963, J. Am. Chem. Soc. 85:2149-54). In vitro protein synthesis may be performed using manual techniques or by automation. Various portions of the antibody may be chemically synthesized separately and combined using chemical or enzymatic methods to produce the desired antibody. Alternatively, antibodies may be purified from cells or bodily fluids, such as milk, of a transgenic animal engineered to express the antibody, as disclosed, for example, in U.S. Pat. Nos. 5,545,807 and 5,827,690.

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of antibodies as provided herein, including, for example, antibodies with higher affinities and lower dissociation rates (see, e.g., U.S. Pat. Nos. 5,605,793; 5,811,238; 5,830,721; 5,834,252; and 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

Methods for fusing or conjugating moieties (including polypeptides) to antibodies are known (see, e.g., Amon et al., Monoclonal Antibodies for Immunotargeting of Drugs in Cancer Therapy, in Monoclonal Antibodies and Cancer Therapy 243-56 (Reisfeld et al. eds., 1985); Hellstrom et al., Antibodiesfor Drug Delivery, in Controlled Drug Delivery 623-53 (Robinson et al. eds., 2d ed. 1987); Thorpe, Antibody Carriers of Cytotoxic Agents in Cancer Therapy: A Review, in Monoclonal Antibodies: Biological and Clinical Applications 475-506 (Pinchera et al. eds., 1985); Analysis, Results, and Future Prospective of the Therapeutic Use of Radiolabeled Antibody in Cancer Therapy, in Monoclonal Antibodies for Cancer Detection and Therapy 303-16 (Baldwin et al. eds., 1985); Thorpe et al., 1982, Immunol. Rev. 62:119-58; U.S. Pat. Nos. 5,336,603; 5,622,929; 5,359,046; 5,349,053; 5,447,851; 5,723,125; 5,783,181; 5,908,626; 5,844,095; and 5,112,946; EP 307,434; EP 367,166; EP 394,827; PCT publications WO 91/06570, WO 96/04388, WO 96/22024, WO 97/34631, and WO 99/04813; Ashkenazi et al., 1991, Proc. Natl. Acad. Sci. USA, 88: 10535-39; Traunecker et al., 1988, Nature, 331:84-86; Zheng et al., 1995, J. Immunol. 154:5590-600; and Vil et al., 1992, Proc. Natl. Acad. Sci. USA 89:11337-41).

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of antibodies as provided herein, including, for example, antibodies with higher affinities and lower dissociation rates (see, e.g., U.S. Pat. Nos. 5,605,793; 5,811,238; 5,830,721; 5,834,252; and 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-13). Antibodies, or the encoded antibodies, may be altered by being subjected to random mutagenesis by error-prone PCR, random nucleotide insertion, or other methods prior to recombination. A polynucleotide encoding an antibody provided herein may be recombined with one or more components, motifs, sections, parts, domains, fragments, etc. of one or more heterologous molecules.

Conjugates of the antibody and agent may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that conjugates of antibodies and agents may be prepared using any suitable methods as disclosed in the art (see, e.g., Bioconjugate Techniques (Hermanson ed., 2d ed. 2008)).

Conventional conjugation strategies for antibodies and agents have been based on random conjugation chemistries involving the F-amino group of Lys residues or the thiol group of Cys residues, which results in heterogenous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen binding (see, e.g., Junutula et al., 2008, J. Immunol. Meth. 332: 41-52; and Junutula et al., 2008, Nature Biotechnol. 26:925-32). In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids (see, e.g., Hofer et al., 2008, Proc. Natl. Acad. Sci. USA 105:12451-56; and Hofer et al., 2009, Biochemistry 48(50):12047-57).

### 5.4 Methods of Using the Immunoconjugate Molecules and Compositions

As would be appreciated from the present disclosure, the immunoconjugate molecules according to the present disclosure can be used for delivering a cytokine and/or activating a cytokine activity at a target site of interest in a subject. Without being bound by the theory, it is also contemplated that when systemic exposure to certain cytokine activity can result in toxic side-effect in a subject, the immunoconjugate molecules of the present disclosure can be used for reducing toxicity or other side-effects of the cytokine by preventing the activation of the cytokine-mediated effect in locations other than the target site in the subject.

Accordingly, in one aspect, provided herein is a method for site-specific delivery of a cytokine molecule in a subject, the method comprising incorporating the cytokine into an immunoconjugate molecule according to the present disclosure, and delivering the immunoconjugate molecule to the subject. Particularly, in certain embodiments, the immunoconjugate molecule comprises the cytokine and an anchoring moiety capable of binding to a target antigen present at the target site in the subject, such that when the immunoconjugate molecule arrives at the target site, the anchoring moiety binds to the target antigen, thereby immobilizing the immunoconjugate molecule at the target site. In some embodiments, the method results in a higher concentration of the administered immunoconjugate molecule at the target site in the subject as compared to a non-target site.

In a related aspect, provided herein is also a method for site-specific activation of a cytokine activity in a subject, the method comprising incorporating the cytokine into an immunoconjugate molecule according to the present disclosure, and delivering the immunoconjugate molecule to the subject. Particularly, in certain embodiments, the immunoconjugate molecule comprises the cytokine and a masking moiety that binds to and inhibits the cytokine activity via intramolecular interaction. Particularly, the masking moiety is also capable of binding to a target antigen present at the target site, such that when the immunoconjugate molecule arrives at the target site, the masking moiety binds to the target antigen and disassociates from the cytokine, thereby activating the cytokine activity at the target site. In some embodiments, the method result in a higher cytokine activity at the target site in the subject as compared to a non-target site.

In specific embodiments, the immunoconjugate molecule used in the present methods comprises both a masking moiety and an anchoring moiety. In various embodiments, the target antigen recognized by the masking moiety and the anchoring moiety of the immunoconjugate molecule can be the same antigen or different antigens. In specific embodiments, the immunoconjugate molecule used in the present methods further comprises a conjugating moiety that operably connecting one or more of the cytokine moiety, masking moiety and anchoring moiety. In specific embodiments, the immunoconjugate molecule used in the present methods can be any of the immunoconjugate molecules as described in Section 5.3.

In some embodiments, the present methods result in reduced cytokine toxicity to the subject as compared a method that administered an equivalent amount of the cytokine in an unconjugated form. Accordingly, in a related aspect, provided herein is also a method for reducing a side-effect associated with the administration of an unconjugated form of the cytokine to a subject. In particular embodiments, the method comprises administering an immunoconjugate molecule comprising the cytokine to the subject in place of the administration of an unconjugated form of the cytokine. In particular embodiments, the subject is under an ongoing cytokine treatment comprising the administration of the cytokine in an unconjugated form, and the method comprises discontinuing the ongoing cytokine treatment and administering to the subject an immunoconjugate molecule comprising an equivalent amount of the cytokine. In particular embodiments, the side effect is toxicity of the cytokine. In particular embodiments, the side effect is measured by the change in body weight of the subject treated with the cytokine. In particular embodiments, the side effect is measured by the change in life-span of the subject treated with the cytokine. In particular embodiments, the side effect is measured by the change of the level of an immune response in the subject treated with the cytokine. In particular embodiments, the side effects are measured by the change in the level of an inflammatory reaction in the subject treated with the cytokine.

In some embodiments, the cytokine is IL-2, and the cytokine-mediated effect according to the present methods include activation of T cell activity in a subject. A non-limiting example of T cell activation is increased proliferation of T cells. Accordingly, in certain embodiments, provided herein are also a method for promoting T cell proliferation and activity at a target site in a subject by administering an IL-2 containing immunoconjugate molecule according to the present disclosure.

Another non-limiting example of T cell activity is secretion of a cytokine. Accordingly, in certain embodiments, provided herein are also a method for promoting secretion of a cytokine at a target site in a subject by administering an IL-2 containing immunoconjugate molecule according to the present disclosure. In certain embodiments, the cytokine is selected from the group consisting of IL-1, IL-2, IL-6, IL-12, IL-17, IL-22, IL-23, GM-CSF, IFN-γ, and TNF-α. In some embodiments, the cytokine is IL-2, IL-17, IFN-γ, or any combination thereof. In certain embodiments, the cytokine is IL-2. In other embodiments, the cytokine is IL-17. In yet other embodiments, the cytokine is IFN-γ. In certain embodiments, the cytokine is IL-2 and IL-17. In some embodiments, the cytokine is IL-2 and IFN-γ. In yet other embodiments, the cytokine is IL-17 and IFN-γ. In still other embodiments, the cytokine is IL-2, IL-17, and IFN-γ. In certain embodiments, the cytokine is IL-1. In other embodiments, the cytokine is IL-6. In yet other embodiments, the cytokine is IL-12. In still other embodiments, the cytokine is IL-22. In certain embodiments, the cytokine is IL-23. In some embodiments, the cytokine is GM-CSF. In other embodiments, the cytokine is TNF-α. Other combinations of two, three or more of the above-mentioned cytokines are also contemplated.

Exemplary target sites for delivering and/or activating the cytokine activity according to the present methods include but are not limited to a cellular environment, such as a particular type of tissue, a particular organ, a particular population of cells. In some embodiments, the target site of the present methods can be distinguished from a non-target site based on the expression of the target antigen recognized by the immunoconjugate molecule used in the method. Particularly, in some embodiments, the target antigen is present at the target site but is not present in the non-target site. In some embodiments, the target antigen is produced by cells that are present at the target site but are not present at a non-target site. In some embodiments, the target antigen is present at the target site at a higher concentration or in a greater amount as compared to the target antigen at the non-target site. In particular embodiments, the target antigen is present at the target site (but not a non-target site) in a sufficient amount that enables the anchoring moiety of immunoconjugate molecule to immobilize the immunoconjugate molecule at the target site through the binding to the target antigen. In particular embodiments, the target antigen is present at the target site (but not a non-target site) in a sufficient amount that enables the masking moiety of immunoconjugate molecule to disassociate from the cytokine through the binding to the target antigen. In specific embodiments, the target site of the present methods contains a population of cancer cells. In specific embodiments, the target site for the present methods is a tumor microenvironment of a solid tumor. In specific embodiments, the target antigen recognized by the immunoconjugate molecule used in the methods is an antigen expressed by cancer cells, such as a tumor associated antigen (TAA). In other embodiments, the target antigen recognized by the immunoconjugate molecule used in the methods is an antigen expressed by non-cancer cells in a tumor microenvironment, such as stromal cells.

In particular embodiments of the present methods, the cytokine is IL-2. In particular embodiments, the target antigen is fibrosis activation protein (FAP). Hence, in particular embodiments, the immunoconjugate molecule used in the present methods comprises a two-in-one antibody capable of binding to both IL-2 and FAP. In particular embodiments, the two-in-one antibody forming part of the present immunoconjugate molecule comprises VH CDR and VL CDR sequences as listed in Tables 1 and 2. In particular embodiments, the two-in-one antibody forming part of the present immunoconjugate molecule comprises VH and VL sequences as listed in Tables 3 and 4. In particular embodiments, the anchoring moiety of the immunoconjugate molecule is an antibody or antigen binding fragment thereof that bind to FAP. In particular embodiments, the anti-FAP antibody comprises VH CDR and VL CDR sequences as listed in Tables 5 and 6. In particular embodiments, the anti-FAP antibody comprises VH and VL sequences as listed in Tables 7 and 8.

In one aspect, provided herein is a method for activating an IL-2R, the method comprising contacting the IL-2R with an effective amount of an immunoconjugate molecule comprising an IL-2 polypeptide as provided herein. In some embodiments, the IL-2R comprises IL-2Rβ. In some embodiments, the IL-2R comprises IL-2Rα. In some embodiments, the IL-2R comprises IL-2Rγ. In some embodiments, the IL-2R comprises IL-2Rα and IL-2Rβ. In some embodiments, the IL-2R comprises IL-2Rα and IL-2Rγ. In some embodiments, the IL-2R comprises IL-2Rβ and IL-2Rγ. In some embodiments, the IL-2R comprises IL-2Rα, IL-2Rβ, and IL-2Rγ.

In some embodiments, one or more subunits forming the activable IL-2R are expressed on the same cell surface. In some embodiments, one or more subunits forming the activable IL-2R are expressed on surfaces of different cells. In some embodiments, one or more subunits forming the activable IL-2R are soluble.

In particular embodiments, the activable IL-2R comprises the IL-2Rβ, and wherein the IL-2Rβ is expressed on the surface of a first cell. In some embodiments, the activable IL-2R further comprises the IL-2Rγ, and wherein the IL-2Rγ is expressed on the surface of the first cell.

In some embodiments, the activable IL-2R further comprises the IL-2Rα. In some embodiments, the IL-2Rα is associated on a cell surface. In some embodiments, the IL-2Rα is associated on the surface of the first cell (cis-presentation). In some embodiments, the IL-2Rα is associated on the surface of a second cell (trans-presentation). In some embodiments, the IL-2Rα is not associated on a cell surface. In some embodiments, the activable IL-2R does not comprises the IL-2Rα.

In some embodiments, the first cell and/or the second cell expressing the subunit(s) of the activable IL-2R is an immune cell. In some embodiments, upon activation of the IL-2R, the immune cell is activated. In some embodiments, activation of the immune cell is measured as increased proliferation or maturation of the immune cell. In some embodiments, proliferation or maturation of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%. In some embodiments, activation of the immune cell is measured as prolonged survival time of the immune cell. In some embodiments, survival time of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

In some embodiments, the immune cell is an effector T cell, memory T cell, or a combination thereof. In some embodiments, the immune cell is CD4+ T cells, CD8+ T cells, helper T cells, cytotoxic T cells, SLECs (short-lived effector cells), MPEC (memory precursor effector cells), TEs (terminal effector cells), NKs (natural killer cells), NKTs (natural killer T cells), innate lymphoid cells (Types I-III), or a combination thereof.

In some embodiments, the immune cell is a regulatory T cell (Treg). In some embodiments, the immune cell is natural Treg (nTreg) cells, induced Treg (iTreg) cells, or a combination thereof.

In some embodiments, the first cell and/or the second cell expressing the subunit(s) of the activable IL-2R is a diseased cell. In some embodiments, upon activation of the IL-2R, the diseased cell dies. In some embodiments, the diseased cell is a cancer cell. In some embodiments, the diseased cell is a cell infected by an infectious pathogen. In some embodiments, the infectious pathogen is a virus, a bacteria, a fungus, a parasite, or a combination thereof. In some embodiments, the infectious pathogen is a virus. In some embodiments, the infectious pathogen is a bacteria. In some embodiments, the infectious pathogen is a fungus. In some embodiments, the infectious pathogen is a parasite.

In one aspect, provided herein is a method of activating a target cell expressing an IL-2R, comprising contacting the target cell with an effective amount of the immunoconjugate molecule of comprising an IL-2 polypeptide as described herein, wherein upon binding of the IL-2 polypeptide with the IL-2R, the target cell is activated. In some embodiments, the target cell is an immune cell. In some embodiments, the target cell is an effector T cell, memory T cell, regulatory T cell, or a combination thereof. In some embodiments, the target cell is an effector T cell. In some embodiments, the target cell is a memory T cell. In some embodiments, the target cell is a regulatory T cell.

In some embodiments, the target cell is CD4+ T cells, CD8+ T cells, helper T cells, cytotoxic T cells, SLECs (short-lived effector cells), MPEC (memory precursor effector cells), TEs (terminal effector cells), NKs (natural killer cells), NKTs (natural killer T cells), innate lymphoid cells (Types I-III), or a combination thereof. In some embodiments, the target cell is CD4+ T cells. In some embodiments, the target cell is CD8+ T cells. In some embodiments, the target cell is helper T cells. In some embodiments, the target cell is cytotoxic T cells. In some embodiments, the target cell is SLECs (short-lived effector cells). In some embodiments, the target cell is MPEC (memory precursor effector cells). In some embodiments, the target cell is TEs (terminal effector cells). In some embodiments, the target cell is NKs (natural killer cells). In some embodiments, the target cell is NKTs (natural killer T cells). In some embodiments, the target cell is innate lymphoid cells (Types I-III).

In some embodiments, the target cell is natural Treg (nTreg) cells, induced Treg (iTreg) cells, or a combination thereof. In some embodiments, the target cell is natural Treg (nTreg) cells. In some embodiments, the target cell is induced Treg (iTreg) cells.

In some embodiments, activation of the target cell is measured as increased proliferation or maturation of the target cell. In some embodiments, proliferation or maturation of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

In some embodiments, activation of the target cell is measured as prolonged survival time of the target cell. In some embodiments, survival time of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

In some embodiments, wherein the contacting further comprises administering a pharmaceutical composition comprising a pharmaceutically acceptable carrier and immunoconjugate molecule comprising an IL-2 polypeptide as described herein. In some embodiments, the contacting enhances an anti-neoplastic immune response. In some embodiments, the contacting enhances an anti-infection immune response.

In one aspect, provided herein is a method of enhancing an antigen-specific immune response of a population of T cells, comprising contacting the population of T cells with an effective amount of the immunoconjugate molecule comprising an IL-2 polypeptide as described herein. 141 In some embodiments, the contacting enhances proliferation or maturation of antigen-specific effector T cells. In some embodiments, the contacting enhances formation of antigen-specific memory T cells. In some embodiments, the contacting is performed in the presence of the antigen. In some embodiments, the antigen is an antigen of a cancer, tumor, pathogen, or allergen.

In one aspect, provided herein is a method of increasing secretion of pro-inflammatory cytokines by a population of T cells, comprising contacting the population of T cells with an immunoconjugate molecule comprising an IL-2 polypeptide as described herein, wherein said IL-2 polypeptide activates the T cells upon binding. In some embodiments, the cytokine is IL-1, IL-2, IL-6, IL-12, IL-17, IL-22, IL-23, GM-CSF, TNF-α, IFN-γ, or any combination thereof. In some embodiments, the cytokine is IL-1. In some embodiments, the cytokine is IL-2. In some embodiments, the cytokine is IL-6. In some embodiments, the cytokine is IL-12. In some embodiments, the cytokine is IL-17. In some embodiments, the cytokine is IL-22. In some embodiments, the cytokine is IL-23. In some embodiments, the cytokine is GM-CSF. In some embodiments, the cytokine is TNF-α. In some embodiments, the cytokine is IFN-7.

In some embodiments, the cytokine production is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

In one aspect, provided herein is a method of increasing assembly of IL-2R on the surface of a target cell, comprising contacting the target cell with an effective amount of the immunoconjugate molecule comprising an IL-2 polypeptide as described herein. In some embodiments, the IL-2R comprises IL-2Rα, IL-2Rβ, IL-2Rγ, or a combination thereof on the surface of the target cell. In some embodiments, the IL-2R comprises IL-2Rα on the surface of the target cell. In some embodiments, the IL-2R comprises IL-2Rβ on the surface of the target cell. In some embodiments, the IL-2R comprises IL-2Rγ on the surface of the target cell. In some embodiments, the IL-2R comprises IL-2Rα and IL-2Rβ on the surface of the target cell. In some embodiments, the IL-2R comprises IL-2Rα and IL-2Rγ on the surface of the target cell. In some embodiments, the IL-2R comprises IL-2Rβ and IL-2Rγ on the surface of the target cell. In some embodiments, the IL-2R comprises IL-2Rα, IL-2Rβ and IL-2Rγ on the surface of the target cell.

In some embodiments, the IL-2R comprises IL-2Rβ and IL-2Rγ on the surface of the target cell, and IL-2Rα on the surface of a second cell in proximity of the target cell. In some embodiments, the IL-2R comprises IL-2Rβ and IL-2Rγ on the surface of the target cell, and IL-2Rα not associated with a cell surface.

In some embodiments, assembly of IL-2R on the surface of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

In some embodiments, the target cell is an immune cell. In some embodiments, the target cell is an effector T cell, memory T cell, regulatory T cell, or a combination thereof. In some embodiments, the target cell is an effector T cell. In some embodiments, the target cell is a memory T cell. In some embodiments, the target cell is regulatory T cell.

In some embodiments, the target cell is CD4+ T cells, CD8+ T cells, helper T cells, cytotoxic T cells, SLECs (short-lived effector cells), MPEC (memory precursor effector cells), TEs (terminal effector cells), NKs (natural killer cells), NKTs (natural killer T cells), innate lymphoid cells (Types I-III), or a combination thereof. In some embodiments, the target cell is CD4+ T cells. In some embodiments, the target cell is CD8+ T cells. In some embodiments, the target cell is helper T cells. In some embodiments, the target cell is cytotoxic T cells. In some embodiments, the target cell is SLECs (short-lived effector cells). In some embodiments, the target cell is MPEC (memory precursor effector cells). In some embodiments, the target cell is TEs (terminal effector cells). In some embodiments, the target cell is NKs (natural killer cells). In some embodiments, the target cell is NKTs (natural killer T cells). In some embodiments, the target cell is innate lymphoid cells (Types I-III).

In some embodiments, the target cell is natural Treg (nTreg) cells, induced Treg (iTreg) cells, or a combination thereof. In some embodiments, the target cell is natural Treg (nTreg) cells. In some embodiments, the target cell is induced Treg (iTreg) cells.

In one aspect, provided herein is a method of forming a pro-inflammatory milieu in a tissue surrounding a population of diseased cells, comprising contacting the tissue with an effective amount of the immunoconjugate molecule comprising an IL-2 polypeptide as described herein.

In some embodiments, concentration of activated B cells, CD4+ effector T cells, CD8+ effector T cells, dendritic cells, macrophages, natural killer cells, monocytes, granulocytes, eosinophil and/or neutrophils in the tissue is increased. In some embodiments, concentration of activated B cells in the tissue is increased. In some embodiments, concentration of CD4+ effector T cells in the tissue is increased. In some embodiments, concentration of activated B cells in the tissue is increased. In some embodiments, concentration of CD8+ effector T cells in the tissue is increased. In some embodiments, concentration of dendritic cells in the tissue is increased. In some embodiments, concentration of macrophages in the tissue is increased. In some embodiments, concentration of natural killer cells in the tissue is increased. In some embodiments, concentration of monocytes in the tissue is increased. In some embodiments, concentration of granulocytes in the tissue is increased. In some embodiments, concentration of eosinophil in the tissue is increased. In some embodiments, concentration of neutrophils in the tissue is increased. In some embodiments, concentration of regulatory T cells in the tissue is reduced.

In some embodiments, concentration of a pro-inflammatory cytokine is increased in the tissue. In some embodiments, the pro-inflammatory cytokine is IL-1, IL-2, IL-6, IL-12, IL-17, IL-22, IL-23, GM-CSF, TNF-α, IFN-γ, or any combination thereof. In some embodiments, concentration of antibodies binding to antigens originated or derived from the diseased cells is increased in the tissue. In some embodiments, presentation of antigens originated or derived from the diseased cells by antigen presentation cells is increased in the tissue. In some embodiments, phagocytosis of the diseased cells is increased in the tissue. In some embodiments, apoptosis of the diseased cells induced by cell-mediated cytotoxicity is increased in the tissue. In some embodiments, apoptosis of the diseased cells induced by antibody-dependent cellular cytotoxicity is increased in the tissue. In some embodiments, the population of the diseased cells is reduced in the tissue. In some embodiments, the population of the diseased cells is reduced by about 10%, about 20%, about 30%, about 40%, about 45%, about 50%, about 55%, about 60%, about 65%, about 70%, about 75%, about 80%, about 85%, about 90%, about 95%, or about 99% in the tissue. In some embodiments, the population of the diseased cells is reduced by about 0.5% to 10%, about 10% to 20%, about 20% to 30%, about 30% to 40%, about 40% to 45%, about 45% to 50%, about 50% to 55%, about 55% to 60%, about 60% to 65%, about 65% to 70%, about 70% to 75%, about 75% to 80%, about 80% to 85%, about 85% to 90%, about 90% to 95%, or about 95% to 99% in the tissue.

In one aspect, provided herein is a method of eliminating a diseased cell in a subject, comprising administering to the subject an effective amount of the immunoconjugate molecule comprising an IL-2 polypeptide as described herein. In some embodiments, the diseased cell is a cancer cell. In some embodiments, the diseased cell is a cell infected by an infectious pathogen. In some embodiments, the infectious pathogen is a virus, a bacteria, a fungus, a parasite, or a combination thereof.

In one aspect, provided herein is a method of treating cancer in a subject in need thereof, comprising administering to the subject an effective amount of the immunoconjugate molecule comprising an IL-2 polypeptide as described herein. In some embodiments, the treatment enhances an innate, humoral or cell-mediated anti-neoplastic immune response. In some embodiments, the method further comprises co-administration of a second therapy.

In one aspect, provided herein is a method of treating an infection in a subject in need thereof, comprising administering to the subject an effective amount of the immunoconjugate molecule comprising an IL-2 polypeptide as described herein. In some embodiments, the treatment enhances an innate, humoral, or cell-mediated anti-infective immune response. In some embodiments, the subject is co-administered with a vaccine composition for preventing the infection in the subject. In some embodiments, the vaccine composition is co-administered simultaneously or sequentially.

In one aspect, provided herein is a method of increasing the response to an antigen in a subject in need thereof, comprising administering to the subject an effective amount of the immunoconjugate molecule comprising an TL-2 polypeptide as described herein. In some embodiments, the antigen is an antigen of a cancer, tumor, pathogen, or allergen. In some embodiments, the antigen is originated or derived from an infectious pathogen. In some embodiments, the infectious pathogen is a virus, a bacteria, a fungus, a parasite, or a combination thereof. In some embodiments, the antigen is originated or derived from a diseased cell. In some embodiments, the antigen is originated or derived from a cell infected by an infectious pathogen. In some embodiments, the infectious pathogen is a virus, a bacteria, a fungus, a parasite, or a combination thereof. In some embodiments, the antigen is originated or derived from a cancer cell.

In one aspect, provided herein is a method of increasing a response to a vaccine in a subject in need thereof, comprising administering to the subject the vaccine and an effective amount of the immunoconjugate molecule comprising an IL-2 polypeptide as described herein. In some embodiments, the vaccine is a vaccine against a tumor, cancer, pathogen or allergen. In some embodiments, the immunoconjugate molecule is formulated as an adjuvant composition for the vaccine.

In some embodiments of each or any of the above- or below-mentioned embodiments, the present immunoconjugate molecules are used for treating solid tumor cancer. In other embodiments, the present immunoconjugate molecules are used for treating blood cancer. In other embodiments, the disease or disorder is an autoimmune and inflammatory disease. In other embodiments, the disease or disorder is an infectious disease.

In some embodiments of each or any of the above- or below-mentioned embodiments, the disease or disorder is a disease of abnormal cell growth and/or dysregulated apoptosis. Examples of such diseases include, but are not limited to, cancer, mesothelioma, bladder cancer, pancreatic cancer, skin cancer, cancer of the head or neck, cutaneous or intraocular melanoma, ovarian cancer, breast cancer, uterine cancer, carcinoma of the fallopian tubes, carcinoma of the endometrium, carcinoma of the cervix, carcinoma of the vagina, carcinoma of the vulva, bone cancer, colon cancer, rectal cancer, cancer of the anal region, stomach cancer, gastrointestinal (gastric, colorectal and/or duodenal) cancer, chronic lymphocytic leukemia, acute lymphocytic leukemia, esophageal cancer, cancer of the small intestine, cancer of the endocrine system, cancer of the thyroid gland, cancer of the parathyroid gland, cancer of the adrenal gland, sarcoma of soft tissue, cancer of the urethra, cancer of the penis, testicular cancer, hepatocellular (hepatic and/or biliary duct) cancer, primary or secondary central nervous system tumor, primary or secondary brain tumor, Hodgkin's disease, chronic or acute leukemia, chronic myeloid leukemia, lymphocytic lymphoma, lymphoblastic leukemia, follicular lymphoma, lymphoid malignancies of T-cell or B-cell origin, melanoma, multiple myeloma, oral cancer, non-small-cell lung cancer, prostate cancer, small-cell lung cancer, cancer of the kidney and/or ureter, renal cell carcinoma, carcinoma of the renal pelvis, neoplasms of the central nervous system, primary central nervous system lymphoma, non-Hodgkin's lymphoma, spinal axis tumors, brain stem glioma, pituitary adenoma, adrenocortical cancer, gall bladder cancer, cancer of the spleen, cholangiocarcinoma, fibrosarcoma, neuroblastoma, retinoblastoma or a combination thereof.

In some embodiments of each or any of the above- or below-mentioned embodiments, the disease or disorder is selected from the group consisting of bladder cancer, brain cancer, breast cancer, bone marrow cancer, cervical cancer, chronic lymphocytic leukemia, acute lymphocytic leukemia, colorectal cancer, esophageal cancer, hepatocellular cancer, lymphoblastic leukemia, follicular lymphoma, lymphoid malignancies of T-cell or B-cell origin, melanoma, myelogenous leukemia, myeloma, oral cancer, ovarian cancer, non-small-cell lung cancer, prostate cancer, small-cell lung cancer and spleen cancer.

In some embodiments of each or any of the above- or below-mentioned embodiments, the disease or disorder is a hematological cancer, such as leukemia, lymphoma, or myeloma. In some embodiments of each or any of the above- or below-mentioned embodiments, the cancer is selected from a group consisting of Hodgkin's lymphoma, non-Hodgkin's lymphoma (NHL), cutaneous B-cell lymphoma, activated B-cell lymphoma, diffuse large B-cell lymphoma (DLBCL), mantle cell lymphoma (MCL), follicular center lymphoma, transformed lymphoma, lymphocytic lymphoma of intermediate differentiation, intermediate lymphocytic lymphoma (TLL), diffuse poorly differentiated lymphocytic lymphoma (PDL), centrocytic lymphoma, diffuse small-cleaved cell lymphoma (DSCCL), peripheral T-cell lymphomas (PTCL), cutaneous T-Cell lymphoma, mantle zone lymphoma, low grade follicular lymphoma, multiple myeloma (MM), chronic lymphocytic leukemia (CLL), diffuse large B-cell lymphoma (DLBCL), myelodysplastic syndrome (MDS), acute T cell leukemia, acute myeloid leukemia (AML), acute promyelocytic leukemia, acute myeloblastic leukemia, acute megakaryoblastic leukemia, precursor B acute lymphoblastic leukemia, precursor T acute lymphoblastic leukemia, Burkitt's leukemia (Burkitt's lymphoma), acute biphenotypic leukemia, chronic myeloid lymphoma, chronic myelogenous leukemia (CML), and chronic monocytic leukemia. In a specific embodiment, the disease or disorder is myelodysplastic syndromes (MDS). In another specific embodiment, the disease or disorder is acute myeloid leukemia (AML). In another specific embodiment, the disease or disorder is chronic lymphocytic leukemia (CLL). In yet another specific embodiment, the disease or disorder is multiple myeloma (MM).

In other embodiments, the disease or disorder is a solid tumor cancer. In some embodiments of each or any of the above- or below-mentioned embodiments, the solid tumor cancer is selected from a group consisting of a carcinoma, an adenocarcinoma, an adrenocortical carcinoma, a colon adenocarcinoma, a colorectal adenocarcinoma, a colorectal carcinoma, a ductal cell carcinoma, a lung carcinoma, a thyroid carcinoma, a nasopharyngeal carcinoma, a melanoma, a non-melanoma skin carcinoma, a liver cancer and a lung cancer.

In some embodiments of each or any of the above- or below-mentioned embodiments, the cancer is an adrenal cancer. In some embodiments of each or any of the above- or below-mentioned embodiments, the cancer is an anal cancer. In some embodiments of each or any of the above- or below-mentioned embodiments, the cancer is an appendix cancer. In some embodiments of each or any of the above- or below-mentioned embodiments, the cancer is a bile duct cancer. In some embodiments of each or any of the above- or below-mentioned embodiments, the cancer is a bladder cancer. In some embodiments of each or any of the above- or below-mentioned embodiments, the cancer is a bone cancer. In some embodiments of each or any of the above- or below-mentioned embodiments, the cancer is a brain cancer. In some embodiments of each or any of the above- or below-mentioned embodiments, the cancer is a breast cancer. In some embodiments of each or any of the above- or below-mentioned embodiments, the cancer is a cervical cancer. In some embodiments of each or any of the above- or below-mentioned embodiments, the cancer is a colorectal cancer. In some embodiments of each or any of the above- or below-mentioned embodiments, the cancer is an esophageal cancer. In some embodiments of each or any of the above- or below-mentioned embodiments, the cancer is a gallbladder cancer. In some embodiments of each or any of the above- or below-mentioned embodiments, the cancer is a gestational trophoblastic. In some embodiments of each or any of the above- or below-mentioned embodiments, the cancer is a head and neck cancer. In some embodiments of each or any of the above- or below-mentioned embodiments, the cancer is a Hodgkin lymphoma. In some embodiments of each or any of the above- or below-mentioned embodiments, the cancer is an intestinal cancer. In some embodiments of each or any of the above- or below-mentioned embodiments, the cancer is a kidney cancer. In some embodiments of each or any of the above- or below-mentioned embodiments, the cancer is a leukemia. In some embodiments of each or any of the above- or below-mentioned embodiments, the cancer is a liver cancer. In some embodiments of each or any of the above-or below-mentioned embodiments, the cancer is a lung cancer. In some embodiments of each or any of the above- or below-mentioned embodiments, the cancer is a melanoma. In some embodiments of each or any of the above- or below-mentioned embodiments, the cancer is a mesothelioma. In some embodiments of each or any of the above- or below-mentioned embodiments, the cancer is a multiple myeloma (MN). In some embodiments of each or any of the above- or below-mentioned embodiments, the cancer is a neuroendocrine tumor. In some embodiments of each or any of the above- or below-mentioned embodiments, the cancer is a non-Hodgkin lymphoma. In some embodiments of each or any of the above- or below-mentioned embodiments, the cancer is an oral cancer. In some embodiments of each or any of the above- or below-mentioned embodiments, the cancer is an ovarian cancer. In some embodiments of each or any of the above- or below-mentioned embodiments, the cancer is a pancreatic cancer. In some embodiments of each or any of the above- or below-mentioned embodiments, the cancer is a prostate cancer. In some embodiments of each or any of the above- or below-mentioned embodiments, the cancer is a sinus cancer. In some embodiments of each or any of the above- or below-mentioned embodiments, the cancer is a skin cancer. In some embodiments of each or any of the above- or below-mentioned embodiments, the cancer is a soft tissue sarcoma spinal cancer. In some embodiments of each or any of the above- or below-mentioned embodiments, the cancer is a stomach cancer. In some embodiments of each or any of the above- or below-mentioned embodiments, the cancer is a testicular cancer. In some embodiments of each or any of the above- or below-mentioned embodiments, the cancer is a throat cancer. In some embodiments of each or any of the above- or below-mentioned embodiments, the cancer is a thyroid cancer. In some embodiments of each or any of the above- or below-mentioned embodiments, the cancer is a uterine cancer endometrial cancer. In some embodiments of each or any of the above- or below-mentioned embodiments, the cancer is a vaginal cancer. In some embodiments of each or any of the above- or below-mentioned embodiments, the cancer is a vulvar cancer.

In some embodiments of each or any of the above- or below-mentioned embodiments, the adrenal cancer is an adrenocortical carcinoma (ACC), adrenal cortex cancer, pheochromocytoma, or neuroblastoma. In some embodiments of each or any of the above- or below-mentioned embodiments, the anal cancer is a squamous cell carcinoma, cloacogenic carcinoma, adenocarcinoma, basal cell carcinoma, or melanoma. In some embodiments of each or any of the above- or below-mentioned embodiments, the appendix cancer is a neuroendocrine tumor (NET), mucinous adenocarcinoma, goblet cell carcinoid, intestinal-type adenocarcinoma, or signet-ring cell adenocarcinoma. In some embodiments of each or any of the above- or below-mentioned embodiments, the bile duct cancer is an extrahepatic bile duct cancer, adenocarcinomas, hilar bile duct cancer, perihilar bile duct cancer, distal bile duct cancer, or intrahepatic bile duct cancer. In some embodiments of each or any of the above- or below-mentioned embodiments, the bladder cancer is transitional cell carcinoma (TCC), papillary carcinoma, flat carcinoma, squamous cell carcinoma, adenocarcinoma, small-cell carcinoma, or sarcoma. In some embodiments of each or any of the above- or below-mentioned embodiments, the bone cancer is a primary bone cancer, sarcoma, osteosarcoma, chondrosarcoma, sarcoma, fibrosarcoma, malignant fibrous histiocytoma, giant cell tumor of bone, chordoma, or metastatic bone cancer. In some embodiments of each or any of the above- or below-mentioned embodiments, the brain cancer is an astrocytoma, brain stem glioma, glioblastoma, meningioma, ependymoma, oligodendroglioma, mixed glioma, pituitary carcinoma, pituitary adenoma, craniopharyngioma, germ cell tumor, pineal region tumor, medulloblastoma, or primary CNS lymphoma. In some embodiments of each or any of the above- or below-mentioned embodiments, the breast cancer is a breast adenocarcinoma, invasive breast cancer, noninvasive breast cancer, breast sarcoma, metaplastic carcinoma, adenocystic carcinoma, phyllodes tumor, angiosarcoma, HER2-positive breast cancer, triple-negative breast cancer, or inflammatory breast cancer. In some embodiments of each or any of the above- or below-mentioned embodiments, the cervical cancer is a squamous cell carcinoma, or adenocarcinoma. In some embodiments of each or any of the above- or below-mentioned embodiments, the colorectal cancer is a colorectal adenocarcinoma, primary colorectal lymphoma, gastrointestinal stromal tumor, leiomyosarcoma, carcinoid tumor, mucinous adenocarcinoma, signet ring cell adenocarcinoma, gastrointestinal carcinoid tumor, or melanoma. In some embodiments of each or any of the above- or below-mentioned embodiments, the esophageal cancer is an adenocarcinoma or squamous cell carcinoma. In some embodiments of each or any of the above- or below-mentioned embodiments, the gall bladder cancer is an adenocarcinoma, papillary adenocarcinoma, adenosquamous carcinoma, squamous cell carcinoma, small cell carcinoma, or sarcoma. In some embodiments of each or any of the above- or below-mentioned embodiments, the gestational trophoblastic disease (GTD) is a hydatidiform mole, gestational trophoblastic neoplasia (GTN), choriocarcinoma, placental-site trophoblastic tumor (PSTT), or epithelioid trophoblastic tumor (ETT). In some embodiments of each or any of the above- or below-mentioned embodiments, the head and neck cancer is a laryngeal cancer, nasopharyngeal cancer, hypopharyngeal cancer, nasal cavity cancer, paranasal sinus cancer, salivary gland cancer, oral cancer, oropharyngeal cancer, or tonsil cancer. In some embodiments of each or any of the above- or below-mentioned embodiments, the Hodgkin lymphoma is a classical Hodgkin lymphoma, nodular sclerosis, mixed cellularity, lymphocyte-rich, lymphocyte-depleted, or nodular lymphocyte-predominant Hodgkin lymphoma (NLPHL). In some embodiments of each or any of the above- or below-mentioned embodiments, the intestinal cancer is a small intestine cancer, small bowel cancer, adenocarcinoma, sarcoma, gastrointestinal stromal tumors, carcinoid tumors, or lymphoma. In some embodiments of each or any of the above- or below-mentioned embodiments, the kidney cancer is a renal cell carcinoma (RCC), clear cell RCC, papillary RCC, chromophobe RCC, collecting duct RCC, unclassified RCC, transitional cell carcinoma, urothelial cancer, renal pelvis carcinoma, or renal sarcoma. In some embodiments of each or any of the above- or below-mentioned embodiments, the leukemia is an acute lymphocytic leukemia (ALL), acute myeloid leukemia (AML), chronic lymphocytic leukemia (CLL), chronic myeloid leukemia (CMIL), hairy cell leukemia (HCL), or a myelodysplastic syndrome (MDS). In a specific embodiment, the leukemia is AMIL. In some embodiments of each or any of the above- or below-mentioned embodiments, the liver cancer is a hepatocellular carcinoma (HCC), fibrolamellar HCC, cholangiocarcinoma, angiosarcoma, or liver metastasis. In some embodiments of each or any of the above- or below-mentioned embodiments, the lung cancer is a small cell lung cancer, small cell carcinoma, combined small cell carcinoma, non-small cell lung cancer, lung adenocarcinoma, squamous cell lung cancer, large-cell undifferentiated carcinoma, pulmonary nodule, metastatic lung cancer, adenosquamous carcinoma, large cell neuroendocrine carcinoma, salivary gland-type lung carcinoma, lung carcinoid, mesothelioma, sarcomatoid carcinoma of the lung, or malignant granular cell lung tumor. In some embodiments of each or any of the above- or below-mentioned embodiments, the melanoma is a superficial spreading melanoma, nodular melanoma, acral-lentiginous melanoma, lentigo maligna melanoma, amelanotic melanoma, desmoplastic melanoma, ocular melanoma, or metastatic melanoma. In some embodiments of each or any of the above- or below-mentioned embodiments, the mesothelioma is a pleural mesothelioma, peritoneal mesothelioma, pericardial mesothelioma, or testicular mesothelioma. In some embodiments of each or any of the above- or below-mentioned embodiments, the multiple myeloma is an active myeloma or smoldering myeloma. In some embodiments of each or any of the above- or below-mentioned embodiments, the neuroendocrine tumor is a gastrointestinal neuroendocrine tumor, pancreatic neuroendocrine tumor, or lung neuroendocrine tumor. In some embodiments of each or any of the above- or below-mentioned embodiments, the non-Hodgkin's lymphoma is an anaplastic large-cell lymphoma, lymphoblastic lymphoma, peripheral T cell lymphoma, follicular lymphoma, cutaneous T cell lymphoma, lymphoplasmacytic lymphoma, marginal zone B-cell lymphoma, MALT lymphoma, small-cell lymphocytic lymphoma, Burkitt lymphoma, chronic lymphocytic leukemia (CLL), small lymphocytic lymphoma (SLL), precursor T-lymphoblastic leukemia/lymphoma, acute lymphocytic leukemia (ALL), adult T cell lymphoma/leukemia (ATLL), hairy cell leukemia, B-cell lymphomas, diffuse large B-cell lymphoma (DLBCL), primary mediastinal B-cell lymphoma, primary central nervous system (CNS) lymphoma, mantle cell lymphoma (MCL), marginal zone lymphomas, mucosa-associated lymphoid tissue (MALT) lymphoma, nodal marginal zone B-cell lymphoma, splenic marginal zone B-cell lymphoma, lymphoplasmacytic lymphoma, B-cell non-Hodgkin lymphoma, T cell non-Hodgkin lymphoma, natural killer cell lymphoma, cutaneous T cell lymphoma, Alibert-Bazin syndrome, Sezary syndrome, primary cutaneous anaplastic large-cell lymphoma, peripheral T cell lymphoma, angioimmunoblastic T cell lymphoma (AITL), anaplastic large-cell lymphoma (ALCL), systemic ALCL, enteropathy-type T cell lymphoma (EATL), or hepatosplenic gamma/delta T cell lymphoma. In some embodiments of each or any of the above- or below-mentioned embodiments, the oral cancer is a squamous cell carcinoma, verrucous carcinoma, minor salivary gland carcinomas, lymphoma, benign oral cavity tumor, eosinophilic granuloma, fibroma, granular cell tumor, karatoacanthoma, leiomyoma, osteochondroma, lipoma, schwannoma, neurofibroma, papilloma, condyloma acuminatum, verruciform xanthoma, pyogenic granuloma, rhabdomyoma, odontogenic tumors, leukoplakia, erythroplakia, squamous cell lip cancer, basal cell lip cancer, mouth cancer, gum cancer, or tongue cancer. In some embodiments of each or any of the above- or below-mentioned embodiments, the ovarian cancer is a ovarian epithelial cancer, mucinous epithelial ovarian cancer, endometrioid epithelial ovarian cancer, clear cell epithelial ovarian cancer, undifferentiated epithelial ovarian cancer, ovarian low malignant potential tumors, primary peritoneal carcinoma, fallopian tube cancer, germ cell tumors, teratoma, dysgerminoma ovarian germ cell cancer, endodermal sinus tumor, sex cord-stromal tumors, sex cord-gonadal stromal tumor, ovarian stromal tumor, granulosa cell tumor, granulosa-theca tumor, Sertoli-Leydig tumor, ovarian sarcoma, ovarian carcinosarcoma, ovarian adenosarcoma, ovarian leiomyosarcoma, ovarian fibrosarcoma, Krukenberg tumor, or ovarian cyst. In some embodiments of each or any of the above- or below-mentioned embodiments, the pancreatic cancer is a pancreatic exocrine gland cancer, pancreatic endocrine gland cancer, or pancreatic adenocarcinoma, islet cell tumor, or neuroendocrine tumor. In some embodiments of each or any of the above- or below-mentioned embodiments, the prostate cancer is a prostate adenocarcinoma, prostate sarcoma, transitional cell carcinoma, small cell carcinoma, or neuroendocrine tumor. In some embodiments of each or any of the above- or below-mentioned embodiments, the sinus cancer is a squamous cell carcinoma, mucosa cell carcinoma, adenoid cystic cell carcinoma, acinic cell carcinoma, sinonasal undifferentiated carcinoma, nasal cavity cancer, paranasal sinus cancer, maxillary sinus cancer, ethmoid sinus cancer, or nasopharynx cancer. In some embodiments of each or any of the above- or below-mentioned embodiments, the skin cancer is a basal cell carcinoma, squamous cell carcinoma, melanoma, Merkel cell carcinoma, Kaposi sarcoma (KS), actinic keratosis, skin lymphoma, or keratoacanthoma. In some embodiments of each or any of the above- or below-mentioned embodiments, the soft tissue cancer is an angiosarcoma, dermatofibrosarcoma, epithelioid sarcoma, Ewing's sarcoma, fibrosarcoma, gastrointestinal stromal tumors (GISTs), Kaposi sarcoma, leiomyosarcoma, liposarcoma, dedifferentiated liposarcoma (DL), myxoid/round cell liposarcoma (MRCL), well-differentiated liposarcoma (WDL), malignant fibrous histiocytoma, neurofibrosarcoma, rhabdomyosarcoma (RMS), or synovial sarcoma. In some embodiments of each or any of the above- or below-mentioned embodiments, the spinal cancer is a spinal metastatic tumor. In some embodiments of each or any of the above- or below-mentioned embodiments, the stomach cancer is a stomach adenocarcinoma, stomach lymphoma, gastrointestinal stromal tumors, carcinoid tumor, gastric carcinoid tumors, Type I ECL-cell carcinoid, Type II ECL-cell carcinoid, or Type III ECL-cell carcinoid. In some embodiments of each or any of the above- or below-mentioned embodiments, the testicular cancer is a seminoma, non-seminoma, embryonal carcinoma, yolk sac carcinoma, choriocarcinoma, teratoma, gonadal stromal tumor, leydig cell tumor, or sertoli cell tumor. In some embodiments of each or any of the above- or below-mentioned embodiments, the throat cancer is a squamous cell carcinoma, adenocarcinoma, sarcoma, laryngeal cancer, pharyngeal cancer, nasopharynx cancer, oropharynx cancer, hypopharynx cancer, laryngeal cancer, laryngeal squamous cell carcinoma, laryngeal adenocarcinoma, lymphoepithelioma, spindle cell carcinoma, verrucous cancer, undifferentiated carcinoma, or lymph node cancer. In some embodiments of each or any of the above- or below-mentioned embodiments, the thyroid cancer is a papillary carcinoma, follicular carcinoma, H0rthle cell carcinoma, medullary thyroid carcinoma, or anaplastic carcinoma. In some embodiments of each or any of the above- or below-mentioned embodiments, the uterine cancer is an endometrial cancer, endometrial adenocarcinoma, endometroid carcinoma, serous adenocarcinoma, adenosquamous carcinoma, uterine carcinosarcoma, uterine sarcoma, uterine leiomyosarcoma, endometrial stromal sarcoma, or undifferentiated sarcoma. In some embodiments of each or any of the above- or below-mentioned embodiments, the vaginal cancer is a squamous cell carcinoma, adenocarcinoma, melanoma, or sarcoma. In some embodiments of each or any of the above-or below-mentioned embodiments, the vulvar cancer is a squamous cell carcinoma or adenocarcinoma.

In one aspect, provided herein is a method of establishing immune tolerance of an antigen in a tissue surrounding the antigen, comprising contacting the tissue with an effective amount of the immunoconjugate molecule comprising an IL-2 polypeptide as described herein. In some embodiments, concentration of activated B cells, CD4+ effector T cells, CD8+ effector T cells, dendritic cells, macrophages, natural killer cells, monocytes, granulocytes, eosinophil and/or neutrophils in the tissue is reduced. In some embodiments, concentration of regulatory T cells in the tissue is increased. In some embodiments, concentration of a pro-inflammatory cytokine is reduced in the tissue. In some embodiments, the pro-inflammatory cytokine is IL-1, IL-2, IL-6, IL-12, IL-17, IL-22, IL-23, GM-CSF, TNF-α, IFN-γ or any combination thereof. In some embodiments, concentration of antibodies binding to the antigen is reduced in the tissue. In some embodiments, presentation of the antigen by antigen presentation cells is reduced in the tissue. In some embodiments, phagocytosis of cells expressing the antigen is reduced in the tissue. In some embodiments, apoptosis of cells expressing the antigen is reduced in the tissue. In some embodiments, wherein the tissue is in a subject, and wherein the antigen is a self-antigen of the subject. In some embodiments, the subject is suffering from an autoimmune disease.

In yet another aspect, provided herein is a method for treating an autoimmune disease in a subject in need thereof, comprising administering to the subject an effective amount of the immunoconjugate molecule comprising an IL-2 polypeptide as described herein. In some embodiments, the treatment reduces an innate, humoral or cell-mediated immune response towards a self-antigen. In some embodiments, the method further comprises co-administration of a second therapy.

In some embodiments of each or any of the above- or below-mentioned embodiments, the disease or disorder is an immune or autoimmune disorder. Such disorders include autoimmune bullous disease, abetalipoprotemia, acquired immunodeficiency-related diseases, acute immune disease associated with organ transplantation, acquired acrocyanosis, acute and chronic parasitic or infectious processes, acute pancreatitis, acute renal failure, acute rheumatic fever, acute transverse myelitis, adenocarcinomas, aerial ectopic beats, adult (acute) respiratory distress syndrome, AIDS dementia complex, alcoholic cirrhosis, alcohol-induced liver injury, alcohol-induced hepatitis, allergic conjunctivitis, allergic contact dermatitis, allergic rhinitis, allergy and asthma, allograft rejection, alpha-l-antitrypsin deficiency, Alzheimer's disease, amyotrophic lateral sclerosis, anemia, angina pectoris, ankylosing spondylitis-associated lung disease, anterior horn cell degeneration, antibody mediated cytotoxicity, antiphospholipid syndrome, anti-receptor hypersensitivity reactions, aortic and peripheral aneurysms, aortic dissection, arterial hypertension, arteriosclerosis, arteriovenous fistula, arthropathy, asthenia, asthma, ataxia, atopic allergy, atrial fibrillation (sustained or paroxysmal), atrial flutter, atrioventricular block, atrophic autoimmune hypothyroidism, autoimmune haemolytic anaemia, autoimmune hepatitis, type-1 autoimmune hepatitis (classical autoimmune or lupoid hepatitis), autoimmune mediated hypoglycemia, autoimmune neutropenia, autoimmune thrombocytopenia, autoimmune thyroid disease, B-cell lymphoma, bone graft rejection, bone marrow transplant (BMT) rejection, bronchiolitis obliterans, bundle branch block, burns, cachexia, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy-associated disorders, chlamydia, choleosatatis, chronic alcoholism, chronic active hepatitis, chronic fatigue syndrome, chronic immune disease associated with organ transplantation, chronic eosinophilic pneumonia, chronic inflammatory pathologies, chronic mucocutaneous candidiasis, chronic obstructive pulmonary disease (COPD), chronic salicylate intoxication, colorectal common varied immunodeficiency (common variable hypogammaglobulinemia), conjunctivitis, connective tissue disease-associated interstitial lung disease, contact dermatitis, Coombs-positive hemolytic anemia, cor pulmonale, Creutzfeldt-Jakob disease, cryptogenic autoimmune hepatitis, cryptogenic fibrosing alveolitis, culture-negative sepsis, cystic fibrosis, cytokine therapy-associated disorders, Crohn's disease, dementia pugilistica, demyelinating diseases, dengue hemorrhagic fever, dermatitis, dermatitis scleroderma, dermatologic conditions, dermatomyositis/polymyositis-associated lung disease, diabetes, diabetic arteriosclerotic disease, diabetes mellitus, diffuse Lewy body disease, dilated cardiomyopathy, dilated congestive cardiomyopathy, discoid lupus erythematosus, disorders of the basal ganglia, disseminated intravascular coagulation, Down's Syndrome in middle age, drug-induced interstitial lung disease, drug-induced hepatitis, drug-induced movement disorders induced by drugs which block CNS dopamine receptors, drug sensitivity, eczema, encephalomyelitis, endocarditis, endocrinopathy, enteropathic synovitis, epiglottitis, Epstein-Barr virus infection, erythromelalgia, extrapyramidal and cerebellar disorders, familial hematophagocytic lymphohistiocytosis, fetal thymus implant rejection, Friedreich's ataxia, functional peripheral arterial disorders, female infertility, fibrosis, fibrotic lung disease, fungal sepsis, gas gangrene, gastric ulcer, giant cell arteritis, glomerular nephritis, glomerulonephritides, Goodpasture's syndrome, goitrous autoimmune hypothyroidism (Hashimoto's disease), gouty arthritis, graft rejection of any organ or tissue, graft versus host disease, gram-negative sepsis, gram-positive sepsis, granulomas due to intracellular organisms, group B streptococci (GBS) infection, Graves' disease, hemosiderosis-associated lung disease, hairy cell leukemia, Hallerrorden-Spatz disease, Hashimoto's thyroiditis, hay fever, heart transplant rejection, hemachromatosis, hematopoietic malignancies (leukemia and lymphoma), hemolytic anemia, hemolytic uremic syndrome/thrombolytic thrombocytopenic purpura, hemorrhage, Henoch-Schoenlein purpura, hepatitis A, hepatitis B, hepatitis C, HIV infection/HIV neuropathy, Hodgkin's disease, hypoparathyroidism, Huntington's chorea, hyperkinetic movement disorders, hypersensitivity reactions, hypersensitivity pneumonitis, hyperthyroidism, hypokinetic movement disorders, hypothalamic-pituitary-adrenal axis evaluation, idiopathic Addison's disease, idiopathic leucopenia, idiopathic pulmonary fibrosis, idiopathic thrombocytopenia, idiosyncratic liver disease, infantile spinal muscular atrophy, infectious diseases, inflammation of the aorta, inflammatory bowel disease, insulin dependent diabetes mellitus, interstitial pneumonitis, iridocyclitis/uveitis/optic neuritis, ischemia-reperfusion injury, ischemic stroke, juvenile pernicious anemia, juvenile rheumatoid arthritis, juvenile spinal muscular atrophy, Kaposi's sarcoma, Kawasaki's disease, kidney transplant rejection, legionella, leishmaniasis, leprosy, lesions of the corticospinal system, linear IgA disease, lipidema, liver transplant rejection, Lyme disease, lymphederma, lymphocytic infiltrative lung disease, malaria, male infertility idiopathic or NOS, malignant histiocytosis, malignant melanoma, meningitis, meningococcemia, microscopic vasculitis of the kidneys, migraine headache, mitochondrial multisystem disorder, mixed connective tissue disease, mixed connective tissue disease-associated lung disease, monoclonal gammopathy, multiple myeloma, multiple systems degenerations (Mencel, Dejerine-Thomas, Shy-Drager and Machado-Joseph), myalgic encephalitis/Royal Free Disease, myasthenia gravis, microscopic vasculitis of the kidneys, Mycobacterium avium intracellulare, Mycobacterium tuberculosis, myelodyplastic syndrome, myocardial infarction, myocardial ischemic disorders, nasopharyngeal carcinoma, neonatal chronic lung disease, nephritis, nephrosis, nephrotic syndrome, neurodegenerative diseases, neurogenic I muscular atrophies, neutropenic fever, non-alcoholic steatohepatitis, occlusion of the abdominal aorta and its branches, occlusive arterial disorders, organ transplant rejection, orchitis/epidydimitis, orchitis/vasectomy reversal procedures, organomegaly, osteoarthrosis, osteoporosis, ovarian failure, pancreas transplant rejection, parasitic diseases, parathyroid transplant rejection, Parkinson's disease, pelvic inflammatory disease, pemphigus vulgaris, pemphigus foliaceus, pemphigoid, perennial rhinitis, pericardial disease, peripheral atherlosclerotic disease, peripheral vascular disorders, peritonitis, pernicious anemia, phacogenic uveitis, Pneumocystis carinii pneumonia, pneumonia, POEMS syndrome (polyneuropathy, organomegaly, endocrinopathy, monoclonal gammopathy, and skin changes syndrome), post-perfusion syndrome, post-pump syndrome, post-MI cardiotomy syndrome, postinfectious interstitial lung disease, premature ovarian failure, primary biliary cirrhosis, primary sclerosing hepatitis, primary myxoedema, primary pulmonary hypertension, primary sclerosing cholangitis, primary vasculitis, progressive supranuclear palsy, psoriasis, psoriasis type 1, psoriasis type 2, psoriatic arthropathy, pulmonary hypertension secondary to connective tissue disease, pulmonary manifestation of polyarteritis nodosa, post-inflammatory interstitial lung disease, radiation fibrosis, radiation therapy, Raynaud's phenomenon and disease, Raynoud's disease, Refsum's disease, regular narrow QRS tachycardia, Reiter's disease, renal disease NOS, renovascular hypertension, reperfusion injury, restrictive cardiomyopathy, rheumatoid arthritis-associated interstitial lung disease, rheumatoid spondylitis, sarcoidosis, Schmidt's syndrome, scleroderma, senile chorea, senile dementia of Lewy body type, sepsis syndrome, septic shock, seronegative arthropathies, shock, sickle cell anemia, T-cell or FAB ALL, Takayasu's disease/arteritis, telangiectasia, Th2-type and Th1-type mediated diseases, thromboangitis obliterans, thrombocytopenia, thyroiditis, toxicity, toxic shock syndrome, transplants, trauma/hemorrhage, type-2 autoimmune hepatitis (anti-LKM antibody hepatitis), type B insulin resistance with acanthosis nigricans, type III hypersensitivity reactions, type IV hypersensitivity, ulcerative colitic arthropathy, ulcerative colitis, unstable angina, uremia, urosepsis, urticaria, uveitis, valvular heart diseases, varicose veins, vasculitis, vasculitic diffuse lung disease, venous diseases, venous thrombosis, ventricular fibrillation, vitiligo acute liver disease, viral and fungal infections, vital encephalitis/aseptic meningitis, vital-associated hemaphagocytic syndrome, Wegener's granulomatosis, Wernicke-Korsakoff syndrome, Wilson's disease, xenograft rejection of any organ or tissue, yersinia and salmonella-associated arthropathy, acquired immunodeficiency disease syndrome (AIDS), autoimmune lymphoproliferative syndrome, hemolytic anemia, inflammatory diseases, thrombocytopenia, acute and chronic immune diseases associated with organ transplantation, Addison's disease, allergic diseases, alopecia, alopecia areata, atheromatous disease/arteriosclerosis, atherosclerosis, arthritis (including osteoarthritis, juvenile chronic arthritis, septic arthritis, Lyme arthritis, psoriatic arthritis and reactive arthritis), Sjogren's disease-associated lung disease, Sjogren's syndrome, skin allograft rejection, skin changes syndrome, small bowel transplant rejection, sperm autoimmunity, multiple sclerosis (all subtypes), spinal ataxia, spinocerebellar degenerations, spondyloarthropathy, sporadic polyglandular deficiency type I, sporadic polyglandular deficiency type II, Still's disease, streptococcal myositis, stroke, structural lesions of the cerebellum, subacute sclerosing panencephalitis, sympathetic ophthalmia, syncope, syphilis of the cardiovascular system, systemic anaphylaxis, systemic inflammatory response syndrome, systemic onset juvenile rheumatoid arthritis, systemic lupus erythematosus, systemic lupus erythematosus-associated lung disease, lupus nephritis, systemic sclerosis, and systemic sclerosis-associated interstitial lung disease.

### 5.5 Pharmaceutical Compositions

In one aspect, the present disclosure further provides pharmaceutical compositions comprising at least one immunoconjugate molecule of the present disclosure. In some embodiments, a pharmaceutical composition comprises 1) the immunoconjugate molecule, and 2) a pharmaceutically acceptable carrier.

Pharmaceutical compositions comprising an antibody or antibody-containing immunoconjugate molecule are prepared for storage by mixing the antibody or the immunoconjugate molecule having the desired degree of purity with optional physiologically acceptable carriers, excipients, or stabilizers (see, e.g., Remington, Remington's Pharmaceutical Sciences (18th ed. 1980)) in the form of aqueous solutions or lyophilized or other dried forms.

The immunoconjugate molecule of the present disclosure may be formulated in any suitable form for delivery to a target cell/tissue, e.g., as microcapsules or macroemulsions (Remington, supra; Park et al., 2005, Molecules 10:146-61; Malik et al., 2007, Curr. Drug. Deliv. 4:141-51), as sustained release formulations (Putney and Burke, 1998, Nature Biotechnol. 16:153-57), or in liposomes (Maclean et al., 1997, Int. J. Oncol. 11:325-32; Kontermann, 2006, Curr. Opin. Mol. Ther. 8:39-45).

An immunoconjugate molecule provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with an antibody or antibody-containing molecules such as the immunoconjugate molecule as described herein, including, but not limited to, encapsulation in liposomes, microparticles, microcapsules, recombinant cells capable of expressing the antibody, receptor-mediated endocytosis (see, e.g., Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see, e.g., Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., an antibody that binds to PD-1 as described herein) or a composition of the invention (see, e.g., Medical Applications of Controlled Release (Langer and Wise eds., 1974); Controlled Drug Bioavailability, Drug Product Design and Performance (Smolen and Ball eds., 1984); Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see, e.g., Goodson, Medical Applications of Controlled Release Vol. 2, 115-38 (1984)). Controlled release systems are discussed, for example, by Langer, 1990, Science 249:1527-33. Any technique known to one of skill in the art can be used to produce sustained release formulations comprising one or more antibodies that bind to PD-1 as described herein (see, e.g., U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 5.6 Kits

Also provided herein are kits comprising an immunoconjugate molecule as provided herein, or a composition (e.g., a pharmaceutical composition) thereof, packaged into suitable packaging material. A kit optionally includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampoules, vials, tubes, etc.).

Kits provided herein can include labels or inserts. Labels or inserts include “printed matter,” e.g., paper or cardboard, separate or affixed to a component, a kit or packing material (e.g., a box), or attached to, for example, an ampoule, tube, or vial containing a kit component. Labels or inserts can additionally include a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media, or memory type cards. Labels or inserts can include information identifying manufacturer information, lot numbers, manufacturer location, and date.

Kits provided herein can additionally include other components. Each component of the kit can be enclosed within an individual container, and all of the various containers can be within a single package. Kits can also be designed for cold storage. A kit can further be designed to contain antibodies provided herein, or cells that contain nucleic acids encoding the antibodies provided herein. The cells in the kit can be maintained under appropriate storage conditions until ready to use.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

All applications, publications, patents and other references, GenBank citations and ATCC citations cited herein are incorporated by reference in their entirety. In case of conflict, the specification, including definitions, will control.

As used herein, the singular forms “a,” “and,” and “the” include plural referents unless the context clearly indicates otherwise. Thus, for example, reference to “a peptide sequence” includes a plurality of such sequences and so forth.

As used herein, numerical values are often presented in a range format throughout this document. The use of a range format is merely for convenience and brevity and should not be construed as an inflexible limitation on the scope of the invention unless the context clearly indicates otherwise. Accordingly, the use of a range expressly includes all possible subranges, all individual numerical values within that range, and all numerical values or numerical ranges including integers within such ranges and fractions of the values or the integers within ranges unless the context clearly indicates otherwise. This construction applies regardless of the breadth of the range and in all contexts throughout this patent document. Thus, for example, reference to a range of 90-100% includes 91-99%, 92-98%, 93-95%, 91-98%, 91-97%, 91-96%, 91-95%, 91-94%, 91-93%, and so forth. Reference to a range of 90-100% also includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth.

In addition, reference to a range of 1-3, 3-5, 5-10, 10-20, 20-30, 30-40, 40-50, 50-60, 60-70, 70-80, 80-90, 90-100, 100-110, 110-120, 120-130, 130-140, 140-150, 150-160, 160-170, 170-180, 180-190, 190-200, 200-225, 225-250 includes 1, 2, 3, 4, 5, 6, 7, 8, 9, 10, 11, 12, 13, 14, 15, 16, 17, 18, 19, 20, etc. In a further example, reference to a range of 25-250, 250-500, 500-1,000, 1,000-2,500, 2,500-5,000, 5,000-25,000, 25,000-50,000 includes any numerical value or range within or encompassing such values, e.g., 25, 26, 27, 28, 29 . . . 250, 251, 252, 253, 254 . . . 500, 501, 502, 503, 504 . . . , etc.

As also used herein a series of ranges are disclosed throughout this document. The use of a series of ranges include combinations of the upper and lower ranges to provide another range. This construction applies regardless of the breadth of the range and in all contexts throughout this patent document. Thus, for example, reference to a series of ranges such as 5-10, 10-20, 20-30, 30-40, 40-50, 50-75, 75-100, 100-150, includes ranges such as 5-20, 5-30, 5-40, 5-50, 5-75, 5-100, 5-150, and 10-30, 10-40, 10-50, 10-75, 10-100, 10-150, and 20-40, 20-50, 20-75, 20-100, 20-150, and so forth.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

A number of embodiments of the invention have been described. Nevertheless, it will be understood that various modifications may be made without departing from the spirit and scope of the invention. Accordingly, the following examples are intended to illustrate but not limit the scope of invention described in the claims.

### 6. EXAMPLES

The examples in this section (i.e., Section 6) are offered by way of illustration, and not by way of limitation.

### 6.1 Example 1: General Methods

### 6.1.1 Cell Lines and Culturing Conditions

If not indicated differently, all cell culture media and supplements were obtained from Gibco by Thermofisher. HEK 293T cells was purchased from (Fenghui ShengWu, China) and was maintained in DMEM supplemented with 10% fetal bovine serum (FBS), 1% L-glutamine (L-glu), 1% Na Pyruvate, 1% penicillin and streptomycin (P/S). HEK Blue IL2 reporter cell line was purchased from InVivoGen, USA and was maintained in DMEM supplemented with 10% heat-inactivated fetal bovein serum (FBS), 1% L-glutamine (L-glu), 1% Na Pyruvate, 1% penicillin and streptomycin (P/S) with 100 μg/mL Normacin (InVivogen). CTLL-2 cell line was purchased from American Type Culture Collection (ATCC) and cultured with RPMI supplemented with 10% fetal bovein serum (FBS), 1% L-glutamine (L-glu), 1% Na Pyruvate, 1% penicillin and streptomycin (P/S). NK-92 cell line was purchased from Procell, China and was maintained in company provided media consisting of RPMI supplemented with 20 U/mL IL2, MEMa, 0.2 mM Inositol, Folic Acid, 0.1 mM b-mercaptoethanol, 12.5% horse serum, 12.5% fetal bovine serum, 1% P/S. Expi293F (Cat #: A14635). ExpiCHO (Cat #: A29133) cells were purchased from Thermofisher and maintained in manufacture-provided media.

6.1.2 Generation of Stable hFAP-Expressing Cell Line

Adherent HEK 293T cells stably expressing hFAP were generated as described below. hFAP expression vector with hygromycin resistance gene was purchased from Sino Biologics (Cat #: HG10464-UT). The plasmid was transfected into HEK 293T using Lipofectamine 2000 (Thermofisher) system. 24 hrs after the transfection, 150 μg/mL Hygromycin was added into the cell culture, and fresh media was changed when necessary in the following two weeks. The surviving cells were expanded, and the hFAP expression was confirmed using flow cytometer (CYTOFLEX, Beckman Coulter) labeled with anti-FAP mAB (Cat #: BMS168, Thermofisher) and Goat anti-mouse Alexa 488 (Cat #: A32723, Thermofisher). The FAP expression clone was sorted from the pool using BD FACSAria III sorter. A high expression clone designated as HEK 293T-hFAP-E5 was selected and used in the assays as described below, and its receptor density was calibrated around 3×10⁶/cell using the Quantum Alexa Fluor 488 MESF kit (Bangslab, USA).

Suspension ExpiCHO cells expressing hFAP were generated similarly as described above, except that the cells were maintained in suspension and the media was not changed. A high expression clone designated as ExpiCHO-hFAP-G7 was selected and used in the assays as described below.

### 6.1.3 Affinity Determination by Biolayer Interferometry:

Binding kinetics of antibody-antigen interactions were determined by biolayer interferometry using the Gator BLI system. Particularly, biotinylated antigen was immobilized on a sensor coated with streptavidin to a response level of ˜0.5-1.0 nm Candidate antibodies were constructed in the form of a monovalent Fab-Fc fusion protein containing a Knob-in-Hole modification in the Fc portion, and were subjected to serial two-fold dilutions that resulted in final concentrations in the range of 100 nM to 3.1 nM. The antibody sample was applied to the sensor and incubated for up to 240 seconds to allow antibody-antigen association, which was followed by incubating the sensor in PBST-BSA for up to 420 seconds to allow disassociation. Binding constants (kₒₙ, k_{off} and K_{D}) were determined after referencing by subtracting responses from sensors without immobilized antigen. Data were fit globally with Gator software using a 1:1 binding interaction while fitting the responses to an unlinked Rₘₐₓ. All protein samples were diluted in PBST-BSA. Typical protocol for K_{D} determination:

| Step | Time | | Solution |
|---|---|---|---|
| Baseline | 120 | seconds | PBST-BSA |
| Antigen | 180 | seconds | Biotinylated Antigen (10-100 nM) |
| Immobilization | | | |
| Baseline | 120 | seconds | PBST-BSA |
| Association | 240 | seconds | Fab-Fc(Knob-in-Hole), _various |
| | | | concentrations |
| Dissociation | 240-420 | seconds | PBST-BSA |

### 6.2 Example 2: Generation of Antibodies

### 6.2.1 Generation of Anti-FAP Antibodies

The parental FAP mAbs were initially generated by phage display methods using human Fibroblast Activation Protein (FAP) antigen. Fabs were isolated from phage antibody libraries constructed by Kunkel mutagenesis where codon-based sequence diversification of the CDRs was introduced by phosphoramidite trinucleotide-based primers. Three to four rounds phage panning were performed with antigen immobilized on Streptavidin beads. Initial characterization of a phage pool by monoclonal phage ELISA identified 82 subclones producing anti-human FAP antibodies with K_{D} ˜1-100 nM to soluble FAP antigen (data not shown), and were designated as IgG-1 through IgG-82, respectively. Antibodies were subcloned into the IgG1 and Fab-Fc format for further studies.

HEK 293T and HEK 293T-FAP-E5 cells were used for cell binding to confirm selected antibodies were able to bind to the epitope on cell surface, and to exclude antibodies with non-specific binding to cells. HEK 293T-FAP-E5 is a single clone expressing ˜1×10⁶ FAP/cell. It was generated by transient transfection on parental HEK 293T cells, sorted by FACS, selected by hygromycin resistance, and its receptor density was quantified by Quantum MESF 488 (Bangs Laboratories, USA) following the manufacture-provided procedure. Both live and fixed cells were used for cell binding. For cell fixation, HEK 293T and HEK 293T-FAP-E5 cells were detached, washed twice with PBS, fixed with 4% paraformaldehyde, and stored in PBS-1% BSA.

Antibody binding to cells was assayed by flow cytometry with Cytoflex (Beckman Coulter). In brief, 1 μg/mL purified antibody was incubated with 5×10⁴ cells in the volume of 100 uL for 30 minutes. Cells were centrifuged and washed once with PBS-1% BSA. 1 μg/mL secondary antibody goat anti-human Alexa488 (A-11013, Thermofisher) was incubated with washed cells in the volume of 100 μL for 30 minutes. Cells were centrifuged and washed once with PBS-1% BSA. The labeled cells were resuspended in 300 μL and loaded on to Cytoflex using the FTIC settings. The pair of primary polyclonal FAP antibody (PA5-95481, Thermofisher) and secondary goat anti-rabbit Alexa488 (A-11008, Thermofisher) were used as positive control. The pair of antibodies including an isotype antibody DP47GS and secondary antibody goat anti-human Alexa488 (A-11013, Thermofisher) were used as negative controls. All antibodies mentioned in Tables 1 to 8 can bind to both HEK 293T-FAP-E5 cells and not to HEK 293T cells.

A panel of 13 clones (872-2, 872-5, 872-10, 872-11, 872-19, 872-26, 872-39, 872-44, 872-58, 872-59, 872-67, 872-70 and 872-75) were selected and subjected to the epitope binning study.

### 6.2.2 Epitope Binning

To determine whether the selected anti-FAP antibodies share non-overlapping epitopes, epitope binning studies were performed on the Gator™ Biolayer interferometry (BLI) system. Antigen (biotinylated FAP) was immobilized to a response level of ˜0.5-1 nm. After establishing a baseline, sensors were subjected to saturating levels (>1 μM) of anti-FAP IgG antibodies. After a short dissociation period of about 60 seconds, the sensor was incubated in a solution containing a second antibody and the response was monitored. Antibody pairs were considered to have different epitopes if the binding event of the second antibody resulted in a response >50% of the first antibody binding event. Antibody pairs where the second antibody event did not result in any further increase in the signal after the first antibody binding event were considered to share overlapping epitopes. Table 9 below summarizes the results of the epitope binning study.

TABLE 9

| | 2^{nd} Binding Ab | 1^{st} Binding Ab | | | |
|---|---|---|---|---|---|
| | Antibody | IgG 5 | IgG 59 | IgG 67 | IgG 70 |
| 1 | 872-2 | Same | Same | Ambiguous | Ambiguous |
| 2 | 872-5 | Same | Same | Different | Different |
| 3 | 872-10 | Same | Same | Same | Same |
| 4 | 872-11 | Same | Same | Different | Same |
| 5 | 872-19 | Same | Same | Different | Same |
| 6 | 872-26 | Same | Different | Different | Different |
| 7 | 872-39 | Same | Same | Different | Different |
| 8 | 872-44 | Same | Same | Same | Same |
| 9 | 872-58 | Same | Same | Same | Different |
| 10 | 872-59 | Same | Same | Different | Different |
| 11 | 872-67 | Same | Different | Same | Different |
| 12 | 872-70 | Different | Different | Different | Same |
| 13 | 872-75 | Same | Same | Same | Same |

### 6.2.3 Cell-Based FAP Binding Assay

Antibodies with confirmed binding by biolayer interferometry were subsequently screened for the ability to bind to HEK-293 cells expressing human FAP. Three anti-FAP IgG antibodies (produced by Clones IgG 5, IgG 59, and IgG 70, respectively, and designated as antibodies 872-5, 872-59, and 872-70, respectively) that bind to two non-overlapping epitopes of FAP at nM level of dissociation constants (Table 10) were selected as the starting antibodies for generation of anti-IL-2/anti-FAP bispecific antibodies.

TABLE 10

| Antibody | kₒₙ (M⁻¹s⁻¹) | k_{off} (s⁻¹) | K_{D} (nM) |
|---|---|---|---|
| 872-5 | 4.2 × 10⁵ | 2.8 × 10⁻³ | 6.6 |
| 872-59 | 3.6 × 10⁵ | 5.6 × 10⁻³ | 15.5 |
| 872-70 | 1.1 × 10⁵ | Slow | <1 |

### 6.2.4 Generation of Anti-IL-2/Anti-FAP Bispecific Antibodies

Phage displaying libraries were constructed for each of the three starting anti-FAP antibodies. Particularly, Kunkel mutagenesis, each codon encoding an amino acid residue in the 6 complementarity-determining regions (CDR) of a starting antibody was replaced with the degenerate codon NNK, one position at a time. Saturation mutagenesis of each mutated residue within a CDR were pooled for subsequent library preparation and phage panning. DNA of the constructed libraries was electroporated into SS320 cells pre-infected with M13K07 following published procedures. Phage was prepared for panning similarly to selections of naive libraries, with several modifications. 1 pmol of biotinylated antigen was used for phage panning. During the course of the selection, 1 μM of soluble competitor was added to well E of the KingFisher plate. This allowed for “off-rate” selection where soluble antigen can compete for binding to phage once it had dissociated from the antigen-coated beats due to the vast molar excess of soluble competitor. Additionally, a parallel selection was performed with biotinylated anti-CH1 antibody to monitor expression bias within the panning experiment. Three rounds of phage panning were performed and the resulting outputs were prepared for next-generation sequencing. The resulting sequence data were analyzed for amino acid distribution preferences within the CDRs.

Secondary libraries were constructed to introduce amino acid diversity into CDR positions found to be amenable to mutation by saturation mutagenesis-NGS sequencing analysis. Phage libraries were constructed in a similar manner to naive antibody libraries using Kunkel mutagenesis with synthesized primers coupled with electroporation into E. co/i strain SS320 pre-infected with M13K07 helper phage. Phage panning was performed on a number of IL-2 variants resulting in antibodies recognizing at least two non-overlapping epitopes of IL-2.

### 6.2.5 Phage Panning for Bispecific Antibody Isolation:

Constructed antibody libraries were subjected to four rounds of phage panning. Particularly, 500 μL of solution containing the starting phage library was diluted to an A₂₆₈=1 (˜1×10¹² colony forming units/mL) in PBST-BSA (Phosphate buffered saline supplemented with 0.2% Tween 20, 2% Bovine Serum Albumin). The phage library was pre-cleared by incubation with 20 μL M280 Streptavidin Dynabeads for one hour. After pre-clearance, the phage library was incubated with 20 μL Dynabeads coated with 50 pmoles biotinylated IL2 (Acro Biosciences). Samples were incubated at room temperature for −1 hour with gentle mixing. Beads were then sedimented with a magnetic stand to remove unbound phage. Samples were washed three times with 500 μL PBST-BSA and then incubated with 200 μL 0.1 M glycine (pH 2.7) for 15 minutes to elute the phage from the beads. The supernatant of the elution was then separated from the beads, neutralized with 40 μL 1 M HEPES, pH 7.2. The elution and beads were added to 5 mL mid log-phase XL1-blue cells and allowed to incubate at room temperature for 30 minutes. The infected cells were then sub-cultured by addition of 25 mL 2xYT supplemented with Ampicillin (50 pg/mL) and M13K07 helper phage (˜10¹⁰ pfu/mL). Cell cultures were allowed to grow for −16 hours at 37° C. with vigorous shaking.

Rounds 2-4 of phage panning were performed similarly. Particularly, after culturing infected cells overnight, the cells were centrifuged to pellet and removed. The resulting supernatant was precipitated by adding 1/5 volume PEG/NaCl solution and incubated for 30 minutes on ice. After centrifugation at 10,000×g for 15 minutes, the supernatant was removed and the phage pellet was resuspended in 200 μL PBS. The resuspended phage was centrifuged at 14,000×g for 5 minutes to remove insoluble materials. The phage was then transferred to a fresh Eppendorf® tube and precipitated a second time by adding 40 μL PEG/NaCl solution. The samples were placed on ice for −15 minutes before spinning at 10,000×g for 10 minutes. The supernatant was removed and phage was resuspended in 200 μL PBS. Samples were again centrifuged at 14,000×g for 5 minutes to remove insoluble materials. Phage was quantitated and prepared for pre-clearance with streptavidin beads. Here, 250 μL of phage A₂₆₈=0.2-0.5 (round 2 uses A₂₆₈=0.5, rounds 3 and 4 use A₂₆₈=0.2) in PBST-BSA was incubated with 10 μL M280 Streptavidin Dynabeads for 30 minutes. After pre-clearance, the phage was added to Well C of a 200 uL KingFisher™ plate for bead manipulation. The following was added to the KingFisher™ plate for phage panning:

| Well | Solution | Volume |
|---|---|---|
| Well A | Streptavidin beads + Antigen (variable concentration) | 100 μL |
| Well B | 10 μM Biotin solution in PBS | 100 μL |
| Well C | Pre-cleared phage solution | 100 μL |
| Well D | PBST-BSA | 100 μL |
| Well E | PBST-BSA | 100 μL |
| Well F | PBST-BSA | 100 μL |
| Well G | PBST-BSA | 100 μL |
| Well H | 0.1M Glycine pH 2.7 | 100 μL |

The KingFisher™ protocol used for phage panning was the following:

| Well | Solution | Time |
|---|---|---|
| Well A | 15 minutes incubation with fast mixing | 15 min. |
| Well B | Transfer to well B with fast mixing | 5 min. |
| Well C | Transfer to well C with fast mixing | 15 min. |
| Well D | Transfer to well D with fast mixing | 5 min. |
| Well E | Transfer to well E with fast mixing | 1 min. |
| Well F | Transfer to well F with fast mixing | 1 min. |
| Well G | Transfer to well G with fast mixing | 1 min. |
| Well H | 15 minutes with fast mixing | 15 min. |
| Well G | Release beads | N/A |

After the KingFisher™ phage panning protocol, the phage was neutralized with 20 μL 1 M HEPES. 50 μL of the phage elution was added to 500 μL mid log phase XL1 for 30 minutes for infection. The infected cells were then sub-cultured in 2.5 mL 2xYT supplemented with Ampicillin (50 pg/mL) and M13K07 helper phage (˜10¹⁰ pfu/mL). Cell cultures were grown overnight at 37° C. with vigorous shaking to amplify phage. Additionally, phage was quantitated by plating serial dilutions of the infection to monitor the number of colony forming units in the elution of each round.

Phage panning was continued through four rounds. The concentration of antigen used during each round was in the range of 100 nM to 10 nM with lower concentrations used during later rounds. Phage panning experiments were monitored for increases in phage titer in successive rounds of panning.

### 6.2.6 Monoclonal Phage ELISA:

To identify individual clones from the phage panning studies with the desired binding properties (e.g., antibodies capable of binding to both FAP and IL-2), monoclonal phage ELISA study was performed. Phage from the elution was used to infect XL1-blue cells. After 30 minutes, the cells were plated on LB-Ampicillin plates so that individual colonies could be isolated and picked. After incubation overnight at 37° C., the colonies on the plate were picked and placed in a 96-deep well block and incubated with 400 μL 2xYT supplemented with Ampicillin (50 μg/mL) and M13K07 helper phage (˜10¹⁰ pfu/mL). Plates were incubated overnight at 37° C. with vigorous shaking. After −14 hours of incubation, the plates were centrifuged (4000×g for 10 minutes) to pellet cells. The resulting phage-containing supernatant was diluted 10-fold in PBST-BSA. 50 μL of the diluted phage-containing supernatant was incubated in three separate wells of a Maxisorpm ELISA plate. Well #1 contained 2.5 pmoles of immobilized interleukin-2, Well #2 contained 2.5 pmoles of immobilized FAP, and Well #3 was coated with BSA. Phage was incubated for 30 minutes before washing three times with PBST. The plates were then incubated with 50 μL 0.2 μg/mL anti-M13-HRP antibody (SinoBiological, Cat #11973-MM05T-H) for 30 minutes. ELISA plates were again washed three times in PBST. Horseradish Peroxidase activity was detected with 1-Step™ Ultra TMB-ELISA TMB substrate (ThermoFisher). ELISA plates were allowed to develop for approximately five minutes and reactions were quenched with 1 M M Phosphoric acid. Reactions were quantitated by measuring absorbance at 410 nm. Samples with significant signal (>3 times about background) were sent for sequence analysis.

Through the screening of the phage libraries, three variants of the 872-70 parent antibody were identified and designated as D001, D002 and D029 variants. These variants were able to (a) bind to the wild-type IL-2 polypeptide and the IL-2hex mutant that does not bind to the IL-2 receptor CD25. To bias the phage panning selection toward epitopes that would likely impair IL-2 signaling, selections of wild-type IL-2 were performed in the presence of an a-IL-2 antibody NARA which binds coincident to the CD25 epitope of IL-2; (b) inhibiting IL-2 activity; and (c) retaining FAP binding activities.

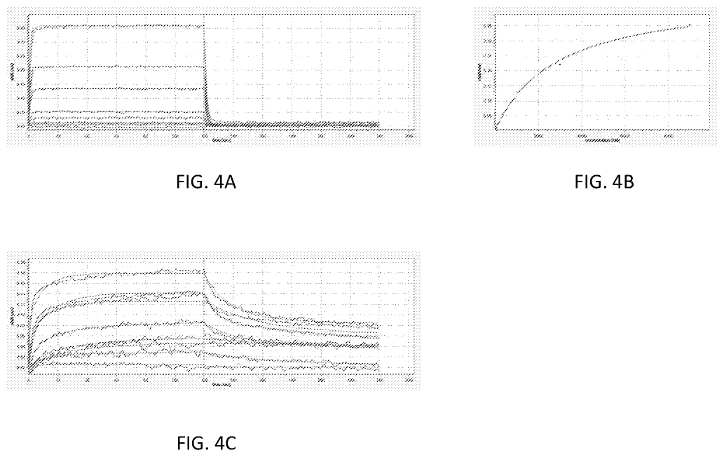

FIG. 4A shows binding kinetics of the monovalent Fab-Fc fusion of D002 to biotinylated IL-2 immobilized on Streptavidin sensor and measured by bio-layer interferometry, and FIG. 4B shows the K_{D} value was 3.4 μM for the interaction of D002 with IL-2, determined by equilibrium binding analysis. FIG. 4C shows binding kinetics of the monovalent Fab-Fc fusion of D002 to FAP immobilized on Streptavidin sensor and measured by bio-layer interferometry. The K_{D} value was 50 nM for the interaction of D002 with FAP (data not shown).

### 6.2.7 Generation of Antibody Variants

In order to examine possible effects of the molecular configuration of the immunoconjugate on the activity of the molecule, Fab and scFv variants were generated for the starting anti-FAP antibodies (872-5, 872-59, and 872-70) and the three variants (D001, D002 and D029), respectively.

Particularly, Fab and scFv variants of the antibodies were recombinantly produced by combining the binding sequences of the parental antibodies. Additionally, a single domain anti-FAP antibody (designated as VHH6) was generated by phage display panning from synthetic VHH phage libraries. Phage panning was performed using the same procedure described for Fab-based phage libraries. Table 11 summarizes the types of variants generated in this study, the epitope bins and binding affinity measured for the generated variants.

TABLE 11
Binding Affinity of Antibody Variants to FAP and IL-2.

| Antibody | Type | Epitope Bin | FAP K_{D} | | IL-2 K_{D} | |
|---|---|---|---|---|---|---|
| 872-5 | Fab/scFv | Different from | 7 | nM | N.B. | |
| | | 872-59, 872-70 | | | | |
| 872-59 | Fab/scFv | Different from | 16 | nM | N.B. | |
| | | 872-59, 872-70 | | | | |
| 872-70 | Fab/scFv | Different from | <1 | nM | N.B. | |
| | | 872-59, 872-70 | | | | |
| VHH6 | Single Domain | N/A | ~μM | | N.B. | |
| D001 | Fab/scFv | Same as 872-70 | 30 | nM | >5 | μM |
| D002 | Fab/scFv | Same as 872-70 | ~50 | nM | 3.4 | μM |

N.B. = no detectable binding
** = estimated value

### 6.2.8 Affinity Maturation

Affinity maturation of the anchoring arm. Affinity maturation of the 872-5 monoclonal antibody was guided by amino acid sequence distributions within the CDRs that was obtained by next-generation sequencing. Briefly, we observed that four positions within the CDRs of 872-5 were enriched in amino acids different from the parent residue after saturation mutagenesis coupled with phage panning. These mutants included VL A91G, VL R92T, VH S52G and VH Q96L. Single mutants were tested for binding to human FAP by biolayer interferometry described in Section 6.1.3 and resulted in improvements in K_{D} of ˜3-fold to 9-fold (Table 12). Single point mutants were then combined to create seven combinations of double, triple and quadruple mutations. The highest affinity observed was less than 100 μM, a greater than 80-fold improvement of the affinity over parent 872-5.

TABLE 12

| 872 Variant | K_{D} | | kₒₙ (M⁻¹s⁻¹) (std. error) | | k_{off} (s⁻¹) (std. error) | |
|---|---|---|---|---|---|---|
| 872-5 | 7.3 | nM | 2.77 × 10⁵ | (8.46 × 10²) | 2.01 × 10⁻³ | (3.53 × 10⁻⁶) |
| 872-5 V_{L} A91G | 780 | pM | 1.30 × 10⁶ | (2.81 × 10³) | 1.02 × 10⁻³ | (1.92 × 10⁻⁶) |
| 872-5 V_{L} T92R | 1.6 | nM | 6.14 × 10⁵ | (4.49 × 10³) | 1.00 × 10⁻³ | (8.10 × 10⁻⁶) |
| 872-5 VH S52G | 2.2 | nM | 6.42 × 10⁵ | (4.01 × 10³) | 1.38 × 10⁻³ | (7.45 × 10⁻⁶) |
| 872-5 VH Q96L | 1.5 | nM | 5.42 × 10⁵ | (1.89 × 10³) | 7.94 × 10⁻⁴ | (3.27 × 10⁻⁶) |
| 872-5 V_{H} | 241 | pM | 7.37 × 10⁵ | (2.03 × 10³) | 1.78 × 10⁻⁴ | (2.03 × 10⁻⁶) |
| S52G/Q96L:V_{L} | | | | | | |
| A91G/T92R | | | | | | |
| 872-5 V_{H} | 251 | pM | 6.45 × 10⁵ | (1.54 × 10³) | 1.61 × 10⁻⁴ | (1.76 × 10⁻⁶) |
| S52G/Q96L | | | | | | |
| 872-5 V_{H} | <100 | pM | 6.25 × 10⁵ | (1.77 × 10³) | Slow | |
| S52G/Q96L/V_{L} | | | | | | |
| A91G (872-5V1) | | | | | | |
| 872-5 V_{H} | 87 | pM | 8.78 × 10⁵ | (2.23 × 10³) | 7.61 × 10⁻⁵ | (1.71 × 10⁻⁶) |
| S52G/Q96L/V_{L} | | | | | | |
| T92R | | | | | | |
| 872-5 V_{L} | 398 | pM | 1.03 × 10⁶ | (3.05 × 10³) | 4.11 × 10⁻⁴ | (2.04 × 10⁻⁶) |
| A91G/T92R | | | | | | |
| 872-5 V_{H} S52G:V_{L} | 2.2 | nM | 6.73 × 10⁵ | (3.44 × 10³) | 1.48 × 10⁻³ | (4.60 × 10⁻⁶) |
| A91G/T92R | | | | | | |
| 872-5 V_{H} Q96L:V_{L} | 707 | pM | 8.12 × 10⁵ | (2.69 × 10³) | 5.71 × 10⁻⁴ | (2.54 × 10⁻⁶) |
| A91G/T92R | | | | | | |

Affinity maturation of bispecific antibody. Affinity maturation of the D029 variant to restore binding to FAP while maintaining binding to IL-2 was guided by next generation sequencing. Comprehensive mutagenesis of the CDRs of mAb D029 was performed by Kunkel mutagenesis similarly to the methods described for mAbs 872-5, 872-59 and 872-70. After phage panning and NGS library preparation and analysis, the sequence distributions for amino acids in the CDRs was compared to 872-70 (the parent monoclonal antibody of D029). Differences between the sequences of mAb D029 and mAb 872-70 were assessed and a series of reversion mutations were created. These mutants were reformatted into monoclonal antibody format, and Fc-Fab format to test binding and for reformatting into immunocytokine constructs. Furthermore, a series of mutations were created that next-generation sequencing indicated were compatible between the two antigens that were not present originally in either 872-70 or D029. These mutants of D029 were tested for binding in small-scale crude lysate and in purified form by biolayer interferometry as described in Section 6.1.3 (Table 13).

TABLE 13A

| | | IL-2 K_{D} |
|---|---|---|
| D029 and Variants | FAP K_{D} (nM) | (nM) |
| D029 | N.B. at 1 μM | 431 |
| D029 Rev1/S55L (D029HVILV1) | weak (crude lysate | N.D. |
| | measurement) | |
| Rev1:Q27G:P94Y/S55L (D029HVILV4) | ~100 (crude lysate | N.D. |
| | measurement) | |
| Rev1:Q27G:P94Y:L96F/S55L | ~100 (crude lysate | N.D. |
| (D029HVILV2) | measurement) | |
| D029 VH-S55L:T30R | ~100 (crude lysate | N.D. |
| | measurement) | |
| Rev1/S55L:T30R (D029 T30R:S55L/LV1) | ~100 (crude lysate | N.D. |
| | measurement) | |
| Rev1:Q27G:P94Y/S55L:T30R (D029VH | ~100 (crude lysate | N.D. |
| T30R:S55L/LV4) | measurement) | |
| Rev1:Q27G:P94Y:L96F/S55L:T30R | ~100 (crude lysate | N.D. |
| (D029VH T30R:S55L/LV2) | measurement) | |
| Rev1:Q27G:P94Y (D029 LV4) | ~100 (crude lysate | N.D. |
| | measurement) | |
| D029L-Rev1/S55L:T30R:S32F (D029 | 1.79 | 427 |
| HV3LV1) | | |
| D029-Rev1:Q27G:P94Y/S55L:T30R:S32F | 1.8 | 1100 |
| (D029 HV3LV4) | | |
| D029L- | 3.58 | 331 |
| Rev1:Q27G:P94Y:L96F/S55L:T3OR:S32F | | |
| (D029 HV3LV2) | | |
| Rev1-Q27G:L96F/S55L:S32F (D029 | 3.66 | 217 |
| HV2LV3) | | |
| Rev1/S55L:S32F (D029 HV2LV1) | 2.31 | 528 |
| Rev1/S55L:T30R:S32A (D029 HV5LV1) | >100 | 100 |
| Rev1-Q27G:P94Y/S55L:S32F (D029 | 1.53 | 1590 |
| HV2LV4) | | |
| Rev1-Q27G:P94Y:L96F/S55L:S32F (D029 | 1.98 | 419 |
| HV2LV2) | | |

N.D. = not determined
N.B. = no detectable binding

TABLE 13B

| | | | EC50 of HEK-Blue IL2 | |
|---|---|---|---|---|
| | | | assay (pM) | |
| | Affinity (nM) | | with HEK | with HEK |
| D029 and Variants | FAP | IL2hex | 293T | 293T-hFAP-E5 |
| D029 | N/A at | 431 | 1512 | 56 |
| | 1 uM | | | |
| Rev1/S55L (D029HVILV1) | 1.8 | 427 | 6058 | 42 |
| Rev1/S55L:T30R:S32F (D029HV3LV1) | weak | 237 | 7376 | 35 |
| Rev1:Q27G:P94Y/S55L:T30R:S32F | 2.3 | 528 | 4123 | 208 |
| (D029HV3LV4) | | | | |
| Rev1:Q27G:P94Y:L96F/S55L:T30R:S32F | 100 | 100 | 5333 | 186 |
| (D029HV3LV2) | | | | |
| Rev1-Q27G:L96F/S55L:S32F | 3.7 | 217 | 5298 | 31 |
| (D029HV2LV3) | | | | |
| Rev1/S55L:T30R:S32A (D029 HV5LV1) | 3.6 | 331 | 5631 | 41 |
| Rev1-Q27G:P94Y/S55L:S32F | 1.5 | 1590 | 5136 | 43 |
| (D029HV2LV4) | | | | |
| Rev1-Q27G:P94Y:L96F/S55L:S32A | 62 | 199 | 4818 | 145 |
| (D029HV6LV2) | | | | |
| Rev1-Q27G:P94Y:L96F/S55L:S32F | 2 | 419 | 6059 | 27 |
| (D029HV2LV2) | | | | |
| Rev1-Q27G/P94Y/L96F:H1V9 | 0.51 | 4500 | 6095 | 40 |
| (D029HV4LV2) | | | | |
| Rev1-Q27G/P94Y/L96F:H1V10 | 10 | 700 | 14752 | 117 |
| Rev1-Q27G/P94Y/L96F:H1V11 | 0.51 | 600 | 9373 | 57 |
| Rev1/S55L:S32F (D029HV2LV1) | 1.8 | 1100 | 9262 | 78 |

“H1V10” = D029 VH domain variant containing the T30S:W31R:S55L mutations
“H1V11’ = D029 VH domain variant containing the W31Y:S32F:S55L mutations

### 6.3 Example 3: Generation of Recombinant Antibody-Cytokine Immunoconjugate

### 6.3.1 Generation of Antibody-Cytokine Immunoconjugate Proteins of Different Molecular Configurations

Antibody-cytokine immunoconjugates having different molecular configurations as illustrated in FIGS. 5B through 5U were recombinantly generated and screened for the ability of shielding and de-shielding (see Table 14). Particularly, DNA sequences of immunoconjugates were codon optimized and cloned into pcDNA3.4 vector (Thermofisher) with a signal peptide as secreted proteins. Each peptide chain was cloned into an independent vector. At the fusion junction, the C-terminal lysine residue of the CH3 domain was removed. Proteins were expressed in Expi293F expression system (Thermofisher), and Fc-containing proteins were purified with MonoA (Genescript) protein A affinity resin. In brief, plasmids of individual chain were combined at equal mass ratio and transfected to Expi293F cells using ExpiFectamine. The cells were fed −18 hours after transfection and the supernatant were harvested within 5-7 days after expression by centrifugation at 4000 rpm for 5 minutes. After MonoA resin was incubated with supernatant and washed, the proteins were eluted by 0.1 acetic acid pH 4.0, neutralized with 1/5 volume of 1 M Tris pH 8.0, and dialyzed in PBS pH7.4.

TABLE 14

| | | | | | Config- | | | Linker | Linker |
|---|---|---|---|---|---|---|---|---|---|
| | | | | | uration | | | to | to |
| | | Expression | | De- | as shown | | | IL2 | Anchor |
| ID | Protein Description* | and Yield | Shielding | shielding | in FIG. 5 | Two-in-One | Anchoring | (# of aa) | (# of aa) |
| | IL2 WT | | | | 1 | | | | |
| | Knob-IL2hex | +++ | | | 2 | | | 15 | |
| 277 | scFv5-Knob-IL2hex x | +++ | Y | Y | 3 | D002 | scFv5 | 15 | 15 |
| | Hole-D002 | | | | | | | | |
| 294 | scFv59-Knob-IL2hex x | +++ | Y | Y | 3 | D002 | scFv59 | 15 | 15 |
| | Hole-D002 | | | | | | | | |
| 295 | scFv70-Knob-IL2hex x | +++ | Y | Y | 3 | D002 | scFv70 | 15 | 15 |
| | Hole-D002 | | | | | | | | |
| 471 | scFv70-sc80-Knob- | +++ | Y | Y | 3 | D029 | scFv70 | 15 | 15 |
| | IL2hex x Hole-D029 | | | | | | | | |
| | (VH) | | | | | | | | |
| 558 | scFv70-sc80-Knob- | +++ | Y | Y | 3 | D029- | scFv70 | 15 | 80 |
| | IL2hex x sHole- | | | | | HVILV1 | | | |
| | D029HV1 x D029LV1 | | | | | | | | |
| 675 | scFv872-5Dbl(S-S)-sc70- | +++ | Y | Y | 3 | D029- | scFv5 | 15 | 70 |
| | sKnob-IL2hex x sHole- | | | | | HV2LV3 | | | |
| | D029HV2 x D029LV3 | | | | | | | | |
| 676 | scFv872-5Dbl(S-S)-sc70- | +++ | Y | Y | 3 | D029- | scFv5 | 15 | 70 |
| | sKnob-IL2hex x sHole- | | | | | HV2LV4 | | | |
| | D029HV2 x D029LV4 | | | | | | | | |
| 278 | VHH6-Knob-IL2hex x | + | Y | Y | 5 | D002 | VHH6 | 15 | 15 |
| | Hole-D002 | | | | | | | | |
| 694 | VHH6-sc30-sKnob- | +++ | Y | Y | 5 | D029- | VHH6 | 15 | 30 |
| | IL2hex x sHole- | | | | | HV2LV4 | | | |
| | D029HV2 x D029LV4 | | | | | | | | |
| 695 | VHH6-sc50-sKnob- | +++ | Y | Y | 5 | D029- | VHH6 | 15 | 50 |
| | IL2hex x sHole- | | | | | HV2LV4 | | | |
| | D029HV2 x D029LV4 | | | | | | | | |
| 696 | VHH6-sc70-sKnob- | +++ | Y | Y | 5 | D029- | VHH6 | 15 | 70 |
| | IL2hex x sHole- | | | | | HV2LV4 | | | |
| | D029HV2 x D029LV4 | | | | | | | | |
| 353 | FAP59-LC x FAP59- | + | Y | Y | 6 | D002 | mAB59 | 15 | 15 |
| | (VH)-Knob-IL2hex x | | | | | | | | |
| | Hole-D002 (scFv) | | | | | | | | |
| 354 | FAP70-LC x FAP70- | + | Y | Y | 6 | D002 | mAB70 | 15 | 15 |
| | (VH)-Knob-IL2hex x | | | | | | | | |
| | Hole-D002 (scFv) | | | | | | | | |
| 355 | FAP5-LC x FAP5-(VH)- | + | Y | Y | 6 | D002 | mAB59 | 15 | 15 |
| | Knob-IL2hex x Hole- | | | | | | | | |
| | D002 (scFv) | | | | | | | | |
| 371 | Knob-D002-(VH) x | ++ | Y | Y | 7 | D002 | scFv5 | 50 | 15 |
| | IL2hex-sc50-D002-(VL) | | | | | | | | |
| | x scFv5-Hole | | | | | | | | |
| 372 | Knob-D002-(VH) x | + | moderate | Y | 7 | D002 | scFv59 | 15 | 80 |
| | IL2hex-sc50-D002-(VL) | | | | | | | | |
| | x scFv59-Hole | | | | | | | | |
| 280 | mAB70-Knob-IL2hex x | − | Y | N | 8 | D002 | mAB70 | 15 | 15 |
| | Hole-sc50-D002 | | | | | | | | |
| 292 | scFv5-Knob x Hole- | − | | | 9 | D002 | scFv5 | 15 | 15 |
| | IL2hex-D002 (VL) | | | | | | | | |
| 293 | scFv59-Knob x Hole- | − | | | 9 | D002 | scFv59 | 15 | 15 |
| | IL2hex-D002 (VL) | | | | | | | | |
| 472 | scFv70-Knob x Hole- | +++ | Y | N | 9 | D029 | scFv70 | 15 | 15 |
| | D029 (VH) x IL2hex- | | | | | | | | |
| | sc80-D029 (VL) | | | | | | | | |
| 473 | scFv5-Knob x Hole-D029 | +++ | Y | N | 9 | D029 | scFv5 | 15 | 15 |
| | (VH) x IL2hex-sc80- | | | | | | | | |
| | D029 (VL) | | | | | | | | |
| 526 | scFv5-Knob x Hole-D029 | − | | | 9 | D029 | scFv5 | 15 | 15 |
| | (VH) x IL2hex-sc30- | | | | | | | | |
| | D029 (VL) | | | | | | | | |
| 527 | scFv70-Knob x Hole- | − | | | 9 | D029 | scFv70 | 15 | 15 |
| | D029 (VH) x IL2hex- | | | | | | | | |
| | sc30-D029 (VL) | | | | | | | | |
| 547 | IL2hex-sc30-D029HV1- | +++ | Y | moderate | 10 | D029HVILV1 | mAB70 | 15 | 15 |
| | sKnob x mAB70 (VH)- | | | | | | | | |
| | sHole x D029LV1 | | | | | | | | |
| 548 | IL2hex-sc80-D029HV1- | +++ | Y | moderate | 10 | D029- | mAB70 | 15 | 15 |
| | sKnob x mAB70 (VH)- | | | | | | | | |
| | sHole x D029L V1 | | | | | HVILV1 | | | |
| 314 | Knob-IL2hex x scFv5- | +++ | Y | Y | 11 | D002 | scFv5 | 15 | 50 |
| | sc50-Hole-D002 | | | | | | | | |
| 315 | Knob-IL 2hex x scFv59- | +++ | Y | Y | 11 | D002 | scFv59 | 15 | 50 |
| | sc50-Hole-D002 | | | | | | | | |
| 316 | Knob-IL2hex x scFv70- | +++ | Y | Y | 11 | D002 | scFv70 | 15 | 50 |
| | sc50-Hole-D002 | | | | | | | | |
| 317 | Knob-IL 2hex x scFv5- | +++ | Y | Y | 11 | D002 | scFv5 | 15 | 15 |
| | sc 15-Hole-D002 | | | | | | | | |
| 318 | Knob-IL2hex x scFv59- | − | | | 11 | D002 | scFv59 | 15 | 15 |
| | sc 15-Hole-D002 | | | | | | | | |
| 319 | Knob-IL2hex x scFv70- | +++ | Y | Y | 11 | D002 | scFv70 | 15 | 15 |
| | sc 15-Hole-D002 | | | | | | | | |
| 320 | Knob-IL2hex x Hole- | − | | | 12 | D002 | scFv5 | 15 | 50 |
| | D002-sc50-scFv5 | | | | | | | | |
| 321 | Knob-IL 2hex x Hole- | +++ | Y | Y | 12 | D002 | scFv59 | 15 | 50 |
| | D002-sc50-scFv59 | | | | | | | | |
| 322 | Knob-IL 2hex x Hole- | − | | | 12 | D002 | scFv70 | 15 | 50 |
| | D002-sc50-scFv70 | | | | | | | | |
| 323 | Knob-IL2hex x Hole- | − | | | 12 | D002 | scFv5 | 15 | 15 |
| | D002-sc15-scFv5 | | | | | | | | |
| 324 | Knob-IL2hex x Hole- | +++ | Y | Y | 12 | D002 | scFv59 | 15 | 15 |
| | D002-sc15-scFv59 | | | | | | | | |
| 325 | Knob-IL2hex x Hole- | +++ | Y | Y | 12 | D002 | scFv70 | 15 | 15 |
| | D002-sc15-scFv70 | | | | | | | | |
| 677 | IL2hex-872- | +++ | moderate | moderate | 13 | D029- | mAB70 | 80 | 15 |
| | 70(Rev1/P94Y)-hole x | | | | | HVILV5 | | | |
| | D029HVILV5-knob | | | | | | | | |
| 621 | IL2hex-sc80-D029HV1- | +++ | Y | N | 14 | D029- | VHH6 | 15 | 30 |
| | sKnob x VHH6-sc30- | | | | | HVILV1 | | | |
| | Hole | | | | | | | | |
| 622 | IL2hex-sc80-D029HV1- | +++ | Y | N | 14 | D029- | VHH6 | 15 | 50 |
| | sKnob x VHH6-sc50- | | | | | HVILV1 | | | |
| | Hole | | | | | | | | |
| 623 | IL2hex-sc80-D029HV1- | +++ | Y | N | 14 | D029- | VHH6 | 15 | 70 |
| | sKnob x VHH6-sc70- | | | | | HVILV1 | | | |
| | Hole | | | | | | | | |
| 624 | IL2hex-sc30-D029HV1- | +++ | Y | N | 14 | D029- | VHH6 | 15 | 30 |
| | sKnob x VHH6-sc30- | | | | | HVILV1 | | | |
| | Hole | | | | | | | | |
| 625 | IL2hex-sc30-D029HV1- | +++ | Y | N | 14 | D029- | VHH6 | 15 | 50 |
| | sKnob x VHH6-sc50- | | | | | HVILV1 | | | |
| | Hole | | | | | | | | |
| 626 | IL2hex-sc30-D029HV1- | +++ | Y | N | 14 | D029- | VHH6 | 15 | 70 |
| | sKnob x VHH6-sc70- | | | | | HVILV1 | | | |
| | Hole | | | | | | | | |
| 707 | IL2hex-sc80-D029HV2- | +++ | Y | Y | 15 | D029- | scFv5 | 80 | 15 |
| | sKnob x D029LV4 x | | | | | HV2LV4 | | | |
| | scFv872-5-Hole | | | | | | | | |
| 359 | scFv5-Knob x IL2hex- | − | | | 16 | D001 | scFv5 | 15 | 15 |
| | D001(VH)-Hole x D001 | | | | | | | | |
| | (VL) | | | | | | | | |
| 360 | scFv5-Knob x IL2hex- | − | | | 16 | D001 | scFv5 | 50 | 15 |
| | sc50-D001(VH)-Hole x | | | | | | | | |
| | D001 (VL) | | | | | | | | |
| 361 | scFv5-Knob x IL2hex- | +++ | Y | Y | 16 | D002 | scFv5 | 15 | 15 |
| | D002 (VH)-Hole x D002 | | | | | | | | |
| | (VL) | | | | | | | | |
| 362 | scFv5-Knob x IL2hex- | +++ | Y | Y | 16 | D002 | scFv5 | 50 | 15 |
| | sc50-D002(VH)-Hole x | | | | | | | | |
| | D002 (VL) | | | | | | | | |
| 336 | scFv59-Knob x IL2hex- | ++ | Y | N | 17 | D001 | scFv59 | 15 | 15 |
| | D001 (VL)-Hole | | | | | | | | |
| 337 | scFv70-Knob x IL2hex- | ++ | | | 17 | D001 | scFv70 | 15 | 15 |
| | D001 (VL)-Hole | | | | | | | | |
| 338 | scFv5-Knob x IL2hex- | ++ | | | 17 | D001 | scFv5 | 15 | 15 |
| | D001 (VL)-Hole | | | | | | | | |
| 367 | D001 (VH)-Knob x | − | | | 18 | D001 | scFv5 | 15 | 15 |
| | IL2hex-D001(VL) x | | | | | | | | |
| | scFv5-Hole | | | | | | | | |
| 368 | D002 (VH)-Knob x | − | | | 18 | D002 | scFv5 | 50 | 15 |
| | IL2hex-sc50-D002(VL) x | | | | | | | | |
| | scFv5-Hole | | | | | | | | |
| 369 | D001(VH)-Knob x | − | | | 18 | D001 | scFv59 | 50 | 15 |
| | IL2hex-D001(VL) x | | | | | | | | |
| | scFv59-Hole | | | | | | | | |
| 370 | D002 (VH)-Knob x | + | | | 18 | D002 | scFv59 | 50 | 15 |
| | IL2hex-sc50-D002 (VL) x | | | | | | | | |
| | scFv59-Hole | | | | | | | | |
| 677 | D029HV1-sKnob x | ++ | | | 19 | D029- | mAB70 | 80 | 15 |
| | IL2hex-872-70H-Rev1 | | | | | VH1LV5 | | | |
| | P94Y-sHole | | | | | | | | |
| 791 | IL2hex-sc80-D029HV2- | +++ | Y | N | 20 | D029- | | 80 | |
| | sKnob x sHole | | | | | HV2LV4 | | | |
| 674 | scFv872-5Dbl(S-S)-sc70- | +++ | Y | Y | 3 | D029- | | 15 | 70 |
| | sKnob-IL2hex x sHole- | | | | | HV3LV2 | | | |
| | D029HV3 x D029LV2 | | | | | | | | |
| 225 | Knob-IL 2hex x Hole- | +++ | | | 2 | N/A | None | 15 | N/A |
| | 155 01 (VH) | | | | | | | | |
| 245 | Knob-IL2hex x Hole- | +++ | | | 2 | D002 | None | 15 | N/A |
| | D002 | | | | | | | | |
| 476 | sKnob x sHole-D047(VH) | ++ | | | 4 | D047 | None | 80 | N/A |
| | x IL2hex-sc80-D047(VL) | | | | | | | | |
| 559 | IL2HEX-SC80-D029HV1 | +++ | | | 20 | D029-HV1 | None | 80 | N/A |
| | -sKnob x D029 (VL) x | | | | | | | | |
| | sHole | | | | | | | | |
| 244 | Knob-IL2hex x Hole- | +++ | | | 2 | D001 | None | 15 | N/A |
| | D001 | | | | | | | | |
| 246 | Knob-IL2hex x Hole- | +++ | | | 2 | D003 | None | 15 | N/A |
| | D003 | | | | | | | | |
| 228 | Knob-sc30-IL2hex x | +++ | | | 2 | N/A | None | 30 | N/A |
| | Hole-155 01 (VH) | | | | | | | | |
| 232 | Knob x Hole-155_01 | ++ | | | 4 | N/A | None | 15 | N/A |
| | (VH) x IL2hex-(VL) | | | | | | | | |
| 255 | FcKnob-sc50-IL2hex x | +++ | | | 2 | D002 | None | 50 | N/A |
| | FcHole-D002 | | | | | | | | |
| 387 | scFv5-Knob-IL2hex x | +++ | | | 3 | D029 | scFv5 | 15 | 15 |
| | Hole-D029 (VH) | | | | | | | | |
| 392 | scFv70-Knob-IL2hex x | +++ | | | 3 | D029 | scFv70 | 15 | 15 |
| | Hole-D029 (VH) | | | | | | | | |
| 604 | sKnob-IL2hex x sHole- | +++ | Y | N | 2 | D029- | None | 15 | N/A |
| | D029HV2 x D029LV4 | | | | | HV2LV4 | | | |
| 626 | IL2hex-sc30-D029HV1- | ++ | | | 14 | D029- | VHH6 | 30 | 70 |
| | sKnob x VHH6-sc70- | | | | | HVILV1 | | | |
| | Hole | | | | | | | | |
| 675 | scFv872-5Dbl(S-S)-sc70- | +++ | | | 3 | D029- | scFv872- | 15 | 70 |
| | sKnob-IL2hex x sHole- | | | | | HV2LV3 | 5V1 | | |
| | D029HV2 x D029LV3 | | | | | | | | |
| 676 | scFv872-5Dbl(S-S)-sc70- | +++ | Y | Y | 3 | D029- | scFv872- | 15 | 70 |
| | sKnob-IL2hex x sHole- | | | | | HV2LV4 | 5V1 | | |
| | D029HV2 x D029LV4 | | | | | | | | |
| 707 | IL2hex-sc80-D029HV2- | +++ | Y | Y | 18 | D029- | scFv872- | 15 | 80 |
| | sKnob x D029LV4 x | | | | | HV2LV4 | 5V1 | | |
| | scFv872-5-Hole | | | | | | | | |
| 794 | IL2hex-sc105-D029HV2 | +++ | Y | Y | 18 | D029- | scFv872- | 105 | 70 |
| | -sKnob x D029L V3 x | | | | | HV2LV3 | 5V1 | | |
| | scfv872-5Dbl(S-S)-sc70- | | | | | | | | |
| | sHole | | | | | | | | |
| 801 | IL2hex-sc105-D029HV2- | +++ | Y | Y | 18 | D029- | scFv872- | 105 | 70 |
| | sKnob x D029LV4 x | | | | | HV2LV4 | 5V1 | | |
| | scfv872-5Dbl(S-S)-sc70- | | | | | | | | |
| | sHole | | | | | | | | |
| 818 | IL2hex(K35E)-sc80- | +++ | Y | Y | 18 | D029- | scFv872- | 80 | 70 |
| | D029HV4-sKnob x | | | | | HV4LV2 | 5V1 | | |
| | D029LV2 x scFv872- | | | | | | | | |
| | 5Dbl(S-S)-sc70-sHole | | | | | | | | |
| 834 | scFv872-5Dbl(S-S)- | +++ | Y | Y | 3 | D029- | scFv872- | 105 | 15 |
| | sKnob-sc105-IL2hex x | | | | | HV4LV2 | 5V1 | | |
| | sHole-D029HV4 x | | | | | | | | |
| | D029LV2 | | | | | | | | |
| 598 | scFv70-sc80-sKnob- | +++ | Y | Y | 3 | D029- | scFv872-70 | 15 | 80 |
| | IL2hex x sHole- | | | | | HV3LV1 | | | |
| | D029HV3 x D029LV1 | | | | | | | | |
| 599 | scFv70-sc80-sKnob- | +++ | Y | Y | 3 | D029- | scFv872-70 | 15 | 80 |
| | IL2hex x sHole- | | | | | HV3LV4 | | | |
| | D029HV3 x D029LV4 | | | | | | | | |
| 600 | scFv70-sc80-sKnob- | +++ | Y | Y | 3 | D029- | scFv872-70 | 15 | 80 |
| | IL2hex x sHole- | | | | | HV3LV2 | | | |
| | D029HV3 x D029LV2 | | | | | | | | |
| 601 | scFv70-sc80-sKnob- | +++ | Y | Y | 3 | D029- | scFv872-70 | 15 | 80 |
| | IL2hex x sHole- | | | | | HV2LV3 | | | |
| | D029HV2 x D029LV3 | | | | | | | | |
| 607 | scFv70-sc80-sKnob- | +++ | Y | Y | 3 | D029- | scFv872-70 | 15 | 80 |
| | IL2hex x sHole- | | | | | HV2LV1 | | | |
| | D029HV2 x D029LV1 | | | | | | | | |
| 608 | scFv70-sc80-sKnob- | +++ | Y | Y | 3 | D029- | scFv872-70 | 15 | 80 |
| | IL2hex x sHole- | | | | | HV5LV1 | | | |
| | D029HV5 x D029LV1 | | | | | | | | |
| 609 | scFv70-sc80-sKnob- | +++ | Y | Y | 3 | D029- | scFv872-70 | 15 | 80 |
| | IL2hex x sHole- | | | | | HV2LV4 | | | |
| | D029HV2 x D029LV4 | | | | | | | | |
| 610 | scFv70-sc80-sKnob- | +++ | Y | Y | 3 | D029- | scFv872-70 | 15 | 80 |
| | IL2hex x sHole- | | | | | HV6LV2 | | | |
| | D029HV6 x D029LV2 | | | | | | | | |
| 611 | scFv70-sc80-sKnob- | +++ | Y | Y | 3 | D029- | scFv872-70 | 15 | 80 |
| | IL2hex x sHole- | | | | | HV2LV2 | | | |
| | D029HV2 x D029LV2 | | | | | | | | |
| 449 | sKnob-IL2hex x Hole- | +++ | Y | N | 2 | D049 | None | 15 | N/A |
| | D049 (VH) | | | | | | | | |
| 476 | sKnob x sHole-D047 | +++ | Y | N | 2 | D047 | None | 15 | N/A |
| | (VH) x IL2hex-sc80- | | | | | | | | |
| | D047 (VL) | | | | | | | | |
| 314 | Knob-IL2hex x scFv5- | +++ | Y | N | 11 | D002 | scFv872-5 | 15 | 50 |
| | sc50-Hole-D002 | | | | | | | | |
| 1097 | IL2hex(K35E)-(G₄S)₁₆ | | | | 15 | D029H- | scFv872-5- | 80 | N/A |
| | D029H-siKnob x D029L | | | | | H1Var11- | Var4H1- | | |
| | x scFv872-5-siHole | | | | | D029L-Rev1 | Var1- | | |
| | | | | | | | H2Var1 | | |
| 1112 | IL2(F42A)-(G₄S)₁₆ | | | | 15 | D029H- | scFv872-5- | 80 | N/A |
| | D029H-siKnob x D029L | | | | | H1Var11- | Var4H1- | | |
| | x scFv872-5-siHole | | | | | D029L-Rev1 | Var1- | | |
| | | | | | | | H2Var1 | | |
| 1125 | IL2(D20T)-(G₄S)₁₆-B10- | | | | 15 | B10 | scFv872-5- | 80 | N/A |
| | siKnob x scFv872-5- | | | | | | Var4H1- | | |
| | sHole | | | | | | Var1- | | |
| | | | | | | | H2Var1 | | |
| 1150 | IL2WT(D20T K35E)- | +++ | Y | Y | 14 | B10 | VHH E33 | 70 | 70 |
| | 14×(G4S)-VH(B10-H2- | | | | | | | | |
| | VAR3)-CH1-siKnob x | | | | | | | | |
| | VHH E33-14×(G₄S)- | | | | | | | | |
| | siHole | | | | | | | | |
| A | IL2hex(K35E)-(G₄S)₁₆ | | | | 15 | FL78-H1Var1 | L2Var3- | 80 | |
| | FL 78-H1 Varl-sKnob x | | | | | | MOC31 scFv | | |
| | FL95-L2Var3 | | | | | | S-S | | |
| | MOC31scFv-SG4S-Hole | | | | | | | | |

*“IL2 WT” = wild type IL-2; “IL2hex” = mutant IL-2 of IL-2hex as defined herein; “IL2hex(K35E)” = mutant IL-2 having the mutations of T3A, K35E, F42A, Y45A, L72G, C125S; “IL2(F42A)” = mutant IL-2 having the mutation of T3A, F42A and C125S; “IL2WT(D20T)” = mutant IL-2 having the mutation of T3A, D20T, and C125S; “scFv5” “scFv59” “scFv70” or “MOC31scFv” = single chain variable fragment derived from antibody 872-5, 872-59, 872-70, or MOC31 respectively; “D002” “D029” “D047” “D049” “mAB70” “155 01” “FL78” “FL95” = Fab derived from antibody D002, D029, D047, D049, mAB70, or 155_01, FL75, or FL95, respectively; “(VH)” = antibody heavy chain variable domain; “(VL)” = antibody light chain variable domain; “Knob” = Fc subunit containing the knob modification; “sKnob” or “siKnob” = Fc subunit containing P329G, L234A, L235A in addition to the knob modification; “Hole” = Fc subunit containing the knob modification; “sHole” or “siHole” = Fc subunit containing P329G, L234A, L235A in addition to the hole modification; “sc15” “sc30”, “sc50”, “sc70”, “sc80”, or “sc105” = repeats of G4S linker at the specified length (for example, sc15 consists of three GGGGS linker at 15 amino acids length as GGGGS GGGGS GGGGS (SEQ ID NO: 100)); “(G₄S)₁₆” = refers to sc90; “5Dbl(S-S)-” = 872-5V1 scFv with a disulfide linker between the VH (residue 44) and VL (residue 100); “x” separating different peptidic chains forming part of a synaptokine molecule; “−” linker or connection between different moieties; moieties in a peptidic chain are arranged according to the N to C orientation in the peptide chain.

6.3.2 Generation of Fc variants

The heterodimeric Fe in the immunoconjugate molecules was modified by introducing knob-in-hole mutations. Particularly, in some embodiments, the mutations were S354C and T366W in one Fc subunit, and Y349C, T366S, L368A and Y407V in the other Fc subunit. Furthermore, to reduce the Fc effector activity for the purpose of screening, a set of mutations P329G, L234A and L235A were introduced to both Fc subunits.

6.3.3 Determination of mutant Fc Antibody Binding to Fc Receptors

In order to determine the impact of Fc mutations on binding with Fc receptors, a biolayer interferometry (BLI) assay was established. Briefly, Avi-tagged Fc receptor (CD16a (V176) or CD64, Acro Bio) were diluted to 100 nM in PBST-BSA and immobilized on a Streptavidin sensor on the Gator BLI instrument to an immobilization level of 1-2 nm depending upon the experiment. After establishing a baseline with PBST-BSA, the sensors were incubated with Certolizumab IgG or Certolizumab IgG Fc mutants complexed with TNFα (500 nM IgG+500 nM TNFα subunits). This association step proceeded for 180 seconds, followed by 180 seconds of dissociation in PBST-BSA. The binding of the Fc mutants to the Fc receptors were normalized as a percentage of binding of the wild-type Certolizumab-IgG1.

A set of Fc mutants were evaluated for the ability of such mutations to abolish Fc binding to FcR receptor as shown in Table 15. The triple mutations P329G, L234A and L235A were incorporated in the Fc domain for subsequent testing.

TABLE 15

| | | | | | | | | | | | | | | | | CD16a | CD64- |
|---|---|---|---|---|---|---|---|---|---|---|---|---|---|---|---|---|---|
| Fc position | 232 | 233 | 234 | 235 | 236 | 237 | 238 | 239 | 265 | 297 | 329 | 330 | 331 | 332 | 333 | Binding | Binding |
| WT | P | E | L | L | G | G | P | S | D | N | P | A | P | I | E | 100.0 | 100.0 |
| Fc mutant 1 | P | P | V | A | — | G | P | S | D | N | P | S | S | I | E | 74.4 | 74.1 |
| Fc mutant 2 | P | E | A | A | G | G | P | S | D | N | P | S | S | I | E | 43.6 | 125.9 |
| Fc mutant 3 | P | P | V | A | — | G | P | S | D | N | E | S | S | I | E | 0.0 | 0.9 |
| Fc mutant 4 | P | E | A | A | G | G | P | S | D | N | E | S | S | I | E | 0.0 | 55.6 |
| Fc mutant 5 | P | P | V | A | — | G | P | S | D | N | E | A | P | I | E | 0.0 | 9.3 |
| Fc mutant 6 | P | E | A | A | G | G | P | S | D | N | E | A | P | I | E | 1.8 | 74.1 |
| Fc mutant 7 | P | P | V | A | — | G | P | S | G | N | P | S | S | I | E | 1.3 | 5.9 |
| Fc mutant 8 | P | E | A | A | G | G | P | S | G | N | P | S | S | I | E | 0.0 | 1.5 |
| Fc mutant 9 | P | P | V | A | — | G | P | S | G | N | E | S | S | I | E | 0.0 | 1.5 |
| Fc mutant 10 | P | E | A | A | G | G | P | S | G | N | E | S | S | I | E | 1.3 | 7.4 |
| Fc mutant 11 | P | P | V | A | — | G | P | S | G | N | E | A | P | I | E | 0.0 | 1.5 |
| Fc mutant 12 | P | E | A | A | G | G | P | S | G | N | E | A | P | I | E | 0.3 | 3.7 |

### 6.4 Example 4

### 6.4.1 Biophysical Properties

Differential scan fluorimetry was determined by the fluorescence change while fluorophore binds to denatured protein induced by the rising temperature. In brief, 2-20 μM protein was mixed with 1X SYPRO Orange (Thermofisher cat: 56650) to a total volume of 25 μL in buffer PBS. The fluorescence was monitored by a QPCR instrument Roche Light Cycler 480 while increases the temperature from 25° C. to 95° C. at a speed of 0.02° C./s. The first derivative of the fluorescence intensity was plotted against temperature, and the temperature of negative peak was the melting temperature and indicates the process of protein denaturation. The higher melting temperature, the more stable the protein is.

Hydrophobic interaction chromatography was performed on an Agilent 1200 HPLC system with a TSKgel Butyl-NPR (14947, TOSH Bioscience) column. In brief, 5 pg protein samples (1 mg/mL) were mixed with a mobile phase A solution (1.8 M ammonium sulfate and 0.1 M sodium phosphate at pH 6.5) to achieve a final ammonium sulfate concentration of about 1 M before analysis. A linear gradient of mobile phase A and mobile phase B solution (0.1 M sodium phosphate, pH 6.5) over 20 min at a flow rate of 1 mL/min UV absorbance monitoring at 280 nm.

Size exclusion chromatography was performed on an Agilent 1200 HPLC system with a TSKgel G3000SW (05789, TOSH Bioscience) column. A flow rate of 0.35 mL/mL with PBS as running buffer was used, and retention time for each sample was assigned based on the major peak.

SMAC assay was performed on an Agilent 1200 HPLC system with a Zenix SEC-300 column (213300-4630, Sepax Technologies). A flow rate of 0.35 mL/min with PBS as running buffer was used, and retention time for each sample was assigned based on the major peak.

Biolayer interferometry (BLI) was used to measure the protein-protein interactions using Gator system (ProbeLife, USA). In brief, an optic fiber was coated with capture reagent such as streptavidin, anti-human Fc antibody etc. The instrument can precisely measure the light interference in terms of wavelength shift when refractive index changes up protein binding at the tip of optical fiber. The kinetics and amplitude of wavelength shift directly reflect the mode of protein-protein interaction. For example, FIG. 15 shows a five-step experiments. In the first step, the optic fiber coated with streptavidin was dipped into PBST-0.5% BSA for equilibration. In the second step, the optic fiber was dipped into 50 nM biotinylated 5UTZ molecule to load 5UTZ onto the surface of sensor. In the third step, the optic fiber was dipped into PBST-0.5% BSA for equilibration. In the fourth step, the optic fiber was dipped into protein mixes such as 100 nM FB-604+100 nM Fc-hFAP. In the fifth step, the optic fiber was dipped into PBST-0.5% BSA for dissociation.

Four immunoconjugate molecules FB-604, FB-675, FB-676, FB-626 were tested for their capability of binding to 5UTZ in the absence or presence of soluble Fc-hFAP. As shown in FIG. 15, in the absence of soluble Fc-hFAP, none of the four immunoconjugate molecules were able to bind 5UTZ, suggesting the cytokine IL-2hex was shielded by the two-in-one antibody through intra-molecular interactions. In the presence of soluble Fc-hFAP, three immunoconjugate molecules, FB-604, FB-675 and FB-676, became able of binding to 5UTZ, indicating soluble FAP compete with IL-2 for binding with the two-in-one antibody thereby releasing the IL-2 from intra-molecular interaction and becoming capable of binding to 5UTZ. As shown in FIG. 16, 5UTZ binds specifically to IL2hex, but not hFAP.

### 6.4.2 In Vivo Half-Life

The pharmacokinetics of interested molecules were measured in health C57BL/6 mice. Mice were injected with desired amounts of molecules (50 pg to 900 pg) in a volume of 150 μL in the tail vein using a slow push. At various time points, small blood samples (20-100 μL) were taken by retro-orbital bleeding and collected in tubes coated with heparin to prevent clotting. After centrifugation to remove the cells, the plasma was assayed by ELISA with Goat anti-human IgG, IgM, IgA (H+L) antibody (A18849, Invitrogen) as capture antibody and Goat anti-human IgG Fc Cross-Absorbed HRP (A18823, Invitrogen) as detection antibody. Results were normalized to the initial concentration in the serum of each mouse taken immediately after injection. As shown in FIG. 7A, the half-life of the control molecule (Knob-IL2Hex), which contains IL-2 fused to the Fc domain, was 1.4 days. Both immunoconjugate molecules tested had the half-life extended to about 5 to 10 days, which was comparable to that of the human IgG. The maximum serum concentration and half-life were analyzed and listed in the table. The serum concentration of 900 pg dose (equivalent to 45 mg/kg in mice) scaled up proportionally from 90 pg dose, suggesting the 90 pg dose exceeded the target-mediated drug disposition (TMDD) and the presence of two-in-one antibody within the immunocytokine molecule effectively masked the cytokine polypeptide IL2hex from binding with its receptors in vivo.

| | Cmax (ug/mL) | Th (days) |
|---|---|---|
| CTRL-50 | 21.1 | 1.4 |
| #476-90 | 46.7 | 10.0 |
| #476-900 | 473.4 | 5.0 |
| #559-90 | 52.0 | 4.9 |
| #559-900 | 380.5 | 7.7 |

### 6.5 Example 5: Activity Assays

### 6.5.1 Cell-Based IL-2 Signaling Assay

HEK Blue IL-2 reporter cell line (Cat #: hkb-il2, InVivogen) was engineered with high affinity human IL-2 receptors (CD25, CD122 and CD132) on surfaces. Its dose-dependent response to IL-2 correlated with the level of secreted embryonic alkaline phosphatase (SEAP) in the supernatant of the cell culture, which was then assayed using an enzymatic assay. In this study, IL-2 activity was assayed using the QUANTI-Blue buffer and substrate following manufacture-provided instructions. The EC₅₀ concentration was calculated using least squares analysis (TREND analysis from Excel).

Particularly, to assay IL-2 activity, 20,000 HEK Blue IL-2 cells was cultured in flat bottom 96-well plates, and naked IL-2 polypeptide or IL-2 containing immunoconjugate molecules were added to the cell culture at the indicated gradient of concentrations. After 20-hour incubation, 20 μL supernatant of the cell culture was added into 180 μL QUANTI-Blue buffer (Cat #: rep-qbs, InVivoGen) and the reaction was incubated at 37° C. for 1-3 hrs. The absorbance at 635 nm (A635) was determined using a TECAN plate reader, which reflected the SEAP level and dose-dependent response to IL-2.

To assay the influence of soluble human Fibroblast Activation Protein (hFAP) on the potency of the IL-2 containing immunoconjugate molecules, 20,000 HEK Blue IL-2 cells was cultured in flat bottom 96-well plates. Soluble hFAP was added to the cell culture to the tested concentration of 200 nM or 2 μM, and IL-2 containing immunoconjugate molecules were added to the cell culture at the indicated gradient of concentrations. After 20-hour incubation, 20 μL supernatant of the cell culture was added into 180 μL QUANTI-Blue buffer (Cat #: rep-qbs, InVivoGen) and the reaction was incubated at 37° C. for 1-3 hrs. The absorbance at 635 nm (A635) was determined using a TECAN plate reader, which reflected the SEAP level and dose-dependent response to IL-2.

To assay the influence of hFAP expressing cells on the potency of the IL-2 containing immunoconjugate molecules, 20,000 HEK Blue IL-2 cells was co-cultured with either 20,000 HEK293T cells or 20,000 HEK293T cells expressing hFAP on the surface (HEK 293T-hFAP-E5 cells) into flat bottom 96-well plates. IL-2 containing immunoconjugate molecules were added to the cell culture at the indicated gradient of concentrations. After 20-hour incubation, 20 μL of supernatant was added into 180 μL QUANTI-Blue buffer (Cat #: rep-qbs, InVivoGen) and the reaction was incubated at 37° C. for 1-3 hrs. The absorbance at 635 nm (A635) was determined using a TECAN plate reader, which reflected the SEAP level and dose-dependent response to IL2.

### 6.5.1.1 Inhibition of Cytokine Activity Via Intramolecular Interaction in an Immunoconjugate Molecule

In order to examine intramolecular inhibition of the cytokine activity in the immunoconjugate molecule according to the present disclosure, IL-2 containing immunoconjugate molecules of configuration 1 and configuration 2 as shown in FIGS. 5B and 5C (or FIGS. 8B and 8C) were constructed and subjected to the cell-based IL-2 signaling assay as described above, and the results are shown in FIG. 8A.

Particularly, in this study, all immunoconjugate molecules contained an Fc domain having two non-identical subunits with knob-into-hole modifications that promoted dimerization of the two polypeptide chains. Immunoconjugate molecules of configuration 1 (circle) contained an IL-2 polypeptide fused to the C-terminus of one of the Fc subunits. Immunoconjugate molecules of configuration 2 (circle) contained an IL-2 polypeptide fused to the C-terminus of one Fc subunit, and an anti-IL-2/anti-FAP bispecific Fab (or a control antibody) fused to the C-terminus of the other Fc subunit. Particularly, four different immunoconjugate molecules of configuration 2 were constructed in this study, two of which containing different anti-IL-2/anti-FAP bispecific Fab molecules derived from the D001 antibody (down triangle) and the D002 antibody (diamond), respectively. As a positive control, a third immunoconjugate molecule of configuration 2 contained a specific anti-IL-2 Fab antibody (155-01; up triangle) capable of inhibiting IL-2 signaling (data not shown) in lieu of the bispecific antibody, and as a negative control, a fourth immunoconjugate molecule of configuration 2 contained a Fab molecule (D003; left triangle) that did not exhibit detectable binding to either IL-2 or FAP (data not shown) in lieu of the bispecific antibody. A sample containing the naked IL-2 polypeptide (Sino Biological, Beijing, China) was also included as a negative control (square).

As shown in FIG. 8A, naked IL-2 polypeptide (square) and the tested immunoconjugate molecule of configuration 1 (circle) elicited similar dose-dependent responses to IL-2 in the reporter cell line, which results were consistent with the lack of the masking moiety in immunoconjugate molecule. In contrast, each of the tested immunoconjugate molecules of configuration 2 (up triangle; down triangle; diamond) significantly inhibited IL-2 activity, suggesting the presence of intramolecular binding between the bispecific antibody and IL-2 in these immunoconjugate molecules.

The above data demonstrate that the cytokine in the immunoconjugate molecule of the present disclosure retains its function in activating cell-surface receptors and eliciting cellular responses. Furthermore, the bispecific antibody (i.e., the masking moiety) in the immunoconjugate molecule is capable of binding with the cytokine, thereby inhibiting the cytokine activity.

### 6.5.1.2 Molecular Configuration of an Immunoconjugate Molecule Influences the Effectiveness of Intracellular Inhibition of Cytokine

In order to examine whether the molecular configuration, including the orientations, arrangements, and formats of the different components, of an immunoconjugate molecule according to the present disclosure would impact the observed intramolecular inhibition of the cytokine activity, immunoconjugates having configuration 1, configuration 2, or configuration 4 as shown in FIGS. 5B, 5C and 5E (or FIGS. 9B, 9C and 9D) were constructed and subjected to the cell-based IL-2 signaling assay as described above, and the results are shown in FIG. 9A.

Particularly, in this study, all immunoconjugate molecules contained an Fc domain having two non-identical subunits with knob-into-hole modifications that promoted dimerization of the two polypeptide chains. Immunoconjugate molecules of configuration 1 (square) contained an IL-2 polypeptide fused to the C-terminus of one of the Fc subunits. Immunoconjugate molecules of configuration 2 (circle) contained an IL-2 polypeptide fused to the C-terminus of one Fc subunit, and an anti-IL-2/anti-FAP bispecific Fab fused to the C-terminus of the other Fc subunit. Immunoconjugate molecules having configuration 4 (down triangle) contained an anti-IL-2/anti-FAP bispecific Fab, where the N-terminus of the Fab heavy chain was fused to the C-terminus of one of the Fc subunits, and an IL-2 polypeptide was fused to the N-terminus of the Fab light chain. Particularly, in this study, the anti-IL-2/anti-FAP bispecific Fab in both configuration 2 and configuration 4 was derived from the D001 antibody.

As shown in FIG. 9A, the immunoconjugate of configuration 1 (square) elicited a dose-dependent response to IL-2 in the reporter cell line. In contrast, the immunoconjugates of configuration 2 (circle) and configuration 4 (down triangle) both exhibited significant inhibition of IL-2 activity. Moreover, the immunoconjugate of configuration 4 (down triangle) was more effective in blocking IL-2 activity as compared to configuration 2 (circle). These data suggest that while the molecular configuration of the immunoconjugates may impact the effectiveness of intramolecular interaction between the masking moiety and the cytokine, the observed cytokine inhibition also does not require the particular molecular configuration that was tested in this study.

### 6.5.1.3 Intracellular Inhibition of Cytokine Reduces Cytokine's Toxicity In Vivo

Intramolecular interaction of two-in-one antibody to cytokine can inhibit its potency in vitro as demonstrated in HEK Blue IL2 assay, CTLL2 proliferation assay, and human CD4+ proliferation assay. To determine how relevant this functional inhibition to the in vivo, acute toxicity was examined in mice.

It has been reported that high dose IL-2 treatment can be lethal to mice. First, both C57BL/6J mice and CB-17 SCID mice were dosed daily for five days a week for two weeks with naked cytokine Knob-IL2hex. The observed toxicity by the death and body weight loss was consistent with results reported in the literature (e.g., Clin Cancer Res 17(11) 3673-85, 2011). To simplify the comparison, C57BL/6J mice were chosen for subsequent acute toxicity studies. Second, two immunoconjugate molecules (#449 and #476) together with Knob-IL2hex and a commercial control #439 Akrevia-IL2hex were evaluated for their toxicity in C57BL/6J. The Knob-IL2hex showed incremental toxicity from 25 μg/dose/day to 50 μg/dose/day in a week, while all other three molecules did not show any sign of toxicity at 180 μg/dose/day which is 4× molar equivalence of 25 μg/dose/day. Although this experiment has not reached the maximum tolerated doses for all these four immunoconjugate molecules, it was demonstrated that the two-in-one antibody in the immunoconjugate molecule significantly inhibited toxicity of IL-2 (FIG. 30). Taken together with the pharmacokinetics data demonstrating that the half-life of immunoconjugate molecules were extended for about 5 times as compared to Knob-IL2hex, no sign of toxicity at 4× molar equivalent dose showed greater than 10 folds improvement in the safety profile.

6.5.1.4 Soluble Antigen does not Activate Cytokine Activity in Non-Anchored Immunoconjugate Molecules

To demonstrate activation of cytokine activity in the immunoconjugate molecules of the present disclosure, first, it was examined whether soluble antigens can compete for binding with the masking moiety, and release the cytokine in an unbound form to activate the activity. In one study, immunoconjugate molecules having configuration 1 and configuration 2 as shown in FIGS. 5B and 5C (or FIGS. 10B and 10C) were constructed and subjected to the cell-based IL-2 signaling assay in the presence of soluble human Fibroblast Activation Protein (hFAP), and the results are shown in FIG. 10A.

Particularly, in this study, all immunoconjugate molecules contained an Fc domain having two non-identical subunits with knob-into-hole modifications that promoted dimerization of the two polypeptide chains. Immunoconjugate molecules of configuration 1 (open square) contained an IL-2 polypeptide fused to the C-terminus of one of the Fc subunits. Immunoconjugate molecules of configuration 2 contained an IL-2 polypeptide fused to the C-terminus of one Fc subunit, and an anti-IL-2/anti-FAP bispecific Fab fused to the C-terminus of the other Fc subunit. Particularly, two different immunoconjugates of configuration 2 were constructed, containing the anti-IL-2/anti-FAP bispecific Fab derived from the 155_01 antibody (open square with a cross) and the D002 antibody (blue square), respectively. Immunoconjugate molecules containing the D002 Fab were tested in the absence of soluble hFAP (blue square), or in the presence of 200 nM (pink square) or 2 μM (red square) soluble hFAP. A sample containing the naked IL-2 polypeptide (Sino Biological, Beijing, China) (closed square) was included as the positive control, and a sample containing soluble hFAP (open square dashed line) were included as a negative control.

As shown in FIG. 10A, in this study, immunoconjugate molecules of configuration 2 tested under all conditions exhibited significant inhibition of IL-2 activity as compared to naked IL-2 or immunoconjugate of configuration 1 that lacked the masking moiety. Soluble hFAP did not produce observable activation of IL-2 at the tested concentrations of 200 nM and 2 μM, suggesting that soluble FAP is a weak competitor for binding with the anti-IL-2/anti-FAP bispecific masking moiety, and thus was less effective in activating IL-2 activity under intramolecular inhibition as compared to hFAP expressed on cellular surface. These data also demonstrate that immunoconjugate molecules of the present disclosure can effectively inhibit the cytokine activity via strong intracellular self-interaction between the cytokine and the masking moiety, and therefore effectively prevent off-target activation of the cytokine activity and ensuing side-effects.

### 6.5.1.5 Anchored Immunoconjugates Exhibit Antigen-Dependent Activation of Cytokine Activity

Next, antigen-dependent activation of the cytokine activity in the immunoconjugate molecules was examined using cells expressing the antigen on the cell surface. Particularly, immunoconjugate molecules having configuration 1 and configuration 3 as shown in FIGS. 5B and 5D (or FIGS. 11B and 11C) were constructed and subjected to the cell-based IL-2 signaling assay in the presence of HEK293T cells expressing human Fibroblast Activation Protein (hFAP) on the surface, and the results are shown in FIG. 11A.

Particularly, in this study, all immunoconjugate molecules contained an Fc domain having two non-identical subunits with knob-into-hole modifications that promoted dimerization of the two polypeptide chains. Immunoconjugate molecules of configuration 1 (square) contained an IL-2 polypeptide fused to the C-terminus of one of the Fc subunits. Immunoconjugate molecules of configuration 3 contained (a) an IL-2 polypeptide fused to the C-terminus of one Fc subunit; (b) an anti-IL-2/anti-FAP bispecific Fab derived from the D002 antibody and was fused to the C-terminus of the other Fc subunit; and (c) an anti-FAP scFv antibody derived from the 872-5 antibody and was fused to the N-terminus of one of the Fc subunits. The immunoconjugate of configuration 1 was tested in the absence of FAP-expressing cells (square); and the immunoconjugates of configuration 3 were tested in the presence of unmodified HEK293T cells (circle) or HEK293T cells expressing hFAP on the surface (triangle).

As shown in FIG. 11A, in the absence of FAP-expressing cells, the immunoconjugate of configuration 3 (circle) exhibited significant inhibition of IL-2 activity as compared to the immunoconjugate of configuration 1 that lacked the masking moiety (square). Activation of IL-2 activity was observed when the immunoconjugate of configuration 3 was in contact with FAP-expressing cells (triangle), suggesting that the cell surface antigen is capable of shifting the bispecific masking moiety towards disassociating from the cytokine, thereby releasing the cytokine in an unbound form to activate its activity.

### 6.5.1.6 Antigen-Dependent Activation of Cytokine Activity is Facilitated by Immobilization of Immunoconjugate Molecules in a Cellular Environment Enriched of the Antigen.

Next, to examine whether the observed cytokine activation requires binding of the immunoconjugate molecules to the antigen-expressing cell, cytokine activation was measured using the cell-based IL-2 signaling assay as described above while soluble FAP or competing antibodies were added to the reaction system to disrupt the binding, and the results were shown in FIGS. 11D and 11E.

Particularly, in one study, the immunoconjugate of configuration 1 was tested in the absence of FAP-expressing cells (square). Immunoconjugates of configuration 3 were tested in the presence of unmodified HEK293T cells (circle), in the presence of HEK293T cells expressing hFAP on the surface (blue triangle), in the presence of HEK293T cells expressing hFAP on the surface and soluble hFAP at the same concentration as the tested immunoconjugate molecules (red triangle), or in the presence of HEK293T cells expressing hFAP on the surface and soluble hFAP at the concentrations of 2 nM (hexagon size 1), 20 nM (hexagon size 2), 200 nM (hexagon size 3), and 2 μM (hexagon size 4), respectively. A reaction containing added unmodified HEK293T cells alone was included as the negative control (upper triangle).

As shown in FIG. 11D, titration of soluble FAP in the presence of FAP-expressing cells exhibited dose-dependent inhibition of the IL-2 activity, suggesting that the soluble antigen molecules compete with the cell surface antigen molecules for binding with the immunoconjugate, thereby interfering with the binding of the immunoconjugate molecules to the cells and inhibiting the cytokine activity.

In a second study, the immunoconjugate of configuration 1 was tested in the absence of FAP-expressing cells (square). Immunoconjugates of configuration 3 were tested in the presence of unmodified HEK293T cells (circle), in the presence of HEK293T cells expressing hFAP on the surface (down triangle), or in the presence of HEK293T cells expressing hFAP on the surface and 200 nM non-binding antibody (diamond), 200 nM 872-5 anti-FAP antibody (hexagon), or 200 nM 872-70 anti-FAP antibody (pentagon), respectively. A reaction containing added unmodified HEK293T cells alone was included as the negative control (upper triangle).

As shown in FIG. 11E, presence of the 872-70 (pentagon) and 872-5 (hexagon) antibodies both reduced IL-2 activity as compared to IL-2 activity measured in the absence of anti-FAP antibodies (down triangle). Inhibition was not observed for the reaction with added non-binding antibody (diamond). These data suggest that the anti-FAP antibodies compete with the immunoconjugate molecules for binding with cell surface FAP, thereby interfering with binding of the immunoconjugate molecules to the cells and inhibiting the cytokine activity.

The above studies suggest that antigen-dependent activation of cytokine activity in an immunoconjugate molecule of the present disclosure can occur when the immunoconjugate molecules bind to antigen-expressing cells. Next, to examine whether the binding is mediated by the binding of the anchoring moiety to cell surface antigen molecules, in a third study, cytokine activation in an immunoconjugate molecule lacking the anchoring moiety was measured. Particularly, immunoconjugate molecules having configuration 1 and configuration 2 as shown in FIGS. 5B and 5C (FIGS. 12B and 12C) were constructed and subjected to the cell-based IL-2 signaling assay as described above, and the results are shown in FIG. 12A.

Particularly, in this study, all immunoconjugate molecules contained an Fc domain having two non-identical subunits with knob-into-hole modifications that promoted dimerization of the two polypeptide chains. Immunoconjugate molecules of configuration 1 contained either a wild-type IL-2 polypeptide (closed square) or the mutant IL-2hex polypeptide (open square) fused to the C-terminus of one of the Fc subunits. The immunoconjugate molecule having configuration 2 (open triangle; closed triangle) contained an IL-2 polypeptide fused to the C-terminus of one of the Fc subunits, and an anti-IL-2/anti-FAP bispecific Fab fused to the C terminus of the other Fc subunit. The two types of immunoconjugates of configuration 1 were tested in the presence of unmodified HEK 293T cells (open square; closed square). The immunoconjugates of configuration 2 were tested in the presence of unmodified HEK 293T cells (open triangle) or HEK 293T cells expressing hFAP on the cell surface (closed triangle).

As shown in FIG. 12A, both types of immunoconjugates of configuration 1 triggered dose-dependent responses to IL-2 in the reporter cell line, which was consistent with the lack of the masking moiety in these molecules. Immunoconjugates of configuration 2 tested with or without FAP-expressing cells both exhibited significant inhibition of IL-2 activity, indicating intramolecular interaction and inhibition of IL-2 by the anti-IL-2/anti-FAP bispecific Fab. Notably, there was no significant difference between the inhibition observed with (closed triangle) or without (open triangle) FAP-expressing cells, suggesting that the lack of the anchoring moiety in these molecules abolishes antigen-dependent IL-2 activation.

These studies suggest that binding of the anchoring moiety of the immunoconjugate molecule to cell surface antigens can immobilize the immunoconjugate molecule in a cellular environment that is enriched of the antigen, thereby shifting the bispecific masking moiety of the immunoconjugate molecule towards binding with the antigen and releasing the cytokine to activate its cellular function in such cellular environment.

6.5.1.7 the Impact of the Affinities within the Two-In-One Antibody on Inhibition and Activation of Cytokine Activity

The affinity of D002 to IL2hex is relatively weak (K_{D}=−3.4 μM), yet D002 was able to effectively inhibit the cytokine activity while the cytokine binds of greater than about 300 times tighter to its receptor at K_{D} of about 1 nM in immunoconjugate molecule having configuration 2. Intramolecular interaction dominates over intermolecular interactions, and the affinity requirement for effective intramolecular inhibition for cytokine activity is relatively low, and the K_{D} value in the pM range appears to be enough for configuration 2. Although the D002 binds to hFAP at higher affinity (K_{D}=−50 nM), the immunoconjugate molecule having D002 cannot be activated by hFAP-expressing cells, and an anchor moiety such as in configuration 3 is needed to create a sudo-intramolecular interaction: immobilized hFAP—anchoring moiety—D002 to hFAP, to compete off the inhibiting intramolecular interaction between D002 and IL2hex, thereby activating the cytokine activity.

The hypothesis is supported by another exemplary bispecific two-in-one antibody D029 which showed no apparent binding to hFAP at 1 μM concentration while bound to IL2hex at K_{D} of about 431 nM. In reference to D002 with K_{D} of about 3.4 μM to IL2hex, it is expected that D029 can inhibit IL2 as well in the format of immunoconjugate of configuration 2. However, somewhat unexpectedly, the immunoconjugate having D029 as the masking moiety and an anchoring moiety in configuration 3 can activate cytokine activity in presence of hFAP expressing cells. Two different anchoring moieties containing scFv70 and scFv5 with comparable affinity to hFAP, but to different epitopes of hFAP were tested. Particularly, scFv70 binds at the same epitope as D029 and scFv5 binds on a distinct epitope. As shown in FIG. 13A, Different anchoring moieties did not appear to affect the activation of cytokine activity in the D029 containing immunoconjugate molecules of configuration 3.

For the effectiveness of inhibition and activation of cytokine activities in an immunoconjugate molecule, satisfying logic requirement of intramolecular interaction seems more important, and the affinity of the two-in-one antibody for binding with the activation signal (e.g., a tumor associated antigen in the tumor microenvironment) or the intramolecular cytokine appears to be less important. Nevertheless, there should be a range for optimal affinity to either the activation signal or the cytokine. For example, the extremely high binding affinity to the cytokine can permanently inhibit cytokine's affinity, while extremely low affinity to the cytokine may not be able to effectively inhibit cytokine activity even in the absence of the activation signal. Hence, functional consequences of the affinity to activation signal and cytokine of D029 were tested, by generating a set of D029 mutants with differed affinity to hFAP in the range of 1 nM to 10 μM and affinity to IL2hex in the range of 100 nM to 10 μM, using configuration 3 of the immunoconjugate. The K_{D} and EC₅₀ values in the presence or absence of hFAP-expressing cells for the set of D029 mutants are shown in Table 13B.

### 6.5.1.8 Activation of Cytokine Activity in Immunoconjugate Molecules by Soluble Antigens

Without being bound by theory, it is contemplated that as far as the immunoconjugate molecule can bind to the same Fc-hFAP dimer, it will suffice the intramolecular interaction which should be able to release the cytokine. Practically, if the anchoring moiety and the two-in-one masking antibody bind at distinct epitopes on hFAP, a long linker would enable simultaneous engagement onto the same Fc-hFAP molecule. A few immunoconjugate molecules were constructed and examined for whether the inhibited cytokine activity can be released by contacting the immunoconjugate molecule with soluble Fc-hFAP. The tested immunoconjugate molecules include FB-604, FB-675, FB-676 and FB-626.

The test started with biophysical characterization by Biolayer Interferometry. An IL-2 binding molecule 5UTZ was used as a reagent. 5UTZ can bind to free IL-2 but not to the IL-2 in above immunoconjugate molecules where the epitope recognized by 5UTZ is shielded by the two-in-one antibody. The biotinylated 5UTZ was immobilized onto the sensor first. Then the immunoconjugate molecule alone or in complex of soluble Fc-hFAP were applied to examine whether 5UTZ can bind to the IL-2. As shown in FIGS. 17 to 20, for all four tested immunoconjugate molecules at 50 nM alone, none can bind to 5UTZ at a detectable level and this result confirms that the IL-2 were effectively shielded by the two-in-one antibody. In complex with 50 nM, three immunoconjugate molecules, namely FB-604, FB-675 and FB-675, showed significant binding to 5UTZ, suggesting the inhibition by two-in-one antibody was competed off by the soluble Fc-hFAP. Since the FB-604 was in configuration 2 and without the anchoring moiety, the current experiments didn't answer the question regarding to the anchor. But one synaptokine FB-626 didn't demonstrate the effect of de-shielding, it has barely appreciable binding to hFAP.

This set of experiments show that the soluble hFAP can induce de-shielding of the cytokine as far as hFAP affinity is not too low.

### 6.5.1.9 Activation of IL-2 Activity in Multi-Epitopic Immunoconjugate

Next, to examine whether antigen-dependent activation of the cytokine activity in an anchored immunoconjugate molecule requires binding of the anchoring moiety (e.g., the anti-FAP antibody) and the masking moiety (e.g., the anti-IL-2/anti-FAP bispecific antibody) to the same epitope of the antigen (e.g., FAP), immunoconjugate molecules having configuration 1 and configuration 3 as shown in FIGS. 5B and 5D (or FIGS. 21B and 21C) were constructed and subjected to the cell-based IL-2 signaling assay as described above, and the results are shown in FIG. 21A.

Particularly, in this study, all immunoconjugate molecules contained an Fc domain having two non-identical subunits with knob-into-hole modifications that promoted dimerization of the two polypeptide chains. Immunoconjugate molecules of configuration 1 contained an IL-2 polypeptide fused to the C-terminus of one of the Fc subunits. Immunoconjugate molecules of configuration 3 contained (a) an IL-2 polypeptide fused to the C-terminus of one of the Fc subunits; (b) an anti-IL-2/anti-FAP bispecific Fab derived from the D002 antibody that was fused to the C-terminus of the other Fc subunit; and (c) an anti-FAP scFv antibody fused to the N-terminus of one of the Fc subunits. Two different anti-FAP scFv antibodies derived respectively from the 872-5 and 872-70 antibodies were used to generate the immunoconjugate molecules used in this study. Particularly, as shown in Table 9, the bispecific D002 Fab and the 872-70 scFv bind to the same epitope of FAP, while the 872-5 scFv binds to a different epitope of FAP. Immunoconjugate of configuration 1 was tested without cells expressing hFAP (square); immunoconjugates of configuration 3 were tested with (open circle: 872-5 scFv; open triangle: 872-70 scFv) or without (closed circle: 872-5 scFv; closed triangle: 872-70 scFv) FAP-expressing cells.

As shown in FIG. 21A, in the absence of FAP-expressing cells, the immunoconjugate of configuration 1 triggered a dose-dependent response to IL-2 in the reporter cell line (square). In contrast, both types of immunoconjugates of configuration 3 exhibited significant inhibition of IL-2 activity (closed circle; closed triangle). Potent activation of IL-2 activity (with enhanced EC₅₀ values up to 200 folds; data not shown) was observed for both types of immunoconjugates of configuration 3 when the molecules were in contact with FAP-expressing cells (open circle; open triangle).

In this study, both the mono-epitopic immunoconjugate (i.e. the anchoring moiety and masking moiety bind to the same epitope) and the bi-epitopic immunoconjugate exhibited potent cytokine activation, indicating that the antigen-dependent activation of cytokine does not require the anchoring and the masking moieties of the immunoconjugate molecule to recognize and bind to the same epitope or different epitopes of the antigen.

As shown in FIGS. 22 to 24, three anchoring moieties comprising scFv872-5, scFv872-59 and scFv-70, respectively, bind to distinct epitopes of hFAP. As shown in the figures, all tested immunoconjugate molecules had similar masking effect on the cytokine in the absence of hFAP expression cells. Further, both immunoconjugate molecules were able to de-shield and activate the cytokine activity in the presence of hFAP expression cells. Hence, these experiments demonstrated that epitope specificity does not appear impact the ability of shielding/de-shielding cytokine activity by a masking moiety in the immunoconjugate molecule.

### 6.5.1.10 Antigen-Dependent Activation of Cytokine Activity Occurs in Immunoconjugate Molecules of Diversified Configurations

The following studies were performed to examine whether antigen-dependent activation of cytokine activity in the immunoconjugate molecules requires any particular molecular configuration of the molecule.

Particularly, in one study, immunoconjugate molecules having configuration 1 and configuration 5 as shown in FIGS. 5B and 5F (or FIGS. 25B and 25C) were constructed and subjected to the cell-based IL-2 signaling assay as described above, and the results are shown in FIG. 25A.

In this study, all immunoconjugate molecules contained an Fc domain having two non-identical subunits with knob-into-hole modifications that promoted dimerization of the two polypeptide chains. Immunoconjugates of configuration 1 contained either a wild-type IL-2 polypeptide (circle) or the mutant IL-2hex polypeptide (square) fused to the C-terminus of one of the Fc subunits. Immunoconjugates of configuration 5 (open diamond; closed diamond) contained (a) a IL-2 polypeptide fused to the C-terminus of one of the Fc subunits, (b) an anti-IL-2/anti-FAP bispecific Fab antibody fused to the C-terminus of the other Fc subunit, and (c) an anti-FAP single domain antibody fused to the N terminus of one of the Fc subunits. The two types of immunoconjugates of configuration 1 were tested without cells expressing hFAP. Immunoconjugates of configuration 5 were tested either in the presence of unmodified HEK293T cells (open diamond), or in the presence of HEK293T cells expressing hFAP (closed diamond).

As shown in FIG. 25A, immunoconjugates of configuration 1 triggered dose-dependent response to IL-2 in the report cell line (square; circle), which was consistent with the lack of the masking moiety in these molecules. In the absence of FAP-expressing cells, immunoconjugates of configuration 5 exhibited significant inhibition of IL-2 activity (open diamond), and IL-2 activation was observed when the immunoconjugate molecules were in contact with FAP-expressing cells (closed diamond).

In another study, immunoconjugate molecules having configuration 1 and configuration 6 as shown in FIGS. 5B and 5G (or FIGS. 26B and 26C) were constructed and subjected to the cell-based IL-2 signaling assay as describe above, and the results are shown in FIGS. 26A and 26D.

In this study, all immunoconjugate molecules contained an Fc domain having two non-identical subunits with knob-into-hole modifications that promoted dimerization of the two polypeptide chains. Immunoconjugates of configuration 1 contained either a wild-type IL-2 polypeptide (circle) or the mutant IL-2hex polypeptide (square) fused to the C-terminus of one of the Fc subunits. Immunoconjugates of configuration 6 contained (a) a IL-2 polypeptide fused to the C-terminus of one of the Fc subunits, (b) an anti-IL-2/anti-FAP bispecific scFv antibody fused to the C-terminus of the other Fc subunit, and (c) an anti-FAP Fab antibody fused to the N-terminus of one of the Fc subunits. Particularly, the anti-IL-2/anti-FAP bispecific scFv antibody used to construct the immunoconjugates of configuration 6 was derived from the D002 antibody, and three different anti-FAP Fab antibodies derived respectively from the 872-5, 872-59, and 872-70 antibodies were used to construct the immunoconjugates of configuration 6 used in this study. Particularly, as shown in Table 9, the D002 scFv binds to the same FAP epitope as the 872-59 Fab and the 872-70 Fab, and the 872-5 Fab binds to a different FAP epitope. The two types of immunoconjugates of configuration 1 were tested without cells expressing hFAP (square; circle). The immunoconjugates of configuration 6 were tested either in the presence of unmodified HEK293T cells (closed down triangle; closed diamond; closed left triangle), or in the presence of HEK293T cells expressing hFAP (open down triangle; open diamond; open left triangle).

As shown in FIGS. 26A and 26D, all three types of immunoconjugates of configuration 6 tested in this study exhibited significant inhibition of IL-2 activity in the absence of FAP-expressing cells (closed diamond, closed down triangle, closed left triangle). In contrast, when in contact with FAP-expressing cells, these molecules all exhibited activation of IL-2 activity that was comparable to that of the immunoconjugate of configuration 1 (open diamond, open down triangle, open left triangle).

The above studies demonstrate that antigen-dependent activation of cytokine activity in immunoconjugate molecules of the present disclosure can occur in molecules of diversified configurations. For example, the anchoring moiety and the masking moiety of an immunoconjugate can recognize the same or different antigenic epitope for the molecule to provide masking and activation of cytokine activities under respective conditions. Furthermore, the anchoring moiety and the masking moiety of an immunoconjugate molecule can independently select from various forms of antibodies or antigen binding fragments thereof, such as the Fab, scFv, single domain antibodies. Although exemplary embodiments of the immunoconjugates tested in the studies described herein may share certain common structural features (e.g., an Fc domain containing a knob-in-hole modification), based on the present disclosure, those of ordinary skill in the art would be able to envision possible variations to the molecular configurations exemplified herein and those alternative embodiments should be considered part of the present disclosure.

Without being bound by the theory, it is contemplated that to create the inhibiting effect by intramolecular interaction, the masking moiety and the cytokine moiety of the present immunoconjugate molecule are to be in the proximity of one another. Hence, alternative configurations of the immunoconjugate molecules having both the cytokine and the masking moieties fused to the N-terminus of the Fc domain were created and tested.

Among the tested configurations, several demonstrated comparable shielding and de-shielding ability. For example, the FB-707 in configuration 15 containing the same anchoring moiety and the two-in-one antibody as FB-676 in configuration 3. As shown in FIGS. 27A to 27C, both molecules behaved similarly in the effect of shielding and deshielding in presence of hFAP-expressing cells.

### 6.5.2 IL-2R Signaling Via Phosphorylation of STAT5

Actively growing primary mouse T cells were first starved overnight in mouse T cell media lacking IL-2 followed by a 2 hr starve in mouse T cell media lacking both IL-2 and FBS, both at 37° C. Cells were pelleted and plated at a density of 5×10⁵ cells per well of an ultra-low binding 96-well round bottom plate in 50 μL warm media. Cells were stimulated by addition of 50 μL solution of serial dilutions of wild-type or mutant IL-2 for 20 min at 37° C. and the reaction was terminated by fixation with 1.5% paraformaldehyde for 10 min at room temperature (RT) with agitation. Cells were pelleted, decanted, and permeabilized with 200 μL of 100% ice-cold methanol for at least 30 min on ice or incubation at −80° C. overnight. Fixed, permeabilized cells were washed three times with FACS buffer and intracellular phosphorylated STAT5 was detected with Alexa647 labeled anti-STAT5 pY694 (Cat. 612601, BD Biosciences) diluted 1:50 in FACS buffer and incubated for 1 hr at 4° C. in the dark. Cells were washed and analyzed on a CytoFLEX equipped with a high-throughput autosampler (Beckman Coulter). Data represent the mean fluorescence intensity normalized to the maximal intensity for wild-type IL-2, and points were fit to a log(agonist) vs. response (three parameters) model.

Human CD4+ T cells were purchased in frozen format (Saily Bio, China) by negatively selected from human PBMC. The human CD4+ cells were pre-activated as previously described (Smith G A et al. Science Signaling 10 eaan4931 (2017)). In brief, 10 million frozen human CD4+ cells were thaw and preactivated on 6-well plates coated with 5 μg/mL anti-CD3 antibody OKT3 (MA1-10176, Thermofisher) and 0.5 ug/mL anti-CD28 antibody (14-0289-82, Thermofisher) for 72 hours. The cells were harvested and cultured with 100 U/mL IL2 for 36 hours, and then cultured without IL2 for 36 hours before pSTAT5 activation and proliferation assay. The protocol for both pSTAT5 staining and proliferation is the same as above for CTLL2 cells.

### 6.5.2.1 T Cell Activation by Immunoconjugate Molecule Following Activation of Cytokine Activity by Soluble Antigens

Without being bound by the theory, the immuno-oncology potential of IL-2 largely arises from its capability to stimulate T cells and NK cells. To explore the therapeutic relevance of optimized molecules and the mechanism of action, the following studies were performed to determine the extent of inhibition and de-shielding in presence of hFAP.

Particularly, the ability of immunoconjugate molecules FB-604, FB-674, FB-675 and FB-676 to stimulate pre-activated human CD4+ cells were measured in the presence or absence of 200 nM Fc-hFAP. As shown FIG. 28A, the potency of IL2hex increased about 2 folds with immunoconjugate molecule FB-604 that does not have an anchoring moiety, and for about 10 folds for all other tested immunoconjugate molecules that have an anchoring moiety.

FIG. 28B shows human CD4+ T cell activation with immunoconjugate molecules of the present disclosure as measured using a pSTAT5 staining assay. The ability of immunoconjugate molecule FB-801, FB-794, FB-818 and FB-834 to stimulate pre-activated human CD4+ cells were measured in the presence or absence of 200 nM Fc-hFAP. As shown in the figure, the potency of IL2hex increased about 30 folds for all tested immunoconjugate molecules that have an anchoring moiety.

FIG. 29A shows human CD4+ T cell activation with immunoconjugate molecules of the present disclosure as measured using a pSTAT5 staining assay. The ability of immunoconjugate molecules FB-611, FB-610, FB-609, FB-608, FB-607, FB-601, FB-600, FB-599, FB-598, FB-676, FB-675, FB-674 and FB-604 to stimulate pre-activated human CD4+ cells were measured in presence or absence of 200 nM Fc-hFAP. FIG. 29B shows quantitation of the EC₅₀ values as measured by the assay of FIG. 29A.

### 6.5.2.2 IL-2 Induced T Cell Proliferation Assays.

Consistent with pSTAT5 activation assay in human CD4+ cells, IL-2hex mutant had about 100 holds lower potency as measured in EC₅₀ as compared to wild-type IL-2, and FB-794 had about 100 folds lower potency than IL-2hex. While soluble Fc-hFAP provided about 5 times increase in potency, the presence of fixed Expi-CHO-hFAP-B7 significantly elevated the potency in the 100 μM to 10 nM range. 50 k Expi-CHO-hFAP-B7 in 100 μL corresponded to ˜1 nM hFAP. It was consistent that potency of FB-794 could be enhanced by both deshielding and immobilization by Expi-CHO-hFAP-B7, and the enhancement was much more effective than comparable amount of soluble Fc-hFAP. When there is excess FB-794 (>>1 nM) which exceed the effective capacity of Expi-CHO-hFAP-B7 the proliferation was dominated by soluble FB-794. It was reasonable to expect FB-794 will be highly potent in the confined environment consisting of high hFAP expressing cells, IL-2 sensitive immune cells and high local concentration of FB-794.

### 6.5.3 In Vivo Toxicity Study

In vivo toxicity of immunoconjugate molecule was evaluated using C57BL/6J and CB-17 SCID mice. Knob-IL2hex contains a silent Knob-in-Hole domain fused with monovalent IL2hex at the C-terminal of the Fc-Knob through the 3X(GGGGS) linker, having a molecular weight 66.8 kDa. Knob-IL2hex was administered at 0, 10 μg, 25 pg, 50 pg/dose to C57BL/6J mice at days 1, 2, 3, 4, 5 of the week for two weeks, by intravenous infusion through vein in the tail at the volume of 150 μL. Death was monitored everyday, and body weight was monitored during weekdays. Death occurred in 25 μg and 50 μg dosage groups, but not in 0 and 10 μg doses. Significant weight loss was observed in all 10 g, 25 μg and 50 μg dosage groups. The results were plotted in FIG. 30 upper panel.

Knob-IL2hex was administered at 0, 5 μg, 10 μg, 30 μg/dose to CB-17 SCID mice at days 1, 2, 3, 4, 5 of the week for two weeks. CB-17 SCID immuno-compromised mice contains a defect in V(D)J recombination, lacking both T and B cells. Death occurred in all 5 μg, 10 μg and 30 μg dosage groups, and significant weight loss was in 10 g, and 30 g doses. The results were shown in FIG. 30 lower panel.

Based on the above study, the scheme of 25 μg/dose at days 1, 2, 3, 4, 5 of every week in C57BL/6J mice was chosen to study the acute toxicity of immunoconjugate molecules of interest.

Four samples were used to evaluate in vivo toxicity of immunoconjugate molecules, including Control (Knob-IL2hex, MW=66.8 kDa), FB-439 (MW=92.3 kDa), FB-449 (MW=120 kDa), and FB-476 (MW=116 kDa). Particularly, Control contains an unmasked IL2hex fused at C-terminal of Fc-Knob; FB-439 contains CD122 as the masking moiety and IL2hex is fused at C-terminal of Fc-Knob; FB-449 contains D049 masking moiety and the IL2hex is fused at the C-terminal of Fc-Knob; FB-476 contains D047 masking moiety and IL2hex is fused at N-terminal of light chain of D047. As shown in FIG. 31A, the immunoconjugate molecules in the samples were pure, intact containing each of the conjugating domains as expected.

The potency of the three immunoconjugate molecules, together with Knob-IL2WT, were assayed by CTLL2 proliferation assay, NK92 proliferation assay and HEK Blue IL2 activation assay. As shown in FIGS. 31B to 31D, all three molecules FB-439, FB-449 and FB-476 showed significant potency shift from Knob-IL2hex in all three assays for about 10 to 1000 folds. D047 in format of FB-476 showed comparable shielding effect as CD122 in FB-439.

The four samples were administered to C57BL/6J mice at days 1, 2, 3, 4, 5 and 6. 25 μg/dose and administration of Knob-IL2hex produced acute toxicity with significant weight loss and death within a week. As shown in FIG. 32, the three immunoconjugate molecules having the masking moiety (FB-439, FB-449 and FB-476) administered at 4-fold excess in molarity showed neither any sign of weight loss nor death, indicating the presence of the masking moiety significantly reduced the acute toxicity in mice. Notably, as demonstrated earlier in FIG. 7A, FB-449 showed about 8 times longer half-life than Knob-IL2hex in this dosing range. The combined toxicity profile of FB-449 has increased the immunocytokine exposure greater than 30-fold without evidence of toxicity.

In another in vivo toxicity study, IL-2 containing immunoconjugate molecules were administered to female C57BL/6J mice through tail vein injection on five consecutive days. Samples included IL-2 Fc fusion protein (10 or 50 μg), FB-439 (140 μg), FB-476 (180 μg), and were compared to a PBS control. Toxicity of the immunocytokines was monitored by measurement of body weight and mouse survival daily. As shown in FIG. 32B, administration of 10 pg IL-2 Fc fusion (sKnob-IL2hex) led to obvious weight loss immediately following the administration, while administration of up to 180 pg IL-2 containing immunoconjugate molecules according to the present disclosure did not result in observable body weight change, demonstrating significantly reduced IL-2 toxicity of the immunoconjugate molecules described herein.

### 6.5.4 In Vitro Antigen-Dependent Activation of Cytokine Activity by IL-2 Containing Immunoconjugate Molecules Having Mutations in IL2 Receptor (IL-2R) Binding Sites

The following studies were performed to examine whether IL-2-induced cellular activities can be fine-tuned by modulating binding of the IL-2 moiety of the immunoconjugate molecule to the different subunits of a functional IL-2R. Three IL-2 containing immunoconjugate molecules (#1097, 1112, 1150 and 1125) that contain different mutations in the IL-2 moiety, different two-in-one antibodies and different anchor arms were designed.

Immunoconjugate molecule 1150 have Configuration 14 as shown in FIGS. 50, and 1097, 1112 and 1125 have Configuration 15 as shown in FIG. 5P. Specifically, in 1112, the IL-2 moiety contains multiple point mutations (T3A, K35E, F42A, C125S) where the F42A mutation impacts the IL-2Rα binding site, and binding of the IL-2 moiety to IL-2Rα is attenuated. The masking moiety is an IL-2/FAP two-in-one antibody that binds to the IL-2 moiety and blocks its binding to IL-2Rβ. The anchor arm is scFv-872-5. In 1150, the IL-2 moiety contains multiple point mutations (D20T, K35E, C125S) where the D20T mutations resides in the IL-2Rβ binding site, and binding of the IL-2 moiety to IL-2Rβ is attenuated. The masking moiety is an IL-2/FAP two-in-one antibody that binds to the IL-2 moiety and blocks its binding to IL-2Rα. The anchor arm is VHH-E33. In 1097, the IL-2 moiety contains multiple point mutations (T3A, K35E, F42A, Y45A, L72G, C125S) where F42A, Y45A and L72G reside in the IL-2Rα binding site, and binding of the IL-2 moiety to IL-2Rα is abolished. The masking moiety is an IL-2/FAP two-in-one antibody that binds to the IL-2 moiety and blocks its binding to IL-2Rβ. The anchor arm is scFv-872-5 In 1125, the IL-2 moiety contains multiple point mutations (T3A, D20T, K35E, C125S) where D20T resides in the IL-2Rβ binding site, and binding of the IL-2 moiety to IL-2Rβ is attenuated. The masking moiety is an IL-2/FAP two-in-one antibody that binds to the IL-2 moiety and blocks its binding to IL-2Rα. The anchor arm is scFv872-5.

IL2-Fc fusion proteins of Configuration 1 containing either a wild-type IL-2 polypeptide (Knob-IL2) or the mutant IL-2hex polypeptide (Knob-IL2hex) and were used as positive controls. Immunoconjugate molecules 1097, 1112, 1150 and 1125 and control molecules were constructed and subjected to cell-based IL-2 signaling assays in the presence of cells expressing hFAP (HEK 293T-hFAP-E5) or cells that did not express hFAP (HEK 293T) as described above. The results are shown in FIG. 34A, FIG. 35A, FIG. 36A and FIG. 37A.

As shown in FIG. 34A, immunoconjugate molecules 1097 exhibited a strong inhibition of IL-2 in the absence of FAP-expressing cells (up triangle; open circle), and strong IL-2 activity in the presence of FAP-expressing cells (down triangle) which activity level was comparable to the positive controls (circle; square). Similarly, as shown in FIG. 36A and FIG. 37A, immunoconjugate molecules 1150 and 1125 also exhibited a strong inhibition of IL-2 in the absence of FAP-expressing cells (up triangle; diamond), and exhibited strong IL-2 activity in the presence of FAP-expressing cells (down triangle) which activity level was comparable to the positive controls (circle; square). In contrast and as shown in FIG. 35A, the masking effect was less prominent in immunoconjugate molecule 1112. Specifically, this molecule exhibited similar IL-2 activities in the presence or absence of FAP, and similar to the control molecules that did not have the masking moiety.

### 6.5.5 In Vivo Anti-Tumor Activity of IL-2 Containing Immunoconjugate Molecules

Next, in vivo anti-tumor activity and toxicity of IL-2 containing immunoconjugate molecules 1097, 1112, 1150 and 1125 were evaluated using tumor-bearing mice. Particularly, a MC38-FAP tumor model was created by implanting 1.5×10⁶ MC-38 mouse colon adenocarcinoma cells ectopically expressing FAP (B-FAP-MC38, Biocytogen) subcutaneously in the flank of female C57BL/6J mice. Tumors size was monitored by caliper (Tumor volume (mm³)=(length (mm)×width(mm)²)/2). Tumors were allowed to grow to −100 mm³ before beginning treatment. Dosing of PBS, IL-2 Fc fusions (12.5 or 25 μg) and immunoconjugate molecules 1097 (55 μg or 220 μg), 1112 (55 μg or 220 μg), 1150 (55 μg) and 1125 (55 μg) was performed on days 0, 3, and 6d via intravenous injection through tail vein. Dosing was terminated if weight loss exceeded 15% of bodyweight or there was a death in the group. Tumor size was measured every 2-3 days and body weight was measured daily. To serve as controls, the IL-2 Fc fusion used for assessing 1097 was IL-2hex containing mutations T3A/F42A/Y45A/L72G/C125S, the IL-2 Fc fusion used for assessing 1112 was a mutant IL-2 containing the mutations T3A/F42A/K35E/C125S, and the IL-2 Fc fusion used for assessing 1150 and 1125 was a mutant IL-2 containing the mutations T3A/D20T/K35E/C125S. The results are shown in FIGS. 34C, 35C, 36C and 37D.

As shown in FIG. 34C, administration of immunoconjugate molecule 1097 (“FB-1097”) at dosage 220 μg suppressed tumor growth in C57BL/6J mice comparable to mutant IL-2hex (CTRLhex) administered at the 25 μg dosage. Specifically, female C57BL/6 mice (n=3 per treatment group) were inoculated with 1 million MC38-FAP cells subcutaneously in the right flank of each mouse. Treatment was initiated when tumors reached 80-100 mm³. Vehicle (PBS), 25 μg CTRL-IL2hex, 55 μg FB-1097 and 220 μg FB-1097 were dosed at day 0, 3, and 6 post inoculation. The 25 μg CTRL-IL2hex and 220 μg FB-1097 groups showed appreciable and similar tumor regression in reference to vehicle. The 25 μg CTRL-hex group showed significant weight loss up to ˜20% while 220 μg FB-1097 did not show any appreciable weight loss. These data show that FB-1097 can match the efficacy of its corresponding IL2 mutant, together with significant toxicity reduction.

As shown in FIG. 34D, administration of FB-1097 at dosage 220 μg did not show any changes in immune cells in the peripheral blood of MC38-FAP C57BL/6 mice compared to mice administered PBS. Specifically, C57BL/6 mice were administered vehicle (PBS), 12.5 μg CTRL-IL2 WT, 12.5 μg CTRL-IL2hex, and 220 μg FB-1097 were dosed at days 0 and 3. The absolute cells in blood were counted on day 5. Both the 12.5 μg CTRL-IL2WT and 12.5 μg CTRL-IL2hex groups show a significant expansion of immune cells in peripheral blood. The 220 μg FB-1097 group did not show any changes in immune cells.

As shown in FIG. 34E, administration of immunoconjugate molecule 1097 at dosage 220 μg did not show any changes in lung weight in C57BL/6 mice compared to mice administered PBS. Specifically, C57BL/6 mice were administered vehicle (PBS), 12.5 μg CTRL-IL2 WT, 12.5 μg CTRL-IL2hex, and 220 μg FB-1097 were dosed at days 0 and 3. Lungs were weighed on day 5. Both the 12.5 μg CTRL-IL2WT and 12.5 μg CTRL-IL2hex groups show a significant increase in lung edema as shown by lung weight. The 220 μg FB-1097 group did not show any changes in lung weight. These data show the administration immunoconjugate molecule 1097 does not lead to edema.

As shown in FIG. 35C, administration of immunoconjugate molecule 1112 (“FB-1112”) at dosage 220 μg suppressed tumor growth in C57BL/6J mice comparable to g of mutant IL-2 having the F42A mutation (CTRLF42A), and both group of mice exhibited tumor rejection (100% CR) at the end of the observing period. Specifically, female C57BL/6 mice (n=3 per treatment group) were inoculated with 1 million MC38-FAP cells subcutaneously in the right flank of each mouse. Treatment was initiated when tumors reached 80-100 mm³. Vehicle (PBS), 25 μg CTRL-IL2hex and 55 μg FB-1112, or 220 μg FB-1112 were dosed at day 0, 3, and 6 post inoculation. The 25 μg CTRL-IL2hex and 220 g FB-1112 groups showed complete tumor regression and remained tumor free after a rechallenge with 1 million MC38-FAP cells. The 25 μg CTRL-F42A showed significant weight loss up to ˜10% while 220 μg FB-1112 didn't show appreciable weight loss. These data show that FB-1112 can match the efficacy of its corresponding IL2 mutant, together with significant toxicity reduction.

As shown in FIG. 36C, administration of the immunoconjugate molecule 1150 (“FB-1150”) at 55 μg dosage suppressed tumor growth in C57BL/6J mice. Specifically, female C57BL/6 mice (n=3 per treatment group) were inoculated with 1 million MC38-FAP subcutaneously in the right flank of each mouse. Treatment was initiated when tumors reached 80-100 mm³. Vehicle (PBS), 25 μg, CTRL-IL2D20T, or 55 μg FB-1150 were dosed at day 0, 3, and 6 post inoculation. The 25 μg CTRL-IL2D20T group showed completed tumor regression with minimal weight loss. These data show that 55 μg FB-1150 showed significant tumor regression (TGI>50%) and no weight loss.

As shown in FIG. 36D, administration of FB-1150 at dosage 55 μg did not show any mortality in MC38-FAP C57BL/6 mice while 12.5 μg CTRL-IL2D20T 25% showed mortality.

As shown in FIG. 36E, administration of immunoconjugate molecule 1150 at dosage 55 μg did not show any changes in body weight in MC38-FAP C57BL/6 mice.

As shown in FIG. 37C, administration of immunoconjugate molecule 1125 (FB-1125) at dosage 220 μg in the absence of FAP did not inhibit tumor growth in MC38 C57BL/6 mice compared to mice administered 12.5 μg CTRL D20T. Specifically, C57BL/6 mice were administered with 1 million MC38 cells subcutaneously in the right flank of each mouse. Treatment was initiated when tumors reached 80-100 mm³. Vehicle (PBS), 12.5 μg CTRL-D20T, or 220 μg FB-1125 were dosed at days 0, 3, and 6 post inoculation. The 12.5 g CTRL-IL2D20T group showed blunted tumor growth. In contrast, the 220 μg FB-1125 group did not show any effect in blunting tumor growth as the tumor volume was similar to the PBS treated group. These data show that FB-1125 is not effective in blunting tumor growth in the absence of FAP expression.

As shown in FIG. 37D, administration of immunoconjugate molecule 1125 at dosage 55 μg was able to blunt tumor volume growth in MC38-FAP C57BL/6 mice. Specifically, C57BL/6 mice were administered with 1 million MC38-FAP subcutaneously in the right flank of each mouse. Treatment was initiated when tumors reached 80-100 mm³. 12.5 μg CTRL-D20T, 55 μg FB-1125, or 55 μg FB-1125 and 100 μg si-4B9 were dosed at days 0, 3, and 6 post inoculation. The FB-1125 group showed blunted tumor growth. In contrast, FB-1125 in the presence of a FAP mAB (si-4B9) was not able to blunt tumor growth. These data show that FB-1125 was able to blunt tumor growth in the MC38-hFAP model, but the efficacy can be compromised in the presence of a FAP mAB which can compete with both the anchoring moiety and the masking moiety of the FB-1125 molecule.

The above in vitro and in vivo activities of molecules 1097, 1112, 1125 and 1150 demonstrates that IL-2 immunoconjugate having (a) mutations in the IL-2 moiety that attenuates IL-2 binding to one of the IL-2R a and R subunits, (b) the masking moiety targeting the binding site of the other one of IL-2R a and R subunits can significantly reduce IL-2 toxicity by effectively shielding IL-2 activity in cellular environment lacking FAP, while retaining strong anti-tumor efficacy by de-shielding IL-2 in proximity of the cancer cells where FAP is present. These studies validated the designing strategy for the immunoconjugate molecules described herein which combines mutational strategy with tailored masking targets in the cytokine to fine tune in vivo activity and toxicity of the immunoconjugate molecules.

### 6.5.6 In Vivo Anti-Tumor Activity of IL-2 Containing Immunoconjugate Molecules

Immunoconjugate molecules 1150 (FB-1150) was another IL-2 containing immunoconjugate molecule constructed to evaluate in vivo anti-tumor activity of the IL-2 containing immunoconjugate molecules described herein. Particularly, FB-1150 has the Configuration 14 as shown in FIG. 50. Specifically, in 1150, the IL-2 moiety contains a point mutations (D20T) in the IL-2Rβbinding site, and binding of the IL-2 moiety to IL-2Rβ is attenuated. The masking moiety is an IL-2/FAP two-in-one antibody that binds to the IL-2 moiety and blocks its binding to IL-2Rα.

Specifically, C57BL/6 mice were administered with 1 million MC38-FAP cells subcutaneously in the right flank of each mouse. Treatment was initiated when tumors reached 80-100 mm³. Vehicle (PBS), 12.5 μg sKnob-IL2D20T, or 55 μg FB-1150 were dosed at days 0, 3, and 6 post inoculation. As shown in FIG. 36C to 36E, administration of immunoconjugate molecule 1150 (FB-1150) at dosage 55 μg inhibited tumor growth in MC38-FAP C57BL/6 mice similar to mice administered 12.5 μg sKnob-IL2D20T. The 12.5 g CTRL-IL2D20T and 55 μg FB-1150 group showed blunted tumor growth. Further, the FB-1150 group showed no changes in survival changes while the CTRL-IL2D20T showed a decreased in survival percentage. None of the groups showed any changes in bodyweight. These data show that FB-1150 inhibited tumor volume without causing intolerable side effects or toxicity, as reflected in measurement of mortality rate or bodyweight.

6.5.7 Activation of Immunoconjugate Molecules Containing a Two-In-One Antibody that Binds IL-2 and EpCAM

The following studies were performed to examine whether antigen-dependent activation of cytokine activity occurs in immunoconjugate molecules containing variants of IL-2/Ep-CAM two-in-one antibodies.

Specifically, immunoconjugate molecules containing the IL-2/Ep-CAM two-in-one antibodies were of configuration 15 as shown in FIG. 5P. These immunoconjugate molecules were constructed and subjected to cell-based IL-2 signaling assays as described above.

For this study, immunoconjugate molecules contained an Fc domain having two non-identical subunits with knob-into-hole modifications that promoted dimerization of the two polypeptide chains. Specifically, these immunoconjugate molecules contained: (a) the IL-2hex polypeptide, that contained the mutations as described above as well as a K35E mutation (IL2hex/K35E), was fused to the C-terminus of one of the Fc subunits, (b) an anti-IL2/anti-EpCAM two-in-one Fab antibody fused to the C-terminus of the other Fc subunit, and (c) an anti-FAP single domain antibody fused to the N-terminus of one of the Fc subunits. Also, immunoconjugate polypeptides of Configuration 1 (IL-2 Fc fusion) that contained either a wild-type IL-2 polypeptide or the mutant IL-2hex polypeptide were used as controls. The control immunoconjugate molecules and immunoconjugate molecules containing variants of IL-2/Ep-CAM two-in-one antibodies were tested in cells expressing hFAP (hek 293T cells) and in cells that have high expression of EpCAM.

FIG. 38A shows Immunoconjugate molecule A having the configuration depicted in FIG. 38B demonstrating a strong shielding and deshielding effect. A low concentration of IL-2 controls increased absorbance at 635 nm (AU635) determined using a TECAN plate reader, which reflected secreted embryonic alkaline phosphatase (SEAP) level and responses to IL-2. In contrast, high concentrations of immunoconjugate molecule A showed increased responses to IL-2 in the presence of HEK 293T cells. Further, low concentrations of immunoconjugate molecule A showed increased IL-2 activity in the presence of Ep-CAM expressing cells. These results show immunoconjugate molecule A containing an IL2/Ep-CAM two-in-one antibody shows strong deshielding of IL2 activity in the presence of FAP. The above studies demonstrate that immunoconjugate molecules containing IL2/Ep-CAM two-in-one antibodies show deshielding of IL2 activity in the presence of FAP.

FIG. 38C shows Biolayer interferometry (BLI) binding curves of immobilized EpCAM and IL2 variant hex/K35E molecules to a Fab-Fc knob-into-hole monovalent construct of the EpCAM and IL2 dual specific molecule. In order to determine whether immunoconjugate molecule A can bind to 1) IL2hex containing a K35E mutation and 2) EpCAM, a biolayer interferometry (BLI) assay was established. Briefly, IL2hex/K35E and EpCAM were diluted to 15.6 nM in PBST-BSA and immobilized on a Streptavidin sensor on the Gator BLI instrument to an immobilization level of 1-2 nm depending upon the experiment. After establishing a baseline with PBST-BSA, the sensors were incubated with immunoconjugate molecule A (500 nM). This association step proceeded for 180 seconds, followed by 180 seconds of dissociation in PBST-BSA. The binding of immunoconjugate molecule A to either IL2hex/K35E or EpCAM was normalized by subtraction of a baseline where the antibody analyte was subjected to an empty BLI sensor containing no EpCAM. These data indicate that immunoconjugate molecule A binds to both IL2hex/K35E or EpCAM.

What is claimed:

1. An immunoconjugate molecule comprising
(a) a cytokine moiety comprising a cytokine polypeptide having a cytokine activity;
(b) a masking moiety; and
wherein the masking moiety comprises a bispecific antibody or antigen binding fragment thereof capable of binding to the cytokine polypeptide and a first target antigen;
wherein when binding to the cytokine polypeptide, the masking moiety reduces or inhibits the cytokine activity; and
wherein when binding to the first target antigen, the masking moiety disassociates from the cytokine polypeptide, thereby activating the cytokine activity.

2. The immunoconjugate molecule of claim 1, wherein the masking moiety comprises an intact antibody, a Fab, a Fab′, a F(ab′)₂, a Fv, a scFv, a dsFv, a diabody, a triabody, a tetrabody, or a VHH formed from antibody fragments.

3. The immunoconjugate molecule of claim 1 or 2, wherein the bispecific antibody is a two-in-one antibody.

4. The immunoconjugate molecule of any one of claims 1 to 3, wherein the first target antigen is not the cytokine polypeptide.

5. The immunoconjugate molecule of any one of claims 1 to 4, wherein the first target antigen is expressed on a cell surface.

6. The immunoconjugate molecule of claim 1, wherein the cell is a cancer cell or a cell in a tumor microenvironment.

7. The immunoconjugate molecule of any one of claims 1 to 6, wherein the first target antigen is soluble.

8. The immunoconjugate molecule of any one of claims 1 to 5, wherein the first target antigen is a tumor associated antigen.

9. The immunoconjugate molecule of any one of claims 1 to 8, wherein the first target antigen is fibrosis activation protein (FAP).

10. The immunoconjugate molecule of any one of claims 1 to 9, wherein the cytokine moiety comprises wild-type or mutant interleukin-2 (IL-2), and optionally human IL-2.

11. The immunoconjugate molecule of any one of claims 1 to 10, further comprising:
(c) an anchoring moiety comprising an antibody or antigen binding fragment thereof that specifically binds to a second target antigen.

12. The immunoconjugate molecule of claim 11, wherein the second target antigen is expressed on a cell surface.

13. The immunoconjugate molecule of claim 11 or 12, wherein the cell is a cancer cell or a cell in a tumor microenvironment.

14. The immunoconjugate molecule of any one of claims 11 to 13, wherein the second target antigen is soluble.

15. The immunoconjugate molecule of any one of claims 11 to 14, wherein the second target antigen is a tumor associated antigen.

16. The immunoconjugate molecule of any one of claims 11 to 15, wherein the first and second target antigens are the same.

17. The immunoconjugate molecule of claim 16, wherein the bispecific masking moiety and the anchoring moiety bind to the same epitope of the first or second target antigen.

18. The immunoconjugate molecule of claim 16, wherein the bispecific masking moiety and the anchoring moiety bind to different epitopes of the first or second target antigen.

19. The immunoconjugate molecule of any one of claims 11 to 18, wherein the second target antigen is fibrosis activation protein (FAP).

20. The immunoconjugate molecule of any one of claims 11 to 15, wherein the first target antigen and second target antigens are different.

21. The immunoconjugate molecule of any one of claims 11 to 20, wherein the anchoring moiety comprises an intact antibody, a Fab, a Fab′, a F(ab′)₂, a Fv, a scFv, a dsFv, a diabody, a triabody, a tetrabody, or a VHH formed from antibody fragments.

22. The immunoconjugate molecule of any one of claims 1 to 21, wherein the bispecific antibody or antigen binding fragment of the masking moiety is a Fab, ScFv or VHH.

23. The immunoconjugate molecule of any one of claims 1 to 22, wherein the antibody or antigen binding fragment thereof of the anchoring moiety is a Fab, ScFv or VHH.

24. The immunoconjugate molecule of any one of claims 1 to 23, further comprising: (d) a conjugating moiety, wherein the conjugating moiety operably connects two or more of the cytokine moiety, the masking moiety, and the anchoring moiety.

25. The immunoconjugate molecule of claim 24, wherein the conjugating moiety comprises an immunoglobulin Fc domain or a mutant thereof.

26. The immunoconjugate molecule of claim 25, wherein the Fc domain comprises a first subunit and a second subunit that are two non-identical polypeptide chains; and wherein the Fc domain comprises a first modification promoting hetero-dimerization of the two non-identical polypeptide chains.

27. The immunoconjugate molecule of claim 26, wherein the first modification is a knob-into-hole modification comprising a knob modification in the first subunit and a hole modification in the second subunit.

28. The immunoconjugate molecule of any one of claims 25 to 27, wherein the Fc domain comprises a second modification, wherein the Fc domain has reduced binding affinity to an Fc receptor compared to a native Fc domain without said second modification.

29. The immunoconjugate molecule of claim 28, wherein the Fc domain has reduced binding affinity to a Fcγ receptor as compared to the native Fc domain without said second modification.

30. The immunoconjugate molecule of claim 29, wherein the Fcγ receptor is an FcγRIIIa, FcγRI or FcγRIIa receptor.

31. The immunoconjugate molecule of any one of claims 28 to 30, wherein the Fc domain has reduced binding affinity to a complement component as compared to the native Fc domain without said second modification.

32. The immunoconjugate molecule of claim 31, wherein the complement component is C1q.

33. The immunoconjugate molecule of claim 28, wherein the Fc domain has reduced Fc effector function as compared to an Fc domain without said second modification.

34. The immunoconjugate molecule of claim 33, wherein the reduced Fc effector function is selected from complement dependent cytotoxicity (CDC), antibody-dependent cell-mediated cytotoxicity (ADCC), antibody-dependent cellular phagocytosis (ADCP), cytokine secretion, downregulation of cell surface receptors, and B cell activation.

35. The immunoconjugate molecule of any one of claims 28 to 34, wherein the second modification comprises one or more mutations selected from S228P, E233P, L234V, L234A, L235A, L235E, AG236, D265G, N297A, N297D, P329E, P329S, P329A, P329G, A330S, or P331S, wherein the numbering is that of the EU index as in Kabat.

36. The immunoconjugate molecule of any one of claims 28 to 35, wherein the second modification comprises one or more mutations selected from E233P, L234V, L234A, L235A, AG236, D265G, P327E, A328S, P329E, A330S, or P331S, wherein the numbering is that of the EU index as in Kabat.

37. The immunoconjugate molecule of any one of claims 24 to 36, wherein the cytokine moiety is connected to the C-terminus of one of the first and second subunits of the Fc domain, and the masking moiety is connected to the C-terminus of the other of the first and second subunits of the Fc domain.

38. The immunoconjugate molecule of claim 37, wherein the anchoring moiety is connected to the N-terminus of one of the first and second subunits of the Fc domain.

39. The immunoconjugate molecule of claim 38, wherein the anchoring moiety and the cytokine moiety are connected to the same subunit of the Fc domain.

40. The immunoconjugate molecule of claim 38, wherein the anchoring moiety and the masking moiety are connected to the same subunit of the Fc domain.

41. The immunoconjugate molecule of any one of claims 24 to 36, wherein the masking moiety is connected to the C-terminus of one of the first and second subunits of the Fc domain; and wherein the cytokine moiety is connected to the masking moiety.

42. The immunoconjugate molecule of claim 41, wherein the anchoring moiety is connected to the N-terminus of one of the first and second subunits of the Fc domain.

43. The immunoconjugate molecule of claim 42, wherein the anchoring moiety and the masking moiety are connected to the same subunit of the Fc domain; or wherein the anchoring moiety and the masking moiety are connected to different subunits of the Fc domain.

44. The immunoconjugate molecule of any one of claims 24 to 36, wherein the masking moiety is connected to the N-terminus of one of the first and second subunits of the Fc domain, and the cytokine moiety is connected to the masking moiety.

45. The immunoconjugate molecule of any one of claims 24 to 36, wherein the masking moiety is connected to the N-terminus of one of the first and second subunits of the Fc domain, and wherein the anchoring moiety is connected to the N-terminus of the other one of the first and second subunits of the Fc domain.

46. The immunoconjugate molecule of claim 45, wherein the cytokine moiety is connected to the masking moiety.

47. The immunoconjugate molecule of claim 45, wherein the cytokine moiety is connected to the anchoring moiety.

48. The immunoconjugate molecule of any one of claims 37 to 47, wherein the two-in-one antibody or antigen binding fragment thereof of the masking moiety is a Fab, a ScFv or a VHH.

49. The immunoconjugate molecule of any one of claims 37 to 48, wherein the antibody or antigen binding fragment thereof of the anchoring moiety is a Fab, a ScFv, or a VHH.

50. The immunoconjugate molecule of any one of claims 24 to 49, wherein the connection between two or more of the cytokine moiety, the masking moiety, the anchoring moiety and the conjugating moiety is via a peptidic linker.

51. The immunoconjugate of any one of claims 1 to 50, wherein the cytokine is IL-2 polypeptide having SEQ ID NOS: 1, 3, 7 to 15, and 107-110.

52. The immunoconjugate of any one of claims 1 to 51, wherein the first target antigen and the second target antigen are Fibroblast Activation Protein (FAP).

53. The immunoconjugate of any one of claims 1 to 52, wherein the masking moiety comprises an antibody or antigen binding fragment thereof that binds to Fibroblast Activation Protein (FAP) and interleukin-2 (IL-2), wherein the antibody or antigen binding fragment comprises
(a) a light chain variable region (VH) comprising VL complementarity determining region 1 (CDR1), VL CDR2, and VL CDR3 of any one of antibodies D001, D002, D029, D029LV1, D029LV2, D029LV3, D029LV4, D029LV5, D003, D047, D049, or B10 as set forth in Table 1; and/or
(b) a heavy chain variable region (VH) comprising VH complementarity determining region 1 (CDR1), VH CDR2, and VH CDR3 of any one of antibodies D001, D002, D029, D029HV1, D029HV2, D029HV3, D029HV4, D029HV5, D029HV6, D003, D047, D049, or B10 as set forth in Table 2.

54. The antibody or antigen binding fragment of claim 53, wherein
(a) the VL CDR1, VL CDR2, and VL CDR3 comprise amino acid sequences of SEQ ID NOS:16, 17, and 18, respectively, and the VH CDR1, VH CDR2, and VH CDR3 comprise amino acid sequences of SEQ ID NOS:36, 37, and 38, respectively;
(b) the VL CDR1, VL CDR2, and VL CDR3 comprise amino acid sequences of SEQ ID NOS:19, 17, and 20, respectively, and the VH CDR1, VH CDR2, and VH CDR3 comprise amino acid sequences of SEQ ID NOS:36, 39, and 38, respectively;
(c) the VL CDR1, VL CDR2, and VL CDR3 comprise amino acid sequences of SEQ ID NOS:21, 22, and 23, respectively, and the VH CDR1, VH CDR2, and VH CDR3 comprise amino acid sequences of SEQ ID NOS:40, 41, and 38, respectively;
(d) the VL CDR1, VL CDR2, and VL CDR3 comprise amino acid sequences of SEQ ID NOS:30, 17, and 31, respectively, and the VH CDR1, VH CDR2, and VH CDR3 comprise amino acid sequences of SEQ ID NOS:46, 47, and 48, respectively;
(e) the VL CDR1, VL CDR2, and VL CDR3 comprise amino acid sequences of SEQ ID NOS:32, 17, and 33, respectively, and the VH CDR1, VH CDR2, and VH CDR3 comprise amino acid sequences of SEQ ID NOS:49, 50, and 51, respectively;
(f) the VL CDR1, VL CDR2, and VL CDR3 comprise amino acid sequences of SEQ ID NOS:34, 17, and 35, respectively, and the VH CDR1, VH CDR2, and VH CDR3 comprise amino acid sequences of SEQ ID NOS:52, 53, and 51, respectively;
(g) the VL CDR1, VL CDR2, and VL CDR3 comprise amino acid sequences of SEQ ID NOS:24, 25, and 23, respectively, and the VH CDR1, VH CDR2, and VH CDR3 comprise amino acid sequences of SEQ ID NOS:40, 42, and 38, respectively;
(h) the VL CDR1, VL CDR2, and VL CDR3 comprise amino acid sequences of SEQ ID NOS:26, 25, and 28, respectively, and the VH CDR1, VH CDR2, and VH CDR3 comprise amino acid sequences of SEQ ID NOS:43, 42, and 38, respectively;
(i) the VL CDR1, VL CDR2, and VL CDR3 comprise amino acid sequences of SEQ ID NOS:26, 25, and 29, respectively, and the VH CDR1, VH CDR2, and VH CDR3 comprise amino acid sequences of SEQ ID NOS:43, 42, and 38, respectively;
(j) the VL CDR1, VL CDR2, and VL CDR3 comprise amino acid sequences of SEQ ID NOS:24, 25, and 29, respectively, and the VH CDR1, VH CDR2, and VH CDR3 comprise amino acid sequences of SEQ ID NOS:40, 42, and 38, respectively;
(k) the VL CDR1, VL CDR2, and VL CDR3 comprise amino acid sequences of SEQ ID NOS:26, 25, and 27, respectively, and the VH CDR1, VH CDR2, and VH CDR3 comprise amino acid sequences of SEQ ID NOS:43, 42, and 38, respectively;
(1) the VL CDR1, VL CDR2, and VL CDR3 comprise amino acid sequences of SEQ ID NOS:26, 25, and 27, respectively, and the VH CDR1, VH CDR2, and VH CDR3 comprise amino acid sequences of SEQ ID NOS:44, 42, and 38, respectively;
(m) the VL CDR1, VL CDR2, and VL CDR3 comprise amino acid sequences of SEQ ID NOS:26, 25, and 27, respectively, and the VH CDR1, VH CDR2, and VH CDR3 comprise amino acid sequences of SEQ ID NOS:45, 42, and 38, respectively; or
(n) the VL CDR1, VL CDR2, and VL CDR3 comprise amino acid sequences of SEQ ID NOS:103, 17, and 104, respectively, and the VH CDR1, VH CDR2, and VH CDR3 comprise amino acid sequences of SEQ ID NOS:105, 106, and 38, respectively.

55. The immunoconjugate of claim 53, wherein the antibody or antigen-binding fragment comprises:
(a) a light chain variable region (VL) comprising VL of any one of antibodies D001, D002, D029, D029LV1, D029LV2, D029LV3, D029LV4, D029LV5, D003, D047, D049, or B10 as set forth in Table 3; and/or
(b) a heavy chain variable region (VH) comprising VH of any one of antibodies D001, D002, D029, D029LV1, D029LV2, D029LV3, D029LV4, D029LV5, D003, D047, D049, or B10 as set forth in Table 4.

56. The antibody or antigen-binding fragment of claim 53, wherein the antibody or antigen-binding fragment thereof comprises a VL comprising an amino acid sequence of SEQ ID NO: 68, SEQ ID NO: 69, SEQ ID NO: 70, SEQ ID NO: 71, SEQ ID NO: 72, SEQ ID NO:73, SEQ ID NO: 74, SEQ ID NO: 75, SEQ ID NO: 76, SEQ ID NO: 77, SEQ ID NO: 78, or SEQ ID NO: 101.

57. The antibody or antigen-binding fragment of claim 54, wherein the antibody or antigen-binding fragment thereof comprises a VH comprising an amino acid sequence of SEQ ID NO: 79, SEQ ID NO: 80, SEQ ID NO: 81, SEQ ID NO: 82, SEQ ID NO: 83, SEQ ID NO:84, SEQ ID NO: 85, SEQ ID NO: 86, SEQ ID NO: 87, SEQ ID NO: 88, SEQ ID NO: 89, SEQ ID NO: 90, or SEQ ID NO: 102.

58. The antibody or antigen-binding fragment of claim 54, wherein the antibody or antigen-binding fragment thereof comprises
(a) a VL comprising an amino acid sequence of SEQ ID NO: 68; and
a VH comprising an amino acid sequence of SEQ ID NO: 79;
(b) a VL comprising an amino acid sequence of SEQ ID NO: 69; and
a VH comprising an amino acid sequence of SEQ ID NO: 80;
(c) a VL comprising an amino acid sequence of SEQ ID NO: 70; and
a VH comprising an amino acid sequence of SEQ ID NO: 81;
(d) a VL comprising an amino acid sequence of SEQ ID NO: 76; and
a VH comprising an amino acid sequence of SEQ ID NO: 88;
(e) a VL comprising an amino acid sequence of SEQ ID NO: 77; and
a VH comprising an amino acid sequence of SEQ ID NO: 89;
(f) a VL comprising an amino acid sequence of SEQ ID NO: 78; and
a VH comprising an amino acid sequence of SEQ ID NO: 90;
(g) a VL comprising an amino acid sequence of SEQ ID NO: 71; and
a VH comprising an amino acid sequence of SEQ ID NO: 82;
(h) a VL comprising an amino acid sequence of SEQ ID NO: 73; and
a VH comprising an amino acid sequence of SEQ ID NO: 83;
(i) a VL comprising an amino acid sequence of SEQ ID NO: 74; and
a VH comprising an amino acid sequence of SEQ ID NO: 83;
(j) a VL comprising an amino acid sequence of SEQ ID NO: 75; and
a VH comprising an amino acid sequence of SEQ ID NO: 82;
(k) a VL comprising an amino acid sequence of SEQ ID NO: 72; and
a VH comprising an amino acid sequence of SEQ ID NO: 84;
(1) a VL comprising an amino acid sequence of SEQ ID NO: 72; and
a VH comprising an amino acid sequence of SEQ ID NO: 85;
(m) a VL comprising an amino acid sequence of SEQ ID NO: 72; and
a VH comprising an amino acid sequence of SEQ ID NO: 87; or
(n) a VL comprising an amino acid sequence of SEQ ID NO: 101; and
a VH comprising an amino acid sequence of SEQ ID NO: 102.

59. The immunoconjugate of any one of claims 1 to 58, wherein the anchoring moiety comprises an antibody or antigen binding fragment thereof that binds to Fibroblast Activation Protein (FAP), wherein the antibody or antigen binding fragment comprises
(a) a light chain variable region (VH) comprising VL complementarity determining region 1 (CDR1), VL CDR2, and VL CDR3 of any one of antibodies 872-5, 872-59, 872-70, or 872-5V1 as set forth in Table 5; and/or
(b) a heavy chain variable region (VH) comprising VH complementarity determining region 1 (CDR1), VH CDR2, and VH CDR3 of any one of antibodies 872-5, 872-59, 872-70, 872-5V1, or VHH6 as set forth in Table 6.

60. The antibody or antigen binding fragment of claim 59, wherein
(a) the VL CDR1, VL CDR2, and VL CDR3 comprise amino acid sequences of SEQ ID NOS:30, 17, and 54, respectively, and the VH CDR1, VH CDR2, and VH CDR3 comprise amino acid sequences of SEQ ID NOS:58, 59, and 60, respectively;
(b) the VL CDR1, VL CDR2, and VL CDR3 comprise amino acid sequences of SEQ ID NOS:30, 17, and 55, respectively, and the VH CDR1, VH CDR2, and VH CDR3 comprise amino acid sequences of SEQ ID NOS:61, 62, and 48, respectively;
(c) the VL CDR1, VL CDR2, and VL CDR3 comprise amino acid sequences of SEQ ID NOS:30, 17, and 56, respectively, and the VH CDR1, VH CDR2, and VH CDR3 comprise amino acid sequences of SEQ ID NOS:36, 63, and 38, respectively;
(d) the VL CDR1, VL CDR2, and VL CDR3 comprise amino acid sequences of SEQ ID NOS:30, 17, and 57, respectively, and the VH CDR1, VH CDR2, and VH CDR3 comprise amino acid sequences of SEQ ID NOS:58, 64, and 51, respectively; or
(e) the antibody is an VHH comprising the VH CDR1, VH CDR2, and VH CDR3 comprise amino acid sequences of SEQ ID NOS:65, 66, and 67, respectively.

61. The antibody or antigen-binding fragment thereof of claim 59, wherein the antibody or antigen-binding fragment comprises:
(a) a light chain variable region (VL) comprising VL of any one of antibodies 872-5, 872-59, 872-70, or 872-5V1 as set forth in Table 7; and/or
(b) a heavy chain variable region (VH) comprising VH of any one of antibodies 872-5, 872-59, 872-70, 872-5V1, or VHH6 as set forth in Table 8.

62. The antibody or antigen-binding fragment of claim 59, wherein the antibody or antigen-binding fragment thereof comprises a VL comprising an amino acid sequence of SEQ ID NO: 91, SEQ ID NO: 92, SEQ ID NO: 93, or SEQ ID NO: 94.

63. The antibody or antigen-binding fragment of claim 59, wherein the antibody or antigen-binding fragment thereof comprises a VH comprising an amino acid sequence of SEQ ID NO: 95, SEQ ID NO: 96, SEQ ID NO: 97, SEQ ID NO: 98, or SEQ ID NO: 99.

64. The antibody or antigen-binding fragment of claim 59, wherein the antibody or antigen-binding fragment thereof comprises
(a) a VL comprising an amino acid sequence of SEQ ID NO: 91; and
a VH comprising an amino acid sequence of SEQ ID NO: 95;
(b) a VL comprising an amino acid sequence of SEQ ID NO: 92; and
a VH comprising an amino acid sequence of SEQ ID NO: 96;
(c) a VL comprising an amino acid sequence of SEQ ID NO: 93; and
a VH comprising an amino acid sequence of SEQ ID NO: 97;
(d) a VL comprising an amino acid sequence of SEQ ID NO: 94; and
a VH comprising an amino acid sequence of SEQ ID NO: 98; or
(e) an VHH comprising an amino acid sequence of SEQ ID NO: 99.

65. A composition comprising the immunoconjugate molecule of any one of claims 1 to 64, and a pharmaceutical acceptable carrier.

66. A polynucleotide encoding the immunoconjugate molecule of any one of any one of claims 1 to 64, or a fragment thereof.

67. The polynucleotide of claim 66, wherein the polynucleotide is operably linked to a promoter.

68. A vector comprising the polynucleotide of claim 66 or 67.

69. A cell comprising the polynucleotide of any one of claims 65 to 67.

70. A cell comprising the vector of claim 68.

71. An isolated cell producing the immunoconjugate molecule of any one of claims 1 to 64.

72. A kit comprising the immunoconjugate molecule of any one of claims 1 to 64.

73. A method of making an immunoconjugate molecule, comprising culturing the cell of any one of claims 57 to 71 to express the immunoconjugate molecule.

74. A method of making an immunoconjugate molecule, comprising expressing the polynucleotide of claim 66 or 67.

75. A method for activating a cytokine-mediated effect at a target site, the method comprising delivering to the target site an immunoconjugate molecule comprising the cytokine and a masking moiety;
wherein the masking moiety comprises a two-in-one antibody or antigen binding fragment thereof that binds to the cytokine through intramolecular interaction and inhibits the cytokine-mediated effect;
wherein the two-in-one antibody or antigen binding fragment is capable of binding to a first target antigen in the target site;
wherein when the immunoconjugate molecule is at the target site, the two-in-one antibody binds to the first target antigen and disassociate from the cytokine; and
wherein the cytokine-mediated effect is activated at the target site.

76. The method of claim 75, wherein the immunoconjugate molecule further comprises a anchoring moiety; wherein the anchoring moiety comprises an antibody or antigen binding fragment thereof capable of binding to a second target antigen in the target site.

77. The method of claim 75, wherein when the immunoconjugate molecule is at the target site, the antibody or antigen binding fragment of the anchoring moiety binds to the second target antigen; and wherein the immunoconjugate molecule is immobilized at the target site.

78. The method of any one of claims 75 to 77, wherein delivering the immunoconjugate molecule to the target site comprises administering the immunoconjugate molecule to a subject.

79. The method of claim 78, wherein the cytokine activity is at least about 10%, 20%, 30%, 40%, 45%, 50%, 55%, 60%, 65%, 70%, 75%, 80%, 85%, 90%, 95%, or 98% lower at a non-target site as compared to the cytokine activity at the target site after administration the immunoconjugate molecule to a subject.

80. A method for enriching a cytokine at a target site, the method comprising delivering to the target site an immunoconjugate molecule comprising the cytokine and an anchoring moiety;
wherein the anchoring moiety comprises an antibody or antigen binding fragment thereof capable of binding to a second target antigen in the target site;
wherein when the immunoconjugate molecule is at the target site, the anchoring moiety binds to the second target antigen; and
wherein the cytokine is distributed at a higher concentration at the target site compared to a non-target site.

81. The method of claim 80, wherein delivering the immunoconjugate molecule to the target site comprises administering the immunoconjugate molecule to a subject.

82. The method of claim 81, wherein the cytokine concentration is at least about 10%, 20%, 30%, 40%, 45%, 50%, 55%, 60%, 65%, 70%, 75%, 80%, 85%, 90%, 95%, or 98% lower at a non-target site as compared to the cytokine activity at the target site after administration the immunoconjugate molecule to a subject.

83. The method of claim 78, 79, 81 or 82, wherein a toxicity or side-effect associated with the cytokine in the subject is reduced.

84. The method of claim 83, wherein cytokine toxicity or side-effect is reduced at least about 10%, 20%, 30%, 40%, 45%, 50%, 55%, 60%, 65%, 70%, 75%, 80%, 85%, 90%, 95%, or 98% as compared to administration to the subject an equivalent amount of the cytokine in an unconjugated form.

85. The method of claim 83 or 84, wherein the reduction in toxicity or side-effect is measured as the elongation of life span of the administered subject.

86. The method of claim 83 or 84, wherein reduction in toxicity or side-effect associated with the cytokine is measured as reduction in loss of body weight of the administered subject.

87. The method of claim 83 or 84, wherein the reduction in toxicity or side-effect associated with the cytokine is measured as change in the level of an immune response in the administered subject.

88. The method of claim 83 or 84, wherein the reduction in toxicity or side-effect associated with the cytokine is measured as a change in an inflammatory response in the administered subject.

89. The method of claim 88, wherein the immunoconjugate molecule further comprises a masking moiety;
wherein the masking moiety comprises a two-in-one antibody or antigen binding fragment thereof that binds to the cytokine through intramolecular interaction and inhibits an cytokine-mediated effect;
wherein the two-in-one antibody or antigen binding fragment is capable of binding to a first target antigen in the target site;
wherein when the immunoconjugate molecule is at the target site, the two-in-one antibody binds to the first target antigen and disassociate from the cytokine; and
wherein the cytokine-mediated effect is activated at the target site.

90. The method of claim 76, 77, or 89 wherein the first antigen and second antigen are the same antigen or different antigens.

91. The method of any one of claims 75 to 90, wherein the target site is tumor microenvironment.

92. The method of any one of claims 75 to 90, wherein the target site is a cancerous cell.

93. The method of claim 91 or 92, wherein the first and/or second antigen is expressed on the surface of cancer cells.

94. The method of claim 91, wherein the first and/or second antigen is expressed by cells in the tumor microenvironment.

95. The method of claim 94, wherein the first and/or second antigen is fibrosis activation protein (FAP).

96. The method of any one of claims 75 to 95, wherein the immunoconjugate molecule further comprises conjugating moiety configured for operably connecting two or more of the cytokine polypeptide, the masking moiety and the anchoring moiety.

97. The method of claim 96, wherein the conjugating moiety is an immunoglobulin Fc domain comprising a first subunit and a second subunit that are two non-identical polypeptide chains; and wherein the Fc domain comprises a first modification promoting hetero-dimerization of the two non-identical polypeptide chains.

98. The method of claim 97, wherein the immunoglobulin domain comprises a second modification, wherein the Fc domain has reduced binding affinity to an Fc receptor compared to a native Fc domain without said second modification.

99. The method of any one of claims 75 to 98, wherein the immunoconjugate molecule is the immunoconjugate molecule of any one of claims 1 to 64.

100. A two-in-one antibody or antigen binding fragment thereof that binds to Fibroblast Activation Protein (FAP) and interleukin-2 (IL-2), wherein the antibody or antigen binding fragment comprises
(a) a light chain variable region (VH) comprising VL complementarity determining region 1 (CDR1), VL CDR2, and VL CDR3 of any one of antibodies D001, D002, D029, D029LV1, D029LV2, D029LV3, D029LV4, D029LV5, D003, D047, D049, or B10 as set forth in Table 1; and/or
(b) a heavy chain variable region (VH) comprising VH complementarity determining region 1 (CDR1), VH CDR2, and VH CDR3 of any one of antibodies D001, D002, D029, D029HV1, D029HV2, D029HV3, D029HV4, D029HV5, D029HV6, D003, D047, D049, or B10 as set forth in Table 2.

101. The antibody or antigen binding fragment of claim 100, wherein
(a) the VL CDR1, VL CDR2, and VL CDR3 comprise amino acid sequences of SEQ ID NOS:16, 17, and 18, respectively, and the VH CDR1, VH CDR2, and VH CDR3 comprise amino acid sequences of SEQ ID NOS:36, 37, and 38, respectively;
(b) the VL CDR1, VL CDR2, and VL CDR3 comprise amino acid sequences of SEQ ID NOS:19, 17, and 20, respectively, and the VH CDR1, VH CDR2, and VH CDR3 comprise amino acid sequences of SEQ ID NOS:36, 39, and 38, respectively;
(c) the VL CDR1, VL CDR2, and VL CDR3 comprise amino acid sequences of SEQ ID NOS:21, 22, and 23, respectively, and the VH CDR1, VH CDR2, and VH CDR3 comprise amino acid sequences of SEQ ID NOS:40, 41, and 38, respectively;
(d) the VL CDR1, VL CDR2, and VL CDR3 comprise amino acid sequences of SEQ ID NOS:30, 17, and 31, respectively, and the VH CDR1, VH CDR2, and VH CDR3 comprise amino acid sequences of SEQ ID NOS:46, 47, and 48, respectively;
(e) the VL CDR1, VL CDR2, and VL CDR3 comprise amino acid sequences of SEQ ID NOS:32, 17, and 33, respectively, and the VH CDR1, VH CDR2, and VH CDR3 comprise amino acid sequences of SEQ ID NOS:49, 50, and 51, respectively;
(f) the VL CDR1, VL CDR2, and VL CDR3 comprise amino acid sequences of SEQ ID NOS:34, 17, and 35, respectively, and the VH CDR1, VH CDR2, and VH CDR3 comprise amino acid sequences of SEQ ID NOS:52, 53, and 51, respectively;
(g) the VL CDR1, VL CDR2, and VL CDR3 comprise amino acid sequences of SEQ ID NOS:24, 25, and 23, respectively, and the VH CDR1, VH CDR2, and VH CDR3 comprise amino acid sequences of SEQ ID NOS:40, 42, and 38, respectively;
(h) the VL CDR1, VL CDR2, and VL CDR3 comprise amino acid sequences of SEQ ID NOS:26, 25, and 28, respectively, and the VH CDR1, VH CDR2, and VH CDR3 comprise amino acid sequences of SEQ ID NOS:43, 42, and 38, respectively;
(i) the VL CDR1, VL CDR2, and VL CDR3 comprise amino acid sequences of SEQ ID NOS:26, 25, and 29, respectively, and the VH CDR1, VH CDR2, and VH CDR3 comprise amino acid sequences of SEQ ID NOS:43, 42, and 38, respectively;
(j) the VL CDR1, VL CDR2, and VL CDR3 comprise amino acid sequences of SEQ ID NOS:24, 25, and 29, respectively, and the VH CDR1, VH CDR2, and VH CDR3 comprise amino acid sequences of SEQ ID NOS:40, 42, and 38, respectively;
(k) the VL CDR1, VL CDR2, and VL CDR3 comprise amino acid sequences of SEQ ID NOS:26, 25, and 27, respectively, and the VH CDR1, VH CDR2, and VH CDR3 comprise amino acid sequences of SEQ ID NOS:43, 42, and 38, respectively;
(l) the VL CDR1, VL CDR2, and VL CDR3 comprise amino acid sequences of SEQ ID NOS:26, 25, and 27, respectively, and the VH CDR1, VH CDR2, and VH CDR3 comprise amino acid sequences of SEQ ID NOS:44, 42, and 38, respectively;
(m) the VL CDR1, VL CDR2, and VL CDR3 comprise amino acid sequences of SEQ ID NOS:26, 25, and 27, respectively, and the VH CDR1, VH CDR2, and VH CDR3 comprise amino acid sequences of SEQ ID NOS:45, 42, and 38, respectively; or
(n) the VL CDR1, VL CDR2, and VL CDR3 comprise amino acid sequences of SEQ ID NOS:103, 17, and 104, respectively, and the VH CDR1, VH CDR2, and VH CDR3 comprise amino acid sequences of SEQ ID NOS:105, 106, and 38, respectively.

102. The antibody or antigen-binding fragment thereof of claim 100, wherein the antibody or antigen-binding fragment comprises:
(a) a light chain variable region (VL) comprising VL of any one of antibodies D001, D002, D029, D029LV1, D029LV2, D029LV3, D029LV4, D029LV5, D003, D047, D049, or B10 as set forth in Table 3; and/or
(b) a heavy chain variable region (VH) comprising VH of any one of antibodies D001, D002, D029, D029LV1, D029LV2, D029LV3, D029LV4, D029LV5, D003, D047, D049, or B10 as set forth in Table 4.

103. The antibody or antigen-binding fragment of claim 100, wherein the antibody or antigen-binding fragment thereof comprises a VL comprising an amino acid sequence of SEQ ID NO: 68, SEQ ID NO: 69, SEQ ID NO: 70, SEQ ID NO: 71, SEQ ID NO: 72, SEQ ID NO:73, SEQ ID NO: 74, SEQ ID NO: 75, SEQ ID NO: 76, SEQ ID NO: 77, SEQ ID NO: 78, or SEQ ID NO: 101.

104. The antibody or antigen-binding fragment of claim 100, wherein the antibody or antigen-binding fragment thereof comprises a VH comprising an amino acid sequence of SEQ ID NO: 79, SEQ ID NO: 80, SEQ ID NO: 81, SEQ ID NO: 82, SEQ ID NO: 83, SEQ ID NO:84, SEQ ID NO: 85, SEQ ID NO: 86, SEQ ID NO: 87, SEQ ID NO: 88, SEQ ID NO: 89, SEQ ID NO: 90, or SEQ ID NO: 102.

105. The antibody or antigen-binding fragment of claim 100, wherein the antibody or antigen-binding fragment thereof comprises
(a) a VL comprising an amino acid sequence of SEQ ID NO: 68; and
a VH comprising an amino acid sequence of SEQ ID NO: 79;
(b) a VL comprising an amino acid sequence of SEQ ID NO: 69; and
a VH comprising an amino acid sequence of SEQ ID NO: 80;
(c) a VL comprising an amino acid sequence of SEQ ID NO: 70; and
a VH comprising an amino acid sequence of SEQ ID NO: 81;
(d) a VL comprising an amino acid sequence of SEQ ID NO: 76; and
a VH comprising an amino acid sequence of SEQ ID NO: 88;
(e) a VL comprising an amino acid sequence of SEQ ID NO: 77; and
a VH comprising an amino acid sequence of SEQ ID NO: 89;
(f) a VL comprising an amino acid sequence of SEQ ID NO: 78; and
a VH comprising an amino acid sequence of SEQ ID NO: 90;
(g) a VL comprising an amino acid sequence of SEQ ID NO: 71; and
a VH comprising an amino acid sequence of SEQ ID NO: 82;
(h) a VL comprising an amino acid sequence of SEQ ID NO: 73; and
a VH comprising an amino acid sequence of SEQ ID NO: 83;
(i) a VL comprising an amino acid sequence of SEQ ID NO: 74; and
a VH comprising an amino acid sequence of SEQ ID NO: 83;
(j) a VL comprising an amino acid sequence of SEQ ID NO: 75; and
a VH comprising an amino acid sequence of SEQ ID NO: 82;
(k) a VL comprising an amino acid sequence of SEQ ID NO: 72; and
a VH comprising an amino acid sequence of SEQ ID NO: 84;
(l) a VL comprising an amino acid sequence of SEQ ID NO: 72; and
a VH comprising an amino acid sequence of SEQ ID NO: 85;
(m) a VL comprising an amino acid sequence of SEQ ID NO: 72; and
a VH comprising an amino acid sequence of SEQ ID NO: 87; or
(n) a VL comprising an amino acid sequence of SEQ ID NO: 101; and
a VH comprising an amino acid sequence of SEQ ID NO: 102.

106. An immunoconjugate molecule comprising the two-in-one antibody or antigen binding fragment of any one of claims 100 to 105 and an IL-2 polypeptide.

107. The immunoconjugate molecule of claim 106, wherein the IL-2 polypeptide is wild-type or mutant IL-2.

108. An antibody or antigen binding fragment thereof that binds to Fibroblast Activation Protein (FAP), wherein the antibody or antigen binding fragment comprises
(a) a light chain variable region (VH) comprising VL complementarity determining region 1 (CDR1), VL CDR2, and VL CDR3 of any one of antibodies 872-5, 872-59, 872-70, or 872-5V1 as set forth in Table 5; and/or
(b) a heavy chain variable region (VH) comprising VH complementarity determining region 1 (CDR1), VH CDR2, and VH CDR3 of any one of antibodies 872-5, 872-59, 872-70, 872-5V1, or VHH6 as set forth in Table 6.

109. The antibody or antigen binding fragment of claim 108, wherein
(a) the VL CDR1, VL CDR2, and VL CDR3 comprise amino acid sequences of SEQ ID NOS:30, 17, and 54, respectively, and the VH CDR1, VH CDR2, and VH CDR3 comprise amino acid sequences of SEQ ID NOS:58, 59, and 60, respectively;
(b) the VL CDR1, VL CDR2, and VL CDR3 comprise amino acid sequences of SEQ ID NOS:30, 17, and 55, respectively, and the VH CDR1, VH CDR2, and VH CDR3 comprise amino acid sequences of SEQ ID NOS:61, 62, and 48, respectively;
(c) the VL CDR1, VL CDR2, and VL CDR3 comprise amino acid sequences of SEQ ID NOS:30, 17, and 56, respectively, and the VH CDR1, VH CDR2, and VH CDR3 comprise amino acid sequences of SEQ ID NOS:36, 63, and 38, respectively;
(d) the VL CDR1, VL CDR2, and VL CDR3 comprise amino acid sequences of SEQ ID NOS:30, 17, and 57, respectively, and the VH CDR1, VH CDR2, and VH CDR3 comprise amino acid sequences of SEQ ID NOS:58, 64, and 51, respectively; or
(e) the antibody is an VHH comprising the VH CDR1, VH CDR2, and VH CDR3 comprise amino acid sequences of SEQ ID NOS:65, 66, and 67, respectively.

110. The antibody or antigen-binding fragment thereof of claim 108, wherein the antibody or antigen-binding fragment comprises:
(a) a light chain variable region (VL) comprising VL of any one of antibodies 872-5, 872-59, 872-70, or 872-5V1 as set forth in Table 7; and/or
(b) a heavy chain variable region (VH) comprising VH of any one of antibodies 872-5, 872-59, 872-70, 872-5V1, or VHH6 as set forth in Table 8.

111. The antibody or antigen-binding fragment of claim 108, wherein the antibody or antigen-binding fragment thereof comprises a VL comprising an amino acid sequence of SEQ ID NO: 91, SEQ ID NO: 92, SEQ ID NO: 93, or SEQ ID NO: 94.

112. The antibody or antigen-binding fragment of claim 108, wherein the antibody or antigen-binding fragment thereof comprises a VH comprising an amino acid sequence of SEQ ID NO: 95, SEQ ID NO: 96, SEQ ID NO: 97, SEQ ID NO: 98, or SEQ ID NO: 99.

113. The antibody or antigen-binding fragment of claim 108, wherein the antibody or antigen-binding fragment thereof comprises
(a) a VL comprising an amino acid sequence of SEQ ID NO: 91; and
a VH comprising an amino acid sequence of SEQ ID NO: 95;
(b) a VL comprising an amino acid sequence of SEQ ID NO: 92; and
a VH comprising an amino acid sequence of SEQ ID NO: 96;
(c) a VL comprising an amino acid sequence of SEQ ID NO: 93; and
a VH comprising an amino acid sequence of SEQ ID NO: 97;
(d) a VL comprising an amino acid sequence of SEQ ID NO: 94; and
a VH comprising an amino acid sequence of SEQ ID NO: 98; or
(e) an VHH comprising an amino acid sequence of SEQ ID NO: 99.

114. An immunoconjugate molecule comprising the antibody or antigen binding fragment of any one of claims 108 to 113, wherein the immunoconjugate molecule further comprises an IL-2 polypeptide.

115. The immunoconjugate molecule of claim 114, wherein the IL-2 polypeptide is wild-type or mutant IL-2.

116. An immunoconjugate molecule comprising an IL-2 polypeptide conjugated to a masking moiety,
wherein the masking moiety comprises a two-in-one antibody or antigen binding fragment thereof capable of binding to the IL-2 polypeptide and a first target antigen;
wherein when binding to the IL-2 polypeptide, the masking moiety blocks binding of the IL-2 polypeptide to a first IL-2 receptor (IL-2R) subunit; and
wherein when binding to the first target antigen, the masking moiety disassociates from the IL-2 polypeptide, thereby releasing the IL-2 polypeptide for binding with the first IL-2R subunit.

117. The immunoconjugate molecule of claim 116, wherein the IL-2 polypeptide comprises one or more mutations that attenuate binding of the IL-2 polypeptide to a second IL-2R subunit.

118. The immunoconjugate molecule of claim 116 or 117, wherein the first IL-2R subunit is the IL-2R a-chain (IL-2Rα), and the second IL-2R subunit is the IL-2R β-chain (IL-2Rβ).

119. The immunoconjugate molecule of claim 118, wherein binding of the IL-2 polypeptide to the second IL-2R subunit is reduced about 10%, about 20%, about 30%, about 40%, about 45%, about 50%, about 55%, about 60%, about 65%, about 70%, about 75%, about 80%, about 85%, about 90%, about 95%, or about 99% comparing to wild-type IL-2.

120. The immunoconjugate molecule of claim 118 or 119, wherein the one or more mutations that attenuate binding of the IL-2 polypeptide to IL-2Rβ are selected from D20T, D20G, D20A, H16E, H16R, H16A, N88D, N88S, N88R, V91G, V91A, V91R, and V91S, or a combination thereof.

121. The immunoconjugate molecule of any one of claims 118 to 120, wherein the masking moiety binds to an epitope of IL-2 comprising one or more of the residues P34, K35, R38, T41, F42, K43, F44, Y45, E61, E62, K64, P65, E68, V69, N71, L72, Q74, Y107, and D109 of IL-2.

122. The immunoconjugate molecule of any one of claims 118 to 120, wherein the masking moiety
(a) binds to an epitope of IL-2 recognized by an antibody comprising a light chain variable region having an amino acid sequence of SEQ ID NO: 101 and a heavy chain variable region having an amino acid sequence of SEQ ID NO:102;
(b) competes for binding with IL-2 with an antibody comprising a light chain variable region having an amino acid sequence of SEQ ID NO: 101 and a heavy chain variable region having an amino acid sequence of SEQ ID NO:102.

123. The immunoconjugate molecule of any one of claims 118 to 120, wherein the masking moiety comprises
(a) a light chain variable region (VL) comprising VL complementarity determining region 1 (CDR1), VL CDR2, and VL CDR3 of antibody B10 as set forth in Table 1; and/or
(b) a heavy chain variable region (VH) comprising VH complementarity determining region 1 (CDR1), VH CDR2, and VH CDR3 of antibody B10 as set forth in Table 2.

124. The immunoconjugate molecule of claim 123, wherein the masking moiety comprises
(a) the VL CDR1, VL CDR2, and VL CDR3 comprising amino acid sequences of SEQ ID NOS:103, 17, and 104, respectively, and
(b) the VH CDR1, VH CDR2, and VH CDR3 comprising amino acid sequences of SEQ ID NOS:105, 106, and 38, respectively.

125. The immunoconjugate molecule of claim 123, wherein the masking moiety comprises:
(a) a light chain variable region (VL) comprising VL of antibody B10 as set forth in Table 3; and/or
(b) a heavy chain variable region (VH) comprising VH of antibody B10 as set forth in Table 4.

126. The immunoconjugate molecule of claim 123, wherein the masking moiety comprises a VL comprising an amino acid sequence of SEQ ID NO: 101.

127. The immunoconjugate molecule of claim 123, wherein the masking moiety comprises a VH comprising an amino acid sequence of SEQ ID NO: 102.

128. The immunoconjugate molecule of claim 123, wherein the masking moiety comprises
(a) a VL comprising an amino acid sequence of SEQ ID NO: 101; and
(b) a VH comprising an amino acid sequence of SEQ ID NO: 102.

129. The immunoconjugate molecule of claim 116 or 117, wherein the first IL-2R subunit is the IL-2Rβ, and the second IL-2R subunit is the IL-2Rα.

130. The immunoconjugate molecule of claim 129, wherein binding of the IL-2 polypeptide to the IL-2Rα is reduced about 10%, about 20%, about 30%, about 40%, about 45%, about 50%, about 55%, about 60%, about 65%, about 70%, about 75%, about 80%, about 85%, about 90%, about 95%, or about 99% comparing to wild-type IL-2.

131. The immunoconjugate molecule of claim 129 or 130, wherein the one or more mutations that attenuate binding of the IL-2 polypeptide to IL-2Rα are selected from K35E, R38A, R38E, R38D, F42A, F42K, K43E, Y45A, E61R, E62A, L72G, or a combination thereof,
optionally wherein the one or more mutations that attenuate binding of the IL-2 polypeptide to IL-2Rα are
(a) F42A; or
(b) K35E and F42A.

132. The immunoconjugate molecule of any one of claims 129 to 131, wherein the masking moiety binds to an epitope of IL-2 comprising one or more of the residues L12, Q13, E15, H16, L19, D20, M23, R81, D84, D87, N88, V91, 192, and E95 or IL-2.

133. The immunoconjugate molecule of any one of claims 129 to 131, wherein the masking moiety
(a) binds to an epitope of IL-2 recognized by the antibody 5UTZ; or
(b) competes for binding with IL-2 with antibody 5UTZ.

134. The immunoconjugate molecule of any one of claims 116 to 133, wherein the IL-2 polypeptide further comprises one or more mutations that modifying binding of the IL-2 polypeptide to IL-2R y-chain (IL-2Rγ), wherein optionally the one or more mutations modifying binding of the IL-2 polypeptide to IL-2Rγ is selected from L18R, Q22E, T123A, Q126T, 1129V, S130A, S130R, or a combination thereof.

135. The immunoconjugate molecule of any one of claims 116 to 134, further comprising an anchoring moiety, wherein the anchoring moiety comprises an antibody or antigen binding fragment thereof that specifically binds to a second target antigen.

136. The immunoconjugate molecule of any one of claims 116 to 135, wherein the masking moiety disassociate from the IL-2 polypeptide in the presence of the first target antigen expressed on the surface of a first cell.

137. The immunoconjugate molecule of claim 136, wherein the second target antigen is expressed on the surface of the first cell or a second cell in proximity of the first cell.

138. The immunoconjugate molecule of claim 137, wherein the first target antigen and the second target antigen are the same or different.

139. The immunoconjugate molecule of any one of claims 116 to 138, wherein the first target antigen and/or the second target antigen is a tumor associated antigen.

140. The immunoconjugate molecule of any one of claims 116 to 139, wherein the first target antigen and the second target antigen are each independently selected from FAP, Her2, Her3, CD19, CD20, BCMA, PSMA, CEA, cMET, EGFR, CA-125, MUC-1, EpCAM, or Trop-2.

141. The immunoconjugate molecule of claim 140, wherein the first target antigen is FAP.

142. A method for activating an IL-2R comprising contacting the IL-2R with an effective amount of an immunoconjugate molecule of any one of claims 116 to 141.

143. The method of claim 142, wherein the IL-2R comprises IL-2Rβ.

144. The method of claim 142 or 143, wherein the TL-2R comprises IL-2Rα.

145. The method of any one of claims 142 to 144, wherein the IL-2R comprises IL-2Rγ.

146. The method of claim 142, wherein the IL-2R comprises the IL-2Rβ, and wherein the IL-2Rβ is expressed on the surface of a first cell.

147. The method of claim 146, wherein the IL-2R further comprises the IL-2Rγ, and wherein the IL-2Rγ is expressed on the surface of the first cell.

148. The method of claim 146 or 147, wherein the IL-2R further comprises the IL-2Rα;
optionally wherein the IL-2Rα is associated on a cell surface; optionally wherein the IL-2Rα is associated on the surface of the first cell (cis-presentation); or optionally wherein the IL-2Rα is associated on the surface of a second cell (trans-presentation);
optionally wherein the IL-2Rα is not associated on a cell surface.

149. The method of claim 146 or 147, wherein the IL-2R does not comprises the IL-2Rα.

150. The method of any one of claims 146 to 149, wherein the first cell and/or the second cell is an immune cell, and wherein upon activation of the IL-2R, the immune cell is activated.

151. The method of claim 150, wherein activation of the immune cell is measured as:
(a) increased proliferation or maturation of the immune cell;
optionally wherein proliferation or maturation of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%; or
(b) prolonged survival time of the immune cell;
optionally wherein survival time of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

152. The method of claim 150 or 151, wherein the immune cell is an effector T cell, memory T cell, or a combination thereof.

153. The method of claim 152, wherein immune cell is CD4+ T cells, CD8+ T cells, helper T cells, cytotoxic T cells, SLECs (short-lived effector cells), MPEC (memory precursor effector cells), TEs (terminal effector cells), NKs (natural killer cells), NKTs (natural killer T cells), innate lymphoid cells (Types I-III), or a combination thereof.

154. The method of claim 150 or 151, wherein the immune cell is a regulatory T cell (Treg).

155. The method of claim 154, wherein the immune cell is natural Treg (nTreg) cells, induced Treg (iTreg) cells, or a combination thereof.

156. The method of any one of claims 146 to 149, wherein the first cell and/or the second cell is a diseased cell, and wherein upon activation of the IL-2R, the diseased cell dies.

157. The method of claim 156, wherein
(a) the diseased cell is a cancer cell; or
(d) the diseased cell is a cell infected by an infectious pathogen;
optionally wherein the infectious pathogen is a virus, a bacteria, a fungus, a parasite, or a combination thereof.

158. A method of activating a target cell expressing an IL-2R, comprising contacting the target cell with an effective amount of the immunoconjugate molecule of any one of claims 116 to 141, wherein upon binding of the IL-2 polypeptide with the IL-2R, the target cell is activated,
optionally wherein the target cell is an immune cell;
optionally wherein the target cell is an effector T cell, memory T cell, regulatory T cell, or a combination thereof,
optionally wherein the target cell is CD4+ T cells, CD8+ T cells, helper T cells, cytotoxic T cells, SLECs (short-lived effector cells), MPEC (memory precursor effector cells), TEs (terminal effector cells), NKs (natural killer cells), NKTs (natural killer T cells), innate lymphoid cells (Types I-III), or a combination thereof;
optionally wherein the target cell is natural Treg (nTreg) cells, included Treg (iTreg) cells, or a combination thereof;
optionally wherein activation of the target cell is measured as:
(a) increased proliferation or maturation of the target cell;
optionally wherein proliferation or maturation of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%; or
(b) prolonged survival time of the target cell;
optionally wherein survival time of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

159. The method of claim 158, wherein the contacting further comprises administering a pharmaceutical composition comprising a pharmaceutically acceptable carrier and immunoconjugate molecule of any one of claims 116 to 141, optionally wherein the contacting enhances an anti-neoplastic immune response;
optionally wherein the contacting enhances an anti-infection immune response.

160. A method of enhancing an antigen-specific immune response of a population of T cells, comprising contacting the population of T cells with an effective amount of the immunoconjugate molecule of any one of claims 116 to 141;
optionally wherein the contacting enhances proliferation or maturation of antigen-specific effector T cells;
optionally wherein the contacting enhances formation of antigen-specific memory T cells;
optionally wherein the contacting is performed in the presence of the antigen; and optionally wherein the antigen is an antigen of a cancer, tumor, pathogen, or allergen.

161. A method of increasing secretion of pro-inflammatory cytokines by a population of T cells, comprising contacting the population of T cells with an immunoconjugate molecule of any one of claims 116 to 141, wherein said IL-2 polypeptide activates the T cells upon binding;
optionally wherein the cytokine is IL-1, IL-2, IL-6, IL-12, IL-17, IL-22, IL-23, GM-CSF, TNF-α, IFN-γ, or any combination thereof,
optionally wherein the cytokine production is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%.

162. A method of increasing assembly of IL-2R on the surface of a target cell, comprising contacting the target cell with an effective amount of the immunoconjugate molecule of any one of claims 116 to 141,
optionally wherein the IL-2R comprises IL-2Rα, IL-2Rβ, IL-2Rγ, or a combination thereof on the surface of the target cell;
optionally wherein the IL-2R comprises IL-2Rβ and IL-2Rγ on the surface of the target cell, and IL-2Rα on the surface of a second cell in proximity of the target cell;
optionally wherein the IL-2R comprises IL-2Rβ and IL-2Rγ on the surface of the target cell, and IL-2Rα not associated with a cell surface;
optionally wherein assembly of IL-2R on the surface of the target cell is increased by about 10%, about 20%, about 30%, about 40%, about 45%, about 50%, about 55%, about 60%, about 65%, about 70%, about 75%, about 80%, about 85%, about 90%, about 95%, about 100%, about 125%, about 150%, about 175%, about 200%, about 250%, about 300%, about 400%, about 500%, about 600%, about 700%, about 800%, about 900% or about 1000%;
optionally wherein the target cell is an immune cell;
optionally wherein the target cell is an effector T cell, memory T cell, regulatory T cell, or a combination thereof,
optionally wherein the target cell is CD4+ T cells, CD8+ T cells, helper T cells, cytotoxic T cells, SLECs (short-lived effector cells), MPEC (memory precursor effector cells), TEs (terminal effector cells), NKs (natural killer cells), NKTs (natural killer T cells), innate lymphoid cells (Types I-III), or a combination thereof;
optionally wherein the target cell is natural Treg (nTreg) cells, included Treg (iTreg) cells, or a combination thereof.

163. A method of forming a pro-inflammatory milieu in a tissue surrounding a population of diseased cells, comprising contacting the tissue with an effective amount of the immunoconjugate molecule of any one of claims 116 to 141;
optionally wherein:
(a) concentration of activated B cells, CD4+ effector T cells, CD8+ effector T cells, dendritic cells, macrophages, natural killer cells, monocytes, granulocytes, eosinophil and/or neutrophils in the tissue is increased;
(b) concentration of regulatory T cells in the tissue is reduced;
(c) concentration of a pro-inflammatory cytokine is increased in the tissue;
optionally wherein the pro-inflammatory cytokine is IL-1, IL-2, IL-6, IL-12, IL-17, IL-22, IL-23, GM-CSF, TNF-α, IFN-γ, or any combination thereof,
(d) concentration of antibodies binding to antigens originated or derived from the diseased cells is increased in the tissue;
(e) presentation of antigens originated or derived from the diseased cells by antigen presentation cells is increased in the tissue;
(f) phagocytosis of the diseased cells is increased in the tissue;
(g) apoptosis of the diseased cells induced by cell-mediated cytotoxicity is increased in the tissue;
(h) apoptosis of the diseased cells induced by antibody-dependent cellular cytotoxicity is increased in the tissue; and/or
(i) the population of the diseased cells is reduced in the tissue;
optionally wherein the population of the diseased cells is reduced by about 10%, about 20%, about 30%, about 40%, about 45%, about 50%, about 55%, about 60%, about 65%, about 70%, about 75%, about 80%, about 85%, about 90%, about 95%, or about 99% in the tissue.

164. A method of eliminating a diseased cell in a subject, comprising administering to the subject an effective amount of the immunoconjugate molecule of any one of claims 116 to 141;
optionally wherein:
(a) the diseased cell is a cancer cell; or
(d) the diseased cell is a cell infected by an infectious pathogen;
optionally wherein the infectious pathogen is a virus, a bacteria, a fungus, a parasite, or a combination thereof.

165. A method of treating cancer in a subject in need thereof, comprising administering to the subject an effective amount of the immunoconjugate molecule of any one of claims 116 to 141;
optionally wherein
(a) the treatment enhances an innate, humoral or cell-mediated anti-neoplastic immune response; and/or
(b) the method further comprises co-administration of a second therapy.

166. A method of treating an infection in a subject in need thereof, comprising administering to the subject an effective amount of the immunoconjugate molecule of any one of claims 116 to 141;
optionally wherein:
(a) the treatment enhances an innate, humoral, or cell-mediated anti-infective immune response;
(b) the subject is co-administered with a vaccine composition for preventing the infection in the subject;
optionally wherein, the vaccine composition is co-administered simultaneously or sequentially.

167. A method of increasing the response to an antigen in a subject in need thereof, comprising administering to the subject an effective amount of the immunoconjugate molecule of any one of claims 116 to 141;
optionally wherein the antigen is an antigen of a cancer, tumor, pathogen, or allergen, optionally wherein the antigen is originated or derived from
(a) an infectious pathogen;
optionally wherein the infectious pathogen is a virus, a bacteria, a fungus, a parasite, or a combination thereof;
(b) a diseased cell;
(c) a cell infected by an infectious pathogen;
optionally wherein the infectious pathogen is a virus, a bacteria, a fungus, a parasite, or a combination thereof, or
(d) a cancer cell.

168. A method of increasing a response to a vaccine in a subject in need thereof, comprising administering to the subject the vaccine and an effective amount of the immunoconjugate molecule of any one of claims 116 to 141;
optionally wherein the vaccine is a vaccine against a tumor, cancer, pathogen or allergen;
optionally wherein the immunoconjugate molecule is formulated as an adjuvant composition for the vaccine.

169. A method of establishing immune tolerance of an antigen in a tissue surrounding the antigen, comprising contacting the tissue with an effective amount of the immunoconjugate molecule of any one of claims 116 to 141;
optionally wherein:
(a) concentration of activated B cells, CD4+ effector T cells, CD8+ effector T cells, dendritic cells, macrophages, natural killer cells, monocytes, granulocytes, eosinophil and/or neutrophils in the tissue is reduced;
(b) concentration of regulatory T cells in the tissue is increased;
(c) concentration of a pro-inflammatory cytokine is reduced in the tissue;
optionally wherein the pro-inflammatory cytokine is IL-1, IL-2, IL-6, IL-12, IL-17, IL-22, IL-23, GM-CSF, TNF-α, IFN-γ or any combination thereof,
(d) concentration of antibodies binding to the antigen is reduced in the tissue;
(e) presentation of the antigen by antigen presentation cells is reduced in the tissue;
(f) phagocytosis of cells expressing the antigen is reduced in the tissue; and/or
(g) apoptosis of cells expressing the antigen is reduced in the tissue.

170. The method of claim 169, wherein the tissue is in a subject, and wherein the antigen is a self-antigen of the subject; optionally wherein the subject is suffering from an autoimmune disease.

171. A method for treating an autoimmune disease in a subject in need thereof, comprising administering to the subject an effective amount of the immunoconjugate molecule of any one of claims 116 to 141;
optionally wherein
(a) the treatment reduces an innate, humoral or cell-mediated immune response towards a self-antigen; and/or
(b) the method further comprises co-administration of a second therapy.
